(19) United States
(12) Patent Application Publication
(10) Pub. No.: US 2026/0124286 A1
(43) Pub. Date: May 7, 2026

(54) E. COLI FIMH MUTANTS AND USES THEREOF

(71) Applicant: Pfizer Inc., New York, NY (US)

(72) Inventors: Ye Che, Niantic, CT (US); Laurent Oliver Chorro, New York, NY (US); Robert George Konrad Donald, South Orange, NJ (US); Matthew Curtis Griffor, North Stonington, CT (US); Natalie Clare Silmon de Monerri, New York, NY (US)

(73) Assignee: Pfizer Inc., New York, NY (US)

(21) Appl. No.: 19/241,786

(22) Filed: Jun. 18, 2025

Related U.S. Application Data
(62) Division of application No. 17/554,424, filed on Dec. 17, 2021, now Pat. No. 12,357,681.
(60) Provisional application No. 63/130,153, filed on Dec. 23, 2020.
(60) Provisional application No. 63/185,425, filed on May 7, 2021.
(60) Provisional application No. 63/282,244, filed on Nov. 23, 2021.

(51) Int. Cl. A61K 39/108; A61K 39/00; C07K 14/245; C07K 16/1232

(52) U.S. Cl. CPC A61K 39/0258; A61K 39/0266; C07K 14/245; C07K 16/1232; A61K 2039/55566

(57) ABSTRACT

This disclosure relates to the design of E. coli mutated FimH polypeptides that result in improved biochemical properties and immunogenicity, compositions comprising such polypeptides, and uses thereof.

### RELATED APPLICATIONS

This application claims priority to U.S. Provisional Application No. 63/130,153 filed Dec. 23, 2020, U.S. Provisional Application No. 63/185,425 filed May 7, 2021 and U.S. Provisional Application No. 63/282,244 filed Nov. 23, 2021. The entire content of each of the foregoing applications is herein incorporated by reference in its entirety.

### REFERENCE TO SEQUENCE LISTING

This application is being filed electronically via EFS-Web and includes an electronically submitted sequence listing in .xml format. The .xml file contains a sequence listing entitled “PC072713A Sequence Listing.xml” created on Mar. 4, 2024 and having a size of 220,025 bytes. The sequence listing contained in this .xml file is part of the specification and is incorporated herein by reference in its entirety.

### TECHNICAL FIELD OF THE INVENTION

The present disclosure relates to mutated Escherichia coli FimH polypeptides and methods of their use.

### BACKGROUND OF THE INVENTION

Urinary tract infections (UTI) affect 1 in 5 women at least once during their lifetime and are responsible for significant morbidity and mortality, resulting in a substantial burden on healthcare systems. While several different bacteria can cause UTI, the most common cause (90-95% of cases) is the Gram-negative bacteria Escherichia coli (E. coli). Most E. coli UTI are caused by uropathogenic E. coli (UPEC) that colonise the gastrointestinal tract and migrate from the faecal flora to the urogenital tract, where they adhere to host uroepithelial cells, thus establishing a reservoir for ascending infections of the urinary tract. Adhesion is facilitated by fimbrial adhesins including type 1 fimbriae, which bind to mannosylated glycoproteins in the epithelial layer or secreted into the urine. Type 1 fimbriae are highly conserved among clinical UPEC isolates and are encoded by a cluster of genes called fim, which encode accessory proteins (FimC, FimD), various structural subunits (FimE, FimF, FimG) and an adhesin called FimH. FimH is essential for all characteristics of UTI infection in mouse models that mimic aspects of human bladder infection (Hannan et al. PLoS Pathog. 2010 Aug. 12; 6(8):e1001042; doi: 10.1371/journal.ppat.1001042; Schwartz et al. Infect Immun. 2011 October; 79(10):4250-9. doi: 10.1128/IAI.05339-11). Small molecule inhibitors that target FimH by mimicking mannosylated receptors further validate the role of FimH in UTI, and are showing promise as therapeutics in animal models (Cusurnano C K, et al. Sci Transl Med. 2011; 3(109):109ra115. doi:10.1126/scitranslmed.3003021). In addition, FimH is under positive selection in E. coli human cystitis isolates (Chen S L, et al. Proc Natl Acad Sci USA. 2009 Dec. 29; 106(52):22439-44. doi: 10.1073/pnas.0902179106) and positively selected residues may influence virulence in mouse models of cystitis (Schwartz, D. J. et al. Proc Natl Acad Sci USA 110, 15530-15537, doi:10.1073/pnas.1315203110 (2013)).

FimH is composed of two domains, the lectin binding domain (FimH_{LD}) responsible for binding to mannosylated glycoproteins, and the pilin domain. The pilin domain serves to link FimH to other structural subunits of the pilus such as FimG, via a mechanism called donor strand exchange (Le Trong, I et al., J. Struct Biol. 2010 December; 172(3):380-8. doi: 10.1016/j.jsb.2010.06.002). The FimH pilin domain forms an incomplete immunoglobulin fold, resulting in a groove that provides a binding site for the N-terminal β-strand of FimG, forming a strong intermolecular linkage between FimH and FimG. While FimH_{LD} can be expressed in a soluble, stable form, full length FimH is unstable alone (Vetsch, M., et al. J. Mol. Biol. 322:827-840 (2002); Barnhart M M, et al., Proc Natl Acad Sci USA. (2000) July 5; 97(14):7709-14) unless in a complex with the chaperone FimC or complemented with the donor strand peptide of FimG in peptide form or as a fusion protein (Barnhart M M, et al., Proc Natl Acad Sci USA. (2000) July 5; 97(14):7709-14; Sauer M M, et al. Nat Commun. (2016) March 7; 7:10738; Barnhart M M, et al. J Bacteriol. 2003 May; 185(9):2723-30). The design and expression of a full length FimH molecule by linking the FimG donor peptide to full length FimH via a glycine-serine linker has been previously described (PCT Intl. Publication No. WO2021/084429, published May 6, 2021), and is designated FimH-DSG.

FimH_{LD} is thought to be a poor immunogen in terms of its ability to stimulate functional immunogenicity. Some studies suggest that although binding antibody titers can be elicited with FimH_{LD} with and without adjuvant, functional neutralizing titers were only observed in the presence of adjuvant (PCT Intl. Publication No. WO2021/084429, published May 6, 2021). Studies suggest that locking FimH in an open conformation, with reduced affinity for mannoside ligands, improves functional immunogenicity (Kisiela, D. I. et al., Proc Natl Acad Sci USA 110, 19089-19094 (2013). Accordingly, there is a need in the art for novel FimH mutants with reduced affinity for mannoside ligands and improved biochemical properties that result in improved functional immunogenicity relative to wild type FimH.

### SUMMARY OF THE INVENTION

The present disclosure relates to the design of E. coli FimH mutated polypeptides that result in improved biochemical properties and immunogenicity, compositions comprising such polypeptides, and uses thereof. For example, in one aspect the present disclosure provides a mutated FimH polypeptide, which comprises at least one amino acid mutation relative to the amino acid sequence of the wild-type FimH polypeptide, wherein the mutation position is selected from the group consisting of: F1, P12, G14, G15, G16, A18, P26, V27, V28, Q32, N33, L34, V35, R60, S62, Y64, G65, L68, F71, T86, L107, Y108, L109, V112, S113, A115, G116, V118, A119, A127, L129, Q133, F144, V154, V155, V156, P157, T158, V163, and V185, wherein the amino acid positions are numbered according to SEQ ID NO:59.

In a further aspect is a mutated FimH polypeptide comprising at least one mutation selected from the group consisting of: F1I; F1L; F1V; F1M; F1Y; F1W; P12C; G14C; G15A; G15P; G16A; G16P; A18C; P26C; V27A; V27C; V28C; Q32C; N33C; L34C; L34N; L34S; L34T; L34D; L34E; L34K; L34R; V35C; R60P; S62C; Y64C; G65A; L68C; F71C; T86C; L107C; Y108C; L109C; V112C; S113C; A115V; G116C; V118C; A119C; A119N; A119S; A119T; A119D; A119E; A119K; A119R; A127C; L129C; Q133K; F144C; V154C; V156C; P157C; T158C; V1631; and V185I, or any combination thereof. For example, a mutated FimH polypeptide comprising the mutations G15A and G16A. Further, for example, a mutated FimH polypeptide comprising the mutations P12C and A18C. Further for example, a mutated FimH polypeptide comprising the mutations G14C and F144C. Further for example, a mutated FimH polypeptide comprising the mutations P26C and V35C. Further for example, a mutated FimH polypeptide comprising the mutations P26C and V154C. Further for example, a mutated FimH polypeptide comprising the mutations P26C and V156C. Further for example, a mutated FimH polypeptide comprising the mutations V27C and L34C. Further for example, a mutated FimH polypeptide comprising the mutations V28C and N33C. Further for example, a mutated FimH polypeptide comprising the mutations V28C and P157C. Further for example, a mutated FimH polypeptide comprising the mutations Q32C and Y108C. Further for example, a mutated FimH polypeptide comprising the mutations N33C and L109C. Further for example, a mutated FimH polypeptide comprising the mutations N33C and P157C. Further for example, a mutated FimH polypeptide comprising the mutations V35C and L107C. Further for example, a mutated FimH polypeptide comprising the mutations V35C and L109C. Further for example, a mutated FimH polypeptide comprising the mutations S62C and T86C. Further for example, a mutated FimH polypeptide comprising the mutations S62C and L129C. Further for example a mutated FimH polypeptide comprising the mutations Y64C and L68C. Further for example a mutated FimH polypeptide comprising the mutations Y64C and A127C. Further for example a mutated FimH polypeptide comprising the mutations L68C and F71C. Further for example a mutated FimH polypeptide comprising the mutations V112C and T158C. Further for example a mutated FimH polypeptide comprising the mutations S113C and G116C. Further for example a mutated FimH polypeptide comprising the mutations S113C and T158C. Further for example a mutated FimH polypeptide comprising the mutations V118C and V156C. Further for example a mutated FimH polypeptide comprising the mutations A119C and V155C. Further for example a mutated FimH polypeptide comprising the mutations L34N and V27A. Further for example a mutated FimH polypeptide comprising the mutations L34S and V27A. Further for example a mutated FimH polypeptide comprising the mutations L34T and V27A. Further for example, a mutated FimH polypeptide comprising the mutations L34D and V27A. Further for example, a mutated FimH polypeptide comprising the mutations L34E and V27A. Further for example, a mutated FimH polypeptide comprising the mutations L34K and V27A. Further for example, a mutated FimH polypeptide comprising the mutations L34R and V27A. Further for example a mutated FimH polypeptide comprising the mutations A119N and V27A. Further for example a mutated FimH polypeptide comprising the mutations A119S and V27A. Further for example a mutated FimH polypeptide comprising the mutations A119T and V27A. Further for example, a mutated FimH polypeptide comprising the mutations A119D and V27A. Further for example, a mutated FimH polypeptide comprising the mutations A119E and V27A. Further for example, a mutated FimH polypeptide comprising the mutations A119K and V27A. Further for example, a mutated FimH polypeptide comprising the mutations A119R and V27A. Further for example a mutated FimH polypeptide comprising the mutations G15A and V27A. Further for example a mutated FimH polypeptide comprising the mutations G16A and V27A. Further for example a mutated FimH polypeptide comprising the mutations G15P and V27A. Further for example a mutated FimH polypeptide comprising the mutations G16P and V27A. Further for example a mutated FimH polypeptide comprising the mutations G15A, G16A, and V27A. Further for example a mutated FimH polypeptide comprising the mutations G65A and V27A. Further for example a mutated FimH polypeptide comprising the mutations V27A and Q133K. Further for example a mutated FimH polypeptide comprising the mutations G15A, G16A, V27A, and Q133K. Further for example a mutated FimH polypeptide comprising the sequence of any one of SEQ ID NOs: 2-58, and 60-64. Further for example a mutated FimH polypeptide disclosed herein, wherein the polypeptide is isolated.

In a further example, the present disclosure provides a pharmaceutical composition comprising (i) a mutated FimH polypeptide as disclosed herein and (ii) a pharmaceutically acceptable carrier.

In a further example, the present disclosure provides an immunogenic composition comprising a mutated FimH polypeptide as disclosed herein. For example, the immunogenic composition further comprises at least one additional antigen, such as a polysaccharide, or a glycoconjugate, or a protein. Further for example, the immunogenic composition further comprises at least one adjuvant.

In a further example, the present disclosure provides a nucleic acid molecule comprising a nucleotide sequence that encodes an amino acid sequence of a mutated FimH polypeptide as disclosed herein.

In a further example, the present disclosure provides a mutated FimH polypeptide as disclosed herein, wherein the polypeptide is immunogenic.

The present disclosure further provides a recombinant mammalian cell, comprising a polynucleotide encoding a mutated FimH polypeptide as disclosed herein.

The present disclosure further provides a culture comprising the recombinant cell as disclosed herein, wherein said culture is at least 5 liters, at least 10 liters, at least 20 liters, at least 50 liters, at least 100 liters, at least 200 liters, at least 500 liters, at least 1000 liters, or at least 2000 liters in size.

The present disclosure further provides a method for producing a mutated FimH polypeptide as disclosed herein, comprising culturing a recombinant mammalian cell as disclosed herein under suitable conditions, thereby expressing the polypeptide; and harvesting the polypeptide.

The present disclosure further provides a method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of a composition as disclosed herein. In one example, the subject is at risk of developing a urinary tract infection. In a further example, the subject is at risk of developing bacteremia. In a further example, the subject is at risk of developing sepsis. In another example, the subject is at risk of developing Crohn's disease.

The present disclosure further provides a method of eliciting an immune response against E. coli in a mammal, comprising administering to the mammal an effective amount of a composition as disclosed herein. In one example, the immune response comprises opsonophagocytic and/or neutralizing antibodies against E. coli. In a further example, the immune response protects the mammal from an E. coli infection.

The present disclosure further provides a method of preventing, treating or ameliorating a bacterial infection, disease or condition in a subject, comprising administering to the subject an immunologically effective amount of a composition as disclosed herein.

### BRIEF DESCRIPTION OF THE DRAWINGS

FIG. 1A-1B show circular dichroism spectra of FimH_{LD} and FimH-DSG mutants. FIG. 1A shows circular dichroism spectra in the near-UV and FIG. 1B shows circular dichroism spectra in the far-UV regions.

FIG. 2 shows the relative immunogenicity of FimH_{LD} mutants in yeast mannan neutralization assay at PD3.

FIG. 3A-3B show the immunogenicity of FimH_{LD} and FimH-DSG mutants in yeast mannan neutralization assay at PD2 (FIG. 3A) and PD3 (FIG. 3B).

FIG. 4 is a diagram showing the major purification steps utilized for isolation of the wild type and mutated forms of His-tagged FimH-DsG.

FIG. 5A-5B show a purification profile of FimH-DSG WT. FIG. 5A is the elution profile of FimH-DSG WT on SP-SEPHAROSE column, and FIG. 5B is the SDS-PAGE analysis of eluted fractions.

FIG. 6A-6B show a purification profile of the FimH-DSG G15A G16A V27A mutant. FIG. 6A shows the elution profile of the FimH-DSG G15A G16A V27A mutant on SP-SEPHAROSE column, and FIG. 6B shows the SDS-PAGE analysis of eluted fractions.

FIG. 7A-7B show the analytical SEC of FimH-DSG proteins. The analytical SEC of FimH-DSG G15A G16A V27A is shown in FIG. 7A, and that of wild type FimH-DSG WT is shown in FIG. 7B.

FIG. 8 shows a schematic representation of the mechanism of FimH HMW complex formation.

FIG. 9 shows the immunization schedule of non-human primates and subsequent challenge described in Example 21 herein.

FIG. 10 shows a rise in O-antigen serotype-specific antibodies following vaccination of NHPs with FimH-DSG G15A G16A V27A mutant+4-valent E. coli O-antigens (O1a, O2, O6 and O25b). Legend: Placebo (circle); FimH-DSG G15A G16A V27A (square); FimH-DSG G15A G16A V27A+4-valent O-Antigen (triangle).

FIG. 11A-11B show that immunization with FimH-DSG G15A G16A V27A+/−4plex O-antigens elicits potent functional anti-FimH antibody responses. FIG. 11A shows the results from a Direct Luminex FimH IgG assay, and FIG. 11B shows the results from an E. coli binding inhibition assay. As used herein, the term “4plex” has the same meaning, and is interchangeable with, the term “4-valent”.

FIG. 12 shows that bacteriuria is reduced in vaccinated nonhuman primates (NHP) after challenge as described in Example 21. Legend: Placebo (circle); FimH-DSG G15A G16A V27A (square); FimH-DSG G15A G16A V27A+4-valent O-Antigen (triangle); * p≤0.007 compared to placebo group.

FIG. 13A-13C show that biomarkers of infection are reduced in vaccinated NHP after challenge of the three groups: placebo, FimH-DSG G15A G16A V27A alone and FimH-DSG G15A G16A V27A+4-valent O-antigens, as described in Example 21. FIG. 13A shows the quantification of MPO in urine, FIG. 13B shows the quantification of IL-8 in urine, and FIG. 13C shows the percentage of animals that had increased PMN in urine sediments. Legend: Placebo (circle); FimH-DSG G15A G16A V27A (square); FimH-DSG G15A G16A V27A+4-valent O-Antigen (triangle).

### SEQUENCE IDENTIFIERS

SEQ ID NO: 1 sets forth an amino acid sequence for wild type E. coli FimH_{LD} (FimHLD_WT).

SEQ ID NO: 2 sets forth an amino acid sequence for the mutant E. coli FimHLD_G65A_V27A.

SEQ ID NO: 3 sets forth an amino acid sequence for the mutant E. coli FimHLD_F1I.

SEQ ID NO: 4 sets forth an amino acid sequence for the mutant E. coli FimHLD_F1L.

SEQ ID NO: 5 sets forth an amino acid sequence for the mutant E. coli FimHLD_F1V.

SEQ ID NO: 6 sets forth an amino acid sequence for the mutant E. coli FimHLD_F1M.

SEQ ID NO: 7 sets forth an amino acid sequence for the mutant E. coli FimHLD_F1Y.

SEQ ID NO: 8 sets forth an amino acid sequence for the mutant E. coli FimHLD_F1W.

SEQ ID NO: 9 sets forth an amino acid sequence for the mutant E. coli FimHLD_Q133K.

SEQ ID NO: 10 sets forth an amino acid sequence for the mutant E. coli FimHLD_G15A.

SEQ ID NO: 11 sets forth an amino acid sequence for the mutant E. coli FimHLD_G15P.

SEQ ID NO: 12 sets forth an amino acid sequence for the mutant E. coli FimHLD_G16A.

SEQ ID NO: 13 sets forth an amino acid sequence for the mutant E. coli FimHLD_G16P.

SEQ ID NO: 14 sets forth an amino acid sequence for the mutant E. coli FimHLD_G15A_G16A.

SEQ ID NO: 15 sets forth an amino acid sequence for the mutant E. coli FimHLD_R60P.

SEQ ID NO: 16 sets forth an amino acid sequence for the mutant E. coli FimHLD_G65A.

SEQ ID NO: 17 sets forth an amino acid sequence for the mutant E. coli FimHLD_P12C_A18C.

SEQ ID NO: 18 sets forth an amino acid sequence for the mutant E. coli FimHLD_G14C_F144C.

SEQ ID NO: 19 sets forth an amino acid sequence for the mutant E. coli FimHLD_P26C_V35C.

SEQ ID NO: 20 sets forth an amino acid sequence for the mutant E. coli FimHLD_P26C_V154C.

SEQ ID NO: 21 sets forth an amino acid sequence for the mutant E. coli FimHLD_P26C_V156C.

SEQ ID NO: 22 sets forth an amino acid sequence for the mutant E. coli FimHLD_V27C_L34C.

SEQ ID NO: 23 sets forth an amino acid sequence for the mutant E. coli FimHLD_V28C_N33C.

SEQ ID NO: 24 sets forth an amino acid sequence for the mutant E. coli FimHLD_V28C_P157C.

SEQ ID NO: 25 sets forth an amino acid sequence for the mutant E. coli FimHLD_Q32C_Y108C.

SEQ ID NO: 26 sets forth an amino acid sequence for the mutant E. coli FimHLD_N33C_L109C.

SEQ ID NO: 27 sets forth an amino acid sequence for the mutant E. coli FimHLD_N33C_P157C.

SEQ ID NO: 28 sets forth an amino acid sequence for the mutant E. coli FimHLD_V35C_L107C.

SEQ ID NO: 29 sets forth an amino acid sequence for the mutant E. coli FimHLD_V35C_L109C.

SEQ ID NO: 30 sets forth an amino acid sequence for the mutant E. coli FimHLD_S62C_T86C.

SEQ ID NO: 31 sets forth an amino acid sequence for the mutant E. coli FimHLD_S62C_L129C.

SEQ ID NO: 32 sets forth an amino acid sequence for the mutant E. coli FimHLD_Y64C_L68C.

SEQ ID NO: 33 sets forth an amino acid sequence for the mutant E. coli FimHLD_Y64C_A127C.

SEQ ID NO: 34 sets forth an amino acid sequence for the mutant E. coli FimHLD_L68C_F71C.

SEQ ID NO: 35 sets forth an amino acid sequence for the mutant E. coli FimHLD_V112C_T158C.

SEQ ID NO: 36 sets forth an amino acid sequence for the mutant E. coli FimHLD_S113C_G116C.

SEQ ID NO: 37 sets forth an amino acid sequence for the mutant E. coli FimHLD_S113C_T158C.

SEQ ID NO: 38 sets forth an amino acid sequence for the mutant E. coli FimHLD_V118C_V156C.

SEQ ID NO: 39 sets forth an amino acid sequence for the mutant E. coli FimHLD_A119C_V155C.

SEQ ID NO: 40 sets forth an amino acid sequence for the mutant E. coli FimHLD_L34N_V27A.

SEQ ID NO: 41 sets forth an amino acid sequence for the mutant E. coli FimHLD_L34S_V27A.

SEQ ID NO: 42 sets forth an amino acid sequence for the mutant E. coli FimHLD_L34T_V27A.

SEQ ID NO: 43 sets forth an amino acid sequence for the mutant E. coli FimHLD_A119N_V27A.

SEQ ID NO: 44 sets forth an amino acid sequence for the mutant E. coli FimHLD_A119S_V27A.

SEQ ID NO: 45 sets forth an amino acid sequence for the mutant E. coli FimHLD_A119T_V27A.

SEQ ID NO: 46 sets forth an amino acid sequence for the mutant E. coli FimH-DSG_A115V.

SEQ ID NO: 47 sets forth an amino acid sequence for the mutant E. coli FimH-DSG_V1631.

SEQ ID NO: 48 sets forth an amino acid sequence for the mutant E. coli FimH-DSG_V1851.

SEQ ID NO: 49 sets forth an amino acid sequence for the mutant E. coli FimH-DSG_DSG_V31.

SEQ ID NO: 50 sets forth an amino acid sequence for the mutant E. coli FimHLD_G15A_V27A.

SEQ ID NO: 51 sets forth an amino acid sequence for the mutant E. coli FimHLD_G16A_V27A.

SEQ ID NO: 52 sets forth an amino acid sequence for the mutant E. coli FimHLD_G15P_V27A.

SEQ ID NO: 53 sets forth an amino acid sequence for the mutant E. coli FimHLD_G16P_V27A.

SEQ ID NO: 54 sets forth an amino acid sequence for the mutant E. coli FimHLD_G15A_G16A_V27A.

SEQ ID NO: 55 sets forth an amino acid sequence for the mutant E. coli FimHLD_V27A_R60P.

SEQ ID NO: 56 sets forth an amino acid sequence for the mutant E. coli FimHLD_G65A_V27A.

SEQ ID NO: 57 sets forth an amino acid sequence for the mutant E. coli FimHLD_V27A_Q133K.

SEQ ID NO: 58 sets forth an amino acid sequence for the mutant E. coli FimHLD_G15A_G16A_V27A_Q133K.

SEQ ID NO: 59 sets forth an amino acid sequence for wild type E. coli full-length FimH, including the donor strand FimG peptide connected through a linker (FimH-DSG_WT).

SEQ ID NO: 60 sets forth an amino acid sequence for the mutant E. coli FimH-DSG_V27A.

SEQ ID NO: 61 sets forth an amino acid sequence for the mutant E. coli FimH-DSG_G15A_V27A.

SEQ ID NO: 62 sets forth an amino acid sequence for the mutant E. coli FimH DSG_G15A_G16A_V27A.

SEQ ID NO: 63 sets forth an amino acid sequence for the mutant E. coli FimH DSG_V27A_Q133K.

SEQ ID NO: 64 sets forth an amino acid sequence for the mutant E. coli FimH DSG_G15A_G16A_V27A_Q133K.

SEQ ID NO: 65 sets forth an amino acid sequence for the mouse Ig Kappa signal peptide sequence.

SEQ ID NOs: 66-108 set forth amino acid and nucleic acid sequences for a nanostructure-related polypeptide or fragment thereof.

SEQ ID NO: 109—primer for PCR.

SEQ ID NO: 110—primer for PCR.

SEQ ID NO: 111—probe for PCR.

SEQ ID NO: 112 sets forth a O25b 2401 WzzB amino acid sequence.

SEQ ID NO: 113 sets forth a O25a:K5:H1 WzzB amino acid sequence.

SEQ ID NO: 114 sets forth a O25a ETEC ATCC WzzB amino acid sequence.

SEQ ID NO: 115 sets forth a K12 W3110 WzzB amino acid sequence.

SEQ ID NO: 116 sets forth a Salmonella LT2 WzzB amino acid sequence.

SEQ ID NO: 117 sets forth a O25b 2401 FepE amino acid sequence.

SEQ ID NO: 118 sets forth a O25a:K5:H1 FepE amino acid sequence.

SEQ ID NO: 119 sets forth a O25a ETEC ATCC FepE amino acid sequence.

SEQ ID NO: 120 sets forth a O157 FepE amino acid sequence.

SEQ ID NO: 121 sets forth a Salmonella LT2 FepE amino acid sequence.

SEQ ID NO: 122 sets forth a primer sequence for LT2wzzB_S.

SEQ ID NO: 123 sets forth a primer sequence for LT2wzzB_AS.

SEQ ID NO: 124 sets forth a primer sequence for O25bFepE_S.

SEQ ID NO: 125 sets forth a primer sequence for O25bFepE_A.

SEQ ID NO: 126 sets forth a primer sequence for wzzB P1_S.

SEQ ID NO: 127 sets forth a primer sequence for wzzB P2_AS.

SEQ ID NO: 128 sets forth a primer sequence for wzzB P3_S.

SEQ ID NO: 129 sets forth a primer sequence for wzzB P4_AS.

SEQ ID NO: 130 sets forth a primer sequence for O157 FepE_S.

SEQ ID NO: 131 sets forth a primer sequence for O157 FepE_AS.

SEQ ID NO: 132 sets forth a primer sequence for pBAD33_adaptor_S.

SEQ ID NO: 133 sets forth a primer sequence for pBAD33_adaptor_AS.

SEQ ID NO: 134 sets forth a primer sequence for JUMPSTART_r.

SEQ ID NO: 135 sets forth a primer sequence for gnd_f.

SEQ ID NO: 136 sets forth an amino acid sequence for a human IgG receptor FcRn large subunit p51 signal peptide.

SEQ ID NO: 137 sets forth an amino acid sequence for a human IL10 protein signal peptide.

SEQ ID NO: 138 sets forth an amino acid sequence for a human respiratory syncytial virus A (strain A2) fusion glycoprotein F0 signal peptide.

SEQ ID NO: 139 sets forth an amino acid sequence for an influenza A hemagglutinin signal peptide.

SEQ ID NOs: 140-147 set forth SignalP 4.1 (DTU Bioinformatics) sequences from various species used for signal peptide predictions.

### DETAILED DESCRIPTION

The present disclosure relates to E. coli FimH mutated polypeptides (mutants), compositions comprising the FimH mutants, methods for producing and purifying the FimH mutants, nucleic acids that encode the FimH mutants, host cells that comprise such nucleic acids, and methods of using compositions that comprise the FimH mutants.

Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to further illustrate the disclosure and does not pose a limitation on the scope of the claims. No language in the specification should be construed as indicating any non-claimed element essential to the practice of the disclosure.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

As used herein the term “about” means approximately or nearly, and in the context of a numerical value or range set forth herein in one embodiment means ±20%, 10%, 5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

As used herein, the term “wild type” or “WT” or “native” refers to an amino acid sequence that is found in nature, including allelic variations. A wild type amino acid sequence, peptide or protein has an amino acid sequence that has not been intentionally modified.

As used herein, “variants” of an amino acid sequence (peptide, protein or polypeptide), or “mutants’, or reference to a “mutated” polypeptide, comprise amino acid insertion variants/mutants, amino acid addition variants/mutants, amino acid deletion variants/mutants and/or amino acid substitution variants/mutants. The term “variant” or “mutant” includes all mutants, splice variants, posttranslationally modified variants, conformations, isoforms, allelic variants, species variants, and species homologs, in particular those which are naturally occurring. The term “variant” or “mutant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

As used herein, “sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues. The amino acid sequence variants/mutants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one aspect, a fragment or variant/mutant of an amino acid sequence (peptide or protein) is preferably a “functional fragment” or “functional variant”. The term “functional fragment” or “functional variant/mutant” of an amino acid sequence relates to any fragment or variant/mutant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant/mutant”, as used herein, in particular refers to a variant/mutant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

As used herein, “isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

I. E. coli FimH Polypeptides

Fimbrial adhesins, including type 1 fimbriae, bind to mannosylated glycoproteins in the epithelial layer or secreted into the urine. Type 1 fimbriae are highly conserved among clinical UPEC isolates and are encoded by a cluster of genes called fim, which encode accessory proteins (FimC, FimD), various structural subunits (FimE, FimF, FimG) and an adhesin called FimH. FimH is composed of two domains, the lectin binding domain (FimH_{LD}) responsible for binding to mannosylated glycoproteins, and the pilin domain. The pilin domain serves to link FimH to other structural subunits of the pilus such as FimG, via a mechanism called donor strand exchange. The FimH pilin domain forms an incomplete immunoglobulin fold, resulting in a groove that provides a binding site for the N-terminal β-strand of FimG, forming a strong intermolecular linkage between FimH and FimG. While FimH_{LD} can be expressed in a soluble, stable form, full length FimH is unstable alone unless in a complex with the chaperone FimC or complemented with the donor strand peptide of FimG in peptide form or as a fusion protein. Accordingly, the expression of a full length FimH molecule that is stable is possible by linking the FimG donor peptide to the C-terminus of full length FimH via a glycine-serine linker, and is designated FimH-DSG.

In one aspect, the present disclosure provides mutated FimH polypeptides, such as those shown in Table 1. Such mutants provide mutations in the amino acid sequence relative to the amino acid sequence of the corresponding wild-type (WT) FimH polypeptide. In some aspects, such mutants are immunogenic against the wild-type FimH protein or against a bacteria expressing the wild-type FimH polypeptide. In certain aspects, the FimH mutants possess certain beneficial characteristics, such as increased immunogenic properties as compared to the corresponding wild-type FimH polypeptide.

As used herein, the term “FimH polypeptide” refers to any domain of the full-length wild type E. coli FimH polypeptide, any combination of domains of the full-length wild type E. coli FimH polypeptide, or to the full-length E. coli FimH polypeptide, or any fragment thereof. For example, in one embodiment the present disclosure provides a mutated FimH polypeptide that is a mutated FimH_{LD} polypeptide, or a FimH-DSG polypeptide. The present disclosure relates to novel FimH_{LD} and FimH-DSG mutants with reduced affinity for mannoside ligands (verified by biochemical and biophysical analyses), which improves functional immunogenicity and describes the evaluation of neutralizing responses of these mutants relative to wild type FimH_{LD}.

The introduced amino acid mutations in the FimH mutant polypeptides can include amino acid substitutions, deletions, or additions. In some aspects, the only mutations in the amino acid sequence of the FimH polypeptide mutants are amino acid substitutions relative to a wild-type FimH protein.

TABLE 1
FimH wild type and mutant sequences

SEQ ID NO: 1 >FimHLD_WT
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 2 >FimHLD_G65A_V27A
FACKTASGTAIPIGGGSANVYVNLAPAVNVGQNLVVDLSTQIFCHNDY
PETITDYVTLQRGSAYAGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 3 >FimHLD_F1I
IACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 4 >FimHLD_F1L
LACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 5 >FimHLD_F1V
VACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 6 >FimHLD_F1M
MACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 7 >FimHLD_F1Y
YACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPIGG
SEQ ID NO: 8 >FimHLD_F1W
WACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 9 >FimHLD_Q133K
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRKTNNYNSDDFQF
VWNIYANNDVVVPTGG
SEQ ID NO: 10 >FimHLD_G15A
FACKTASGTAIPIGAGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 11 >FimHLD_G15P
FACKTASGTAIPIGPGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPIGG
SEQ ID NO: 12 >FimHLD_G16A
FACKTASGTAIPIGGASANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 13 >FimHLD_G16P
FACKTASGTAIPIGGPSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 14 >FimHLD_G15A_G16A
FACKTASGTAIPIGAASANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 15 >FimHLD_R60P
FACKTASGTAIPIGGGSANVYVNLAPVVNVGQNLVVDLSTQIFCHNDY
PETITDYVTLQPGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 16 >FimHLD_G65A
FACKTASGTAIPIGGGSANVYVNLAPVVNVGQNLVVDLSTQIFCHNDY
PETITDYVTLQRGSAYAGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 17 >FimHLD_P12C_A18C
FACKTASGTAICIGGGSCNVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 18 >FimHLD_G14C_F144C
FACKTASGTAIPICGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQC
VWNIYANNDVVVPIGG
SEQ ID NO: 19 >FimHLD_P26C_V35C
FACKTASGTAIPIGGGSANVYVNLACVVNVGQNLCVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 20 >FimHLD_P26C_V154C
FACKTASGTAIPIGGGSANVYVNLACVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDCVVPTGG
SEQ ID NO: 21 >FimHLD_P26C_V156C
FACKTASGTAIPIGGGSANVYVNLACVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVCPTGG
SEQ ID NO: 22 >FimHLD_V27C_L34C
FACKTASGTAIPIGGGSANVYVNLAPCVNVGQNCVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 23 >FimHLD_V28C_N33C
FACKTASGTAIPIGGGSANVYVNLAPVCNVGQCLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 24 >FimHLD_V28C_P157C
FACKTASGTAIPIGGGSANVYVNLAPVCNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVCTGG
SEQ ID NO: 25 >FimHLD_Q32C_Y108C
FACKTASGTAIPIGGGSANVYVNLAPVVNVGCNLVVDLSTQIFCHNDY
PETITDYVTLQRGSAYGGVLSSFSGTVKYSGSSYPFPTTSETPRVVYN
SRTDKPWPVALCLTPVSSAGGVAIKAGSLIAVLILRQTNNYNSDDFQF
VWNIYANNDVVVPTGG
SEQ ID NO: 26 >FimHLD_N33C_L109C
FACKTASGTAIPIGGGSANVYVNLAPVVNVGQCLVVDLSTQIFCHNDY
PETITDYVTLQRGSAYGGVLSSFSGTVKYSGSSYPFPTTSETPRVVYN
SRTDKPWPVALYCIPVSSAGGVAIKAGSLIAVLILRQTNNYNSDDFQF
VWNIYANNDVVVPTGG
SEQ ID NO: 27 >FimHLD_N33C_P157C
FACKTASGTAIPIGGGSANVYVNLAPVVNVGQCLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVCTGG
SEQ ID NO: 28 >FimHLD_V35C_L107C
FACKTASGTAIPIGGGSANVYVNLAPVVNVGQNLCVDLSTQIFCHNDY
PETITDYVTLQRGSAYGGVLSSFSGTVKYSGSSYPFPTTSETPRVVYN
SRTDKPWPVACYLTPVSSAGGVAIKAGSLIAVLILRQTNNYNSDDFQF
VWNIYANNDVVVPTGG
SEQ ID NO: 29 >FimHLD_V35C_L109C
FACKTASGTAIPIGGGSANVYVNLAPVVNVGQNLCVDLSTQIFCHNDY
PETITDYVTLQRGSAYGGVLSSFSGTVKYSGSSYPFPTTSETPRVVYN
SRTDKPWPVALYCIPVSSAGGVAIKAGSLIAVLILRQTNNYNSDDFQF
VWNIYANNDVVVPTGG
SEQ ID NO: 30 >FimHLD_S62C_T86C
FACKTASGTAIPIGGGSANVYVNLAPVVNVGQNLVVDLSTQIFCHNDY
PETITDYVTLQRGCAYGGVLSSFSGTVKYSGSSYPFPCTSETPRVVYN
SRTDKPWPVALYLTPVSSAGGVAIKAGSLIAVLILRQTNNYNSDDFQF
VWNIYANNDVVVPIGG
SEQ ID NO: 31 >FimHLD_S62C_L129C
FACKTASGTAIPIGGGSANVYVNLAPVVNVGQNLVVDLSTQIFCHNDY
PETITDYVTLQRGCAYGGVLSSFSGTVKYSGSSYPFPTTSETPRVVYN
SRTDKPWPVALYLTPVSSAGGVAIKAGSLIAVCILRQTNNYNSDDFQF
VWNIYANNDVVVPTGG
SEQ ID NO: 32 >FimHLD_Y64C_L68C
FACKTASGTAIPIGGGSANVYVNLAPVVNVGQNLVVDLSTQIFCHNDY
PETITDYVTLQRGSACGGVCSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 33 >FimHLD_Y64C_A127C
FACKTASGTAIPIGGGSANVYVNLAPVVNVGQNLVVDLSTQIFCHNDY
PETITDYVTLQRGSACGGVLSSFSGTVKYSGSSYPFPTTSETPRVVYN
SRTDKPWPVALYLTPVSSAGGVAIKAGSLICVLILRQTNNYNSDDFQF
VWNIYANNDVVVPTGG
SEQ ID NO: 34 >FimHLD_L68C_F71C
FACKTASGTAIPIGGGSANVYVNLAPVVNVGQNLVVDLSTQIFCHNDY
PETITDYVTLQRGSAYGGVCSSCSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 35 >FimHLD_V112C_T158C
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPCSSAGGVAIKAGSLIAVLILRQTNNYNSDDFQF
VWNIYANNDVVVPCGG
SEQ ID NO: 36 >FimHLD_S113C_G116C
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVCSACGVAIKAGSLIAVLILRQTNNYNSDDFQF
VWNIYANNDVVVPTGG
SEQ ID NO: 37 >FimHLD_S113C_T158C
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVCSAGGVAIKAGSLIAVLILRQTNNYNSDDFQF
VWNIYANNDVVVPCGG
SEQ ID NO: 38 >FimHLD_V118C_V156C
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGCAIKAGSLIAVLILRQTNNYNSDDFQF
VWNIYANNDVVCPTGG
SEQ ID NO: 39 >FimHLD_A119C_V155C
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVCIKAGSLIAVLILRQTNNYNSDDFQF
VWNIYANNDVCVPTGG
SEQ ID NO: 40 >FimHLD_L34N_V27A
FACKTASGTAIPIGGGSANVYVNLAPAVNVGQNNVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 41 >FimHLD_L34S_V27A
FACKTASGTAIPIGGGSANVYVNLAPAVNVGQNSVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 42 >FimHLD_L34T_V27A
FACKTASGTAIPIGGGSANVYVNLAPAVNVGQNTVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 43 >FimHLD_A119N_V27A
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVNIKAGSLIAVLILRQTNNYNSDDFQF
VWNIYANNDVVVPTGG
SEQ ID NO: 44 >FimHLD_A119S_V27A
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVSIKAGSLIAVLILRQTNNYNSDDFQF
VWNIYANNDVVVPTGG
SEQ ID NO: 45 >FimHLD_A119T_V27A
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVTIKAGSLIAVLILRQTNNYNSDDFQF
VWNIYANNDVVVPTGG
SEQ ID NO: 46 >FimH-DSG_A115V
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSVGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 47 >FimH-DSG_V163I
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDISARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 48 >FimH-DSG_V185I
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTIYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 49 >FimH-DSG_DSG_V3I
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
ITITVNGKVVAK
SEQ ID NO: 50 >FimHLD_G15A_V27A
FACKTASGTAIPIGAGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 51 >FimHLD_G16A_V27A
FACKTASGTAIPIGGASANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 52 >FimHLD_G15P_V27A
FACKTASGTAIPIGPGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 53 >FimHLD_G16P_V27A
FACKTASGTAIPIGGPSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 54 >FimHLD_G15A_G16A_V27A
FACKTASGTAIPIGAASANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 55 >FimHLD_V27A_R60P
FACKTASGTAIPIGGGSANVYVNLAPAVNVGQNLVVDLSTQIFCHNDY
PETITDYVTLQPGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 56 >FimHLD_G65A_V27A
FACKTASGTAIPIGGGSANVYVNLAPAVNVGQNLVVDLSTQIFCHNDY
PETITDYVTLQRGSAYAGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGG
SEQ ID NO: 57 >FimHLD_V27A_Q133K
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRKTNNYNSDDFQF
VWNIYANNDVVVPTGG
SEQ ID NO: 58 >FimHLD_G15A_G16A_V27A_Q133K
FACKTASGTAIPIGAASANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRKTNNYNSDDFQF
VWNIYANNDVVVPTGG
SEQ ID NO: 59 >FimH-DSG_WT
FACKTASGTAIPIGGGSANVYVNLAPVVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 60 >FimH-DSG_V27A
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 61 >FimH-DSG_G15A_V27A
FACKTASGTAIPIGAGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 62 >FimH-DSG_G15A_G16A_V27A
FACKTASGTAIPIGAASANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRQTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 63 >FimH-DSG_V27A_Q133K
FACKTASGTAIPIGGGSANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRKTNNYNSDDFQF
VWNIYANNDVVVPIGGCDVSARDVIVTLPDYPGSVPIPLIVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 64 >FimH-DSG_G15A_G16A_V27A_Q133K
FACKTASGTAIPIGAASANVYVNLAPAVNVGQNLVVDLSTQIFCHNDY
PETITDYVTLQRGSAYGGVLSSFSGTVKYSGSSYPFPTTSETPRVVYN
SRTDKPWPVALYLTPVSSAGGVAIKAGSLIAVLILRKTNNYNSDDFQF
VWNIYANNDVVVPTGGCDVSARDVTVTLPDYPGSVPIPLTVYCAKSQN
LGYYLSGTTADAGNSIFTNTASFSPAQGVGVQLTRQGTIIPANNTVSL
GAVGTSAVSLGLTANYARTGGQVTAGNVQSIIGVTFVYQGGSSGGGAD
VTITVNGKVVAK
SEQ ID NO: 65 >mouse Ig Kappa signal peptide
ETDTLLLWVLLLWVPGSTG

The amino acid sequence, of the wild type FimH polypeptide is well known in the art. For example, the amino acid sequence of the FimH_{LD} domain is provided herein as SEQ ID NO:1. The full-length wild type FimH polypeptide, including the FimG donor peptide linked to the C-terminus of full length FimH via a glycine-serine linker, is provided herein as SEQ ID NO:59. Nucleic acid sequences encoding such amino acid sequences are also well known in the art.

In one aspect of the disclosure, certain mutated FimH polypeptides result in a locked, open confirmation that results in reduced affinity for mannoside ligands, and leads to improved functional immunogenicity. Accordingly, such FimH mutants can be useful as antigens in an immunogenic composition, such as a vaccine, against E. coli infection. Since wild-type FimH_{LD} is considered to be a poor immunogen in terms of its ability to stimulate functional immunogenicity, such FimH mutants can provide improved antigens to be used in such immunogenic compositions.

In one aspect, as described in Example 1, the FimH mutants were designed in an attempt to lock the FimH lectin domain in an open confirmation in order to reduce the affinity for mannoside ligands. Such mutants can include at least 1, 2, 3, 4, 5, or more mutations. Mutations can include: naturally occurring amino acid substitutions that are common among urinary tract infection isolates (such as V27A); substitutions in the ligand binding side of FimH_{LD} (such as at positions F1, and Q133); glycine switch mutations in FimH_{LD} (such as at positions G15, G16, and G65); introducing cysteine pairs for disulfide bond stabilization in FimH_{LD} (such as at position pairs P12-A18; G14-F144; P26-V35; P26-V154; P26-V156; V27-L34; V28-N33; V28-P157; Q32-Y108; N33-L109; N33-P157; V35-L107; V35-L109; S62-T86; S62-L129; Y64-A127; L68-F71; V112-T158; S113-T158; V118-V 156; and/or A119-V155); nonpolar-to-polar mutations in FimH_{LD} (such as at positions V27, L34, A119, or any combination thereof); cavity filling mutations at the pilin-lectin interface of FimH-DSG (such as at positions A115, V163, V185, or V3 within the DSG sequence); or any combination of the types of mutations and at the amino acid positions noted above. In another aspect, the present disclosure provides FimH mutants as provided in SEQ ID NOs: 2-58, and 60-64, or to any combination of the mutants noted in any of such sequences. In another aspect, the present disclosure provides a FimH mutant according to any of SEQ ID NOs: 23, 50, 51, 52, 53, 54, 60, and 62. In a further aspect, the present disclosure provides a FimH mutant according to SEQ ID NO: 62.

In a further aspect, the present disclosure provides any of the FimH mutants as provided in SEQ ID NOs: 2-58, and 60-64, wherein said mutants are isolated. For example, in one aspect the present disclosure provides a FimH mutant according to any of SEQ ID NOs: 23, 50, 51, 52, 53, 54, 60, and 62 wherein said FimH mutant is isolated. In a further aspect, the present disclosure provides a FimH mutant according to SEQ ID NO: 62, wherein said FimH mutant is isolated.

Accordingly, in some specific aspects, the present disclosure provides a FimH mutant comprising a combination of introduced mutations, wherein the mutant comprises a combination of mutations set forth in any of the mutants provided in Table 1 (i.e. in SEQ ID NOs: 2-58 and 60-64). Any combination of the amino acid substitutions provided in each of the mutants in Table 1 can be made to a wild-type FimH polypeptide sequence to arrive at different FimH mutants. FimH mutants that are based on a native FimH polypeptide sequence of any other subtype or strain and comprise any of the combination of mutations described herein are also within the scope of the present disclosure.

A further aspect of the present disclosure is a polypeptide that is at least 90%, at least 91%, at least 92%, at least 93%, at least 94%, at least 95%, at least 96%, at least 97%, at least 98%, or at least 99% identical to any of SEQ ID NOs: 1-64. In a preferred aspect, the polypeptide is at least 90%, at least 91%, at least 92%, at least 93%, at least 94%, at least 95%, at least 96%, at least 97%, at least 98%, or at least 99% identical to SEQ ID NO: 62. In another aspect of the present invention is a polypeptide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identical to any of SEQ ID NOs: 1-64. In a preferred aspect, the polypeptide is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identical to SEQ ID NO: 62.

The FimH mutants provided by the present disclosure can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx® cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The FimH mutant polypeptides can be isolated using any suitable methods. For example, methods for purifying FimH protein mutant polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the FimH mutant polypeptides can include a “tag” that facilitates purification, such as an epitope tag or a histidine (His) tag. Such tagged polypeptides can conveniently be isolated, for example from conditioned media, by chelating chromatography or affinity chromatography.

The term “antigen” as used herein refers to a molecule that can be recognized by an antibody. Examples of antigens include polypeptides, peptides, lipids, polysaccharides, and nucleic acids containing antigenic determinants, such as those recognized by an immune cell.

### II. Nucleic Acids Encoding FimH Mutants

In another aspect, the present disclosure provides nucleic acid molecules that encode a FimH mutant as disclosed herein. Such nucleic acid molecules include DNA, cDNA, and RNA sequences. In one embodiment, the nucleic acid molecule can be incorporated into a vector, such as an expression vector.

In one aspect, nucleic acids encoding the E. coli FimH mutated polypeptides, or any fragment thereof, are disclosed. One or more nucleic acid constructs encoding the FimH mutant polypeptides, or a fragment thereof, may be used for genomic integration and subsequent expression of the polypeptide. For example, a single nucleic acid construct encoding the FimH mutant polypeptide, or fragment thereof, may be introduced into a host cell. Alternatively, the coding sequences for the polypeptide may be carried by two or more nucleic acid constructs, which are then introduced into a host cell simultaneously or sequentially.

For example, in one exemplary embodiment, a single nucleic acid construct encodes the lectin domain and pilin domain of an E. coli FimH. In another exemplary embodiment, one nucleic acid construct encodes the lectin domain and a second nucleic acid construct encodes the pilin domain of an E. coli FimH. In some aspects, genomic integration is achieved.

The nucleic acid construct may comprise genomic DNA that comprises one or more introns, or cDNA. Some genes are expressed more efficiently when introns are present. In some aspects, the nucleic acid sequence is suitable for the expression of exogenous polypeptides in said mammalian cell.

In some aspects, the nucleic acid encoding the polypeptide or fragment thereof is codon optimized to increase the level of expression in any particular cell.

In some aspects, the nucleic acid construct includes a signal sequence that encodes a peptide that directs secretion of the polypeptide derived from E. coli or a fragment thereof. In some aspects, the nucleic acid includes the native signal sequence of the polypeptide derived from E. coli FimH. In some aspects where the polypeptide derived from E. coli or a fragment thereof includes an endogenous signal sequence, the nucleic acid sequence encoding the signal sequence may be codon optimized to increase the level of expression of the protein in a host cell.

In some aspects, the signal sequence is any one of the following lengths: 15, 16, 17, 18, 19, 20, 21, 22, 23, 24, 25, 26, 27, 28, 29, and 30 amino acids long. In some aspects, the signal sequence is 20 amino acids long. In some aspects, the signal sequence is 21 amino acids long.

In some aspects, where the polypeptide or fragment thereof includes a signal sequence, the endogenous signal sequence naturally associated with the polypeptide may be replaced with a signal sequence not associated with the wild type polypeptide to improve the level of expression of the polypeptide or fragment thereof in cultured cells. Accordingly, in some aspects, the nucleic acid does not include the native signal sequence of the polypeptide derived from E. coli or a fragment thereof. In some aspects, the nucleic acid does not include the native signal sequence of the polypeptide derived from E. coli FimH. In some aspects, the polypeptide derived from E. coli or a fragment thereof may be expressed with a heterologous peptide, which is preferably a signal sequence or other peptide having a specific cleavage site at the N-terminus of the mature protein or polypeptide derived from E. coli or a fragment thereof. For example, the polypeptide derived from E. coli FimH or a fragment thereof may be expressed with a heterologous peptide (e.g., IgK signal sequence), which is preferably a signal sequence or other peptide having a specific cleavage site at the N-terminus of the mature E. coli FimH protein. In preferred aspects, the specific cleavage site at the N-terminus of the mature protein E. coli FimH occurs immediately before the initial phenylalanine residue of the mature E. coli FimH protein. The heterologous sequence selected is preferably one that is recognized and processed (i.e., cleaved by signal peptidase) by the host cell.

In preferred aspects, the signal sequence is an IgK signal sequence. In some aspects, the nucleic acid encodes a polypeptide having the amino acid sequence set forth in any of SEQ ID NOs: 1-64. In some aspects, the nucleic acid encodes the amino acid sequence SEQ ID NO: 23, 50, 51, 52, 53, 54, 60, 61, or 62. In some aspects, the nucleic acid encodes a polypeptide having the amino acid sequence set forth in SEQ ID NO: 62. In preferred aspects, the signal sequence is a mouse IgK signal sequence.

Suitable mammalian expression vectors for producing the FimH mutant polypeptides, or fragments thereof, are known in the art and may be commercially available, such as pSecTag2 expression vector from Invitrogen™. An exemplary mouse Ig Kappa signal peptide sequence includes the sequence ETDTLLLWVLLLWVPGSTG (SEQ ID NO: 65). In some aspects, the vector includes pBudCE4.1 mammalian expression vector from Thermo Fisher. Additional exemplary and suitable vectors include the pcDNA™ 3.1 mammalian expression vector (Thermo Fisher).

In some aspects, the signal sequence does not include a hemagglutinin signal sequence.

In some aspects, the nucleic acid includes the native signal sequence of the FimH polypeptide, or a fragment thereof. In some aspects, the signal sequence is not an IgK signal sequence. In some aspects, the signal sequence includes a hemagglutinin signal sequence.

In one aspect, disclosed herein are vectors that include the coding sequences for the FimH mutant polypeptide, or a fragment thereof. Exemplary vectors include plasmids that are able to replicate autonomously or to be replicated in a mammalian cell. Typical expression vectors contain suitable promoters, enhancers, and terminators that are useful for regulation of the expression of the coding sequence(s) in the expression construct. The vectors may also include selection markers to provide a phenotypic trait for selection of transformed host cells (such as conferring resistance to antibiotics such as ampicillin or neomycin).

Suitable promoters are known in the art. Exemplary promoters include, e.g., CMV promoter, adenovirus, EF1 a, GAPDH metallothionine promoter, SV-40 early promoter, SV-40 later promoter, murine mammary tumor virus promoter, Rous sarcoma virus promoter, polyhedrin promoter, etc. Promoters may be constitutive or inducible. One or more vectors may be used (e.g., one vector encoding all subunits or domains or fragments thereof, or multiple vectors together encoding the subunits or domains or fragments thereof).

Internal ribosome entry site (IRES) and 2A peptide sequences may also be used. IRES and 2A peptide provides alternative approaches for co-expression of multiple sequences. IRES is a nucleotide sequence that allows for translation initiation in the middle of a messenger RNA (mRNA) sequence as part of the greater process of protein synthesis. Usually, in eukaryotes, translation may be initiated only at the 5′ end of the mRNA molecule. IRES elements allow expression of multiple genes in one transcript. IRES-based polycistronic vectors, which express multiple proteins from one transcript, may reduce the escape of non-expressing clones from selection. The 2A peptide allows translation of multiple proteins in a single open reading frame into a polyprotein that is subsequently cleaved into individual proteins through a ribosome-skipping mechanism. 2A peptide may provide more balanced expression of multiple protein products. Exemplary IRES sequences include, e.g., EV71 IRES, EMCV IRES, HCV IRES. For genomic integration, the integration may be site-specific or random. Site-specific recombination may be achieved by introducing homologous sequence(s) into the nucleic acid constructs described herein. Such homologous sequence substantially matches the endogenous sequence at a specific target site in the host genome. Alternatively, random integration may be used. Sometimes, the expression level of a protein may vary depending upon the integration site. Therefore, it may be desirable to select a number of clones according to recombinant protein expression level to identify a clone that achieves the desired level of expression.

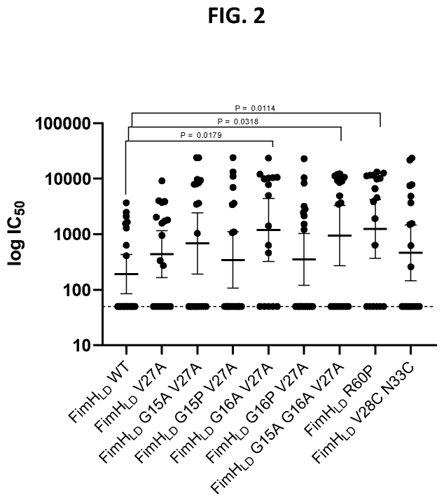

Exemplary nucleic acid constructs are further described in the figures, e.g. FIG. 2A-2T, of PCT Intl. Publication No. WO2021/084429, published May 6, 2021, which is incorporated herein by reference.

In one aspect, the nucleic acid sequence encodes the amino acid sequence having at least 90%, at least 91%, at least 92%, at least 93%, at least 94%, at least 95%, at least 96%, at least 97%, at least 98%, at least 99%, identity to any one of any of SEQ ID NOs: 1-64. In a preferred aspect, the nucleic acid sequence encodes the amino acid sequence having at least 90%, at least 91%, at least 92%, at least 93%, at least 94%, at least 95%, at least 96%, at least 97%, at least 98%, or at least 99% identical to SEQ ID NO: 62. In another aspect of the present invention the nucleic acid sequence encodes the amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identical to any of SEQ ID NOs: 1-64. In a preferred aspect, the nucleic acid sequence encodes the amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9% identical to SEQ ID NO: 62.

In certain aspects of the present disclosure, the RNA is messenger RNA (mRNA) that relates to a RNA transcript which encodes a peptide or protein. As established in the art, mRNA generally contains a 5 untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides. In one aspect, the RNA described herein may have modified nucleosides. In some aspects, the RNA comprises a modified nucleoside in place of at least one (e.g., every) uridine.

In some embodiments, compositions or medical preparations described herein comprise RNA encoding an amino acid sequence comprising a FimH mutant polypeptide. Likewise, methods described herein comprise administration of such RNA. One possible platform for use herein is based on an antigen-coding RNA vaccine to induce robust neutralizing antibodies and accompanying/concomitant T cell response to achieve protective immunization with preferably minimal vaccine doses. The RNA administered is preferably in-vitro transcribed RNA. Three different RNA platforms are particularly preferred, namely non-modified uridine containing mRNA (uRNA), nucleoside modified mRNA (modRNA) and self-amplifying RNA (saRNA). In one particularly preferred aspect, the RNA is in vitro transcribed RNA.

### III. Host Cells

In one aspect, the disclosure relates to cells in which the sequences encoding the FimH mutant polypeptide, or a fragment thereof are expressed in a mammalian host cell. In one embodiment, the polypeptide is transiently expressed in the host cell. In another embodiment, the polypeptide is stably integrated into the genome of the host cells, and, when cultured under a suitable condition, expresses the polypeptide or a fragment thereof. In a preferred embodiment, the polynucleotide sequence is expressed with high efficiency and genomic stability.

Suitable mammalian host cells are known in the art. Preferably, the host cell is suitable for producing protein at industrial manufacturing scale. Exemplary mammalian host cells include any one of the following and derivatives thereof: Chinese Hamster Ovary (CHO) cells, COS cells (a cell line derived from monkey kidney (African green monkey), Vero cells, Hela cells, baby hamster kidney (BHK) cells, Human Embryonic Kidney (HEK) cells, NSO cells (Murine myeloma cell line), and C127 cells (nontumorigenic mouse cell line). Further exemplary mammalian host cells include mouse Sertoli (TM4), buffalo rat liver (BRL 3A), mouse mammary tumor (MMT), rat hepatoma (HTC), mouse myeloma (NSO), murine hybridoma (Sp2/0), mouse thymoma (EL4), Chinese Hamster Ovary (CHO) and CHO cell derivatives, murine embryonic (NIH/3T3, 3T3 Li), rat myocardial (H9c2), mouse myoblast (C2C12), and mouse kidney (miMCD-3). Further examples of mammalian cell lines include NSO/1, Sp2/0, Hep G2, PER.C6, COS-7, TM4, CV1, VERO-76, MDCK, BRL 3A, W138, MMT 060562, TR1, MRC5, and FS4.

Any cell susceptible to cell culture may be utilized in accordance with the present invention. In some aspects, the cell is a mammalian cell. Non-limiting examples of mammalian cells that may be used in accordance with the present invention include BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse 28yophil cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2). In some preferred embodiment, the cells are CHO cells. In some preferred aspects, the cells are GS-cells.

Additionally, any number of commercially and non-commercially available hybridoma cell lines may be utilized in accordance with the present invention. The term “hybridoma” as used herein refers to a cell or progeny of a cell resulting from fusion of an immortalized cell and an antibody-producing cell. Such a resulting hybridoma is an immortalized cell that produces antibodies. Individual cells used to create the hybridoma can be from any mammalian source, including, but not limited to, rat, pig, rabbit, sheep, pig, goat, and human. In some aspects, a hybridoma is a trioma cell line, which results when progeny of heterohybrid myeloma fusions, which are the product of a fusion between human cells and a murine myeloma cell line, are subsequently fused with a plasma cell. In some aspects, a hybridoma is any immortalized hybrid cell line that produces antibodies such as, for example, quadromas (See, e.g., Milstein et al., Nature 537:3053 (1983)). One skilled in the art will appreciate that hybridoma cell lines might have different nutrition requirements and/or might require different culture conditions for optimal growth, and will be able to modify conditions as needed.

In some aspects, the cell comprises a first gene of interest, wherein the first gene of interest is chromosomally-integrated. In some aspects, the first gene of interest comprises a reporter gene, a selection gene, a gene of interest (e.g., encoding a polypeptide derived from E. coli or a fragment thereof), an ancillary gene, or a combination thereof. In some aspects, the gene of therapeutic interest comprises a gene encoding a difficult to express (DtE) protein.

In some aspects, the first gene of interest is located between two of the distinct recombination target sites (RTS) in a site-specific integration (SSI) mammalian cell, wherein two RTS are chromosomally-integrated within the NL1 locus or the NL2 locus. See, for example, United States Patent Application Publication No. 20200002727, for a description of the NL1 locus, the NL2 locus, the NL3 locus, the NL4 locus, the NL5 locus, and the NL6 locus. In some aspects, the first gene of interest is located within the NL1 locus. In some aspects, the cell comprises a second gene of interest, wherein the second gene of interest is chromosomally-integrated. In some aspects, the second gene of interest comprises a reporter gene, a selection gene, a gene of therapeutic interest (such as a FimH mutant polypeptide, or a fragment thereof), an ancillary gene, or a combination thereof. In some aspects, the gene of therapeutic interest comprises a gene encoding a DtE protein. In some aspects, the second gene of interest is located between two of the RTS. In some aspects, the second gene of interest is located within the NL1 locus or the NL2 locus. In some aspects, the first gene of interest is located within the NL1 locus, and the second gene of interest is located within the NL2 locus. In some aspects, the cell comprises a third gene of interest, wherein the third gene of interest is chromosomally-integrated. In some aspects, the third gene of interest comprises a reporter gene, a selection gene, a gene of therapeutic interest (such as a polypeptide derived from E. coli or a fragment thereof), an ancillary gene, or a combination thereof. In some aspects, the gene of therapeutic interest comprises a gene encoding a DtE protein. In some aspects, the third gene of interest is located between two of the RTS. In some aspects, the third gene of interest is located within the NL1 locus or the NL2 locus. In some aspects, the third gene of interest is located within a locus distinct from the NL1 locus and the NL2 locus. In some aspects, the first gene of interest, the second gene of interest, and the third gene of interest are within three separate loci. In some aspects, at least one of the first genes of interest, the second gene of interest, and the third gene of interest is within the NL1 locus, and at least one of the first gene of interest, the second gene of interest, and the third gene of interest is within the NL2 locus. In some aspects, the cell comprises a site-specific recombinase gene. In some aspects, the site-specific recombinase gene is chromosomally-integrated.

In another aspect, the present disclosure provides a mammalian cell comprising at least four distinct RTS, wherein the cell comprises (a) at least two distinct RTS are chromosomally-integrated within the NL1 locus or NL2 locus; (b) a first gene of interest is integrated between the at least two RTS of (a), wherein the first gene of interest comprises a reporter gene, a gene encoding a DtE protein, an ancillary gene or a combination thereof; (c) and a second gene of interest is integrated within a second chromosomal locus distinct from the locus of (a), wherein the second gene of interest comprises a reporter gene, a gene encoding a DtE protein (such as a polypeptide derived from E. coli or a fragment thereof), an ancillary gene or a combination thereof. In some aspects, the present disclosure provides a mammalian cell comprising at least four distinct RTS, wherein the cell comprises (a) at least two distinct RTS are chromosomally-integrated within the Fer1L4 locus; (b) at least two distinct RTS are chromosomally-integrated within the NL1 locus or the NL2 locus; (c) a first gene of interest is chromosomally-integrated within the Fer1L4 locus, wherein the first gene of interest comprises a reporter gene, a gene encoding a DtE protein, an ancillary gene or a combination thereof; and (d) a second gene of interest is chromosomally-integrated within the within the NL1 locus or NL2 locus of (b), wherein the second gene of interest comprises a reporter gene, a gene encoding a DtE protein (such as a polypeptide derived from E. coli or a fragment thereof), an ancillary gene or a combination thereof.

In some aspects, the present disclosure provides a mammalian cell comprising at least six distinct RTS, wherein the cell comprises (a) at least two distinct RTS and a first gene of interest are chromosomally-integrated within the Fer1L4 locus; (b) at least two distinct RTS and a second gene of interest are chromosomally-integrated within the NL1 locus; and (c) at least two distinct RTS and a third gene of interest are chromosomally-integrated within the NL2 locus.

As referred to herein, the terms “in operable combination,” “in operable order,” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. In some aspects, a gene of interest is operably linked to a promoter, wherein the gene of interest is chromosomally-integrated into the host cell. In some aspects, the gene of interest is operably linked to a heterologous promoter; where in the gene of interest is chromosomally-integrated into the host cell. In some aspects, an ancillary gene is operably linked to a promoter, wherein the ancillary gene is chromosomally-integrated into the host cell genome. In some aspects, the ancillary gene is operably linked to a heterologous promoter; where in the ancillary gene is chromosomally-integrated into the host cell genome. In some aspects, a gene encoding a DtE protein is operably linked to a promoter, wherein the gene encoding a DtE protein is chromosomally-integrated into the host cell genome. In some aspects, the gene encoding a DtE protein is operably linked to a heterologous promoter, where in the gene encoding a DtE protein is chromosomally-integrated into the host cell genome. In some aspects, a recombinase gene is operably linked to a promoter, wherein the recombinase gene is chromosomally-integrated into the host cell. In some aspects, the recombinase gene is operably linked to a promoter, where in the recombinase gene is not integrated into the host cell genome. In some aspects, a recombinase gene is operably linked to a heterologous promoter, wherein the recombinase gene is not chromosomally-integrated into the host cell genome. In some aspects, the recombinase gene is operably linked to a heterologous promoter, wherein the recombinase gene is not chromosomally-integrated into the host cell genome.

As referred to herein, the term “chromosomally-integrated” or “chromosomal integration” refers to the stable incorporation of a nucleic acid sequence into the chromosome of a host cell, e.g. a mammalian cell. i.e., a nucleic acid sequence that is chromosomally-integrated into the genomic DNA (gDNA) of a host cell, e.g. a mammalian cell. In some aspects, a nucleic acid sequence that is chromosomally-integrated is stable. In some aspects, a nucleic acid sequence that is chromosomally-integrated is not located on a plasmid or a vector. In some aspects, a nucleic acid sequence that is chromosomally-integrated is not excised. In some aspects, chromosomal integration is mediated by the clustered regularly interspaced short palindromic repeats (CRISPR) and CRISPR associated protein (Cas) gene editing system (CRISPR/CAS).

### IV. Compositions and Formulations

In one aspect, the present disclosure includes a composition that comprises at least one FimH mutant polypeptide or fragment thereof as described herein. In some aspects, the composition elicits an immune response, including antibodies, that may confer immunity to pathogenic species of E. coli.

In some aspects, the composition comprises a FimH mutant polypeptide as the only antigen. In some aspects, the composition does not include a conjugate.

In some aspects, the composition comprises a FimH mutant polypeptide and at least one additional antigen. In some aspects, the composition comprises a FimH mutant polypeptide and an additional E. coli antigen. In some aspects, the composition comprises a FimH mutant polypeptide and a glycoconjugate from E. coli.

In some aspects, the composition comprises a FimH mutant polypeptide and a polypeptide derived from E. coli FimC or a fragment thereof.

In one embodiment, the disclosure includes a composition comprising a FimH mutant polypeptide, and a saccharide comprising a structure selected from any one of the saccharide structures disclosed in PCT Intl. Publication Nos. WO2021/084429, published May 6, 2021, and WO2020/039359, published Feb. 27, 2020, and US Publication No. US2020/0061177, published Feb. 27, 2020, which are each incorporated herein by reference in their entireties. In one aspect, the disclosure includes a composition comprising a FimH mutant polypeptide,; and a saccharide comprising a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187, wherein n is an integer from 1 to 100, preferably from 31 to 90.

In some embodiments, the composition includes any one of the saccharides disclosed herein. In preferred embodiments, the composition includes any one of the conjugates disclosed herein.

In some embodiments, the composition includes at least one glycoconjugate from E. coli serotype O25, preferably serotype O25b. In one embodiment, the composition includes at least one glycoconjugate from E. coli serotype O1, preferably serotype O1a. In one embodiment, the composition includes at least one glycoconjugate from E. coli serotype O2. In one embodiment, the composition includes at least one glycoconjugate from E. coli serotype O6.

In one embodiment, the composition comprises at least one glycoconjugate selected from any one of the following E. coli serotypes O25, O1, O2, and O6, preferably O25b, O1a, O2, and O6. In one embodiment, the composition comprises at least two glycoconjugates selected from any one of the following E. coli serotypes O25, O1, O2, and O6, preferably O25b, O1a, O2, and O6. In another embodiment, the composition comprises at least three glycoconjugates selected from any one of the following E. coli serotypes O25, O1, O2, and O6, preferably O25b, O1a, O2, and O6. In a further embodiment, the composition comprises a glycoconjugate from each of the following E. coli serotypes O25, O1, O2, and O6, preferably O25b, O1a, O2, and O6.

In a preferred embodiment, the glycoconjugate of any of the above compositions is individually conjugated to CRM₁₉₇. In another preferred embodiment, the glycoconjugate of any of the above compositions is individually conjugated to SCP.

Accordingly, in some embodiments, the composition includes a FimH mutant polypeptide, and an O-antigen from at least one E. coli serotype. In a preferred embodiment, the composition includes a FimH mutant polypeptide, and an O-antigen from more than 1 E. coli serotype. For example, the composition may include an O-antigen from two different E. coli serotypes (or “v”, valences) to 12 different serotypes (12v). In one embodiment, the composition includes a FimH mutant polypeptide, and an O-antigen from 3 different serotypes. In one embodiment, the composition includes a FimH mutant polypeptide, and an O-antigen from 4 different E. coli serotypes. In one embodiment, the composition includes a FimH mutant polypeptide, and an O-antigen from 5 different E. coli serotypes. In one embodiment, the composition includes a FimH mutant polypeptide, and an O-antigen from 6 different E. coli serotypes. In one embodiment, the composition includes a FimH mutant polypeptide, and an O-antigen from 7 different E. coli serotypes. In one embodiment, the composition includes a FimH mutant polypeptide, and an O-antigen from 8 different E. coli serotypes. In one embodiment, the composition includes a FimH mutant polypeptide, and an O-antigen from 9 different E. coli serotypes. In one embodiment, the composition includes a FimH mutant polypeptide, and an O-antigen from 10 different E. coli serotypes. In one embodiment, the composition includes a FimH mutant polypeptide, and an O-antigen from 11 different E. coli serotypes. In one embodiment, the composition includes a FimH mutant polypeptide, and an O-antigen from 12 different serotypes. In one embodiment, the composition includes a FimH mutant polypeptide, and an O-antigen from 13 different serotypes. In one embodiment, the composition includes a FimH mutant polypeptide, and an O-antigen from 14 different serotypes. In one embodiment, the composition includes a FimH mutant polypeptide, and an O-antigen from 15 different serotypes. In one embodiment, the composition includes a FimH mutant polypeptide, and an O-antigen from 16 different serotypes. In one embodiment, the composition includes a FimH mutant polypeptide, and an O-antigen from 17 different serotypes. In one embodiment, the composition includes a FimH mutant polypeptide, and an O-antigen from 18 different serotypes. In one embodiment, the composition includes a FimH mutant polypeptide, and an O-antigen from 19 different serotypes. In one embodiment, the composition includes a FimH mutant polypeptide, and an O-antigen from 20 different serotypes.

Preferably, the number of E. coli saccharides can range from 1 serotype (or “v”, valences) to 26 different serotypes (26v). In one embodiment there is one serotype. In one embodiment there are 2 different serotypes. In one embodiment there are 3 different serotypes. In one embodiment there are 4 different serotypes. In one embodiment there are 5 different serotypes. In one embodiment there are 6 different serotypes. In one embodiment there are 7 different serotypes. In one embodiment there are 8 different serotypes. In one embodiment there are 9 different serotypes. In one embodiment there are 10 different serotypes. In one embodiment there are 11 different serotypes. In one embodiment there are 12 different serotypes. In one embodiment there are 13 different serotypes. In one embodiment there are 14 different serotypes. In one embodiment there are 15 different serotypes. In one embodiment there are 16 different serotypes. In one embodiment there are 17 different serotypes. In one embodiment there are 18 different serotypes. In one embodiment there are 19 different serotypes. In one embodiment there are 20 different serotypes. In one embodiment there are 21 different serotypes. In one embodiment there are 22 different serotypes. In one embodiment there are 23 different serotypes. In one embodiment there are 24 different serotypes. In an embodiment there are 25 different serotypes. In one embodiment there are 26 different serotypes. The saccharides are conjugated to a carrier protein to form glycoconjugates as described herein.

In one aspect, the composition includes a FimH mutant polypeptide; and a glycoconjugate that includes an O-antigen from at least one E. coli serogroup, wherein the O-antigen is conjugated to a carrier protein. In one embodiment, the composition includes a FimH mutant polypeptide; and an O-antigen from more than 1 E. coli serotype, wherein each O-antigen is conjugated to a carrier protein. In one embodiment, the composition includes a FimH mutant polypeptide, and an O-antigen from 2 different E. coli serotypes, wherein each O-antigen is conjugated to a carrier protein. In one embodiment, the composition includes a FimH mutant polypeptide, and an O-antigen from 3 different E. coli serotypes, wherein each O-antigen is conjugated to a carrier protein. In one embodiment, the composition includes a FimH mutant polypeptide, and an O-antigen from 4 different E. coli serotypes, wherein each O-antigen is conjugated to a carrier protein. In one embodiment, the composition includes a FimH mutant polypeptide, and an O-antigen from 5 different E. coli serotypes, wherein each O-antigen is conjugated to a carrier protein. In one embodiment, the composition includes a FimH mutant polypeptide, and an O-antigen from 6 different E. coli serotypes, wherein each O-antigen is conjugated to a carrier protein. In one embodiment, the composition includes a FimH mutant polypeptide, and an O-antigen from 7 different E. coli serotypes, wherein each O-antigen is conjugated to a carrier protein. In one embodiment, the composition includes a FimH mutant polypeptide, and an O-antigen from 8 different E. coli serotypes, wherein each O-antigen is conjugated to a carrier protein. In one embodiment, the composition includes a FimH mutant polypeptide, and an O-antigen from 9 different E. coli serotypes, wherein each O-antigen is conjugated to a carrier protein. In one embodiment, the composition includes an O-antigen from a FimH mutant polypeptide, and 10 different E. coli serotypes, wherein each O-antigen is conjugated to a carrier protein. In one embodiment, the composition includes an O-antigen from a FimH mutant polypeptide, and 11 different E. coli serotypes, wherein each O-antigen is conjugated to a carrier protein. In one embodiment, the composition includes a FimH mutant polypeptide, and an O-antigen from 12 different serotypes, wherein each O-antigen is conjugated to a carrier protein. In one embodiment, the composition includes a FimH mutant polypeptide, and an O-antigen from 13 different serotypes, wherein each O-antigen is conjugated to a carrier protein. In one embodiment, the composition includes a FimH mutant polypeptide, and an O-antigen from 14 different serotypes, wherein each O-antigen is conjugated to a carrier protein. In one embodiment, the composition includes a FimH mutant polypeptide, and an O-antigen from 15 different serotypes, wherein each O-antigen is conjugated to a carrier protein. In one embodiment, the composition includes a FimH mutant polypeptide, and an O-antigen from 16 different serotypes, wherein each O-antigen is conjugated to a carrier protein. In one embodiment, the composition includes a FimH mutant polypeptide, and an O-antigen from 17 different serotypes, wherein each O-antigen is conjugated to a carrier protein. In one embodiment, the composition includes a FimH mutant polypeptide, and an O-antigen from 18 different serotypes, wherein each O-antigen is conjugated to a carrier protein. In one embodiment, the composition includes a FimH mutant polypeptide, and an O-antigen from 19 different serotypes, wherein each O-antigen is conjugated to a carrier protein. In one embodiment, the composition includes a FimH mutant polypeptide, and an O-antigen from 20 different serotypes, wherein each O-antigen is conjugated to a carrier protein.

In another aspect, the composition includes an O-polysaccharide from at least one E. coli serotype. In a preferred embodiment, the composition includes an O-polysaccharide from more than 1 E. coli serotype. For example, the composition may include an O-polysaccharide from two different E. coli serotypes to 12 different E. coli serotypes. In one embodiment, the composition includes an O-polysaccharide from 3 different E. coli serotypes. In one embodiment, the composition includes an O-polysaccharide from 4 different E. coli serotypes. In one embodiment, the composition includes an O-polysaccharide from 5 different E. coli serotypes. In one embodiment, the composition includes an O-polysaccharide from 6 different E. coli serotypes. In one embodiment, the composition includes an O-polysaccharide from 7 different E. coli serotypes. In one embodiment, the composition includes an O-polysaccharide from 8 different E. coli serotypes. In one embodiment, the composition includes an O-polysaccharide from 9 different E. coli serotypes. In one embodiment, the composition includes an O-polysaccharide from 10 different E. coli serotypes. In one embodiment, the composition includes an O-polysaccharide from 11 different E. coli serotypes. In one embodiment, the composition includes an O-polysaccharide from 12 different serotypes. In one embodiment, the composition includes an O-polysaccharide from 13 different serotypes. In one embodiment, the composition includes an O-polysaccharide from 14 different serotypes. In one embodiment, the composition includes an O-polysaccharide from 15 different serotypes. In one embodiment, the composition includes an O-polysaccharide from 16 different serotypes. In one embodiment, the composition includes an O-polysaccharide from 17 different serotypes. In one embodiment, the composition includes an O-polysaccharide from 18 different serotypes. In one embodiment, the composition includes an O-polysaccharide from 19 different serotypes. In one embodiment, the composition includes an O-polysaccharide from 20 different serotypes.

In a preferred embodiment, the composition includes an O-polysaccharide from at least one E. coli serotype, wherein the O-polysaccharide is conjugated to a carrier protein. In a preferred embodiment, the composition includes an O-polysaccharide from more than 1 E. coli serotype, wherein each O-polysaccharide is conjugated to a carrier protein. For example, the composition may include an O-polysaccharide from two different E. coli serotypes to 12 different E. coli serotypes, wherein each O-polysaccharide is conjugated to a carrier protein. In one embodiment, the composition includes an O-polysaccharide from 3 different E. coli serotypes, wherein each O-polysaccharide is conjugated to a carrier protein. In one embodiment, the composition includes an O-polysaccharide from 4 different E. coli serotypes, wherein each O-polysaccharide is conjugated to a carrier protein. In one embodiment, the composition includes an O-polysaccharide from 5 different E. coli serotypes, wherein each O-polysaccharide is conjugated to a carrier protein. In one embodiment, the composition includes an O-polysaccharide from 6 different E. coli serotypes, wherein each O-polysaccharide is conjugated to a carrier protein. In one embodiment, the composition includes an O-polysaccharide from 7 different E. coli serotypes, wherein each O-polysaccharide is conjugated to a carrier protein. In one embodiment, the composition includes an O-polysaccharide from 8 different E. coli serotypes, wherein each O-polysaccharide is conjugated to a carrier protein. In one embodiment, the composition includes an O-polysaccharide from 9 different E. coli serotypes, wherein each O-polysaccharide is conjugated to a carrier protein. In one embodiment, the composition includes an O-polysaccharide from 10 different E. coli serotypes, wherein each O-polysaccharide is conjugated to a carrier protein. In one embodiment, the composition includes an O-polysaccharide from 11 different E. coli serotypes, wherein each O-polysaccharide is conjugated to a carrier protein. In one embodiment, the composition includes an O-polysaccharide from 12 different serotypes, wherein each O-polysaccharide is conjugated to a carrier protein. In one embodiment, the composition includes an O-polysaccharide from 13 different serotypes, wherein each O-polysaccharide is conjugated to a carrier protein. In one embodiment, the composition includes an O-polysaccharide from 14 different serotypes, wherein each O-polysaccharide is conjugated to a carrier protein. In one embodiment, the composition includes an O-polysaccharide from 15 different serotypes, wherein each O-polysaccharide is conjugated to a carrier protein. In one embodiment, the composition includes an O-polysaccharide from 16 different serotypes, wherein each O-polysaccharide is conjugated to a carrier protein. In one embodiment, the composition includes an O-polysaccharide from 17 different serotypes, wherein each O-polysaccharide is conjugated to a carrier protein. In one embodiment, the composition includes an O-polysaccharide from 18 different serotypes, wherein each O-polysaccharide is conjugated to a carrier protein. In one embodiment, the composition includes an O-polysaccharide from 19 different serotypes, wherein each O-polysaccharide is conjugated to a carrier protein. In one embodiment, the composition includes an O-polysaccharide from 20 different serotypes, wherein each O-polysaccharide is conjugated to a carrier protein.

In a most preferred embodiment, the composition includes an O-polysaccharide from at least one E. coli serotype, wherein the O-polysaccharide is conjugated to a carrier protein, and wherein the O-polysaccharide includes the O-antigen and core saccharide. In a preferred embodiment, the composition includes an O-polysaccharide from more than 1 E. coli serotype, wherein each O-polysaccharide is conjugated to a carrier protein, and wherein the O-polysaccharide includes the O-antigen and core saccharide. For example, the composition may include an O-polysaccharide from two different E. coli serotypes to 12 different E. coli serotypes, wherein each O-polysaccharide is conjugated to a carrier protein, and wherein the O-polysaccharide includes the O-antigen and core saccharide. In one embodiment, the composition includes an O-polysaccharide from 3 different E. coli serotypes, wherein each O-polysaccharide is conjugated to a carrier protein, and wherein the O-polysaccharide includes the O-antigen and core saccharide. In one embodiment, the composition includes an O-polysaccharide from 4 different E. coli serotypes, wherein each O-polysaccharide is conjugated to a carrier protein, and wherein the O-polysaccharide includes the O-antigen and core saccharide. In one embodiment, the composition includes an O-polysaccharide from 5 different E. coli serotypes, wherein each O-polysaccharide is conjugated to a carrier protein, and wherein the O-polysaccharide includes the O-antigen and core saccharide. In one embodiment, the composition includes an O-polysaccharide from 6 different E. coli serotypes, wherein each O-polysaccharide is conjugated to a carrier protein, and wherein the O-polysaccharide includes the O-antigen and core saccharide. In one embodiment, the composition includes an O-polysaccharide from 7 different E. coli serotypes, wherein each O-polysaccharide is conjugated to a carrier protein, and wherein the O-polysaccharide includes the O-antigen and core saccharide. In one embodiment, the composition includes an O-polysaccharide from 8 different E. coli serotypes, wherein each O-polysaccharide is conjugated to a carrier protein, and wherein the O-polysaccharide includes the O-antigen and core saccharide. In one embodiment, the composition includes an O-polysaccharide from 9 different E. coli serotypes, wherein each O-polysaccharide is conjugated to a carrier protein, and wherein the O-polysaccharide includes the O-antigen and core saccharide. In one embodiment, the composition includes an O-polysaccharide from 10 different E. coli serotypes, wherein each O-polysaccharide is conjugated to a carrier protein, and wherein the O-polysaccharide includes the O-antigen and core saccharide. In one embodiment, the composition includes an O-polysaccharide from 11 different E. coli serotypes, wherein each O-polysaccharide is conjugated to a carrier protein, and wherein the O-polysaccharide includes the O-antigen and core saccharide. In one embodiment, the composition includes an O-polysaccharide from 12 different serotypes, wherein each O-polysaccharide is conjugated to a carrier protein, and wherein the O-polysaccharide includes the O-antigen and core saccharide. In one embodiment, the composition includes an O-polysaccharide from 13 different serotypes, wherein each O-polysaccharide is conjugated to a carrier protein, and wherein the O-polysaccharide includes the O-antigen and core saccharide. In one embodiment, the composition includes an O-polysaccharide from 14 different serotypes, wherein each O-polysaccharide is conjugated to a carrier protein, and wherein the O-polysaccharide includes the O-antigen and core saccharide. In one embodiment, the composition includes an O-polysaccharide from 15 different serotypes, wherein each O-polysaccharide is conjugated to a carrier protein, and wherein the O-polysaccharide includes the O-antigen and core saccharide. In one embodiment, the composition includes an O-polysaccharide from 16 different serotypes, wherein each O-polysaccharide is conjugated to a carrier protein, and wherein the O-polysaccharide includes the O-antigen and core saccharide. In one embodiment, the composition includes an O-polysaccharide from 17 different serotypes, wherein each O-polysaccharide is conjugated to a carrier protein, and wherein the O-polysaccharide includes the O-antigen and core saccharide. In one embodiment, the composition includes an O-polysaccharide from 18 different serotypes, wherein each O-polysaccharide is conjugated to a carrier protein, and wherein the O-polysaccharide includes the O-antigen and core saccharide. In one embodiment, the composition includes an O-polysaccharide from 19 different serotypes, wherein each O-polysaccharide is conjugated to a carrier protein, and wherein the O-polysaccharide includes the O-antigen and core saccharide. In one embodiment, the composition includes an O-polysaccharide from 20 different serotypes, wherein each O-polysaccharide is conjugated to a carrier protein, and wherein the O-polysaccharide includes the O-antigen and core saccharide. In a preferred embodiment, the carrier protein is CRM₁₉₇.

In another preferred embodiment, the composition includes a FimH mutant polypeptide, and an O-polysaccharide conjugated to CRM₁₉₇, wherein the O-polysaccharide includes Formula O25a, wherein n is at least 30, and the core saccharide. In a preferred embodiment, the composition further includes an O-polysaccharide conjugated to CRM₁₉₇, wherein the O-polysaccharide includes Formula O25b, wherein n is at least 40, and the core saccharide. In another embodiment, the composition further includes an O-polysaccharide conjugated to CRM₁₉₇, wherein the O-polysaccharide includes Formula O1a, wherein n is at least 30, and the core saccharide. In another embodiment, the composition further includes an O-polysaccharide conjugated to CRM₁₉₇, wherein the O-polysaccharide includes Formula O2, wherein n is at least 30, and the core saccharide. In another embodiment, the composition further includes an O-polysaccharide conjugated to CRM₁₉₇, wherein the O-polysaccharide includes Formula O6, wherein n is at least 30, and the core saccharide.

In another embodiment, the composition further includes an O-polysaccharide conjugated to CRM₁₉₇, wherein the O-polysaccharide includes Formula O17, wherein n is at least 30, and the core saccharide. In another embodiment, the composition further includes an O-polysaccharide conjugated to CRM₁₉₇, wherein the O-polysaccharide includes Formula O15, wherein n is at least 30, and the core saccharide. In another embodiment, the composition further includes an O-polysaccharide conjugated to CRM₁₉₇, wherein the O-polysaccharide includes Formula O18A, wherein n is at least 30, and the core saccharide. In another embodiment, the composition further includes an O-polysaccharide conjugated to CRM₁₉₇, wherein the O-polysaccharide includes Formula O75, wherein n is at least 30, and the core saccharide. In another embodiment, the composition further includes an O-polysaccharide conjugated to CRM₁₉₇, wherein the O-polysaccharide includes Formula O4, wherein n is at least 30, and the core saccharide. In another embodiment, the composition further includes an O-polysaccharide conjugated to CRM₁₉₇, wherein the O-polysaccharide includes Formula O16, wherein n is at least 30, and the core saccharide. In another embodiment, the composition further includes an O-polysaccharide conjugated to CRM₁₉₇, wherein the O-polysaccharide includes Formula O13, wherein n is at least 30, and the core saccharide. In another embodiment, the composition further includes an O-polysaccharide conjugated to CRM₁₉₇, wherein the O-polysaccharide includes Formula O7, wherein n is at least 30, and the core saccharide.

In another embodiment, the composition further includes an O-polysaccharide conjugated to CRM₁₉₇, wherein the O-polysaccharide includes Formula O8, wherein n is at least 30, and the core saccharide. In another embodiment, the O-polysaccharide includes Formula O8, wherein n is 1-20, preferably 2-5, more preferably 3. In another embodiment, the composition further includes an O-polysaccharide conjugated to CRM₁₉₇, wherein the O-polysaccharide includes Formula O9, wherein n is at least 30, and the core saccharide. In another embodiment, the O-polysaccharide includes Formula O9, wherein n is 1-20, preferably 4-8, more preferably 5. In another embodiment, the O-polysaccharide includes Formula O9a, wherein n is 1-20, preferably 4-8, more preferably 5.

In some embodiments, the O-polysaccharide is selected from any one of Formula O20ab, Formula O20ac, Formula O52, Formula O97, and Formula O101, wherein n is 1-20, preferably 4-8, more preferably 5.

As described above, the composition may include a FimH mutant polypeptide, and any combination of conjugated O-polysaccharides (antigens). In one exemplary embodiment, the composition includes a polysaccharide that includes Formula O25b, a polysaccharide that includes Formula O1a, a polysaccharide that includes Formula O2, and a polysaccharide that includes Formula O6. More specifically, such as a composition that includes: (i) an O-polysaccharide conjugated to CRM₁₉₇, wherein the O-polysaccharide includes Formula O25b, wherein n is at least 30, and the core saccharide; (ii) an O-polysaccharide conjugated to CRM₁₉₇, wherein the O-polysaccharide includes Formula O1a, wherein n is at least 30, and the core saccharide; (iii) an O-polysaccharide conjugated to CRM₁₉₇, wherein the O-polysaccharide includes Formula O2, wherein n is at least 30, and the core saccharide; and (iv) an O-polysaccharide conjugated to CRM₁₉₇, wherein the O-polysaccharide includes Formula O6, wherein n is at least 30, and the core saccharide.

In one embodiment, the composition includes a FimH mutant polypeptide, and at least one O-polysaccharide derived from any E. coli serotype, wherein the serotype is not O25a. For example, in one embodiment, the composition does not include a saccharide that includes the Formula O25a. Such a composition may include, for example, an O-polysaccharide that includes Formula O25b, an O-polysaccharide that includes Formula O1a, an O-polysaccharide that includes Formula O2, and an O-polysaccharide that includes Formula O6.

In one embodiment, the composition includes a FimH mutant polypeptide, and an O-polysaccharide from 2 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 3 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 4 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 5 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 6 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 7 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 8 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 9 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 10 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 11 different E. coli serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 12 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 13 different serotypes, wherein each O-polysaccharide is conjugated to CRM₁₉₇ and wherein the O-polysaccharide includes the O-antigen and core saccharide. In one embodiment, the composition includes a FimH mutant polypeptide, and an O-polysaccharide from 14 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 15 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 16 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 17 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 18 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 19 different serotypes, wherein each O-polysaccharide is conjugated to CRM₁₉₇, and wherein the O-polysaccharide includes the O-antigen and core saccharide. In one embodiment, the composition includes a FimH mutant polypeptide, and an O-polysaccharide from 20 different serotypes, wherein each O-polysaccharide is conjugated to CRM₁₉₇, and wherein the O-polysaccharide includes the O-antigen and core saccharide.

In one aspect, the invention provides a composition that comprises a FimH mutant polypeptide, and a conjugate including a saccharide covalently bound to a carrier protein, wherein the saccharide includes Formula O25b, wherein n is 15±2. In one aspect, the invention relates to a composition that includes a FimH mutant polypeptide, and a conjugate including a saccharide covalently bound to a carrier protein, wherein the saccharide includes Formula O25b, wherein n is 17±2. In one aspect, the invention relates to a composition that includes a FimH mutant polypeptide, and a conjugate including a saccharide covalently bound a carrier protein, wherein the saccharide includes Formula O25b, wherein n is 55±2. In another aspect, the invention relates to a composition that includes a FimH mutant polypeptide, and a conjugate including a saccharide covalently bound a carrier protein, wherein the saccharide includes Formula O25b, wherein n is 51±2. In another aspect, the invention relates to a composition that includes a FimH mutant polypeptide, and a conjugate including a saccharide covalently bound a carrier protein, wherein the saccharide includes Formula O25b, wherein n is an integer greater than 30, preferably n is an integer from 31 to 100. In one embodiment, the saccharide further includes the E. coli R1 core saccharide moiety. In another embodiment, the saccharide further includes the E. coli K12 core saccharide moiety. In another embodiment, the saccharide further includes the KDO moiety. Preferably, the carrier protein is CRM₁₉₇. In one embodiment, the conjugate is prepared by single end linked conjugation. In one embodiment, the conjugate is prepared by reductive amination chemistry, preferably in DMSO buffer. In one embodiment, the saccharide is conjugated to the carrier protein through a (2-((2-oxoethyl)thio)ethyl) carbamate (eTEC) spacer. Preferably, the composition further includes a pharmaceutically acceptable diluent.

In one aspect, the immunogenic composition elicits IgG antibodies in humans, said antibodies being capable of binding an E. coli serotype O25b polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the invention can be conducted and compared for their response to serotype O25b to assess the potential increase of responders. In one embodiment, the immunogenic composition elicits IgG antibodies in humans, said antibodies being capable of killing E. coli serotype O25b as determined by in vitro opsonophagocytic assay. In one embodiment, the immunogenic composition elicits functional antibodies in humans, said antibodies being capable of killing E. coli serotype O25b as determined by in vitro opsonophagocytic assay. In one embodiment, the immunogenic composition of the invention increases the proportion of responders against E. coli serotype O25b (i.e., individual with a serum having a titer of at least 1:8 as determined by in vitro OPA) as compared to the pre-immunized population. In one embodiment, the immunogenic composition elicits a titer of at least 1:8 against E. coli serotype O25b in at least 50% of the subjects as determined by in vitro opsonophagocytic killing assay. In one embodiment, the immunogenic composition of the invention elicits a titer of at least 1:8 against E. coli serotype O25b in at least 60%, 70%, 80%, or at least 90% of the subjects as determined by in vitro opsonophagocytic killing assay. In one embodiment, the immunogenic composition of the invention significantly increases the proportion of responders against E. coli serotypes O25b (i.e., individual with a serum having a titer of at least 1:8 as determined by in vitro OPA) as compared to the pre-immunized population. In one embodiment, the immunogenic composition of the invention significantly increases the OPA titers of human subjects against E. coli serotype O25b as compared to the pre-immunized population.

In one aspect, the invention relates to a composition that includes a FimH mutant polypeptide, and a conjugate including a saccharide covalently bound to a carrier protein, wherein the saccharide includes Formula O1a, wherein n is an integer greater than 30, preferably n is an integer from 31 to 100. In another aspect, the invention relates to a composition that includes a FimH mutant polypeptide, and a conjugate including a saccharide covalently bound a carrier protein, wherein the saccharide includes Formula O1a, wherein n is 39±2. In another aspect, the invention relates to a composition that includes a FimH polypeptide, and a conjugate including a saccharide covalently bound a carrier protein, wherein the saccharide includes Formula O1a, wherein n is 13±2. In one embodiment, the saccharide further includes the E. coli R1 core saccharide moiety. In one embodiment, the saccharide further includes the KDO moiety. Preferably, the carrier protein is CRM₁₉₇. In one embodiment, the conjugate is prepared by single end linked conjugation. In one embodiment, the conjugate is prepared by reductive amination chemistry, preferably in DMSO buffer. In one embodiment, the saccharide is conjugated to the carrier protein through a (2-((2-oxoethyl)thio)ethyl) carbamate (eTEC) spacer. Preferably, the composition further includes a pharmaceutically acceptable diluent.

In one embodiment, the immunogenic composition elicits IgG antibodies in humans, said antibodies being capable of binding an E. coli serotype O1a polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the invention can be conducted and compared for their response to serotype O1a to assess the potential increase of responders. In one embodiment, the immunogenic composition elicits IgG antibodies in humans, said antibodies being capable of killing E. coli serotype O1a as determined by in vitro opsonophagocytic assay. In one embodiment, the immunogenic composition elicits functional antibodies in humans, said antibodies being capable of killing E. coli serotype O1a as determined by in vitro opsonophagocytic assay. In one embodiment, the immunogenic composition of the invention increases the proportion of responders against E. coli serotype O1a (i.e., individual with a serum having a titer of at least 1:8 as determined by in vitro OPA) as compared to the pre-immunized population. In one embodiment, the immunogenic composition elicits a titer of at least 1:8 against E. coli serotype O1a in at least 50% of the subjects as determined by in vitro opsonophagocytic killing assay. In one embodiment, the immunogenic composition of the invention elicits a titer of at least 1:8 against E. coli serotype O1a in at least 60%, 70%, 80%, or at least 90% of the subjects as determined by in vitro opsonophagocytic killing assay. In one embodiment, the immunogenic composition of the invention significantly increases the proportion of responders against E. coli serotypes O1a (i.e., individual with a serum having a titer of at least 1:8 as determined by in vitro OPA) as compared to the pre-immunized population. In one embodiment, the immunogenic composition of the invention significantly increases the OPA titers of human subjects against E. coli serotype O1a as compared to the pre-immunized population.

In one aspect, the invention relates to a composition that includes a FimH mutant polypeptide, and a conjugate including a saccharide covalently bound to a carrier protein, wherein the saccharide includes Formula O2, wherein n is an integer greater than 30, preferably n is an integer from 31 to 100. In another aspect, the invention relates to a composition that includes a FimH mutant polypeptide, and a conjugate including a saccharide covalently bound to a carrier protein, wherein the saccharide includes Formula O2, wherein n is 43±2. In another aspect, the invention relates to a composition that includes a FimH mutant polypeptide, and a conjugate including a saccharide covalently bound to a carrier protein, wherein the saccharide includes Formula O2, wherein n is 47±2. In another aspect, the invention relates to a composition that includes FimH mutant polypeptide, and a conjugate including a saccharide covalently bound a carrier protein, wherein the saccharide includes Formula O2, wherein n is 17±2. In another aspect, the invention relates to a composition that includes FimH mutant polypeptide, and a conjugate including a saccharide covalently bound to a carrier protein, wherein the saccharide includes Formula O2, wherein n is 18±2. In one embodiment, the saccharide further includes the E. coli R1 core saccharide moiety. In another embodiment, the saccharide further includes the E. coli R4 core saccharide moiety. In another embodiment, the saccharide further includes the KDO moiety. Preferably, the carrier protein is CRM₁₉₇. In one embodiment, the conjugate is prepared by single end linked conjugation. In one embodiment, the conjugate is prepared by reductive amination chemistry, preferably in DMSO buffer. In one embodiment, the saccharide is conjugated to the carrier protein through a (2-((2-oxoethyl)thio)ethyl)carbamate (eTEC) spacer. Preferably, the composition further includes a pharmaceutically acceptable diluent.

In one embodiment, the immunogenic composition elicits IgG antibodies in humans, said antibodies being capable of binding an E. coli serotype O2 polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the invention can be conducted and compared for their response to serotype O2 to assess the potential increase of responders. In one embodiment, the immunogenic composition elicits IgG antibodies in humans, said antibodies being capable of killing E. coli serotype O2 as determined by in vitro opsonophagocytic assay. In one embodiment, the immunogenic composition elicits functional antibodies in humans, said antibodies being capable of killing E. coli serotype O2 as determined by in vitro opsonophagocytic assay. In one embodiment, the immunogenic composition of the invention increases the proportion of responders against E. coli serotype O2 (i.e., individual with a serum having a titer of at least 1:8 as determined by in vitro OPA) as compared to the pre-immunized population. In one embodiment, the immunogenic composition elicits a titer of at least 1:8 against E. coli serotype O2 in at least 50% of the subjects as determined by in vitro opsonophagocytic killing assay. In one embodiment, the immunogenic composition of the invention elicits a titer of at least 1:8 against E. coli serotype O2 in at least 60%, 70%, 80%, or at least 90% of the subjects as determined by in vitro opsonophagocytic killing assay. In one embodiment, the immunogenic composition of the invention significantly increases the proportion of responders against E. coli serotypes O2 (i.e., individual with a serum having a titer of at least 1:8 as determined by in vitro OPA) as compared to the pre-immunized population. In one embodiment, the immunogenic composition of the invention significantly increases the OPA titers of human subjects against E. coli serotype O2 as compared to the pre-immunized population.

In one aspect, the invention relates to a composition that includes a FimH mutant polypeptide, and a conjugate including a saccharide covalently bound to a carrier protein, wherein the saccharide includes Formula O6, wherein n is an integer greater than 30, preferably n is an integer from 31 to 100. In one aspect, the invention relates to a composition that includes a FimH mutant polypeptide, and a conjugate including a saccharide covalently bound to a carrier protein, wherein the saccharide includes Formula O6, wherein n is 42±2. In another aspect, the invention relates to a composition that includes a FimH mutant polypeptide, and a conjugate including a saccharide covalently bound to a carrier protein, wherein the saccharide includes Formula O6, wherein n is 50±2. In another aspect, the invention relates to a composition that includes a FimH mutant polypeptide, and a conjugate including a saccharide covalently bound to a carrier protein, wherein the saccharide includes Formula O6, wherein n is 17±2. In another aspect, the invention relates to a composition that includes a FimH mutant polypeptide, and a conjugate including a saccharide covalently bound to a carrier protein, wherein the saccharide includes Formula O6, wherein n is 18±2. In one embodiment, the saccharide further includes the E. coli R1 core saccharide moiety. In one embodiment, the saccharide further includes the KDO moiety. Preferably, the carrier protein is CRM₁₉₇. In one embodiment, the conjugate is prepared by single end linked conjugation. In one embodiment, the conjugate is prepared by reductive amination chemistry, preferably in DMSO buffer. In one embodiment, the saccharide is conjugated to the carrier protein through a (2-((2-oxoethyl)thio)ethyl) carbamate (eTEC) spacer. Preferably, the composition further includes a pharmaceutically acceptable diluent.

In one embodiment, the immunogenic composition elicits IgG antibodies in humans, said antibodies being capable of binding an E. coli serotype O6 polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the invention can be conducted and compared for their response to serotype O6 to assess the potential increase of responders. In one embodiment, the immunogenic composition elicits IgG antibodies in humans, said antibodies being capable of killing E. coli serotype O6 as determined by in vitro opsonophagocytic assay. In one embodiment, the immunogenic composition elicits functional antibodies in humans, said antibodies being capable of killing E. coli serotype O6 as determined by in vitro opsonophagocytic assay. In one embodiment, the immunogenic composition of the invention increases the proportion of responders against E. coli serotype O6 (i.e., individual with a serum having a titer of at least 1:8 as determined by in vitro OPA) as compared to the pre-immunized population. In one embodiment, the immunogenic composition elicits a titer of at least 1:8 against E. coli serotype O6 in at least 50% of the subjects as determined by in vitro opsonophagocytic killing assay. In one embodiment, the immunogenic composition of the invention elicits a titer of at least 1:8 against E. coli serotype O6 in at least 60%, 70%, 80%, or at least 90% of the subjects as determined by in vitro opsonophagocytic killing assay. In one embodiment, the immunogenic composition of the invention significantly increases the proportion of responders against E. coli serotypes O6 (i.e., individual with a serum having a titer of at least 1:8 as determined by in vitro OPA) as compared to the pre-immunized population. In one embodiment, the immunogenic composition of the invention significantly increases the OPA titers of human subjects against E. coli serotype O6 as compared to the pre-immunized population.

In one aspect, the composition includes a FimH mutant polypeptide, and a conjugate including a saccharide covalently bound to a carrier protein, wherein the saccharide includes a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187, wherein n is an integer from 1 to 100, preferably from 31 to 90. In one embodiment, the saccharide further includes the E. coli R1 core saccharide moiety. In one embodiment, the saccharide further includes the E. coli R2 core saccharide moiety. In one embodiment, the saccharide further includes the E. coli R3 core saccharide moiety. In another embodiment, the saccharide further includes the E. coli R4 core saccharide moiety. In one embodiment, the saccharide further includes the E. coli K12 core saccharide moiety. In another embodiment, the saccharide further includes the KDO moiety. Preferably, the carrier protein is CRM₁₉₇. In one embodiment, the conjugate is prepared by single end linked conjugation. In one embodiment, the conjugate is prepared by reductive amination chemistry, preferably in DMSO buffer. In one embodiment, the saccharide is conjugated to the carrier protein through a (2-((2-oxoethyl)thio)ethyl)carbamate (eTEC) spacer. Preferably, the composition further includes a pharmaceutically acceptable diluent. In one embodiment, the composition further includes at least 1, 2, 3, 4, 5, 6, 7, 8, 9, 10, 11, 12, 13, 14, 15, 16, 17, 18, 19, 20, 21, 22, 23, 24, 25, 26, 27, 28, or 29 additional conjugates to at most 30 additional conjugates, each conjugate including a saccharide covalently bound to a carrier protein, wherein the saccharide includes a structure selected from any one of said Formulas.

### A. Saccharides

### 1. Saccharides and O-Polysaccharides

In one embodiment, the saccharide is produced by expression (not necessarily overexpression) of different Wzz proteins (e.g., WzzB) to control of the size of the saccharide.

As used herein, the term “saccharide” refers to a single sugar moiety or monosaccharide unit as well as combinations of two or more single sugar moieties or monosaccharide units covalently linked to form disaccharides, oligosaccharides, and polysaccharides. The saccharide may be linear or branched.

In one embodiment, the saccharide is produced in a recombinant Gram-negative bacterium. In one embodiment, the saccharide is produced in a recombinant E. coli cell. In one embodiment, the saccharide is produced in a recombinant Salmonella cell. Exemplary bacteria include E. coli O25K5H1, E. coli BD559, E. coli GAR2831, E. coli GAR865, E. coli GAR868, E. coli GAR869, E. coli GAR872, E. coli GAR878, E. coli GAR896, E. coli GAR1902, E. coli O25a ETC NR-5, E. coli O157:H7:K-, Salmonella enterica serovar Typhimurium strain LT2, E. coli GAR2401, Salmonella enterica serotype Enteritidis CVD 1943, Salmonella enterica serotype Typhimurium CVD 1925, Salmonella enterica serotype Paratyphi A CVD 1902, and Shigella flexneri CVD 1208S. In one embodiment, the bacterium is not E. coli GAR2401. This genetic approach towards saccharide production allows for efficient production of O-polysaccharides and O-antigen molecules as vaccine components.

The term “wzz protein,” as used herein, refers to a chain length determinant polypeptide, such as, for example, wzzB, wzz, wzz_{SF}, wzz_{ST}, fepE, wzz_{fepE}, wzzI and wzz2. The GenBank accession numbers for the exemplary wzz gene sequences are AF011910 for E4991/76, AF011911 for F186, AF011912 for M70/1-1, AF011913 for 79/311, AF011914 for Bi7509-41, AF011915 for C664-1992, AF011916 for C258-94, AF011917 for C722-89, and AF011919 for EDL933. The GenBank accession numbers for the G7 and Bi316-41 wzz genes sequences are U39305 and U39306, respectively. Further GenBank accession numbers for exemplary wzz gene sequences are NP_459581 for Salmonella enterica subsp. Enterica serovar Typhimurium str. LT2 FepE; AIG66859 for E. coli O157:H7 Strain EDL933 FepE; NP_461024 for Salmonella enterica subsp. Enterica serovar Typhimurium str. LT2 WzzB. NP_416531 for E. coli K-12 substr. MG1655 WzzB, NP_415119 for E. coli K-12 substr. MG1655 FepE. In preferred aspects, the wzz family protein is any one of wzzB, wzz, WZZ_{SF}, WZZ_{ST}, fepE, WZZ_{fepE}, wzz1 and wzz2, most preferably wzzB, more preferably fepE.

Exemplary wzzB sequences include sequences set forth in SEQ ID Nos: 112-116. Exemplary FepE sequences include sequences set forth in SEQ ID Nos: 117-121.

In some aspects, a modified saccharide (modified as compared to the corresponding wild-type saccharide) may be produced by expressing (not necessarily overexpressing) a wzz family protein (e.g., fepE) from a Gram-negative bacterium in a Gram-negative bacterium and/or by switching off (i.e., repressing, deleting, removing) a second wzz gene (e.g., wzzB) to generate high molecular weight saccharides, such as lipopolysaccharides, containing intermediate or long O-antigen chains, which have an increased number of repeating units as compared to the corresponding wild-type O-polysaccharide. For example, the modified saccharides may be produced by expressing (not necessarily overexpressing) wzz2 and switching off wzzI. Or, in the alternative, the modified saccharides may be produced by expressing (not necessarily overexpressing) wzz/fepE and switching off wzzB. In another embodiment, the modified saccharides may be produced by expressing (not necessarily overexpressing) wzzB but switching off wzz/fepE. In another embodiment, the modified saccharides may be produced by expressing fepE. Preferably, the wzz family protein is derived from a strain that is heterologous to the host cell. Methods of determining the length of saccharides are known in the art. Such methods include, but are not limited to, nuclear magnetic resonance, mass spectroscopy, and size exclusion chromatography. Methods for producing the high molecular weight saccharides described herein, such as lipopolysaccharides, containing intermediate or long O-antigen chains, are described in PCT Intl. Publication No. WO2020/039359 and corresponding US Publication No. US2020/0061177, which are each incorporated herein by reference in their entireties.

In some embodiments, the saccharide is produced by expressing a wzz family protein having an amino acid sequence that is at least 30%, 50%, 70%, 75%, 80%, 85%, 90%, 95%, 98%, 99% or 100% sequence identity to any one of SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, and SEQ ID NO: 121. In one embodiment, the wzz family protein includes a sequence selected from any one of SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, and SEQ ID NO: 121. Preferably, the wzz family protein has at least 30%, 50%, 70%, 75%, 80%, 85%, 90%, 95%, 98%, 99% or 100% sequence identity to SEQ ID NO: 112, SEQ ID NO: 113, SEQ ID NO: 114, SEQ ID NO: 115, SEQ ID NO: 116. In some embodiments, the saccharide is produced by expressing a protein having an amino acid sequence that is at least 30%, 50%, 70%, 75%, 80%, 85%, 90%, 95%, 98%, 99% or 100% sequence identity to an fepE protein.

In one aspect, the invention relates to saccharides produced by expressing a wzz family protein, preferably fepE, in a Gram-negative bacterium to generate high molecular weight saccharides containing intermediate or long O-antigen chains, which have an increase of at least 1, 2, 3, 4, or 5 repeating units, as compared to the corresponding wild-type O-polysaccharide. In one aspect, the invention relates to saccharides produced by a Gram-negative bacterium in culture that expresses (not necessarily overexpresses) a wzz family protein (e.g., wzzB) from a Gram-negative bacterium to generate high molecular weight saccharides containing intermediate or long O-antigen chains, which have an increase of at least 1, 2, 3, 4, or 5 repeating units, as compared to the corresponding wild-type O-antigen. See description of O-polysaccharides and O-antigens below for additional exemplary saccharides having increased number of repeat units, as compared to the corresponding wild-type saccharides. A desired chain length is the one which produces improved or maximal immunogenicity in the context of a given vaccine construct.

In another embodiment, the saccharide includes any one Formula selected from Table A, wherein the number of repeat units n in the saccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide. Methods of determining the length of saccharides are known in the art. Such methods include nuclear magnetic resonance, mass spectroscopy, and size exclusion chromatography.

In a preferred embodiment, the invention relates to a saccharide produced in a recombinant E. coli host cell, wherein the gene for an endogenous wzz O-antigen length regulator (e.g., wzzB) is deleted and is replaced by a (second) wzz gene from a Gram-negative bacterium heterologous to the recombinant E. coli host cell (e.g., Salmonella fepE) to generate high molecular weight saccharides, such as lipopolysaccharides, containing intermediate or long O-antigen chains. In some embodiments, the recombinant E. coli host cell includes a wzz gene from Salmonella, preferably from Salmonella enterica.

In one embodiment, the host cell includes the heterologous gene for a wzz family protein as a stably maintained plasmid vector. In another embodiment, the host cell includes the heterologous gene for a wzz family protein as an integrated gene in the chromosomal DNA of the host cell. Methods of stably expressing a plasmid vector in an E. coli host cell and methods of integrating a heterologous gene into the chromosome of an E. coli host cell are known in the art. In one embodiment, the host cell includes the heterologous genes for an O-antigen as a stably maintained plasmid vector. In another embodiment, the host cell includes the heterologous genes for an O-antigen as an integrated gene in the chromosomal DNA of the host cell. Methods of stably expressing a plasmid vector in an E. coli host cell and a Salmonella host cell are known in the art. Methods of integrating a heterologous gene into the chromosome of an E. coli host cell and a Salmonella host cell are known in the art.

In one aspect, the recombinant host cell is cultured in a medium that comprises a carbon source. Carbon sources for culturing E. coli are known in the art. Exemplary carbon sources include sugar alcohols, polyols, aldol sugars or keto sugars including but not limited to arabinose, cellobiose, fructose, glucose, glycerol, inositol, lactose, maltose, mannitol, mannose, rhamnose, raffinose, sorbitol, sorbose, sucrose, trehalose, pyruvate, succinate and methylamine. In a preferred embodiment, the medium includes glucose. In some embodiments, the medium includes a polyol or aldol sugar, for example, mannitol, inositol, sorbose, glycerol, sorbitol, lactose and arabinose as the carbon source. All of the carbon sources may be added to the medium before the start of culturing, or it may be added step by step or continuously during culturing.

An exemplary culture medium for the recombinant host cell includes an element selected from any one of KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O. Preferably, the medium includes KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O.

The medium used herein may be solid or liquid, synthetic (i.e. man-made) or natural, and may include sufficient nutrients for the cultivation of the recombinant host cell. Preferably, the medium is a liquid medium.

In some embodiments, the medium may further include suitable inorganic salts. In some embodiments, the medium may further include trace nutrients. In some embodiments, the medium may further include growth factors. In some embodiments, the medium may further include an additional carbon source. In some embodiments, the medium may further include suitable inorganic salts, trace nutrients, growth factors, and a supplementary carbon source. Inorganic salts, trace nutrients, growth factors, and supplementary carbon sources suitable for culturing E. coli are known in the art.

In some embodiments, the medium may include additional components as appropriate, such as peptone, N-Z Amine, enzymatic soy hydrosylate, additional yeast extract, malt extract, supplemental carbon sources and various vitamins. In some embodiments, the medium does not include such additional components, such as peptone, N-Z Amine, enzymatic soy hydrosylate, additional yeast extract, malt extract, supplemental carbon sources and various vitamins.

Illustrative examples of suitable supplemental carbon sources include, but are not limited to other carbohydrates, such as glucose, fructose, mannitol, starch or starch hydrolysate, cellulose hydrolysate and molasses; organic acids, such as acetic acid, propionic acid, lactic acid, formic acid, malic acid, citric acid, and fumaric acid; and alcohols, such as glycerol, inositol, mannitol and sorbitol.

In some embodiments, the medium further includes a nitrogen source. Nitrogen sources suitable for culturing E. coli are known in the art. Illustrative examples of suitable nitrogen sources include, but are not limited to ammonia, including ammonia gas and aqueous ammonia; ammonium salts of inorganic or organic acids, such as ammonium chloride, ammonium nitrate, ammonium phosphate, ammonium sulfate and ammonium acetate; urea; nitrate or nitrite salts, and other nitrogen-containing materials, including amino acids as either pure or crude preparations, meat extract, peptone, fish meal, fish hydrolysate, corn steep liquor, casein hydrolysate, soybean cake hydrolysate, yeast extract, dried yeast, ethanol-yeast distillate, soybean flour, cottonseed meal, and the like.

In some embodiments, the medium includes an inorganic salt. Illustrative examples of suitable inorganic salts include, but are not limited to salts of potassium, calcium, sodium, magnesium, manganese, iron, cobalt, zinc, copper, molybdenum, tungsten and other trace elements, and phosphoric acid.

In some embodiments, the medium includes appropriate growth factors. Illustrative examples of appropriate trace nutrients, growth factors, and the like include, but are not limited to coenzyme A, pantothenic acid, pyridoxine-HCl, biotin, thiamine, riboflavin, flavine mononucleotide, flavine adenine dinucleotide, DL-6,8-thioctic acid, folic acid, Vitamin B12, other vitamins, amino acids such as cysteine and hydroxyproline, bases such as adenine, uracil, guanine, thymine and cytosine, sodium thiosulfate, p- or r-aminobenzoic acid, niacinamide, nitriloacetate, and the like, either as pure or partially purified chemical compounds or as present in natural materials. The amounts may be determined empirically by one skilled in the art according to methods and techniques known in the art.

In another embodiment, the modified saccharide (as compared to the corresponding wild-type saccharide) described herein is synthetically produced, for example, in vitro. Synthetic production or synthesis of the saccharides may facilitate the avoidance of cost- and time-intensive production processes. In one embodiment, the saccharide is synthetically synthesized, such as, for example, by using sequential glycosylation strategy or a combination of sequential glycosylations and [3+2] block synthetic strategy from suitably protected monosaccharide intermediates. For example, thioglycosides and glycosyl trichloroacetimidate derivatives may be used as glycosyl donors in the glycosylations. In one embodiment, a saccharide that is synthetically synthesized in vitro has the identical structure to a saccharide produced by recombinant means, such as by manipulation of a wzz family protein described above.

The saccharide produced (by recombinant or synthetic means) includes a structure derived from any E. coli serotype including, for example, any one of the following E. coli serotypes: O1 (e.g., O1A, O1B, and O1C), O2, O3, O4 (e.g., O4:K52 and O4:K6), O5 (e.g., O5ab and O5ac (strain 180/C3)), O6 (e.g., O6:K2; K13; K15 and O6:K54), O7, O8, O9, O10, O11, O12, O13, O14, O15, O16, O17, O18 (e.g., O18A, O18ac, O18A1, O18B, and O18B1), O19, O20, O21, O22, O23 (e.g., O23A), O24, O25 (e.g., O25a and O25b), O26, O27, O28, O29, O30, O32, O33, O34, O35, O36, O37, O38, O39, O40, O41, O42, O43, O44, O45 (e.g., O45 and O45rel), O46, O48, O49, O50, O51, O52, O53, O54, O55, O56, O57, O58, O59, O60, O61, O62, 62D1, O63, O64, O65, O66, O68, O69, O70, O71, O73 (e.g., O73 (strain 73-1)), O74, O75, O76, O77, O78, O79, O80, O81, O82, O83, O84, O85, O86, O87, O88, O89, O90, O91, O92, O93, O95, O96, O97, O98, O99, O100, O101, O102, O103, O104, O105, O106, O107, O108, O109, O110, O111, O112, O113, O114, O115, O116, O117, O118, O119, O120, O121, O123, O124, O125, O126, O127, O128, O129, O130, O131, O132, O133, O134, O135, O136, O137, O138, O139, O140, O141, O142, O143, O144, O145, O146, O147, O148, O149, O150, O151, O152, O153, O154, O155, O156, O157, O158, O159, O160, O161, O162, O163, O164, O165, O166, O167, O168, O169, O170, O171, O172, O173, O174, O175, O176, O177, O178, O179, O180, O181, O182, O183, O184, O185, O186, and O187.

The individual polysaccharides are typically purified (enriched with respect to the amount of polysaccharide-protein conjugate) through methods known in the art, such as, for example, dialysis, concentration operations, diafiltration operations, tangential flow filtration, precipitation, elution, centrifugation, precipitation, ultra-filtration, depth filtration, and/or column chromatography (ion exchange chromatography, multimodal ion exchange chromatography, DEAE, and hydrophobic interaction chromatography). Preferably, the polysaccharides are purified through a method that includes tangential flow filtration.

Purified polysaccharides may be activated (e.g., chemically activated) to make them capable of reacting (e.g., either directly to the carrier protein or via a linker such as an eTEC spacer) and then incorporated into glycoconjugates of the invention, as further described herein.

In one preferred embodiment, the saccharide of the invention is derived from an E. coli serotype, wherein the serotype is O25a. In another preferred embodiment, the serotype is O25b. In another preferred embodiment, the serotype is O1A. In another preferred embodiment, the serotype is O2. In another preferred embodiment, the serotype is O6. In another preferred embodiment, the serotype is O17. In another preferred embodiment, the serotype is O15. In another preferred embodiment, the serotype is O18A. In another preferred embodiment, the serotype is O75. In another preferred embodiment, the serotype is O4. In another preferred embodiment, the serotype is O16. In another preferred embodiment, the serotype is O13. In another preferred embodiment, the serotype is O7. In another preferred embodiment, the serotype is O8. In another preferred embodiment, the serotype is O9.

As used herein, reference to any of the serotypes listed above, refers to a serotype that encompasses a repeating unit structure (O-unit, as described below) known in the art and is unique to the corresponding serotype. For example, the term “O25a” serotype (also known in the art as serotype “O25”) refers to a serotype that encompasses Formula O25 shown in Table A. As another example, the term “O25b” serotype refers to a serotype that encompasses Formula O25b shown in Table A.

As used herein, the serotypes are referred generically herein unless specified otherwise such that, for example, the term Formula “O18” refers generically to encompass Formula O18A, Formula O18ac, Formula 18A1, Formula O18B, and Formula O18B1. As used herein, the term “O1” refers generically to encompass the species of Formula that include the generic term “O1” in the Formula name according to Table A, such as any one of Formula O1A, Formula O1A1, Formula O1B, and Formula O1C, each of which is shown in Table A. Accordingly, an “O1 serotype” refers generically to a serotype that encompasses any one of Formula O1A, Formula O1A1, Formula O1B, and Formula O1C.

As used herein, the term “O6” refers generically to species of Formula that include the generic term “O6” in the Formula name according to Table A, such as any one of Formula O6:K2; K13; K15; and O6:K54, each of which is shown in Table A. Accordingly, an “O6 serotype” refers generically to a serotype that encompasses any one of Formula O6:K2; K13; K15; and O6:K54. Other examples of terms that refer generically to species of a Formula that include the generic term in the Formula name according to Table A include: “O4”, “O5”, “O18”, and “O45”. As used herein, the term “O2” refers to Formula O2 shown in Table A. The term “O2 O-antigen” refers to a saccharide that encompasses Formula O2 shown in Table A.

As used herein, reference to an O-antigen from a serotype listed above refers to a saccharide that encompasses the formula labeled with the corresponding serotype name. For example, the term “O25B O-antigen” refers to a saccharide that encompasses Formula O25B shown in Table A.

As another example, the term “O1 O-antigen” generically refers to a saccharide that encompasses a Formula including the term “O1,” such as the Formula O1A, Formula O1A1, Formula O1B, and Formula O1C, each of which are shown in Table A.

As another example, the term “O6 O-antigen” generically refers to a saccharide that encompasses a Formula including the term “O6,” such as Formula O6:K2; Formula O6:K13; Formula O6:K15 and Formula O6:K54, each of which are shown in Table A.

As used herein, the term “O-polysaccharide” refers to any structure that includes an O-antigen, provided that the structure does not include a whole cell or Lipid A. For example, in one embodiment, the O-polysaccharide includes a lipopolysaccharide wherein the Lipid A is not bound. The step of removing Lipid A is known in the art and includes, as an example, heat treatment with addition of an acid. An exemplary process includes treatment with 1% acetic acid at 100° C. for 90 minutes. This process is combined with a process of isolating Lipid A as removed. An exemplary process for isolating Lipid A includes ultracentrifugation.

In one embodiment, the O-polysaccharide refers to a structure that consists of the O-antigen, in which case, the O-polysaccharide is synonymous with the term O-antigen. In one preferred embodiment, the O-polysaccharide refers to a structure that includes repeating units of the O-antigen, without the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety. In another preferred embodiment, the O-polysaccharide refers to a structure that includes an O-antigen and a core saccharide. In another embodiment, the O-polysaccharide refers to a structure that includes an O-antigen, a core saccharide, and a KDO moiety.

Methods of purifying an O-polysaccharide, which includes the core oligosaccharide, from LPS are known in the art. For example, after purification of LPS, purified LPS may be hydrolyzed by heating in 1% (v/v) acetic acid for 90 minutes at 100 degrees Celsius, followed by ultracentrifugation at 142,000×g for 5 hours at 4 degrees Celsius. The supernatant containing the O-polysaccharide is freeze-dried and stored at 4 degrees Celsius. In certain embodiments, deletion of capsule synthesis genes to enable simple purification of O-polysaccharide is described.

The O-polysaccharide can be isolated by methods including, but not limited to mild acid hydrolysis to remove lipid A from LPS. Other embodiments may include use of hydrazine as an agent for O-polysaccharide preparation. Preparation of LPS can be accomplished by known methods in the art.

In certain embodiments, the O-polysaccharides purified from wild-type, modified, or attenuated Gram-negative bacterial strains that express (not necessarily overexpress) a Wzz protein (e.g., wzzB) are provided for use in conjugate vaccines. In preferred embodiments, the O-polysaccharide chain is purified from the Gram-negative bacterial strain expressing (not necessarily overexpressing) wzz protein for use as a vaccine antigen either as a conjugate or complexed vaccine.

In one embodiment, the O-polysaccharide has a molecular weight that is increased by about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold or more, as compared to the corresponding wild-type O-polysaccharide. In a preferred embodiment, the O-polysaccharide has a molecular weight that is increased by at least 1-fold and at most 5-fold, as compared to the corresponding wild-type O-polysaccharide. In another embodiment, the O-polysaccharide has a molecular weight that is increased by at least 2-fold and at most 4-fold, as compared to the corresponding wild-type O-polysaccharide. An increase in molecular weight of the O-polysaccharide, as compared to the corresponding wild-type O-polysaccharide, is preferably associated with an increase in number of O-antigen repeat units. In one embodiment, the increase in molecular weight of the O-polysaccharide is due to the wzz family protein.

In one embodiment, the O-polysaccharide has a molecular weight that is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 kDa or more, as compared to the corresponding wild-type O-polysaccharide. In one embodiment, the O-polysaccharide of the invention has a molecular weight that is increased by at least 1 and at most 200 kDa, as compared to the corresponding wild-type O-polysaccharide. In one embodiment, the molecular weight is increased by at least 5 and at most 200 kDa. In one embodiment, the molecular weight is increased by at least 10 and at most 200 kDa. In one embodiment, the molecular weight is increased by at least 12 and at most 200 kDa. In one embodiment, the molecular weight is increased by at least 15 and at most 200 kDa. In one embodiment, the molecular weight is increased by at least 18 and at most 200 kDa. In one embodiment, the molecular weight is increased by at least 20 and at most 200 kDa. In one embodiment, the molecular weight is increased by at least 21 and at most 200 kDa. In one embodiment, the molecular weight is increased by at least 22 and at most 200 kDa. In one embodiment, the molecular weight is increased by at least 30 and at most 200 kDa. In one embodiment, the molecular weight is increased by at least 1 and at most 100 kDa. In one embodiment, the molecular weight is increased by at least 5 and at most 100 kDa. In one embodiment, the molecular weight is increased by at least 10 and at most 100 kDa. In one embodiment, the molecular weight is increased by at least 12 and at most 100 kDa. In one embodiment, the molecular weight is increased by at least 15 and at most 100 kDa. In one embodiment, the molecular weight is increased by at least 20 and at most 100 kDa. In one embodiment, the molecular weight is increased by at least 1 and at most 75 kDa. In one embodiment, the molecular weight is increased by at least 5 and at most 75 kDa. In one embodiment, the molecular weight is increased by at least 10 and at most 75 kDa. In one embodiment, the molecular weight is increased by at least 12 and at most 75 kDa. In one embodiment, the molecular weight is increased by at least 15 and at most 75 kDa. In one embodiment, the molecular weight is increased by at least 18 and at most 75 kDa. In one embodiment, the molecular weight is increased by at least 20 and at most 75 kDa. In one embodiment, the molecular weight is increased by at least 30 and at most 75 kDa. In one embodiment, the molecular weight is increased by at least 10 and at most 90 kDa. In one embodiment, the molecular weight is increased by at least 12 and at most 85 kDa. In one embodiment, the molecular weight is increased by at least 10 and at most 75 kDa. In one embodiment, the molecular weight is increased by at least 10 and at most 70 kDa. In one embodiment, the molecular weight is increased by at least 10 and at most 60 kDa. In one embodiment, the molecular weight is increased by at least 10 and at most 50 kDa. In one embodiment, the molecular weight is increased by at least 10 and at most 49 kDa. In one embodiment, the molecular weight is increased by at least 10 and at most 48 kDa. In one embodiment, the molecular weight is increased by at least 10 and at most 47 kDa. In one embodiment, the molecular weight is increased by at least 10 and at most 46 kDa. In one embodiment, the molecular weight is increased by at least 20 and at most 45 kDa. In one embodiment, the molecular weight is increased by at least 20 and at most 44 kDa. In one embodiment, the molecular weight is increased by at least 20 and at most 43 kDa. In one embodiment, the molecular weight is increased by at least 20 and at most 42 kDa. In one embodiment, the molecular weight is increased by at least 20 and at most 41 kDa. Such an increase in molecular weight of the O-polysaccharide, as compared to the corresponding wild-type O-polysaccharide, is preferably associated with an increase in number of O-antigen repeat units. In one embodiment, the increase in molecular weight of the O-polysaccharide is due to the wzz family protein.

In another embodiment, the O-polysaccharide includes any one Formula selected from Table A, wherein the number of repeat units n in the O-polysaccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide.

### 2. O-Antigen

The O-antigen is part of the lipopolysaccharide (LPS) in the outer membrane of Gram-negative bacteria. The O-antigen is on the cell surface and is a variable cell constituent. The variability of the O-antigen provides a basis for serotyping of Gram-negative bacteria. The current E. coli serotyping scheme includes O-polysaccharides 1 to 181.

The O-antigen includes oligosaccharide repeating units (O-units), the wild type structure of which usually contains two to eight residues from a broad range of sugars. The O-units of exemplary E. coli O-antigens are described Table A and in PCT Intl. Publication No. WO2021/084429, published May 6, 2021, which is incorporated herein by reference in its entirety. In some embodiments, the present disclosure includes a composition comprising at least one FimH mutant polypeptide and at least one of the O-antigens as described Table A and in PCT Intl. Publication No. WO2021/084429, published May 6, 2021, which is incorporated herein by reference in its entirety.

In one embodiment, the saccharide of the invention may be one oligosaccharide unit. In one embodiment, the saccharide of the invention is one repeating oligosaccharide unit of the relevant serotype. In such embodiments, the saccharide may include a structure selected from any one of Formula O1a, Formula O2, Formula O6, Formula O8, Formula O9a, Formula O9, Formula O20ab, Formula O20ac, Formula O25b, Formula O52, Formula O97, and Formula O101. In a further embodiment, the saccharide may include a structure selected from any one of Formula O1a, Formula O2, Formula O6, and Formula O25b.

In one embodiment, the saccharide of the invention may be oligosaccharides. Oligosaccharides have a low number of repeat units (typically 5-15 repeat units) and are typically derived synthetically or by hydrolysis of polysaccharides. In such embodiments, the saccharide may include a structure selected from any one of Formula O1a, Formula O2, Formula O6, Formula O8, Formula O9a, Formula O9, Formula O20ab, Formula O20ac, Formula O25b, Formula O52, Formula O97, and Formula O101. In a further embodiment, the saccharide may include a structure selected from any one of Formula O1a, Formula O2, Formula O6, and Formula O25b.

Preferably, all of the saccharides of the present invention and in the immunogenic compositions of the present invention are polysaccharides. High molecular weight polysaccharides may induce certain antibody immune responses due to the epitopes present on the antigenic surface. The isolation and purification of high molecular weight polysaccharides are preferably contemplated for use in the conjugates, compositions and methods of the present invention.

In some embodiments, the number of repeat O units in each individual O-antigen polymer (and therefore the length and molecular weight of the polymer chain) depends on the wzz chain length regulator, an inner membrane protein. Different wzz proteins confer different ranges of modal lengths (4 to >100 repeat units). The term “modal length” refers to the number of repeating O-units. Gram-negative bacteria often have two different Wzz proteins that confer two distinct Oag modal chain lengths, one longer and one shorter. The expression (not necessarily the overexpression) of wzz family proteins (e.g., wzzB) in Gram-negative bacteria may allow for the manipulation of O-antigen length, to shift or to bias bacterial production of O-antigens of certain length ranges, and to enhance production of high-yield large molecular weight lipopolysaccharides. In one embodiment, a “short” modal length as used herein refers to a low number of repeat O-units, e.g., 1-20. In one embodiment, a “long” modal length as used herein refers to a number of repeat O-units greater than 20 and up to a maximum of 40. In one embodiment, a “very long” modal length as used herein refers to greater than 40 repeat O-units.

In one embodiment, the saccharide produced has an increase of at least 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, or 100 repeating units, as compared to the corresponding wild-type O-polysaccharide.

In another embodiment, the saccharide of the invention has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide. See, for example, Table 21. Methods of determining the length of saccharides are known in the art. Such methods include nuclear magnetic resonance, mass spectroscopy, and size exclusion chromatography, as described in Example 13.

Methods of determining the number of repeat units in the saccharide are also known in the art. For example, the number of repeat units (or “n” in the Formula) may be calculated by dividing the molecular weight of the polysaccharide (without the molecular weight of the core saccharide or KDO residue) by the molecular weight of the repeat unit (i.e., molecular weight of the structure in the corresponding Formula, shown for example in Table 1, which may be theoretically calculated as the sum of the molecular weight of each monosaccharide within the Formula). The molecular weight of each monosaccharide within the Formula is known in the art. The molecular weight of a repeat unit of Formula O25b, for example, is about 862 Da. The molecular weight of a repeat unit of Formula O1a, for example, is about 845 Da. The molecular weight of a repeat unit of Formula O2, for example, is about 829 Da. The molecular weight of a repeat unit of Formula O6, for example, is about 893 Da. When determining the number of repeat units in a conjugate, the carrier protein molecular weight and the protein:polysaccharide ratio is factored into the calculation. As defined herein, “n” refers to the number of repeating units (represented in brackets in Table 1) in a polysaccharide molecule. As is known in the art, in biological macromolecules, repeating structures may be interspersed with regions of imperfect repeats, such as, for example, missing branches. In addition, it is known in the art that polysaccharides isolated and purified from natural sources such as bacteria may be heterogenous in size and in branching. In such a case, n may represent an average or median value for n for the molecules in a population.

In one embodiment, the O-polysaccharide has an increase of at least one repeat unit of an O-antigen, as compared to the corresponding wild-type O-polysaccharide. The repeat units of O-antigens are shown in Table 1. In one embodiment, the O-polysaccharide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more total repeat units. Preferably, the saccharide has a total of at least 3 to at most 80 repeat units. In another embodiment, the O-polysaccharide has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide.

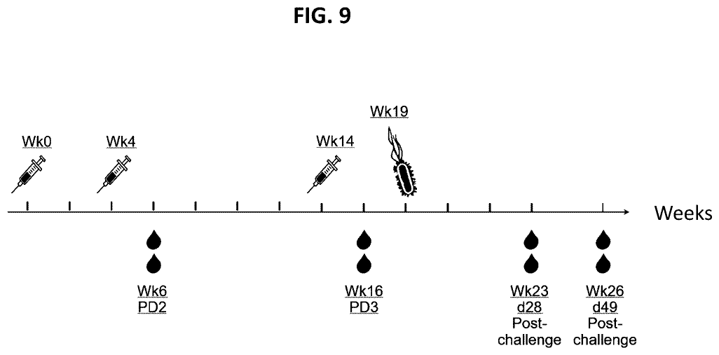

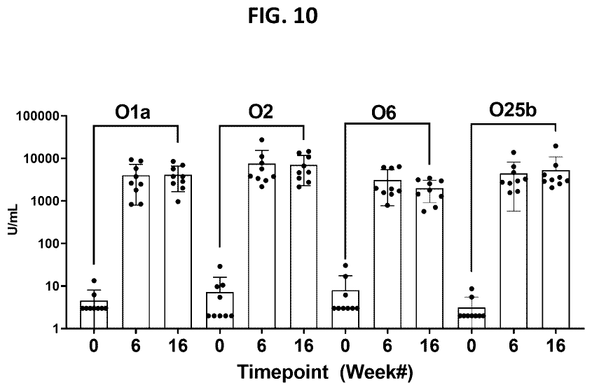

In one embodiment, the saccharide includes an O-antigen wherein n in any of the O-antigen formulas (such as, for example, the Formulas shown in Table 1 (see also FIG. 9A-9C and FIG. 10A-10B)) is an integer of at least 1, 2, 3, 4, 5, 10, 20, 21, 22, 23, 24, 25, 26, 27, 28, 29, 30, 31, 32, 33, 34, 35, 36, 37, 38, 39, 40, and at most 200, 100, 99, 98, 97, 96, 95, 94, 93, 92, 91, 90, 89, 88, 87, 86, 81, 80, 79, 78, 77, 76, 75, 74, 73, 72, 71, 70, 69, 68, 67, 66, 65, 60, 59, 58, 57, 56, 55, 54, 53, 52, 51, or 50. Any minimum value and any maximum value may be combined to define a range. Exemplary ranges include, for example, at least 1 to at most 1000; at least 10 to at most 500; and at least 20 to at most 80, preferably at most 90. In one preferred embodiment, n is at least 31 to at most 90. In a preferred embodiment, n is 40 to 90, more preferably 60 to 85.

In one embodiment, the saccharide includes an O-antigen wherein n in any one of the O-antigen Formulas is at least 1 and at most 200. In one embodiment, n in any one of the O-antigen Formulas is at least 5 and at most 200. In one embodiment, n in any one of the O-antigen Formulas is at least 10 and at most 200. In one embodiment, n in any one of the O-antigen Formulas is at least 25 and at most 200. In one embodiment, n in any one of the O-antigen Formulas is at least 50 and at most 200. In one embodiment, n in any one of the O-antigen Formulas is at least 75 and at most 200. In one embodiment, n in any one of the O-antigen Formulas is at least 100 and at most 200. In one embodiment, n in any one of the O-antigen Formulas is at least 125 and at most 200. In one embodiment, n in any one of the O-antigen Formulas is at least 150 and at most 200. In one embodiment, n in any one of the O-antigen Formulas is at least 175 and at most 200. In one embodiment, n in any one of the O-antigen Formulas is at least 1 and at most 100. In one embodiment, n in any one of the O-antigen Formulas is at least 5 and at most 100. In one embodiment, n in any one of the O-antigen Formulas is at least 10 and at most 100. In one embodiment, n in any one of the O-antigen Formulas is at least 25 and at most 100. In one embodiment, n in any one of the O-antigen Formulas is at least 50 and at most 100. In one embodiment, n in any one of the O-antigen Formulas is at least 75 and at most 100. In one embodiment, n in any one of the O-antigen Formulas is at least 1 and at most 75. In one embodiment, n in any one of the O-antigen Formulas is at least 5 and at most 75. In one embodiment, n in any one of the O-antigen Formulas is at least 10 and at most 75. In one embodiment, n in any one of the O-antigen Formulas is at least 20 and at most 75. In one embodiment, n in any one of the O-antigen Formulas is at least 25 and at most 75. In one embodiment, n in any one of the O-antigen Formulas is at least 30 and at most 75. In one embodiment, n in any one of the O-antigen Formulas is at least 40 and at most 75. In one embodiment, n in any one of the O-antigen Formulas is at least 50 and at most 75. In one embodiment, n in any one of the O-antigen Formulas is at least 30 and at most 90. In one embodiment, n in any one of the O-antigen Formulas is at least 35 and at most 85. In one embodiment, n in any one of the O-antigen Formulas is at least 35 and at most 75. In one embodiment, n in any one of the O-antigen Formulas is at least 35 and at most 70. In one embodiment, n in any one of the O-antigen Formulas is at least 35 and at most 60. In one embodiment, n in any one of the O-antigen Formulas is at least 35 and at most 50. In one embodiment, n in any one of the O-antigen Formulas is at least 35 and at most 49. In one embodiment, n in any one of the O-antigen Formulas is at least 35 and at most 48. In one embodiment, n in any one of the O-antigen Formulas is at least 35 and at most 47. In one embodiment, n in any one of the O-antigen Formulas is at least 35 and at most 46. In one embodiment, n in any one of the O-antigen Formulas is at least 36 and at most 45. In one embodiment, n in any one of the O-antigen Formulas is at least 37 and at most 44. In one embodiment, n in any one of the O-antigen Formulas is at least 38 and at most 43. In one embodiment, n in any one of the O-antigen Formulas is at least 39 and at most 42. In one embodiment, n in any one of the O-antigen Formulas is at least 39 and at most 41.

For example, in one embodiment, n in the saccharide is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, most preferably 40. In another embodiment, n is at least 35 to at most 60. For example, in one embodiment, n is any one of 35, 36, 37, 38, 39, 40, 41, 42, 43, 44, 45, 46, 47, 48, 49, 50, 51, 52, 53, 54, 55, 56, 57, 58, 59, and 60, preferably 50. In another preferred embodiment, n is at least 55 to at most 75. For example, in one embodiment, n is 55, 56, 57, 58, 59, 60, 61, 62, 63, 64, 65, 66, 67, 68, or 69, most preferably 60.

The saccharide structure may be determined by methods and tools known art, such as, for example, NMR, including 1D, 1H, and/or 13C, 2D TOCSY, DQF-COSY, NOESY, and/or HMQC.

In some embodiments, the purified polysaccharide before conjugation has a molecular weight of between 5 kDa and 400 kDa. In other such embodiments, the saccharide has a molecular weight of between 10 kDa and 400 kDa; between 5 kDa and 400 kDa; between 5 kDa and 300 kDa; between 5 kDa and 200 kDa; between 5 kDa and 150 kDa; between 10 kDa and 100 kDa; between 10 kDa and 75 kDa; between 10 kDa and 60 kDa; between 10 kDa and 40 kDa; between 10 kDa and 100 kDa; 10 kDa and 200 kDa; between 15 kDa and 150 kDa; between 12 kDa and 120 kDa; between 12 kDa and 75 kDa; between 12 kDa and 50 kDa; between 12 and 60 kDa; between 35 kDa and 75 kDa; between 40 kDa and 60 kDa; between 35 kDa and 60 kDa; between 20 kDa and 60 kDa; between 12 kDa and 20 kDa; or between 20 kDa and 50 kDa. In further embodiments, the polysaccharide has a molecular weight of between 7 kDa to 15 kDa; 8 kDa to 16 kDa; 9 kDa to 25 kDa; 10 kDa to 100; 10 kDa to 60 kDa; 10 kDa to 70 kDa; 10 kDa to 160 kDa; 15 kDa to 600 kDa; 20 kDa to 1000 kDa; 20 kDa to 600 kDa; 20 kDa to 400 kDa; 30 kDa to 1,000 Kda; 30 kDa to 60 kDa; 30 kDa to 50 kDa or 5 kDa to 60 kDa. Any whole number integer within any of the above ranges is contemplated as an embodiment of the disclosure.

As used herein, the term “molecular weight” of polysaccharide or of carrier protein-polysaccharide conjugate refers to molecular weight calculated by size exclusion chromatography (SEC) combined with multiangle laser light scattering detector (MALLS).

A polysaccharide can become slightly reduced in size during normal purification procedures. Additionally, as described herein, polysaccharide can be subjected to sizing techniques before conjugation. Mechanical or chemical sizing maybe employed. Chemical hydrolysis may be conducted using acetic acid. Mechanical sizing may be conducted using High Pressure Homogenization Shearing. The molecular weight ranges mentioned above refer to purified polysaccharides before conjugation (e.g., before activation).

TABLE A
E. coli serogroups/serotypes and O-unit moieties

| | | Moiety structure |
|---|---|---|
| Serogroup/ | | referred to |
| Serotype | Moiety Structure (O-unit) | herein as: |
| O1A, O1A1 | [→3)-α-L-Rha-(1→3)-α-L-Rha-(1→3)-β-L-Rha-(1→4)-β-D-GlcNAc- | Formula O1A |
| | (1→ \| β-D-ManNAc-(1→2) ]ₙ | |
| O1B | [→3)-α-L-Rha-(1→2)-α-L-Rha-(1→2)-α-D-Gal-(1→3)-β-D-GlcNAc- | Formula O1B |
| | (1→\|β-D-ManNAc-(1→2) ]ₙ | |
| O1C | [→3)-α-L-Rha-(1→2)-α-L-Rha-(1→3)-α-D-Gal-(1→3)-β-D-GlcNAc- | Formula O1C |
| | (1→\|β-D-ManNAc-(1→2) ]ₙ | |
| O2 | [→3)-α-L-Rha-(1→2)-α-L-Rha-(1→3)-β-L-Rha-(1→4)-β-D-GlcNAc- | Formula O2 |
| | (1→ \| α-D-ManNAc-(1→2) ]ₙ | |
| O3 | [β-L-RhaNAc(1→4)α-D-Glc-(1→4)\| \| →3)-β-D-GlcNAc-(1→3)-α-D- | Formula O3 |
| | Gal-(1→3)-β-D-GlcNAc-(1→ ]ₙ | |
| O4:K52 | [→2)-α-L-Rha-(1→6)-α-D-Glc-(1→3)-α-L-FucNAc-(1→3)-β-D- | Formula O4:K52 |
| | GlcNAc(1→ ]ₙ | |
| O4:K6 | [α-D-Glc-(1→3) \| →2)-α-L-Rha-(1→6)-α-D-Glc-(1→3)-α-L-FucNAc- | Formula O4:K6 |
| | (1→3)-β-D-GlcNAc(1→ ]ₙ | |
| O5ab | [→4)-β-D-Qui3Nac-(1→3)-β-D-Ribf-(1→4)-β-D-Gal-(1→3)-α-D- | Formula O5ab |
| | GalNAc(1→]ₙ | |
| O5ac (strain | [→2)-β-D-Qui3Nac-(1→3)-β-D-Ribf-(1→4)-β-D-Gal-(1→3)-α-D- | Formula O5ac |
| 180/C3) | GalNAc(1→ ]ₙ | (strain 180/C3) |
| O6:K2; K13; | [→4)-α-D-GalNAc-(1→3)-β-D-Man-(1→4)-β-D-Man-(1→3)-α-D- | Formula O6:K2; |
| K15 | GlcNAc-(1→ \| β-D-Glc-(1→2) ]ₙ | K13; K15 |
| O6:K54 | [→4)-α-D-GalNAc-(1→3)-β-D-Man-(1→4)-β-D-Man-(1→3)-α-D- | Formula O6:K54 |
| | GlcNAc-(1→\|β-D-GlcNAc-(1→2) ]ₙ | |
| O7 | [α-L-Rha-(1→3) \| →3)-β-D-Qui4Nac-(1→2)-α-D-Man-(1→4)-β-D- | Formula O7 |
| | Gal-(1→3)-α-D-GlcNAc-(1→ ]ₙ | |
| O10 | [→3)-α-L-Rha-(1→3)-α-L-Rha-(1→3)-α-D-Gal-(1→3)-β-D-GlcNAc- | Formula O10 |
| | (1→ \| α-D-Fuc4Nacyl-(1→2) Acyl = acetyl (60%) or l-3- | |
| | hydroxybutyryl (40%) ]ₙ | |
| O16 | [→2)-β-D-Galf-(1→6)-α-D-Glc-(1→3)-α-L-Rha2Ac-(1→3)-α-D- | Formula O16 |
| | GlcNAc-(1→ ]ₙ | |
| O17 | [α-D-Glc-(1→6) \| →6)-α-D-Man-(1→2)-α-D-Man-(1→2)-β-D-Man- | Formula O17 |
| | (1→3)-α-D-GlcNAc(1→ ]ₙ | |
| O18A, O18ac | [→2)-α-L-Rha-(1→6)-α-D-Glc-(1→4)-α-D-Gal-(1→3)-α-D-GlcNAc- | Formula O18A, |
| | (1→ \| β-D-GlcNAc-(1→3) ]ₙ | Formula O18ac |
| O18A1 | [α-D-Glc-(1→6) \| →2)-α-L-Rha-(1→6)-α-D-Glc-(1→4)-α-D-Gal- | Formula O18A1 |
| | (1→3)-α-D-GlcNAc-(1→ \| β-D-GlcNAc-(1→3) ]ₙ | |
| O18B | [→3)-α-L-Rha-(1→6)-α-D-Glc-(1→4)-α-D-Gal-(1→3)-α-D-GlcNAc- | Formula O18B |
| | (1→ \| β-D-Glc-(1→3) ]ₙ | |
| O18B1 | [α-D-Glc-(1→4) \| →3)-α-L-Rha-(1→6)-α-D-Glc-(1→4)-α-D-Gal- | Formula O18B1 |
| | (1→3)-α-D-GlcNAc-(1→ \| β-D-Glc-(1→3) ]ₙ | |
| O21 | [(β-D-Gal-(1→4) \| →3)-β-D-Gal-(1→4)-β-D-Glc-(1→3)-β-D-GalNAc- | Formula O21 |
| | (1→ \| β-D-GlcNAc-(1→2) ]ₙ | |
| O23A | [α-D-Glc-(1→6) \| →6)-α-D-Glc-(1→4)-β-D-Gal-(1→3)-α-D-GalNAc- | Formula O23A |
| | (1→3)-β-D-GlcNAc-(1→ \| β-D-GlcNAc(1→3) ]ₙ | |
| O24 | [→7)-α-Neu5Ac-(2→3)-β-D-Glc-(1→3)-β-D-GalNAc-(1→ \| α-D-Glc- | Formula O24 |
| | (1→2) ]ₙ | |
| O25/O25a | [β-D-Glc-(1→6) \| →4)-α-D-Glc-(1→3)-α-L-FucNAc-(1→3)-β-D- | Formula O25a |
| | GlcNAc-(1→ \| α-L-Rha-(1→3) ]ₙ | |
| O25b | 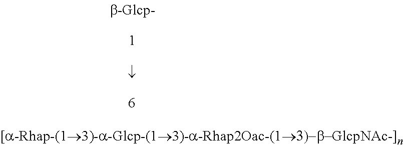 | Formula O25b |
| O26 | [ →3)-α-L-Rha-(1→4)-α-L-FucNAc-(1→3)-β-D-GlcNAc-(1→ ]ₙ | Formula O26 |
| O28 | [ →2)-l-Gro-1-P→4)-β-D-GlcNAc-(1→3)-β-D-Galf2Ac-(1→3)-α-D- | Formula O28 |
| | GlcNAc-(1→ ]ₙ | |
| O36 | 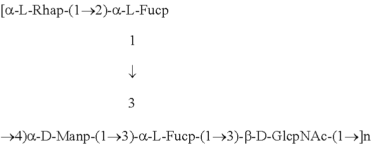 | Formula O36 |
| O44 | [ α-D-Glc-(1→4) \| →6)-α-D-Man-(1→2)-α-D-Man-(1→2)-β-D-Man- | Formula O44 |
| | (1→3)-α-D-GlcNAc(1→ ]ₙ | |
| O45 | [ →2)-β-D-Glc-(1→3)-α-L-6dTal2Ac-(1→3)-α-D-FucNAc-(1→ ]ₙ | Formula O45 |
| O45rel | [ →2)-β-D-Glc-(1→3)-α-L-6dTal2Ac-(1→3)-β-D-GlcNAc-(1→ ]ₙ | Formula O45rel |
| O54 | [→4)-α-d-GalpA-(1 → 2)-α-l-Rhap-(1 → 2)-β-d-Ribf- | Formula O54 |
| | (1 → 4)-β-d-Galp-(1 → 3)-β-d-GlapNAc-(1→]n | |
| O55 | [ →6)-β-D-GlcNAc-(1→3)-α-D-Gal-(1→3)-β-D-GalNAc-(1→ \| α-Col- | Formula O55 |
| | (1→2)-β-D-Gal-(1→3) ]ₙ | |
| O56 | [ →7)-α-Neu5Ac-(2→3)-β-D-Glc-(1→3)-β-D-GlcNAc-(1→ \| α-D- | Formula O56 |
| | Gal-(1→2) ]ₙ | |
| O57 | 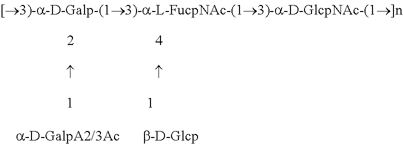 | Formula O57 |
| O58 | [ 3-O-[l-1-carboxyethyl]-α-L-Rha -(1→3) \| →4)-α-D-Man-(1→4)- | Formula O58 |
| | α-D-Man2Ac-(1→3)-β-D-GlcNAc-(1→ ]ₙ | |
| O64 | [ β-D-Gal-(1→6) \| →3)-α-D-ManNAc-(1→3)-β-D-GlcA-(1→3)-β-D- | Formula O64 |
| | Gal-(1→3)-β-D-GlcNAc(1→ ]ₙ | |
| O68 | 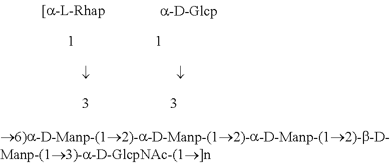 | Formula O68 |
| O69 | [ →2)-α-L-Rha-(1→2)-α-L-Rha-(1→2)-α-D-Gal-(1→3)-β-D-GlcNAc- | Formula O69 |
| | (1→ ]ₙ | |
| O73 (Strain | [ α-D-Glc-(1→3) \| →4)-α-D-Man-(1→2)-α-D-Man-(1→2)-β-D-Man- | Formula O73 |
| 73-1) | (1→3)-α-D-GalNAc(1→ ]ₙ | (Strain 73-1) |
| O74 | 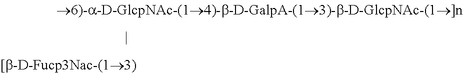 | Formula O74 |
| O75 | [ β-D-Man-(1→4) \| →3)-α-D-Gal-(1→4)-α-L-Rha-(1→3)-β-D- | Formula O75 |
| | GlcNAc-(1→ ]ₙ | |
| O76 | [→4)-β-D-GlcpA-(1→4)-β-D-GalpNAc3Ac-(1→4)-α-D-GalpNAc- | Formula O76 |
| | (1→3)-β-D-GalpNAc-(1→]n | |
| O77 | [ →6)-α-D-Man-(1→2)-α-D-Man-(1→2)-α-D-Man-(1→3)-α-D- | Formula O77 |
| | GlcNAc(1→ ]ₙ | |
| O78 | [ →4)-β-D-GlcNAc-(→4)-β-D-Man-(1→4)-α-D-Man-(1→3)-β-D- | Formula O78 |
| | GlcNAc-(1→ ]ₙ | |
| O86 | [ α-D-Gal-(1→3) \| →4)-α-L-Fuc-(1→2)-β-D-Gal-(1→3)-α-D- | Formula O86 |
| | GalNAc-(1→3)-β-D-GalNAc-(1→ ]ₙ | |
| O88 | [ α-L-6dTal-(1→3) \| →4)-α-D-Man-(1→3)-α-D-Man-(1→3)-β-D- | Formula O88 |
| | GlcNAc-(1→ ]ₙ | |
| O90 | [ →4)-α-L-Fuc2/3Ac-(1→2)-β-D-Gal-(1→3)-α-D-GalNAc-(1→3)-β- | Formula O90 |
| | D-GalNAc-(1→ ]ₙ | |
| O98 | [ →3)-α-L-QuiNAc-(1→4)-α-D-GalNAcA-(1→3)-α-L-QuiNAc- | Formula O98 |
| | (1→3)-β-D-GlcNAc-(1→ ]ₙ | |
| O104 | [ →4)-α-D-Gal-(1→4)-α-Neu5,7,9Ac₃-(2→3)-β-D-Gal-(1→3)-β-D- | Formula O104 |
| | GalNAc-(1→]ₙ | |
| O111 | [ α-Col-(1→6) \| →4)-α-D-Glc-(1→4)-α-D-Gal-(1→3)-β-D-GlcNAc- | Formula O111 |
| | (1→ \| α-Col-(1→3) ]ₙ | |
| O113 | [ →4)-α-D-GalNAc-(1→4)-α-D-GalA-(1→3)-α-D-Gal-(1→3)-β-D- | Formula O113 |
| | GlcNAc-(1→ \| β-D-Gal-(1→3) ]ₙ | |
| O114 | [ →4)-β-D-Qui3N(N-acetyl-L-seryl)-(1→3)-β-D-Ribf-(1→4)-β-D- | Formula O114 |
| | Gal-(1→3)-α-D-GlcNAc(1→ ]ₙ | |
| O119 | [ β-D-RhaNAc3Nfo-(1→3) \| →2)-β-D-Man-(1→3)-α-D-Gal-(1→4)- | Formula O119 |
| | α-L-Rha-(1→3)-α-D-GlcNAc-(1→ ]ₙ | |
| O121 | [ →3)-β-D-Qui4N(N-acetyl-glycyl)-(1→4)-α-D-GalNAc3AcA6N- | Formula O121 |
| | (1→4)-α-D-GalNAcA-(1→3)-α-D-GlcNAc-(1→ ]ₙ | |
| O124 | [ 4-O-[l-1-carboxyethyl]-β-D-Glc-(1→6)-α-D-Glc(1→4) \|→3)-α-D- | Formula O124 |
| | Gal-(1→6)-β-D-Galf-(1→3)-β-D-GalNAc-(1→ ]ₙ | |
| O125 | [ α-D-Glc-(1→3) \| →4)-β-D-GalNAc-(1→2)-α-D-Man-(1→3)-α-L- | Formula O125 |
| | Fuc-(1→3)-α-D-GalNAc-(1→\| β-D-Gal-(1→3) ]ₙ | |
| O126 | [ →)-β-D-Man-(1→3)-β-D-Gal-(1→3)-αD-GlcNAc-(1→3)-β-D- | Formula O126 |
| | GlcNAc-(1→ \| α-L-Fuc-(1→2) ]ₙ | |
| O127 | [ →2)-α-L-Fuc-(1→2)-β-D-Gal-(1→3)-α-D-GalNAc-(1→3)-α-D- | Formula O127 |
| | GalNAc-(1→ ]ₙ | |
| O128 | [ α-L-Fuc-(1→2) \| →6)-β-D-Gal-(1→3)-β-D-GalNAc-(1→4)-α-D- | Formula O128 |
| | Gal-(1→3)-β-D-GalNAc-(1→ ]ₙ | |
| O136 | [ →4)-β-Pse5Ac7Ac-(2→4)-β-D-Gal-(1→4)-β-D-GlcNAc-(1→β- | Formula O136 |
| | Pse5Ac7Ac = 5,7-diacetamido-3,5,7,9-tetradeoxy-L-glycero-β- | |
| | L-manno-nonulosonic acid ]ₙ | |
| O138 | [ →2)-α-L-Rha-(1→3)-α-L-Rha-(1→4)-α-D-GalNAcA-(1→3)-β-D- | Formula O138 |
| | GlcNAc-(1→ ]ₙ | |
| O140 | 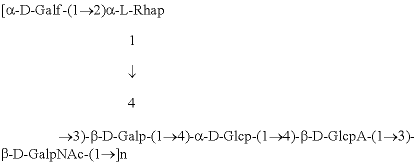 | Formula O140 |
| O141 | [ α-L-Rha-(1→3) \|→4)-α-D-Man-(1→3)-α-D-Man6Ac-(1→3)-β-D- | Formula O141 |
| | GlcNAc-(1→ \| β-D-GlcA-(1→2) ]ₙ | |
| O142 | [ →2)-α-L-Rha-(1→6)-α-D-GalNAc-(1→4)-α-D-GalNAc-(1→3)-α-D- | Formula O142 |
| | GalNAc-(1→ \| β-D-GlcNAc-(1→3) ]ₙ | |
| O143 | [ →2)-β-D-GalA6R3,4Ac-(1→3)-α-D-GalNAc-(1→4)-β-D-GlcA- | Formula O143 |
| | (1→3)-β-D-GlcNAc-(1→ R = 1,3-dihydroxy-2-propylamino ]ₙ | |
| O147 | [ →2)-α-L-Rha-(1→2)-α-L-Rha-(1→4)-β-D-GalA-(1→3)-β-D- | Formula O147 |
| | GalNAc-(1→ ]ₙ | |
| O149 | [ →3)-β-D-GlcNAc-(S)-4,6Py-(1→3)-β-L-Rha-(1→4)-β-D-GlcNAc- | Formula O149 |
| | (1→ (S)-4,6Py = 4,6-O-[(S)-1-carboxyethylidene]- ]ₙ | |
| O152 | [ β-L-Rha-(1→4) \| →3)-α-D-GlcNAc-(1-P→6)-α-D-Glc-(1→2)-β-D- | Formula O152 |
| | Glc-(1→3)-β-D-GlcNAc-(1→ ]ₙ | |
| O157 | [ →2)-α-D-Rha4Nac-(1→3)-α-L-Fuc-(1→4)-β-D-Glc-(1→3)-α-D- | Formula O157 |
| | GalNAc-(1→ ]ₙ | |
| O158 | [ α-D-Glc-(1→6) \| →4)-α-D-Glc-(1→3)-α-D-GalNAc-(1→3)-β-D- | Formula O158 |
| | GalNAc-(1→ \| α-L-Rha-(1→3) ]ₙ | |
| O159 | [ α-L-Fuc-(1→4) \| →3)-β-D-GlcNAc-(1→4)-α-D-GalA-(1→3)-α-L- | Formula O159 |
| | Fuc-(1→3)-β-D-GlcNAc-(1→ ]ₙ | |
| O164 | [ β-D-Glc-(1→6)-α-D-Glc(1→4) \| →3)-β-D-Gal-(1→6)-β-D-Galf- | Formula O164 |
| | (1→3)-β-D-GalNAc-(1→ ]ₙ | |
| O173 | [ α-L-Fuc-(1→4) \| →3)-α-D-Glc-(1-P→6)-α-D-Glc-(1→2)-β-D-Glc- | Formula O173 |
| | (1→3)-β-D-GlcNAc-(1→]ₙ | |
| 62D₁ | [ α-D-Gal(1→6) \| →2)-β-D-Qui3Nac-(1→3)-α-L-Rha-(1→3)-β-D- | Formula 62D₁ |
| Suggested as | Gal-(1→3)-α-D-FucNAc-(1→ ]ₙ | |
| Erwinia | | |
| herbicola | | |
| O22 | [ →6)-α-D-Glc-(1→4)-β-D-GlcA-(1→4)-β-D-GalNAc3Ac-(1→3)-α- | Formula O22 |
| | D-Gal-(1→3)-β-D-GalNAc-(1→]ₙ | |
| O35 | [ →3)-α-L-Rha-(1→2)-α-L-Rha-(1→3)-α-L-Rha-(1→2)-α-L-Rha- | Formula O35 |
| | (1→3)-β-D-GlcNAc-(1→ \| α-D-GalNAcA6N-(1→2) ]ₙ | |
| O65 | [ →2)-β-D-Qui3Nac-(1→4)-α-D-GalA6N-(1→4)-α-D-GalNAc- | Formula O65 |
| | (1→4)-β-D-GalA-(1→3)-α-D-GlcNAc-(1→ ]ₙ | |
| O66 | [ →2)-β-D-Man-(1→3)-α-D-GlcNAc-(1→2)-β-D-Glc3Ac-(1→3)-α-L- | Formula O66 |
| | 6dTal-(1→3)-α-D-GlcNAc(1→ ]ₙ | |
| O83 | [ →6)-α-D-Glc-(1→4)-β-D-GlcA-(1→6)-β-D-Gal-(1→4)-β-D-Gal- | Formula O83 |
| | (1→4)-β-D-GlcNAc-(1→ ]ₙ | |
| O91 | [ →4)-α-D-Qui3Nacyl-(1→4)-β-D-Gal-(1→4)-β-D-GlcNAc-(1→4)- | Formula O91 |
| | β-D-GlcA6Ngly-(1→3)-β-D-GlcNAc-(1→ Acyl = l-3- | |
| | hydroxybutyryl ]ₙ | |
| O105 | [ β-D-Ribf-(1→3) \| →4)-α-D-GlcA2Ac3Ac-(1→2)-α-L-Rha4Ac- | Formula O105 |
| | (1→3)-β-L-Rha-(1→4)-β-L-Rha-(1→3)-β-D-GlcNAc6Ac-(1→ ]ₙ | |
| O116 | [ →2)-β-D-Qui4Nac-(1→6)-α-D-GlcNAc-(1→4)-α-D-GalNAc- | Formula O116 |
| | (1→4)-α-D-GalA-(1→3)-β-D-GlcNAc-(1→ ]ₙ | |
| O117 | [ →4)-β-D-GalNAc-(1→3)-α-L-Rha-(1→4)-α-D-Glc-(1→4)-β-D-Gal- | Formula O117 |
| | (1→3)-α-D-GalNAc-(1→]ₙ | |
| O139 | [ β-D-Glc-(1→3) \| →3)-α-L-Rha-(1→4)-α-D-GalA-(1→2)-α-L-Rha- | Formula O139 |
| | (1→3)-α-L-Rha-(1→2)-α-L-Rha-(1→3)-α-D-GlcNAc-(1→ ]ₙ | |
| O153 | [ →2)-β-D-Ribf-(1→4)-β-D-Gal-(1→4)-α-D-GlcNAc-(1→4)-β-D- | Formula O153 |
| | Gal-(1→3)-α-D-GlcNAc-(1→ ]ₙ | |
| O167 | [ α-D-Galf-(1→4) \| →2)-β-D-GalA6N(L)Ala-(1→3)-α-D-GlcNAc- | Formula O167 |
| | (1→2)-β-D-Galf-(1→5)-β-D-Galf-(1→3)-β-D-GlcNAc-(1→ ]ₙ | |
| O172 | [ →3)-α-L-FucNAc-(1→4)-α-D-Glc6Ac-(1-P→4)-α-D-Glc-(1→3)-α- | Formula O172 |
| | L-FucNAc-(1→3)-α-D-GlcNAc-(1→ ]ₙ | |
| O8 | [ →2)-α-D-Man-(1→2)-α-D-Man-(1→3)-β-D-Man-(1→ ]ₙ | Formula O8 |
| O9a | [ →2)-α-D-Man-(1→2)-α-D-Man-(1→3)-α-D-Man-(1→3)-α-D- | Formula O9a |
| | Man-(1→ ]ₙ | |
| O9 | [ →2)-[α-D-Man-(1→2)]₂-α-D-Man-(1→3)-α-D-Man-(1→3)- | Formula O9 |
| | α-D-Man-(1→ ]ₙ | |
| O20ab | [ →2)-β-D-Ribf-(1→4)-α-D-Gal-(1→ ]ₙ | Formula O20ab |
| O20ac | [ α-D-Gal-(1→3) \| →2)-β-D-Ribf-(1→4)-α-D-Gal-(1→ ]ₙ | Formula O20ac |
| O52 | [ →3)-β-D-Fucf-(1→3)-β-D-6dmanHep2Ac-(1→ ]ₙ | Formula O52 |
| O97 | [ →3)-α-L-Rha-(1→3)-β-L-Rha-(1→ \|\| β-D-Xulf-(2→2)β-D- | Formula O97 |
| | Xulf-(2→2) ]ₙ | |

† β-D-6dmanHep2Ac is 2-O-acetyl-6-deoxy-β-D-manno-heptopyranosyl.
‡ β-D-Xulf is β-D-threo-pentofuranosyl.

### 3. Core Oligosaccharide

The core oligosaccharide is positioned between Lipid A and the O-antigen outer region in wild-type E. coli LPS. More specifically, the core oligosaccharide is the part of the polysaccharide that includes the bond between the O-antigen and the lipid A in wild type E. coli. This bond includes a ketosidic bond between the hemiketal function of the innermost 3-deoxy-d-manno-oct-2-ulosonic acid (KDO)) residue and a hydroxyl-group of a GlcNAc-residue of the lipid A. The core oligosaccharide region shows a high degree of similarity among wild-type E. coli strains. It usually includes a limited number of sugars. The core oligosaccharide includes an inner core region and an outer core region.

More specifically, the inner core is composed primarily of L-glycero-D-manno-heptose (heptose) and KDO residues. The inner core is highly conserved. A KDO residue includes the following Formula KDO:

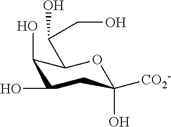

The outer region of the core oligosaccharide displays more variation than the inner core region, and differences in this region distinguish the five chemotypes in E. coli: R1, R2, R3, R4, and K-12. The generalized structures of the carbohydrate backbone of the outer core oligosaccharides of the five known chemotypes are well-known in the art. HepII is the last residue of the inner core oligosaccharide. While all of the outer core oligosaccharides share a structural theme, with a (hexose)₃ carbohydrate backbone and two side chain residues, the order of hexoses in the backbone and the nature, position, and linkage of the side chain residues can all vary. The structures for the R1 and R4 outer core oligosaccharides are highly similar, differing in only a single β-linked residue.

The core oligosaccharides of wild-type E. coli are categorized in the art based on the structures of the distal oligosaccharide, into five different chemotypes: E. coli R1, E. coli R2, E. coli R3, E. coli R4, and E. coli K12.

In a preferred embodiment, the compositions described herein include glycoconjugates in which the O-polysaccharide includes a core oligosaccharide bound to the O-antigen. In one embodiment, the composition induces an immune response against at least any one of the core E. coli chemotypes E. coli R1, E. coli R2, E. coli R3, E. coli R4, and E. coli K12. In another embodiment, the composition induces an immune response against at least two core E. coli chemotypes. In another embodiment, the composition induces an immune response against at least three core E. coli chemotypes. In another embodiment, the composition induces an immune response against at least four core E. coli chemotypes. In another embodiment, the composition induces an immune response against all five core E. coli chemotypes.

In another preferred embodiment, the compositions described herein include glycoconjugates in which the O-polysaccharide does not include a core oligosaccharide bound to the O-antigen. In one embodiment, such a composition induces an immune response against at least any one of the core E. coli chemotypes E. coli R1, E. coli R2, E. coli R3, E. coli R4, and E. coli K12, despite the glycoconjugate having an O-polysaccharide that does not include a core oligosaccharide.

E. coli serotypes may be characterized according to one of the five chemotypes. Table B lists exemplary serotypes characterized according to chemotype. The serotypes in bold represent the serotypes that are most commonly associated with the indicated core chemotype. Accordingly, in a preferred embodiment, the composition induces an immune response against at least any one of the core E. coli chemotypes E. coli R1, E. coli R2, E. coli R3, E. coli R4, and E. coli K12, which includes an immune response against any one of the respective corresponding E. coli serotypes.

TABLE B
Core Chemotype and associated E. coli Serotype

| Core chemotype | Serotype |
|---|---|
| R1 | O25a, O6, O2, O1, O75, O4, O16, O8, |
| | O18, O9, O13, O20, O21, O91, and O163. |
| R2 | O21, O44, O11, O89, O162, O9 |
| R3 | O25b, O15, O153, O21, O17, O11, |
| | O159, O22 O86, O93 |
| R4 | O2, O1, O86, O7, O102, O160, O166 |
| K-12 | O25b, O16 |

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O8, Formula O18, Formula O9, Formula O13, Formula O20, Formula O21, Formula O91, and Formula O163, wherein n is 1 to 100. In some embodiments, the saccharide in said composition further includes an E. coli R1 core moiety.

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O18, Formula O13, Formula O20, Formula O21, Formula O91, and Formula O163, wherein n is 1 to 100, preferably 31 to 100, preferably from 31 to 90 more preferably 35 to 90, most preferably 35 to 65. In some embodiments, the saccharide in said composition further includes an E. coli R1 core moiety in the saccharide.

In some embodiments, the composition includes a saccharide that includes a structure derived from a serotype having an R2 chemotype, e.g., selected from a saccharide having Formula O21, Formula O44, Formula O11, Formula O89, Formula O162, and Formula O9, wherein n is 1 to 100, preferably 31 to 100, preferably from 31 to 90, more preferably 35 to 90, most preferably 35 to 65. In some embodiments, the saccharide in said composition further includes an E. coli R2 core moiety.

In some embodiments, the composition includes a saccharide that includes a structure derived from a serotype having an R3 chemotype, e.g., selected from a saccharide having Formula O25b, Formula O15, Formula O153, Formula O21, Formula O17, Formula O11, Formula O159, Formula O22, Formula O86, and Formula O93, wherein n is 1 to 100, preferably 31 to 100, preferably from 31 to 90, more preferably 35 to 90, most preferably 35 to 65. In some embodiments, the saccharide in said composition further includes an E. coli R3 core moiety.

In some embodiments, the composition includes a saccharide that includes a structure derived from a serotype having an R4 chemotype, e.g., selected from a saccharide having Formula O2, Formula O1, Formula O86, Formula O7, Formula O102, Formula O160, and Formula O166, wherein n is 1 to 100, preferably 31 to 100, preferably from 31 to 90, more preferably 35 to 90, most preferably 35 to 65. In some embodiments, the saccharide in said composition further includes an E. coli R4 core moiety.

In some embodiments, the composition includes a saccharide that includes a structure derived from a serotype having an K-12 chemotype (e.g., selected from a saccharide having Formula O25b and a saccharide having Formula O16), wherein n is 1 to 1000, preferably 31 to 100, preferably from 31 to 90, more preferably 35 to 90, most preferably 35 to 65. In some embodiments, the saccharide in said composition further includes an E. coli K-12 core moiety.

In some embodiments, the saccharide includes the core saccharide. Accordingly, in one embodiment, the O-polysaccharide further includes an E. coli R1 core moiety. In another embodiment, the O-polysaccharide further includes an E. coli R2 core moiety. In another embodiment, the O-polysaccharide further includes an E. coli R3 core moiety. In another embodiment, the O-polysaccharide further includes an E. coli R4 core moiety. In another embodiment, the O-polysaccharide further includes an E. coli K12 core moiety.

In some embodiments, the saccharide does not include the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety.

### 4. Conjugated O-Antigens

Chemical linkage of O-antigens or preferably O-polysaccharides to protein carriers may improve the immunogenicity of the O-antigens or O-polysaccharides. However, variability in polymer size represents a practical challenge for production. In commercial use, the size of the saccharide can influence the compatibility with different conjugation synthesis strategies, product uniformity, and conjugate immunogenicity. Controlling the expression of a Wzz family protein chain length regulator through manipulation of the O-antigen synthesis pathway allows for production of a desired length of O-antigen chains in a variety of Gram-negative bacterial strains, including E. coli.

In one embodiment, the purified saccharides are chemically activated to produce activated saccharides capable of reacting with the carrier protein. Once activated, each saccharide is separately conjugated to a carrier protein to form a conjugate, namely a glycoconjugate. As used herein, the term “glycoconjugate” refers to a saccharide covalently linked to a carrier protein. In one embodiment a saccharide is linked directly to a carrier protein. In another embodiment, a saccharide is linked to a protein through a spacer/linker.

Conjugates may be prepared by schemes that bind the carrier to the O-antigen at one or at multiple sites along the O-antigen, or by schemes that activate at least one residue of the core oligosaccharide.

In one embodiment, each saccharide is conjugated to the same carrier protein. If the protein carrier is the same for 2 or more saccharides in the composition, the saccharides may be conjugated to the same molecule of the carrier protein (e.g., carrier molecules having 2 or more different saccharides conjugated to it).

In a preferred embodiment, the saccharides are each individually conjugated to different molecules of the protein carrier (each molecule of protein carrier only having one type of saccharide conjugated to it). In said embodiment, the saccharides are said to be individually conjugated to the carrier protein.

The chemical activation of the saccharides and subsequent conjugation to the carrier protein can be achieved by the activation and conjugation methods disclosed herein. After conjugation of the polysaccharide to the carrier protein, the glycoconjugates are purified (enriched with respect to the amount of polysaccharide-protein conjugate) by a variety of techniques. These techniques include concentration/diafiltration operations, precipitation/elution, column chromatography, and depth filtration. After the individual glycoconjugates are purified, they are compounded to formulate the immunogenic composition of the present invention.
a. Activation. The present invention further relates to activated polysaccharides produced from any of the embodiments described herein wherein the polysaccharide is activated with a chemical reagent to produce reactive groups for conjugation to a linker or carrier protein. In some embodiments, the saccharide of the invention is activated prior to conjugation to the carrier protein. In some embodiments, the degree of activation does not significantly reduce the molecular weight of the polysaccharide. For example, in some embodiments, the degree of activation does not cleave the polysaccharide backbone. In some embodiments, the degree of activation does not significantly impact the degree of conjugation, as measured by the number of lysine residues modified in the carrier protein, such as, CRM₁₉₇ (as determined by amino acid analysis). For example, in some embodiments, the degree of activation does not significantly increase the number of lysine residues modified (as determined by amino acid analysis) in the carrier protein by 3-fold, as compared to the number of lysine residues modified in the carrier protein of a conjugate with a reference polysaccharide at the same degree of activation. In some embodiments, the degree of activation does not increase the level of unconjugated free saccharide. In some embodiments, the degree of activation does not decrease the optimal saccharide/protein ratio.

In some embodiments, the activated saccharide has a percentage of activation wherein moles of thiol per saccharide repeat unit of the activated saccharide is between 1-100%, such as, for example, between 2-80%, between 2-50%, between 3-30%, and between 4-25%. The degree of activation is at least 1%, 2%, 3%, 4%, 5%, 6%, 7%, 8%, 9%, 10%, 11%, 12%, 13%, 14%, 15%, 16%, 17%, 18%, 19%, ≥20%, ≥30%, ≥40%, ≥50%, ≥60%, ≥70%, ≥80%, or 90%, or about 100%. Preferably, the degree of activation is at most 50%, more preferably at most 25%. In one embodiment, the degree of activation is at most 20%. Any minimum value and any maximum value may be combined to define a range.

In one embodiment, the polysaccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇ or tetanus toxoid).

For example, the spacer may be cystamine or cysteamine to give a thiolated polysaccharide which could be coupled to the carrier via a thioether linkage obtained after reaction with a maleimide-activated carrier protein (for example using N-[Y-maleimidobutyrloxy]succinimide ester (GMBS)) or a haloacetylated carrier protein (for example using 76yophilized76e, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). In one embodiment, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier.

Other suitable techniques for conjugation use carbodiimides, hydrazides, active esters, norborane, p-nitrobenzoic acid, N-hydroxysuccinimide, S-NHS, EDC, TSTU. Conjugation may involve a carbonyl linker which may be formed by reaction of a free hydroxyl group of the saccharide with CDI followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein (CDI chemistry).
b. Molecular weight. In some embodiments, the glycoconjugate comprises a saccharide having a molecular weight of between 10 kDa and 2,000 kDa. In other embodiments, the saccharide has a molecular weight of between 50 kDa and 1,000 kDa. In other embodiments, the saccharide has a molecular weight of between 70 kDa and 900 kDa. In other embodiments, the saccharide has a molecular weight of between 100 kDa and 800 kDa. In other embodiments, the saccharide has a molecular weight of between 200 kDa and 600 kDa. In further embodiments, the saccharide has a molecular weight of 100 kDa to 1000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1,000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. In one embodiment, the glycoconjugate having such a molecular weight is produced by single-end conjugation. In another embodiment, the glycoconjugate having such a molecular weight is produced by reductive amination chemistry (RAC) prepared in aqueous buffer. Any whole number integer within any of the above ranges is contemplated as an embodiment of the disclosure.

In some embodiments, the glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the glycoconjugate has a molecular weight of between 500 kDa and 10,000 kDa. In other embodiments, glycoconjugate has a molecular weight of between 1,000 kDa and 8,000 kDa. In still other embodiments, the glycoconjugate has a molecular weight of between 2,000 kDa and 8,000 kDa or between 3,000 kDa and 7,000 kDa. In further embodiments, the glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa. In one embodiment, the glycoconjugate having such a molecular weight is produced by eTEC conjugation described herein. In another embodiment, the glycoconjugate having such a molecular weight is produced by reductive amination chemistry (RAC). In another embodiment, the glycoconjugate having such a molecular weight is produced by reductive amination chemistry (RAC) prepared in DMSO.

In further embodiments, the glycoconjugate of the invention has a molecular weight of between 1,000 kDa and 20,000 kDa; between 1,000 kDa and 15,000 kDa; between 2,000 kDa and 10,000 kDa; between 2000 kDa and 7,500 kDa; between 2,000 kDa and 5,000 kDa; between 3,000 kDa and 20,000 kDa; between 3,000 kDa and 15,000 kDa; between 3,000 kDa and 12,500 kDa; between 4,000 kDa and 10,000 kDa; between 4,000 kDa and 7,500 kDa; between 4,000 kDa and 6,000 kDa; or between 5,000 kDa and 7,000 kDa. In one embodiment, the glycoconjugate having such a molecular weight is produced by reductive amination chemistry (RAC). In another embodiment, the glycoconjugate having such a molecular weight is produced by reductive amination chemistry (RAC) prepared in DMSO. In another embodiment, the glycoconjugate having such a molecular weight is produced by eTEC conjugation described herein.

In further embodiments, the glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate may be measured by SEC-MALLS. Any whole number integer within any of the above ranges is contemplated as an embodiment of the disclosure. The glycoconjugates of the invention may also be characterized by the ratio (weight/weight) of saccharide to carrier protein. In some embodiments, the ratio of polysaccharide to carrier protein in the glycoconjugate (w/w) is between 0.5 and 3 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In a preferred embodiment, the ratio of polysaccharide to carrier protein in the conjugate is between 0.9 and 1.1. In some such embodiments, the carrier protein is CRM197.

The glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−Vo)/(Vi−V0).
c. Free saccharide. The glycoconjugates and immunogenic compositions of the invention may include free saccharide that is not covalently conjugated to the carrier protein, but is nevertheless present in the glycoconjugate composition. The free saccharide may be non-covalently associated with (i.e., non-covalently bound to, adsorbed to, or entrapped in or with) the glycoconjugate. In a preferred embodiment, the glycoconjugate comprises at most 50%, 45%, 40%, 35%, 30%, 25%, 20% or 15% of free polysaccharide compared to the total amount of polysaccharide. In a preferred embodiment the glycoconjugate comprises less than about 25% of free polysaccharide compared to the total amount of polysaccharide. In a preferred embodiment the glycoconjugate comprises at most about 20% of free polysaccharide compared to the total amount of polysaccharide. In a preferred embodiment the glycoconjugate comprises at most about 15% of free polysaccharide compared to the total amount of polysaccharide. In another preferred embodiment, the glycoconjugate comprises at most about 20%, 19%, 18%, 17%, 16%, 15%, 14%, 13%, 12%, 11%, 10%, 9%, 8%, 7%, 6%, 5%, 4%, 3%, 2%, or 1% of free polysaccharide compared to the total amount of polysaccharide. In a preferred embodiment the glycoconjugate comprises less than about 8% of free polysaccharide compared to the total amount of polysaccharide. In a preferred embodiment the glycoconjugate comprises at most about 6% of free polysaccharide compared to the total amount of polysaccharide. In a preferred embodiment the glycoconjugate comprises at most about 5% of free polysaccharide compared to the total amount of polysaccharide.
d. Covalent linkage. In other embodiments, the conjugate comprises at least one covalent linkage between the carrier protein and saccharide for every 5 to 10 saccharide repeat units; every 2 to 7 saccharide repeat units; every 3 to 8 saccharide repeat units; every 4 to 9 saccharide repeat units; every 6 to 1 1 saccharide repeat units; every 7 to 12 saccharide repeat units; every 8 to 13 saccharide repeat units; every 9 to 14 saccharide repeat units; every 10 to 15 saccharide repeat units; every 2 to 6 saccharide repeat units, every 3 to 7 saccharide repeat units; every 4 to 8 saccharide repeat units; every 6 to 10 saccharide repeat units; every 7 to 1 1 saccharide repeat units; every 8 to 12 saccharide repeat units; every 9 to 13 saccharide repeat units; every 10 to 14 saccharide repeat units; every 10 to 20 saccharide repeat units; every 4 to 25 saccharide repeat units or every 2 to 25 saccharide repeat units. In frequent embodiments, the carrier protein is CRM197. In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In one embodiment, the carrier protein is CRM197. Any whole number integer within any of the above ranges is contemplated as an embodiment of the disclosure.
e. Lysine residues. Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In one embodiment, the degree of conjugation of the glycoconjugate of the invention is about 2, about 3, about 4, about 5, about 6, about 7, about 8, about 9, about 10, about 1 1, about 12, about 13, about 14 or about 15. In a preferred embodiment, the degree of conjugation of the glycoconjugate of the invention is between 4 and 7. In some such embodiments, the carrier protein is CRM₁₉₇.

The frequency of attachment of the saccharide chain to a lysine on the carrier protein is another parameter for characterizing the glycoconjugates of the invention. For example, in some embodiments, at least one covalent linkage between the carrier protein and the polysaccharide for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide.
f. O-acetylation. In some embodiments, the saccharides of the invention are O-acetylated. In some embodiments, the glycoconjugate comprises a saccharide which has a degree of O-acetylation of between 10-100%, between 20-100%, between 30-100%, between 40-100%, between 50-100%, between 60-100%, between 70-100%, between 75-100%, 80-100%, 90-100%, 50-90%, 60-90%, 70-90% or 80-90%. In other embodiments, the degree of O-acetylation is ≥10%, ≥20%, ≥30%, ≥40%, ≥50%, ≥60%, ≥70%, ≥80%, or ≥90%, or about 100%. By % of O-acetylation it is meant the percentage of a given saccharide relative to 100% (where each repeat unit is fully acetylated relative to its acetylated structure).

In some embodiments, the glycoconjugate is prepared by reductive amination. In some embodiments, the glycoconjugate is a single-end-linked conjugated saccharide, wherein the saccharide is covalently bound to a carrier protein directly. In some embodiments, the glycoconjugate is covalently bound to a carrier protein through a (2-((2-oxoethyl)thio)ethyl) carbamate (eTEC) spacer.
g. Reductive Amination. In one embodiment, the saccharide is conjugated to the carrier protein by reductive amination (such as described in U.S. Patent Appl. Pub. Nos. 2006/0228380, 2007/0231340, 2007/0184071 and 2007/0184072, WO 2006/110381, WO 2008/079653, and WO 2008/143709).

Reductive amination includes (1) oxidation of the saccharide, (2) reduction of the activated saccharide and a carrier protein to form a conjugate. Before oxidation, the saccharide is optionally hydrolyzed. Mechanical or chemical hydrolysis may be employed. Chemical hydrolysis may be conducted using acetic acid.

The oxidation step may involve reaction with periodate. The term “periodate” as used herein refers to both periodate and periodic acid. The term also includes both metaperiodate (IO4-) and orthoperiodate (IO65-) and the various salts of periodate (e.g., sodium periodate and potassium periodate). In one embodiment the polysaccharide is oxidized in the presence of metaperiodate, preferably in the presence of sodium periodate (NaIO4). In another embodiment the polysaccharide is oxidized in the presence of orthoperiodate, preferably in the presence of periodic acid.

In one embodiment, the oxidizing agent is a stable nitroxyl or nitroxide radical compound, such as piperidine-N-oxy or pyrrolidine-N-oxy compounds, in the presence of an oxidant to selectively oxidize primary hydroxyls. In said reaction, the actual oxidant is the N-oxoammonium salt, in a catalytic cycle. In an aspect, said stable nitroxyl or nitroxide radical compound are piperidine-N-oxy or pyrrolidine-N-oxy compounds. In an aspect, said stable nitroxyl or nitroxide radical compound bears a TEMPO (2,2,6,6-tetramethyl-1-piperidinyloxy) or a PROXYL (2,2,5,5-tetramethyl-1-pyrrolidinyloxy) moiety. In an aspect, said stable nitroxyl radical compound is TEMPO or a derivative thereof. In an aspect, said oxidant is a molecule bearing a N-halo moiety. In an aspect, said oxidant is selected from any one of N-ChloroSuccinimide, N-Bromosuccinimide, N-Iodosuccinimide, Dichloroisocyanuric acid, 1,3,5-trichloro-I,3,5-triazinane-2,4,6-trione, Dibromoisocyanuric acid, 1,3,5-tribromo-I,3,5-triazinane-2,4,6-trione, Diiodoisocyanuric acid and 1,3,5-triiodo-I,3,5-triazinane-2,4,6-trione. Preferably said oxidant is N-Chlorosuccinimide.

Following the oxidation step of the saccharide, the saccharide is said to be activated and is referred to as “activated” herein below. The activated saccharide and the carrier protein may be 82yophilized (freeze-dried), either independently (discrete lyophilization) or together (co-lyophilized). In one embodiment the activated saccharide and the carrier protein are co-lyophilized. In another embodiment the activated polysaccharide and the carrier protein are lyophilized independently.

In one embodiment the lyophilization takes place in the presence of a non-reducing sugar, possible non-reducing sugars include sucrose, trehalose, raffinose, stachyose, melezitose, dextran, mannitol, lactitol and palatinit.

The next step of the conjugation process is the reduction of the activated saccharide and a carrier protein to form a conjugate (so-called reductive amination), using a reducing agent. Suitable reducing agents include the cyanoborohydrides, such as sodium cyanoborohydride, sodium triacetoxyborohydride or sodium or zinc borohydride in the presence of Bronsted or Lewis acids), amine boranes such as pyridine borane, 2-Picoline Borane, 2,6-diborane-methanol, dimethylamine-borane, t-BuMe′PrN-BH3, benzylamine-BH3 or 5-ethyl-2-methylpyridine borane (PEMB), borane-pyridine, or borohydride exchange resin. In one embodiment the reducing agent is sodium cyanoborohydride.

In an embodiment, the reduction reaction is carried out in aqueous solvent (e.g., selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, 7.0 and 8.0, or 7.0 and 7.5), in another embodiment the reaction is carried out in aprotic solvent. In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment this capping agent is sodium borohydride (NaBH4). Following the conjugation (the reduction reaction and optionally the capping), the glycoconjugates may be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. The glycoconjugates maybe purified by diafiltration and/or ion exchange chromatography and/or size exclusion chromatography. In an embodiment, the glycoconjugates are purified by diafiltration or ion exchange chromatography or size exclusion chromatography. In one embodiment the glycoconjugates are sterile filtered.

In a preferred embodiment, a glycoconjugate from an E. coli serotype is selected from any one of O25b, O1a, O2, and O6 is prepared by reductive amination. In a preferred embodiment, the glycoconjugates from E. coli serotypes O25b, O1a, O2, and O6 are prepared by reductive amination.

In one aspect, the invention relates to a conjugate that includes a carrier protein, e.g., CRM197, linked to a saccharide of Formula O25B, presented by

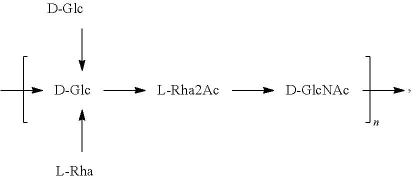

wherein n is any integer greater than or equal to 1. In a preferred embodiment, n is an integer of at least 31, 32, 33, 34, 35, 36, 37, 38, 39, 40, and at most 200, 100, 99, 98, 97, 96, 95, 94, 93, 92, 91, 90, 89, 88, 87, 86, 81, 80, 79, 78, 77, 76, 75, 74, 73, 72, 71, 70, 69, 68, 67, 66, 65, 60, 59, 58, 57, 56, 55, 54, 53, 52, 51, or 50. Any minimum value and any maximum value may be combined to define a range. Exemplary ranges include, for example, at least 1 to at most 1000; at least 10 to at most 500; and at least 20 to at most 80. In one preferred embodiment, n is at least 31 to at most 90, more preferably 40 to 90, most preferably 60 to 85.

In another aspect, the invention relates to a conjugate that includes a carrier protein, e.g., CRM197, linked to a saccharide having any one of the following structures shown in Table A, wherein n is an integer greater than or equal to 1.

Without being bound by theory or mechanism, in some embodiments, a stable conjugate is believed to require a level of saccharide antigen modification that is balanced against preserving the structural integrity of the critical immunogenic epitopes of the antigen.
h. Activation and formation of an Aldehyde. In some embodiments, the saccharide of the invention is activated and results in the formation of an aldehyde. In such embodiments wherein the saccharide is activated, the percentage (%) of activation (or degree of oxidation (DO)) refers to moles of a saccharide repeat unit per moles of aldehyde of the activated polysaccharide. For example, in some embodiments, the saccharide is activated by periodate oxidation of vicinal diols on a repeat unit of the polysaccharide, resulting in the formation of an aldehyde. Varying the molar equivalents (meq) of sodium periodate relative to the saccharide repeat unit and temperature during oxidation results in varying levels of degree of oxidation (DO).

The saccharide and aldehyde concentrations are typically determined by colorimetric assays. An alternative reagent is TEMPO (2,2,6,6-tetramethylpiperidine 1-oxyl radical)-N-chlorosuccinimide (NCS) combination, which results in the formation of aldehydes from primary alcohol groups.

In some embodiments, the activated saccharide has a degree of oxidation wherein the moles of a saccharide repeat unit per moles of aldehyde of the activated saccharide is between 1-100, such as, for example, between 2-80, between 2-50, between 3-30, and between 4-25. The degree of activation is at least 1, 2, 3, 4, 5, 6, 7, 8, 9, 10, 11, 12, 13, 14, 15, 16, 17, 18, 19, ≥20, ≥30, ≥40, ≥50, ≥60, ≥70, ≥80, or ≥90, or about 100. Preferably, the degree of oxidation (DO) is at least 5 and at most 50, more preferably at least 10 and at most 25. In one embodiment, the degree of activation is at least 10 and at most 25. Any minimum value and any maximum value may be combined to define a range. A degree of oxidation value may be represented as percentage (%) of activation. For example, in one embodiment, a DO value of 10 refers to one activated saccharide repeat unit out of a total of 10 saccharide repeat units in the activated saccharide, in which case the DO value of 10 may be represented as 10% activation.

In some embodiments, the conjugate prepared by reductive amination chemistry includes a carrier protein and a saccharide, wherein the saccharide includes a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D₁, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187. In some embodiments, the saccharide in the conjugate includes a Formula, wherein n is an integer from 1 to 1000, from 5 to 1000, preferably 31 to 100 or 31 to 90, more preferably 35 to 90, most preferably 35 to 65.
i. Single-end Linked Conjugates. In some embodiments, the conjugate is single-end-linked conjugated saccharide, wherein the saccharide is covalently bound at one end of the saccharide to a carrier protein. In some embodiments, the single-end-linked conjugated polysaccharide has a terminal saccharide. For example, a conjugate is single-end linked if one of the ends (a terminal saccharide residue) of the polysaccharide is covalently bound to a carrier protein. In some embodiments, the conjugate is single-end linked if a terminal saccharide residue of the polysaccharide is covalently bound to a carrier protein through a linker. Such linkers may include, for example, a cystamine linker (A1), a 3,3′-dithio bis(propanoic dihydrazide) linker (A4), and a 2,2′-dithio-N,N′-bis(ethane-2,1-diyl)bis(2-(aminooxy)acetamide) linker (A6).

In some embodiments, the saccharide is conjugated to the carrier protein through a 3-deoxy-d-manno-oct-2-ulosonic acid (KDO) residue to form a single-end linked conjugate.

In some embodiments, the conjugate is preferably not a bioconjugate. The term “bioconjugate” refers to a conjugate between a protein (e.g., a carrier protein) and an antigen, e.g., an O antigen (e.g., O25B) prepared in a host cell background, wherein host cell machinery links the antigen to the protein (e.g., N-links). Glycoconjugates include bioconjugates, as well as sugar antigen (e.g., oligo- and polysaccharides)-protein conjugates prepared by means that do not require preparation of the conjugate in a host cell, e.g., conjugation by chemical linkage of the protein and saccharide.
j. Thiol Activated Saccharides. In some embodiments, the saccharide of the invention is thiol activated. In such embodiments wherein the saccharide is thiol activated, the percentage (%) of activation refers to moles of thiol per saccharide repeat unit of the activated polysaccharide.

The saccharide and thiol concentrations are typically determined by Ellman's assay for quantitation of sulfhydryls. For example, in some embodiments, the saccharide includes activation of 2-Keto-3-deoxyoctanoic acid (KDO) with a disulfide amine linker. In some embodiments, the saccharide is covalently bound to a carrier protein through a bivalent, heterobifunctional linker (also referred to herein as a “spacer”). The linker preferably provides a thioether bond between the saccharide and the carrier protein, resulting in a glycoconjugate referred to herein as a “thioether glycoconjugate.” In some embodiments, the linker further provides carbamate and amide bonds, such as, for example, (2-((2-oxoethyl)thio)ethyl) carbamate (eTEC).

In some embodiments, the single-end linked conjugate includes a carrier protein and a saccharide, wherein the saccharide includes a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187. In some embodiments, the saccharide in the conjugate includes a Formula, wherein n is an integer from 1 to 1000, from 5 to 1000, preferably 31 to 100, more preferably 35 to 90, most preferably 35 to 65.

For example, in one embodiment, the single-end linked conjugate includes a carrier protein and a saccharide having a structure selected from Formula O8, Formula O9a, Formula O9, Formula O20ab, Formula O20ac, Formula O52, Formula O97, and Formula O101, wherein n is an integer from 1 to 10.

5. eTEC Conjugates

In one aspect, the invention relates generally to glycoconjugates comprising a saccharide derived from E. coli described above covalently conjugated to a carrier protein through a (2-((2-oxoethyl)thio)ethyl)carbamate (eTEC) spacer (as described, for example, in U.S. Pat. No. 9,517,274 and International Patent Application Publication WO2014027302, incorporated by reference herein in their entireties), including immunogenic compositions comprising such glycoconjugates, and methods for the preparation and use of such glycoconjugates and immunogenic compositions. Said glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC spacer includes seven linear atoms (i.e., —C(O)NH(CH2)2SCH2C(O)—) and provides stable thioether and amide bonds between the saccharide and carrier protein.

The eTEC linked glycoconjugates of the invention may be represented by the general formula (I):

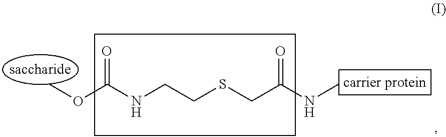

where the atoms that comprise the eTEC spacer are contained in the central box.

In said glycoconjugates of the invention, the saccharide may be a polysaccharide or an oligosaccharide.

The carrier proteins incorporated into the glycoconjugates of the invention are selected from the group of carrier proteins generally suitable for such purposes, as further described herein or known to those of skill in the art. In particular embodiments, the carrier protein is CRM197.

In another aspect, the invention provides a method of making a glycoconjugate comprising a saccharide described herein conjugated to a carrier protein through an eTEC spacer, comprising the steps of a) reacting a saccharide with a carbonic acid derivative in an organic solvent to produce an activated saccharide; b) reacting the activated saccharide with cystamine or cysteamine or a salt thereof, to produce a thiolated saccharide; c) reacting the thiolated saccharide with a reducing agent to produce an activated thiolated saccharide comprising one or more free sulfhydryl residues; d) reacting the activated thiolated saccharide with an activated carrier protein comprising one or more α-haloacetamide groups, to produce a thiolated saccharide-carrier protein conjugate; and e) reacting the thiolated saccharide-carrier protein conjugate with (i) a first capping reagent capable of capping unconjugated α-haloacetamide groups of the activated carrier protein; and/or (ii) a second capping reagent capable of capping unconjugated free sulfhydryl residues of the activated thiolated saccharide; whereby an eTEC linked glycoconjugate is produced.

In frequent embodiments, the carbonic acid derivative is 1,1′-carbonyl-di-(1,2,4-triazole) (CDT) or 1,1′-carbonyldiimidazole (CDI). Preferably, the carbonic acid derivative is CDT and the organic solvent is a polar aprotic solvent, such as dimethylsulfoxide (DMSO). In preferred embodiments, the thiolated saccharide is produced by reaction of the activated saccharide with the bifunctional symmetric thioalkylamine reagent, cystamine or a salt thereof. Alternatively, the thiolated saccharide may be formed by reaction of the activated saccharide with cysteamine or a salt thereof. The eTEC linked glycoconjugates produced by the methods of the invention may be represented by general Formula (I).

In frequent embodiments, the first capping reagent is N-acetyl-L-cysteine, which reacts with unconjugated α-haloacetamide groups on lysine residues of the carrier protein to form an S-carboxymethylcysteine (CMC) residue covalently linked to the activated lysine residue through a thioether linkage.

In other embodiments, the second capping reagent is iodoacetamide (IAA), which reacts with unconjugated free sulfhydryl groups of the activated thiolated saccharide to provide a capped thioacetamide. Frequently, step e) comprises capping with both a first capping reagent and a second capping reagent. In certain embodiments, step e) comprises capping with N-acetyl-L-cysteine as the first capping reagent and IAA as the second capping reagent.

In some embodiments, the capping step e) further comprises reaction with a reducing agent, for example, DTT, TCEP, or mercaptoethanol, after reaction with the first and/or second capping reagent.

The eTEC linked glycoconjugates and immunogenic compositions of the invention may include free sulfhydryl residues. In some instances, the activated thiolated saccharides formed by the methods provided herein will include multiple free sulfhydryl residues, some of which may not undergo covalent conjugation to the carrier protein during the conjugation step. Such residual free sulfhydryl residues are capped by reaction with a thiol-reactive capping reagent, for example, iodoacetamide (IAA), to cap the potentially reactive functionality. Other thiol-reactive capping reagents, e.g., maleimide containing reagents and the like are also contemplated.

In addition, the eTEC linked glycoconjugates and immunogenic compositions of the invention may include residual unconjugated carrier protein, which may include activated carrier protein which has undergone modification during the capping process steps.

In some embodiments, step d) further comprises providing an activated carrier protein comprising one or more α-haloacetamide groups prior to reacting the activated thiolated saccharide with the activated carrier protein. In frequent embodiments, the activated carrier protein comprises one or more α-bromoacetamide groups.

In another aspect, the invention provides an eTEC linked glycoconjugate comprising a saccharide described herein conjugated to a carrier protein through an eTEC spacer produced according to any of the methods disclosed herein.

In some embodiments, the carrier protein is CRM197 and the covalent linkage via an eTEC spacer between the CRM197 and the polysaccharide occurs at least once in every 4, 10, 15 or 25 saccharide repeat units of the polysaccharide.

For each of the aspects of the invention, in particular embodiments of the methods and compositions described herein, the eTEC linked glycoconjugate comprises a saccharide described herein, such as, a saccharide derived from E. coli.

In another aspect, the invention provides a method of preventing, treating or ameliorating a bacterial infection, disease or condition in a subject, comprising administering to the subject an immunologically effective amount of an immunogenic composition of the invention, wherein said immunogenic composition comprises an eTEC linked glycoconjugate comprising a saccharide described herein. In some embodiments, the saccharide is derived from E. coli.

In some embodiments, the eTEC linked glycoconjugate comprises a carrier protein and a saccharide, in which said saccharide comprises a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187. In some embodiments, the saccharide in the conjugate includes a Formula, wherein n is an integer from 1 to 1000, from 5 to 1000, preferably 31 to 100, more preferably 35 to 90, most preferably 35 to 65.

The number of lysine residues in the carrier protein that become conjugated to the saccharide can be characterized as a range of conjugated lysines. For example, in some embodiments of the immunogenic compositions, the CRM₁₉₇ may comprise 4 to 16 lysine residues out of 39 covalently linked to the saccharide. Another way to express this parameter is that about 10% to about 41% of CRM₁₉₇ lysines are covalently linked to the saccharide. In other embodiments, the CRM₁₉₇ may comprise 2 to 20 lysine residues out of 39 covalently linked to the saccharide. Another way to express this parameter is that about 5% to about 50% of CRM₁₉₇ lysines are covalently linked to the saccharide.

In frequent embodiments, the carrier protein is CRM₁₉₇ and the covalent linkage via an eTEC spacer between the CRM₁e7 and the polysaccharide occurs at least once in every 4, 10, 15 or 25 saccharide repeat units of the polysaccharide.

In other embodiments, the conjugate comprises at least one covalent linkage between the carrier protein and saccharide for every 5 to 10 saccharide repeat units; every 2 to 7 saccharide repeat units; every 3 to 8 saccharide repeat units; every 4 to 9 saccharide repeat units; every 6 to 11 saccharide repeat units; every 7 to 12 saccharide repeat units; every 8 to 13 saccharide repeat units; every 9 to 14 saccharide repeat units; every 10 to 15 saccharide repeat units; every 2 to 6 saccharide repeat units, every 3 to 7 saccharide repeat units; every 4 to 8 saccharide repeat units; every 6 to 10 saccharide repeat units; every 7 to 11 saccharide repeat units; every 8 to 12 saccharide repeat units; every 9 to 13 saccharide repeat units; every 10 to 14 saccharide repeat units; every 10 to 20 saccharide repeat units; or every 4 to 25 saccharide repeat units. In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide.

### 6. Carrier Proteins

A component of the glycoconjugate of the invention is a carrier protein to which the saccharide is conjugated. The terms “protein carrier” or “carrier protein” or “carrier” may be used interchangeably herein. Carrier proteins should be amendable to standard conjugation procedures.

One component of the conjugate is a carrier protein to which the O-polysaccharide is conjugated. In one embodiment, the conjugate includes a carrier protein conjugated to the core oligosaccharide of the O-polysaccharide. In one embodiment, the conjugate includes a carrier protein conjugated to the O-antigen of the O-polysaccharide.

The terms “protein carrier” or “carrier protein” or “carrier” may be used interchangeably herein. Carrier proteins should be amendable to standard conjugation procedures.

In a preferred embodiment, the carrier protein of the conjugates is independently selected from any one of TT, DT, DT mutants (such as CRM197), H. influenzae protein D, PhtX, PhtD, PhtDE fusions (particularly those described in WO 01/98334 and WO 03/54007), detoxified pneumolysin, PorB, N19 protein, PspA, OMPC, toxin A or B of C. difficile and PsaA. In an embodiment, the carrier protein of the conjugates of the invention is DT (Diphtheria toxoid). In another embodiment, the carrier protein of the conjugates of the invention is TT (tetanus toxoid). In another embodiment, the carrier protein of the conjugates of the invention is PD (Haemophilus influenzae protein D—see, e.g., EP 0 594 610 B). In some embodiments, the carrier protein includes poly(L-lysine) (PLL).

In a preferred embodiment, the saccharides are conjugated to CRM197 protein. The CRM197 protein is a nontoxic form of diphtheria toxin but is immunologically indistinguishable from the diphtheria toxin. CRM197 is produced by C. diphtheriae infected by the nontoxigenic phage β197tox-created by nitrosoguanidine mutagenesis of the toxigenic corynephage beta. The CRM197 protein has the same molecular weight as the diphtheria toxin but differs therefrom by a single base change (guanine to adenine) in the structural gene. This single base change causes an amino acid substitution glutamic acid for glycine) in the mature protein and eliminates the toxic properties of diphtheria toxin. The CRM197 protein is a safe and effective T-cell dependent carrier for saccharides.

Accordingly, in some embodiments, the conjugates of the invention include CRM₁₉₇ as the carrier protein, wherein the saccharide is covalently linked to CRM₁₉₇.

In a preferred embodiment, the carrier protein of the glycoconjugates is selected in the group consisting of DT (Diphtheria toxin), TT (tetanus toxoid) or fragment C of TT, CRM197 (a nontoxic but antigenically identical variant of diphtheria toxin), other DT mutants (such as CRM176, CRM228, CRM 45 (Uchida et al J. Biol. Chem. 218; 3838-3844, 1973), CRM9, CRM45, CRM102, CRM103 or CRM107; and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711), pneumococcal pneumolysin (Kuo et al (1995) Infect Immun 63; 2706-13) including ply detoxified in some fashion for example dPLY-GMBS (WO 04081515, PCT/EP2005/010258) or dPLY-formol, PhtX, including PhtA, PhtB, PhtD, PhtE (sequences of PhtA, PhtB, PhtD or PhtE are disclosed in WO 00/37105 or WO 00/39299) and fusions of Pht proteins for example PhtDE fusions, PhtBE fusions, Pht A-E (WO 01/98334, WO 03/54007, WO2009/000826), OMPC (meningococcal outer membrane protein—usually extracted from N. meningitidis serogroup B—EP0372501), PorB (from N. meningitidis), PD (Haemophilus influenzae protein D—see, e.g., EP 0 594 610 B), or immunologically functional equivalents thereof, synthetic peptides (EP0378881, EP0427347), heat shock proteins (WO 93/17712, WO 94/03208), pertussis proteins (WO 98/58668, EP0471 177), cytokines, lymphokines, growth factors or hormones (WO 91/01146), artificial proteins comprising multiple human CD4+ T cell epitopes from various pathogen derived antigens (Falugi et al (2001) Eur J Immunol 31; 3816-3824) such as N19 protein (Baraldoi et al (2004) Infect Immun 72; 4884-7) pneumococcal surface protein PspA (WO 02/091998), iron uptake proteins (WO 01/72337), toxin A or B of C. difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Uchida Cameron D M, R J Collier. 1987. J. Bacteriol. 169:4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in Int'l Patent Application No. WO 2004/083251), E. coli LT, E. coli ST, and exotoxin A from Pseudomonas aeruginosa.

In some embodiments, the carrier protein is selected from any one of, for example, CRM197, diphtheria toxin fragment B (DTFB), DTFB C8, Diphtheria toxoid (DT), tetanus toxoid (TT), fragment C of TT, pertussis toxoid, cholera toxoid, or exotoxin A from Pseudomonas aeruginosa; detoxified Exotoxin A of P. aeruginosa (EPA), maltose binding protein (MBP), flagellin, detoxified hemolysin A of S. aureus, clumping factor A, clumping factor B, Cholera toxin B subunit (CTB), Streptococcus pneumoniae Pneumolysin and detoxified variants thereof, C. jejuni AcrA, C. jejuni natural glycoproteins and Streptococcal C5a peptidase (SCP). In one embodiment, the carrier protein is detoxified Pseudomonas exotoxin (EPA). In another embodiment, the carrier protein is not detoxified Pseudomonas exotoxin (EPA). In one embodiment, the carrier protein is flagellin. In another embodiment, the carrier protein is not flagellin.

In a preferred embodiment, the carrier protein of the glycoconjugates is independently selected from the group consisting of TT, DT, DT mutants (such as CRM197), H. influenzae protein D, PhtX, PhtD, PhtDE fusions (particularly those described in WO 01/98334 and WO 03/54007), detoxified pneumolysin, PorB, N19 protein, PspA, OMPC, toxin A or B of C. difficile and PsaA. In an embodiment, the carrier protein of the glycoconjugates of the invention is DT (Diphtheria toxoid). In another embodiment, the carrier protein of the glycoconjugates of the invention is TT (tetanus toxoid). In another embodiment, the carrier protein of the glycoconjugates of the invention is PD (Haemophilus influenzae protein D—see, e.g., EP 0 594 610 B).

In a preferred embodiment, the capsular saccharides of the invention are conjugated to CRM197 protein. The CRM197 protein is a nontoxic form of diphtheria toxin but is immunologically indistinguishable from the diphtheria toxin. CRM197 is produced by C. diphtheriae infected by the nontoxigenic phage β197tox-created by nitrosoguanidine mutagenesis of the toxigenic corynephage beta (Uchida, T. et al. 1971, Nature New Biology 233:8-11). The CRM197 protein has the same molecular weight as the diphtheria toxin but differs therefrom by a single base change (guanine to adenine) in the structural gene. This single base change causes an amino acid substitution glutamic acid for glycine) in the mature protein and eliminates the toxic properties of diphtheria toxin. The CRM197 protein is a safe and effective T-cell dependent carrier for saccharides. Further details about CRM197 and production thereof can be found e.g. in U.S. Pat. No. 5,614,382

Accordingly, in frequent embodiments, the glycoconjugates of the invention comprise CRM197 as the carrier protein, wherein the capsular polysaccharide is covalently linked to CRM197.

In a further embodiment, the carrier protein of the glycoconjugates is SCP (Streptococcal C5a Peptidase). All human isolates of β-hemolytic streptococci produce a highly conserved cell-wall protein SCP (Streptococcal C5a Peptidase) that specifically inactivates C5a. The scp genes encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

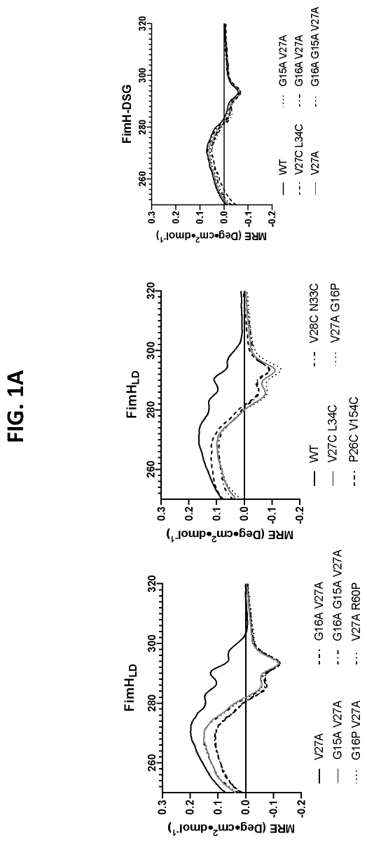

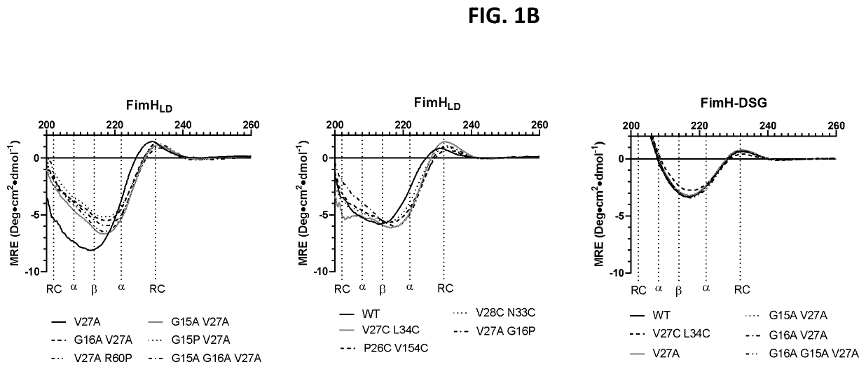

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is split in two part (protease part 1 commonly residues 89-333/334 and protease domain part 2 and commonly residues 467/468-583/584), the protease-associated domain (PA domain) (commonly residues 333/334-467/468), three fibronectin type Ill (Fn) domains (Fn1, commonly residues 583/584-712/713; Fn2, commonly residues 712/713-928/929/930; commonly Fn3, residues 929/930-1029/1030/1031) and a cell wall anchor domain (commonly residues 1029/1030/1031 to the C-terminus).

In an embodiment, the carrier protein of the glycoconjugates of the invention is an SCP from GBS (SCPB). An example of SCPB is provided at SEQ. ID. NO: 3 of WO97/26008. See also SEQ ID NO: 3 of WO00/34487.

In another embodiment, the carrier protein of the glycoconjugate of the invention is an SCP from GAS (SCPA). Examples of SCPA can be found at SEQ. ID. NO:1 and SEQ. ID. NO:2 of WO97/26008. See also SEQ ID Nos: 1, 2 and 23 of WO00/34487.

In a further embodiment, the carrier protein of the glyconjugate of the invention is an SCP as set forth in SEQ ID NO: 150 or 151 of WO2014/136064.

### B. Adjuvants

In some aspects, the immunogenic compositions disclosed herein may further comprise at least one, two or three adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (TWEEN 80), 0.5% w/v sorbitan trioleate (SPAN 85), water-in-oil emulsions such as MONTANIDE, and poly(D,L-lactide-co-glycolide) (PLG) microparticles or nanoparticles.

In one aspect, the immunogenic compositions disclosed herein comprise aluminum salts (alum) as adjuvant (e.g., aluminum phosphate, aluminum sulfate or aluminum hydroxide). In a further aspect, the immunogenic compositions disclosed herein comprise aluminum phosphate or aluminum hydroxide as adjuvant. In one aspect, the immunogenic compositions disclosed herein comprise from 0.1 mg/mL to 1 mg/mL or from 0.2 mg/mL to 0.3 mg/mL of elemental aluminum in the form of aluminum phosphate. In one aspect, the immunogenic compositions disclosed herein comprise about 0.25 mg/mL of elemental aluminum in the form of aluminum phosphate.

Examples of known suitable immune modulatory type adjuvants that can be used in humans include, but are not limited to, saponin extracts from the bark of the Aquilla tree (QS21, Quil A), TLR4 agonists such as MPL (Monophosphoryl Lipid A), 3DMPL (3-O-deacylated MPL) or GLA-AQ, LT/CT mutants, cytokines such as the various interleukins (e.g., IL-2, IL-12) or GM-CSF, AS01, and the like.

Examples of known suitable immune modulatory type adjuvants with both delivery and immune modulatory features that can be used in humans include, but are not limited to, ISCOMS (see, e.g., Sjölander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

For veterinary applications including but not limited to animal experimentation, one can use Complete Freund's Adjuvant (CFA), Freund's Incomplete Adjuvant (IFA), Emulsigen, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/TWEEN 80 emulsion.

Further exemplary adjuvants to enhance effectiveness of the immunogenic compositions disclosed herein include, but are not limited to (1) oil-in-water emulsion formulations (with or without other specific immunostimulating agents such as muramyl peptides (see below) or bacterial cell wall components), such as for example (a) SAF, containing 10% Squalane, 0.4% TWEEN 80, 5% pluronic-blocked polymer L121, and thr-MDP either microfluidized into a submicron emulsion or vortexed to generate a larger particle size emulsion, and (b) RIBI adjuvant system (RAS), (Ribi Immunochem, Hamilton, Mont.) containing 2% Squalene, 0.2% TWEEN 80, and one or more bacterial cell wall components such as monophosphorylipid A (MPL), trehalose dimycolate (TDM), and cell wall skeleton (CWS), preferably MPL+CWS (DETOX); (2) saponin adjuvants, such as QS21, STIMULON (Cambridge Bioscience, Worcester, Mass.), ABISCO (Isconova, Sweden), or ISCOMATRIX (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB2220211, EP0689454) (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+1M2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In another embodiment, the adjuvant is a liposomal Quillaja saponaria-21 (QS21) formulation comprising 5.1 mg/mL QS-21, 5 mM Succinate, 60 mM NaCl, 0.1% PS80, pH 5.6. In a further embodiment, the adjuvant is a liposomal monophosphoryl Lipid A (MPLA, Synthetic, PHAD®, Avanti) formulation comprising 15 mM phosphate buffer, pH 6.1, 4 mg/mL 1,2-dioleoyl-sn-glycero-3-phosphocholine (DOPC), 1 mg/mL Cholesterol, 0.2 mg/mL MPLA (Lot 00714551-0018-2XLipoMPL), having a liposomal particle size of 71 nm determined by dynamic light scattering. In a still further embodiment, the adjuvant is a liposomal MPLA/QS21 formulation comprising 15 mM phosphate buffer, pH 6.1, 4 mg/mL DOPC, 1 mg/mL Cholesterol, 0.2 mg/mL MPLA, and 0.2 mg/mL QS-21 (Lot 00714551-0018-2XlipoMQ), having a particle size of 75 nm for MPLA-QS21 liposomes determined by dynamic light scattering.

In a further aspect of the present disclosure, the immunogenic compositions as disclosed herein comprise a CpG oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

Different classes of CpG immunostimulatory oligonucleotides have been identified. These are referred to as A, B, C and P class, and are described in greater detail at page 3, line 22, to page 12, line 36, of WO 2010/125480. Methods of the present disclosure include the use of these different classes of CpG immunostimulatory oligonucleotides.

### V. Methods of Purification and Production

In one aspect, the present disclosure relates to methods of producing a FimH mutated polypeptide. Such methods can include, for example, culturing a mammalian cell under a suitable condition, thereby expressing the Fim H mutant polypeptide. The method may further include harvesting the polypeptide from the culture. The process may further include purifying the polypeptide.

In some aspects, the method produces the FimH mutant polypeptide at a yield of about 0.1 g/L to 0.5 g/L. In some aspects, the yield of the FimH mutated polypeptide is at least about 1 mg/L, at least about 2 mg/L, at least about 3 mg/L, at least about 4 mg/L, at least about 5 mg/L, at least about 6 mg/L, at least about 7 mg/L, at least about 8 mg/L, at least about 9 mg/L, at least about 10 mg/L, at least about 11 mg/L, at least about 12 mg/L, at least about 13 mg/L, at least about 14 mg/L, at least about 15 mg/L, at least about 16 mg/L, at least about 17 mg/L, at least about 18 mg/L, at least about 19 mg/L, at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 100 mg/L.

In some aspects, a cell culture suitable for the present disclosure is a fed-batch culture. The term “fed-batch culture” as used herein refers to a method of culturing cells in which additional components are provided to the culture at a time or times subsequent to the beginning of the culture process. Such provided components typically comprise nutritional components for the cells which have been depleted during the culturing process. A fed-batch culture is typically stopped at some point and the cells and/or components in the medium are harvested and optionally isolated. In some aspects, the fed-batch culture comprises a base medium supplemented with feed media.

In some aspects, the cells may be grown in batch or fed-batch cultures, where the culture is terminated after sufficient expression of the polypeptide, after which the expressed polypeptide is harvested and optionally isolated. In some aspects, the cells may be grown in perfusion cultures, where the culture is not terminated and new nutrients and other components are periodically or continuously added to the culture, during which the expressed polypeptide is periodically or continuously harvested.

In some aspects, the expression level or activity of the FimH mutant polypeptide is increased by at least 2-fold, at least 3 fold, at least 5 fold, at least 10 fold, at least 20 fold, at least 30 fold, at least 40 fold, at least 50 fold, at least 60 fold, at least 70 fold, at least 75 fold, at least 80 fold, at least 90 fold, at least 100 fold, as compared to expression of the FimH mutant polypeptide in a bacterial cell, such as, for example, an E. coli host cell.

In some aspects, the cells may be grown in small scale reaction vessels to form a cell culture ranging in volume from a few milliliters to several liters. In some aspects, the cells may be grown in large scale commercial bioreactors to form a cell culture, wherein the cell culture may range in volume from approximately at least 1 liter to 10, 100, 250, 500, 1,000, 2,500, 5,000, 8,000, 10,000, 12,000 liters or more, or any volume in between. In some embodiments, the cell culture size may range from 10 L to 5000 L, from 10 L to 10,000 L, from 10 L to 20,000 L, from 10 L to 50,000 L, from 40 L to 50,000 L, from 100 L to 50,000 L, from 500 L to 50,000 L, from 1000 L to 50,000 L, from 2000 L to 50,000 L, from 3000 L to 50,000 L, from 4000 L to 50,000 L, from 4500 L to 50,000 L, from 1000 L to 10,000 L, from 1000 L to 20,000 L, from 1000 L to 25,000 L, from 1000 L to 30,000 L, from 15 L to 2000 L, from 40 L to 1000 L, from 100 L to 500 L, from 200 L to 400 L, or any integer in between.

The temperature of the cell culture will be selected based primarily on the range of temperatures at which the cell culture remains viable, at which a high level of polypeptide is produced, the temperature at which production or accumulation of metabolic waste products is minimized, and/or any combination of these or other factors deemed important by the practitioner.

As one non-limiting example, CHO cells grow well and produce high levels of protein or polypeptide at approximately 37° C. In general, most mammalian cells grow well and/or can produce high levels or protein or polypeptide within a range of about 25° C. to 42° C., although methods taught by the present disclosure are not limited to these temperatures. Certain mammalian cells grow well and/or can produce high levels of protein or polypeptide within the range of about 35° C. to 40° C. In certain aspects, the cell culture is grown at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. at one or more times during the cell culture process.

The terms “culture” and “cell culture” as used herein refer to a cell population that is suspended in a medium under conditions suitable to survival and/or growth of the cell population. As will be clear to those of ordinary skill in the art, in some aspects, these terms as used herein refer to the combination comprising the cell population and the medium in which the population is suspended. In some aspects, the cells of the cell culture comprise mammalian cells.

In some aspects, cells may be grown in one of a variety of chemically defined media, wherein the components of the media are both known and controlled. In some aspects, cells may be grown in a complex medium, in which not all components of the medium are known and/or controlled. Chemically defined growth media for mammalian cell culture have been extensively developed and published over the last several decades. All components of defined media are well characterized, and so defined media do not contain complex additives such as serum or hydrolysates. Early media formulations were developed to permit cell growth and maintenance of viability with little or no concern for protein production. More recently, media formulations have been developed with the express purpose of supporting highly productive recombinant protein producing cell cultures. Such media are preferred for use in the method of the invention. Such media generally comprises high amounts of nutrients and in particular of amino acids to support the growth and/or the maintenance of cells at high density. If necessary, these media can be modified by the skilled person for use in the method of the invention. For example, the skilled person may decrease the amount of phenylalanine, tyrosine, tryptophan and/or methionine in these media for their use as base media or feed media in a method as disclosed herein.

Not all components of complex media are well characterized, and so complex media may contain additives such as simple and/or complex carbon sources, simple and/or complex nitrogen sources, and serum, among other things. In some aspects, complex media suitable for the present invention contains additives such as hydrolysates in addition to other components of defined medium as described herein. In some aspects, defined media typically includes roughly fifty chemical entities at known concentrations in water. Most of them also contain one or more well-characterized proteins such as insulin, IGF-1, transferrin or BSA, but others require no protein components and so are referred to as protein-free defined media. Typical chemical components of the media fall into five broad categories: amino acids, vitamins, inorganic salts, trace elements, and a miscellaneous category that defies neat categorization.

Cell culture medium may be optionally supplemented with supplementary components. The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In some aspects, supplementary components may be added to the initial cell culture. In some aspects, supplementary components may be added after the beginning of the cell culture. Typically, trace elements refer to a variety of inorganic salts included at micromolar or lower levels. For example, commonly included trace elements are zinc, selenium, copper, and others. In some aspects, iron (ferrous or ferric salts) can be included as a trace element in the initial cell culture medium at micromolar concentrations. Manganese is also frequently included among the trace elements as a divalent cation (MnCl2 or MnSO4) in a range of nanomolar to micromolar concentrations. Numerous less common trace elements are usually added at nanomolar concentrations.

In some aspects, the medium used in the method of the invention is a medium suitable for supporting high cell density, such as for example 1×106 cells/mL, 5×106 cells/mL, 1×107 cells/mL, 5×107 cells/mL, 1×108 cells/mL or 5×108 cells/mL, in a cell culture. In some aspects, the cell culture is a mammalian cell fed-batch culture, preferably a CHO cells fed-batch culture.

In some aspects, the cell culture medium comprises phenylalanine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tyrosine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tryptophan at a concentration below 2 mM, below 1 mM, between 0.1 and 2 mM, between 0.1 to 1 mM, between 0.5 and 1.5 mM or between 0.5 to 1 mM. In some aspects, the cell culture medium comprises methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises leucine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises serine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises threonine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises two of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine and tyrosine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine and tryptophan at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine and methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tyrosine and tryptophan at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tyrosine and methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tryptophan and methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises three of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine, tyrosine and tryptophan at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine, tyrosine and methionine at a concentration below 2 mM, below 1 mM, between 0.1 and 2 mM, between 0.1 to 1 mM, between 0.5 and 1.5 mM or between 0.5 to 1 mM.

In some aspects, the cell culture medium comprises phenylalanine, tryptophan and methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises tyrosine, tryptophan and methionine at a concentration below 2 mM, below 1 mM, between 0.1 and 2 mM, between 0.1 to 1 mM, between 0.5 and 1.5 mM or between 0.5 to 1 mM.

In some aspects, the cell culture medium comprises four of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM.

In some aspects, the cell culture medium comprises phenylalanine, tyrosine, tryptophan and methionine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises five of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises six of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises seven of phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium comprises phenylalanine, tyrosine, tryptophan, methionine, leucine, serine, threonine and glycine at a concentration below 2 mM, below 1 mM, between 0.1 and 2 mM, between 0.1 to 1 mM, between 0.5 and 1.5 mM or between 0.5 to 1 mM. In some aspects, the cell culture medium further comprises at least 1, 2, 3, 4, 5, 6, 7, 8, 9, 10, 11, 12 or 13 of glycine, valine, leucine, isoleucine, proline, serine, threonine, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium further comprises at least 5 of glycine, valine, leucine, isoleucine, proline, serine, threonine, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium further comprises glycine, valine, leucine, isoleucine, proline, serine, threonine, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium further comprises at least 1, 2, 3, 4, 5, 6, 7, 8, or 9 of valine, isoleucine, proline, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium further comprises at least 5 of valine, isoleucine, proline, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium further comprises valine, isoleucine, proline, lysine, arginine, histidine, aspartate, glutamate and asparagine at a concentration above 2 mM, 3 mM, 4 mM, 5 mM, 10 mM, 15 mM, preferably 2 mM. In some aspects, the cell culture medium comprises serine at a concentration above 3 mM, 5 mM, 7 mM, 10 mM, 15 mM or 20 mM, preferably 10 mM. In some aspects, the cell culture medium comprises valine at a concentration above 3 mM, 5 mM, 7 mM, 10 mM, 15 mM or 20 mM, preferably 10 mM. In some aspects, the cell culture medium comprises cysteine at a concentration above 3 mM, 5 mM, 7 mM, 10 mM, 15 mM or 20 mM, preferably 10 mM. In some aspects, the cell culture medium comprises isoleucine at a concentration above 3 mM, 5 mM, 7 mM, 10 mM, 15 mM or 20 mM, preferably 10 mM.

In some aspects, the cell culture medium comprises leucine at a concentration above 3 mM, 5 mM, 7 mM, 10 mM, 15 mM or 20 mM, preferably 10 mM. In some aspects, the above cell culture medium is for use in a method as disclosed herein. In some aspects, the above cell culture medium is used in a method as disclosed herein as a base media. In some aspects, the above cell culture medium is used a method as disclosed herein as a feed media.

The methods of the present disclosure may be used with any cell culture method that is amenable to the desired process (e.g., production of a recombinant protein). As a non-limiting example, cells may be grown in batch or fed-batch cultures, where the culture is terminated after sufficient expression of the recombinant protein (e.g., antibody), after which the expressed protein is harvested. Alternatively, as another non-limiting example, cells may be grown in batch-refeed, where the culture is not terminated and new nutrients and other components are periodically or continuously added to the culture, during which the expressed recombinant protein is harvested periodically or continuously. Other suitable methods (e.g., spin-tube cultures) are known in the art and can be used to practice the present invention.

Cells may be grown in any convenient volume chosen by the practitioner. For example, cells may be grown in small scale reaction vessels ranging in volume from a few milliliters to several liters. Alternatively, cells may be grown in large scale commercial Bioreactors ranging in volume from approximately at least 1 liter to 10, 50, 100, 250, 500, 1000, 2500, 5000, 8000, 10,000, 12,000, 15000, 20000 or 25000 liters or more, or any volume in between.

The temperature of a cell culture will be selected based primarily on the range of temperatures at which the cell culture remains viable and the range in which a high level of desired product (e.g., a recombinant protein) is produced. In general, most mammalian cells grow well and can produce desired products (e.g., recombinant proteins) within a range of about 25° C. to 42° C., although methods taught by the present disclosure are not limited to these temperatures. Certain mammalian cells grow well and can produce desired products (e.g., recombinant proteins or antibodies) within the range of about 35° C. to 40° C. In certain aspects, a cell culture is grown at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. at one or more times during the cell culture process. Those of ordinary skill in the art will be able to select appropriate temperature or temperatures in which to grow cells, depending on the particular needs of the cells and the particular production requirements of the practitioner. The cells may be grown for any amount of time, depending on the needs of the practitioner and the requirement of the cells themselves. In some embodiment, the cells are grown at 37° C. In some aspects, the cells are grown at 36.5° C.

In some aspects, the cells may be grown during the initial growth phase (or growth phase) for a greater or lesser amount of time, depending on the needs of the practitioner and the requirement of the cells themselves. In some aspects, the cells are grown for a period of time sufficient to achieve a predefined cell density. In some aspects, the cells are grown for a period of time sufficient to achieve a cell density that is a given percentage of the maximal cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal cell density. In some aspects, the cells are grown until the cell density does not increase by more than 15%, 14%, 13%, 12%, 11%, 10%, 9%, 8%, 7%, 6%, 5%, 4%, 3%, 2% or 1% per day of culture. In some aspects, the cells are grown until the cell density does not increase by more than 5% per day of culture.

In some aspects the cells are allowed to grow for a defined period of time. For example, depending on the starting concentration of the cell culture, the temperature at which the cells are grown, and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days, preferably for 4 to 10 days. In some cases, the cells may be allowed to grow for a month or more. The practitioner of the present invention will be able to choose the duration of the initial growth phase depending on protein production requirements and the needs of the cells themselves.

The cell culture may be agitated or shaken during the initial culture phase in order to increase oxygenation and dispersion of nutrients to the cells. In accordance with the present invention, one of ordinary skill in the art will understand that it can be beneficial to control or regulate certain internal conditions of the bioreactor during the initial growth phase, including but not limited to pH, temperature, oxygenation, etc.

At the end of the initial growth phase, at least one of the culture conditions may be shifted so that a second set of culture conditions is applied and a metabolic shift occurs in the culture. A metabolic shift can be accomplished by, e.g., a change in the temperature, pH, osmolality or chemical inductant level of the cell culture. In one non-limiting embodiment, the culture conditions are shifted by shifting the temperature of the culture. However, as is known in the art, shifting temperature is not the only mechanism through which an appropriate metabolic shift can be achieved. For example, such a metabolic shift can also be achieved by shifting other culture conditions including, but not limited to, pH, osmolality, and sodium butyrate levels. The timing of the culture shift will be determined by the practitioner of the present invention, based on protein production requirements or the needs of the cells themselves.

When shifting the temperature of the culture, the temperature shift may be relatively gradual. For example, it may take several hours or days to complete the temperature change. Alternatively, the temperature shift may be relatively abrupt. For example, the temperature change may be complete in less than several hours. Given the appropriate production and control equipment, such as is standard in the commercial large-scale production of polypeptides or proteins, the temperature change may even be complete within less than an hour.

In some aspects, once the conditions of the cell culture have been shifted as discussed above, the cell culture is maintained for a subsequent production phase under a second set of culture conditions conducive to the survival and viability of the cell culture and appropriate for expression of the desired polypeptide or protein at commercially adequate levels.

As discussed above, the culture may be shifted by shifting one or more of a number of culture conditions including, but not limited to, temperature, pH, osmolality, and sodium butyrate levels. In some aspects, the temperature of the culture is shifted. According to this embodiment, during the subsequent production phase, the culture is maintained at a temperature or temperature range that is lower than the temperature or temperature range of the initial growth phase. As discussed above, multiple discrete temperature shifts may be employed to increase cell density or viability or to increase expression of the recombinant protein.

The term “titer” as used herein refers, for example, to the total amount of recombinantly expressed protein produced by a cell culture in a given amount of medium volume. Titer is typically expressed in units of grams of protein per liter of medium.

In some aspects, cell growth is increased by at least 5%, 10%, 15%, 20% or 25% as compared to the control culture. In some aspects, cell growth is increased by at least 10% as compared to the control culture. In some aspects, cell growth is increased by at least 20% as compared to the control culture.

In some aspects, the productivity is determined by titer and/or volumetric productivity. In some aspects, the productivity is determined by titer. In some aspects, the productivity is increased by at least 5%, 10%, 15%, 20% or 25% as compared to the control culture. In some aspects, the productivity is increased by at least 10% as compared to a control culture. In some aspects, the productivity is increased by at least 20% as compared to a control culture.

### Purification

In some aspects, the method for producing a FimH mutant polypeptide includes isolating and/or purifying the polypeptide. In some aspects, the expressed polypeptide is secreted into the medium and thus cells and other solids may be removed by centrifugation and/or filtration. In a preferred embodiment, the polypeptide or a fragment thereof is soluble.

The FimH mutated polypeptide produced in accordance with the methods described herein may be harvested from host cells and isolated using any suitable method, and are generally known in the art (e.g. Ruiz-Arguello et al., J. Gen. Virol., 85:3677-3687 (2004)). Suitable methods for purifying the polypeptide include precipitation and various types of chromatography, such as hydrophobic interaction, ion exchange, affinity, chelation, and size exclusion, all of which are known in the art. Suitable purification schemes may include two or more of these or other suitable methods. In some aspects, one or more of the polypeptides may include a “tag” that facilitates purification or detection. Examples include, e.g., His tag (binds to metal ion, e.g. hexahistidine), an antibody, maltose-binding protein (MBP) (binds to amylose), glutathione-S-transferase (GST) (binds to glutathione), FLAG tag (binds to anti-flag antibody), Strep tag (binds to streptavidin or a derivative thereof). Such tagged polypeptides may conveniently be isolated, for example from conditioned media, by chelating chromatography or affinity chromatography. Optionally, the tag sequence may be cleaved post-purification. In one aspect, the FimH mutant polypeptide does not include a purification tag.

In one aspect, the FimH mutant polypeptides can be isolated by first obtaining the cell culture supernatant, and then subjecting the supernatant to both ultrafiltration and diafiltration methods. Such filtration methods are known to those of skilled in the art. Following ultrafiltration and diafiltration, the resulting cell-free solution is then subjected to a chromatography step, such as Ni-NTA chromatography using, for example, nickel affinity resin. This step can then be followed by dialysis, which can then be followed by cation exchange chromatography, such as with a SP column. Use of acidic pH (e.g. less than about 6.0, less than about 5.5, less than about 5.0, less than about 4.5, about 4.4, about 4.3, about 4.2, about 4.1 or about 4.0 or less) during the purification on SP-SEPHAROSE can be desirable under certain conditions.

While specific strains of E. coli may be referenced herein, it should be understood that the polypeptide derived from E. coli or a fragment thereof are not limited to specific strains unless specified.

### VI. Uses of the Compositions

In one aspect, the disclosure provides the use of a FimH mutant polypeptide, nucleic acids encoding such mutant, vectors for expressing such mutant, compositions comprising such mutant or nucleic acids as a medicament, or in the manufacture of a medicament, for eliciting an immune response against E. coli infection or for preventing E. coli infection in a subject.

In other aspects, the present disclosure provides a method of eliciting an immune response against E. coli in a subject, such as a human, comprising administering to the subject an effective amount of a FimH mutant polypeptide, a nucleic acid molecule encoding a FimH mutant polypeptide, or a composition comprising a FimH mutant polypeptide or nucleic acid molecule. The present disclosure also provides a method of preventing E. coli infection in a subject, comprising administering to the subject an effective amount of a pharmaceutical composition, such as a vaccine, comprising a FimH mutant polypeptide, a nucleic acid encoding a FimH mutant polypeptide, or a vector expressing a FimH mutant polypeptide. In some particular aspects, the pharmaceutical composition comprises a FimH mutant polypeptide as disclosed herein. In some aspects of the methods provided herein above, the subject is a human.

In other aspects, the present disclosure provides a method for inducing an immune response in a subject against extra-intestinal pathogenic E. coli, or inducing the production of opsonophagocytic and/or neutralizing antibodies in a subject that are specific to extra-intestinal pathogenic E. coli, wherein the method comprises administering to the subject an effective amount of any of the compositions describe herein, such as those comprising a FimH mutant polypeptide as described herein. In a further aspect of such methods, the subject is at risk of developing a urinary tract infection, and/or at risk of developing bacteremia, and/or at risk of developing sepsis.

In a further aspect, the present disclosure provides a method of eliciting an immune response against E. coli in a mammal, comprising administering to the mammal an effective amount of any of the compositions described herein. For example, in one aspect the immune response comprises opsonophagocytic and/or neutralizing antibodies against E. coli. In a further aspect, the immune response protects the mammal from an E. coli infection.

In a further aspect the present disclosure provides a method of preventing, treating or ameliorating a bacterial infection, disease or condition in a subject, comprising administering to the subject an immunologically effective amount of any of the compositions described herein.

In the methods of the present disclosure, the composition may be administered to the subject with or without administration of an adjuvant. The effective amount administered to the subject is an amount that is sufficient to elicit an immune response against an E. coli antigen, such as a FimH protein, in the subject. Subjects that can be selected for treatment include those that are at risk for developing an E. coli infection, such as those at risk of developing a urinary tract infection, and/or at risk of developing bacteremia, and/or at risk of developing sepsis, because of exposure or the possibility of exposure to E. coli.

“As used herein, “subject” means a mammal, preferably a human. In one embodiment, the subject is at risk of any one of the conditions selected from the group consisting of urinary tract infection, cholecystitis, cholangitis, diarrhea, hemolytic uremic syndrome, neonatal meningitis, urosepsis, intra-abdominal infection, meningitis, complicated pneumonia, wound infection, post-prostate biopsy-related infection, neonatal/infant sepsis, neutropenic fever, and other blood stream infection; pneumonia, bacteremia, and sepsis.

Administration of the compositions provided by the present disclosure, such as pharmaceutical compositions, can be carried out using standard routes of administration. Non-limiting embodiments include parenteral administration, such as intradermal, intramuscular, subcutaneous, transcutaneous, mucosal, or oral administration.

The total dose of the composition provided to a subject during one administration can be varied as is known to the skilled practitioner.

It is also possible to provide one or more booster administrations of one or more of the immunogenic compositions. If a boosting vaccination is performed, typically, such a boosting vaccination will be administered to the same subject at a moment between one week and 10 years, preferably between two weeks and six months, after administering the composition to the subject for the first time (which is in such cases referred to as “priming vaccination”). In alternative boosting regimens, it is also possible to administer different vectors, e.g., one or more adenovirus, or other vectors such as modified vaccinia virus of Ankara (MVA), or DNA, or protein, to the subject after the priming vaccination. The immunogenic compositions provided by the present disclosure may be used together with one or more other immunogenic compositions.

### Dosages of the Compositions

Dosage regimens may be adjusted to provide the optimum desired response. For example, a single dose of a mutated FimH polypeptide may be administered, several divided doses may be administered over time, or the dose may be proportionally reduced or increased as indicated by the exigencies of the situation. It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In some aspects, the amount of the FimH mutant polypeptide in the composition, may range from about 10 μg to about 300 μg of each protein antigen. In some aspects, the amount of the FimH mutant polypeptide in the composition may range from about 20 μg to about 200 μg of each protein antigen.

The amount of glycoconjugate(s) in each dose is selected as an amount which induces an immunoprotective response without significant, adverse side effects in typical vaccines. Such amount will vary depending upon which specific immunogen is employed and how it is presented. The amount of a particular glycoconjugate in an immunogenic composition can be calculated based on total polysaccharide for that conjugate (conjugated and non-conjugated). For example, a glycoconjugate with 20% free polysaccharide will have about 80 g of conjugated polysaccharide and about 20 g of non-conjugated polysaccharide in a 100 g polysaccharide dose. The amount of glycoconjugate can vary depending upon the E. coli serotype. The saccharide concentration can be determined by the uronic acid assay.

The “immunogenic amount” of the different polysaccharide components in the immunogenic composition, may diverge and each may comprise about 1.0 g, about 2.0 g, about 3.0 g, about 4.0 g, about 5.0 g, about 6.0 g, about 7.0 g, about 8.0 g, about 9.0 g, about 10.0 g, about 15.0 g, about 20.0 g, about 30.0 g, about 40.0 pg, about 50.0 pg, about 60.0 pg, about 70.0 pg, about 80.0 pg, about 90.0 pg, or about 100.0 g of any particular polysaccharide antigen. Generally, each dose will comprise 0.1 g to 100 g of polysaccharide for a given serotype, particularly 0.5 g to 20 g, more particularly 1 g to 10 g, and even more particularly 2 g to 5 g. Any whole number integer within any of the above ranges is contemplated as an embodiment of the disclosure. In one embodiment, each dose will comprise 1 g, 2 g, 3 g, 4 g, 5 g, 6 g, 7 g, 8 g, 9 g, 10 g, 15 g or 20 g of polysaccharide for a given serotype.

VII. Combination with a Saccharide and/or Polypeptide or Fragment Thereof Derived from Klebsiella pneumoniae

Klebsiella pneumoniae (K. pneumoniae) is a Gram-negative pathogen, known to cause urinary tract infections, bacteremia, and sepsis. Multidrug-resistant K. pneumoniae infections are an increasing cause of mortality in vulnerable populations at risk. The O-antigen serotypes are highly prevalent among strains causing invasive disease globally and derived O-antigen glycoconjugates are attractive as vaccine antigens.

In one aspect, any of the compositions disclosed herein may further comprise at least one saccharide that is, or is derived from, at least one K. pneumoniae serotype selected from O1 (and d-Gal-III variants), O2 (and d-Gal-III variants), O2ac, O3, O4, O5, O7, O8, and O12. In a preferred embodiment, any of the compositions disclosed herein may further comprise a polypeptide derived from K. pneumoniae selected from a polypeptide derived from K. pneumoniae Type I fimbrial protein or an immunogenic fragment thereof; or a polypeptide derived from K. pneumoniae Type Ill fimbrial protein or an immunogenic fragment thereof; or a combination thereof.

As is known in the art, K. pneumoniae O1 and O2 O-antigens and their corresponding v1 and v2 subtypes are polymeric galactans that differ in the structures of their repeat units. K. pneumoniae O1 and O2 antigens contain homopolymer galactose units (or galactans). K. pneumoniae O1 and O2 antigens each contain D-galactan I units (sometimes referred to as the O2a repeat unit), but O1 antigens differ in that O1 antigens have a D-galactan II cap structure. D-galactan III (d-Gal-III) is a variant of D-galactan I. Structures of the base galactans I and Ill that define the two distinct serotype O2 subtypes, O2v1 and O2v2; and structures of the derived chimeras resulting from capping by galactan II which yields subtypes O1v1 and O1v2, are shown in Kelly S D, et al. J Biol Chem 2019; 294:10863-76; and Clarke B R, et al. J Biol Chem 2018; 293:4666-79.

In some embodiments, the saccharide derived from K. pneumoniae O1 includes a repeat unit of [→3)-β-D-Galf-(1→3)-α-D-Galp-(1→]. In some embodiments, the saccharide derived from K. pneumoniae O1 includes a repeat unit of [→3)-α-D-Galp-(1→3)-β-D-Galp-(1→]. In some embodiments, the saccharide derived from K. pneumoniae O1 includes a repeat unit of [→3)-β-D-Galf-(1→3)-α-D-Galp-(1→], and a repeat unit of [→3)-α-D-Galp-(1→3)-β-D-Galp-(1→]. In some embodiments, the saccharide derived from K. pneumoniae O1 includes a repeat unit of →3)-β-D-Galf-(1→3)-[α-D-Galp-(1→4)]-α-D-Galp-(1→] (referred to as the D-Gal-III repeat unit). (Kol O., et al. (1992) Carbohydr. Res. 236, 339-344; Whitfield C., et al. (1991) J. Bacteriol. 173, 1420-1431).

In some embodiments, the saccharide derived from K. pneumoniae O2 includes a repeat unit of [→3)-α-D-Galp-(1→3)-β-D-Galf-(1→](which may be an element of K. pneumoniae serotype O2a antigen). In some embodiments, the saccharide derived from K. pneumoniae O2 includes a repeat unit of [→3)-β-D-GlcpNAc-(1→5)-β-D-Galf-(1→](which may be an element of K. pneumoniae serotype O2c antigen). In some embodiments, the saccharide derived from K. pneumoniae O2 includes a modification of the O2a repeat unit by side chain addition of (1→4)-linked Galp residues (which may be an element of the K. pneumoniae O2afg antigen). In some embodiments, the saccharide derived from K. pneumoniae O2 includes a modification of the O2a repeat unit by side chain addition of (1→2)-linked Galp residues (which may be an element of the K. pneumoniae O2aeh antigen). (Whitfield C., et al. (1992) J. Bacteriol. 174, 4913-4919).

Without being bound by mechanism or theory, O-antigen polysaccharide structure of K. pneumoniae serotypes O3 and O5 are disclosed in the art to be identical to those of E. coli serotypes O9a (Formula O9a) and O8 (Formula O8), respectively.

In some embodiments, the saccharide derived from K. pneumoniae O4 includes a repeat unit of [→4)-α-D-Galp-(1→2)-β-D-Ribf-(1→)]. In some embodiments, the saccharide derived from K. pneumoniae O7 includes a repeat unit of [→2-α-L-Rhap-(1→2)-β-D-Ribf-(1→3)-α-L-Rhap-(1→3)-α-L-Rhap-(1→]. In some embodiments, the saccharide derived from K. pneumoniae O8 serotype includes the same repeat-unit structure as K. pneumoniae O2a, but is nonstoichiometrically O-acetylated. In some embodiments, the saccharide derived from K. pneumoniae O12 serotype includes a repeat unit of [α-Rhap-(1 →3)-β-GlcpNAc]disaccharide repeat unit.

In one aspect, the invention includes a composition including a polypeptide derived from E. coli FimH or a fragment thereof; and at least one saccharide that is, or derived from, at least one K. pneumoniae serotype selected from O1 (and d-Gal-III variants), O2 (and d-Gal-III variants), O2ac, O3, O4, O5, O7, O8, and O12. In some embodiments, the composition includes saccharides from or derived from one or more of serotypes O1, O2, O3, and O5, or a combination thereof. In some embodiments, the composition includes saccharides from or derived from each of serotypes O1, O2, O3, and O5.

In another aspect, the invention includes a composition including at least one saccharide that is, or is derived from, at least one K. pneumoniae serotype selected from O1 (and d-Gal-III variants), O2 (and d-Gal-III variants), O2ac, O3, O4, O5, O7, O8, and O12; and a saccharide derived from an E. coli O-antigen having a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187, wherein n is an integer from 1 to 100. In some embodiments, the composition includes a saccharide from or derived from one or more of K. pneumoniae serotypes O1, O2, O3 and O5, or a combination thereof. In some embodiments, the composition includes a saccharide from or derived from each of K. pneumoniae serotypes O1, O2, O3 and O5. In some embodiments, the composition includes a saccharide derived from an E. coli O-antigen having Formula O9 and does not include a saccharide derived from K. pneumoniae serotype O3. In some embodiments, the composition includes a saccharide derived from an E. coli O-antigen having Formula O8 and does not include a saccharide derived from K. pneumoniae serotype O5.

In another aspect, the invention relates to a composition including a polypeptide derived from E. coli FimH or a fragment thereof; at least one saccharide that is, or derived from, at least one K. pneumoniae serotype selected from O1 (and d-Gal-III variants), O2 (and d-Gal-III variants), O2ac, O3, O4, O5, O7, O8, and O12; and a saccharide having a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187, wherein n is an integer from 1 to 100, preferably from 31 to 90. In some embodiments, the composition includes a saccharide derived from an E. coli O-antigen having Formula O9 and does not include a saccharide derived from K. pneumoniae serotype O3. In some embodiments, the composition includes a saccharide derived from an E. coli O-antigen having Formula O8 and does not include a saccharide derived from K. pneumoniae serotype O5.

In some embodiments, the composition includes at least one saccharide derived from any one K. pneumoniae type selected from the group consisting of O1, O2, O3, and O5.

In some embodiments, the composition includes at least one saccharide derived from K. pneumoniae type O1. In one aspect of this embodiment, the K. pneumoniae O-antigen is selected from subtype v1 (O1v) or subtype v2 (O1v2). In one aspect of this embodiment, the K. pneumoniae O-antigen is selected from subtype v1 (O1v) and subtype v2 (O1v2). In some embodiments, the composition includes at least one saccharide derived from K. pneumoniae type O2. In one aspect of this embodiment, the K. pneumoniae O-antigen is selected from subtype v1 (O2v1) or subtype v2 (O2v2). In one aspect of this embodiment, the K. pneumoniae O-antigen is selected from subtype v1 (O2v1) and subtype v2 (O2v2). In another aspect, the K. pneumoniae O-antigen is selected from the group consisting of: a) serotype O1 subtype v1 (O1v), b) serotype O1 subtype v2 (O1v2), c) serotype O2 subtype v1 (O2v1), and d) serotype O2 subtype v2 (O2v2). In one aspect of this embodiment, the K. pneumoniae O-antigen is subtype v1 (O1v). In one aspect of this embodiment, the K. pneumoniae O-antigen is subtype v2 (O1v2). In one aspect of this embodiment, the K. pneumoniae O-antigen is subtype v1 (O2v1). In one aspect of this embodiment, the K. pneumoniae O-antigen is subtype v2 (O2v2). In another aspect of this embodiment, the composition comprises one, two, three or four K. pneumoniae O-antigen selected from the group consisting of: a) serotype O1 subtype v1 (O1v), b) serotype O1 subtype v2 (O1v2), c) serotype O2 subtype v1 (O2v1), and d) serotype O2 subtype v2 (O2v2). In some embodiments, the composition includes a combination of saccharides derived from K. pneumoniae, wherein a first saccharide is derived from any one of K. pneumoniae types selected from the group consisting of O1, O2, O3, and O5; and a second saccharide is derived from a saccharide is derived from any one of K. pneumoniae types selected from the group consisting of O1 (and d-Gal-III variants), O2 (and d-Gal-III variants), O2ac, O3, O4, O5, O7, O8, and O12. For example, in some embodiments, the composition includes at least one saccharide derived from K. pneumoniae type O1 and at least one saccharide derived from K. pneumoniae type O2. In a preferred embodiment, the saccharide derived from K. pneumoniae is conjugated to a carrier protein; and the saccharide derived from E. coli is conjugated to a carrier protein.

In another aspect, the invention includes a composition including a polypeptide derived from E. coli FimH or a fragment thereof; and at least one saccharide derived from any one K. pneumoniae type selected from the group consisting of O1, O2, O3, and O5.

In another aspect, the invention includes at least one saccharide derived from any one K. pneumoniae type selected from the group consisting of O1, O2, O3, and O5; and at least one saccharide derived from E. coli having a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187. In some embodiments, the composition includes a saccharide derived from an E. coli O-antigen having Formula O9 and does not include a saccharide derived from K. pneumoniae serotype O3. In some embodiments, the composition includes a saccharide derived from an E. coli O-antigen having Formula O8 and does not include a saccharide derived from K. pneumoniae serotype O5.

In some embodiments, the composition includes at least one saccharide derived from K. pneumoniae type O1; and at least one saccharide derived from E. coli having a structure selected from the group consisting of Formula O8 and Formula O9. In another embodiment, the composition includes at least one saccharide derived from K. pneumoniae type O2; and at least one saccharide derived from E. coli having a structure selected from the group consisting of Formula O8 and Formula O9. In another embodiment, the composition includes at least one saccharide derived from K. pneumoniae type O1; at least one saccharide derived from K. pneumoniae type O2; and at least one saccharide derived from E. coli having a structure selected from the group consisting of Formula O8 and Formula O9.

In one embodiment, the invention provides a method of inducing an immune response to K. pneumoniae in a subject comprising administering to the subject an immunologically effective amount of an immunogenic composition comprising at least one glycoconjugate from E. coli serotype O8 or O9, wherein said immunogenic composition does not comprise glycoconjugates from K. pneumoniae serotype O5 or O3. In one aspect, the composition includes a saccharide derived from an E. coli O-antigen having Formula O8 and does not include a saccharide derived from K. pneumoniae serotype O5. In another aspect, the composition includes a saccharide derived from an E. coli O-antigen having Formula O9 and does not include a saccharide derived from K. pneumoniae serotype O3.

In another embodiment, the invention provides a method of inducing an immune response to E. coli in a subject comprising administering to the subject an immunologically effective amount of an immunogenic composition comprising at least one glycoconjugate from K. pneumoniae serotype O5 or O3, or a variant thereof, wherein said immunogenic composition does not comprise glycoconjugates from E. coli serotype O8 or O9. In one aspect, the composition includes a saccharide derived from K. pneumoniae serotype O5 and does not include a saccharide derived from an E. coli O-antigen having Formula O8. In another aspect, the composition includes a saccharide derived from K. pneumoniae serotype O3 and does not include a saccharide derived from an E. coli O-antigen having Formula O9.

In some embodiments, the composition includes at least one saccharide that is, or is derived from, at least one K. pneumoniae serotype selected from O1 (and d-Gal-III variants), O2 (and d-Gal-III variants), O2ac, O3, O4, O5, O7, O8, and O12; at least one saccharide derived from E. coli having a structure selected from the group consisting of Formula O8 and Formula O9. In some embodiments, the composition includes at least one saccharide that is, or derived from, at least one K. pneumoniae serotype selected from O1 (and d-Gal-III variants), O2 (and d-Gal-III variants), O2ac, O3, O4, O5, O7, O8, and O12; at least one saccharide derived from E. coli having a structure selected from the group consisting of Formula O1A, Formula O1B, Formula O2, Formula O6, and Formula O25B.

In some embodiments, the composition further includes a polypeptide derived from K. pneumoniae selected from a polypeptide derived from K. pneumoniae Type I fimbrial protein or an immunogenic fragment thereof; or a polypeptide derived from K. pneumoniae Type Ill fimbrial protein or an immunogenic fragment thereof, or a combination thereof. The sequences of said polypeptides are known in the art.

### VIII. Nanoparticles

In another aspect, disclosed herein is an immunogenic complex that includes 1) a nanostructure; and 2) at least one fimbrial polypeptide antigen or fragment thereof. Preferably, the fimbrial polypeptide or fragment thereof is derived from E. coli fimbrial H (fimH). In a preferred embodiment, the fimbrial polypeptide is selected from any one of the fimbrial polypeptides described above. For example, the fimbrial polypeptide may comprise any one amino acid sequence selected from SEQ ID NOs:1-65.

In some embodiments, the antigen is fused or conjugated to the nanostructure exterior to stimulate development of adaptive immune responses to the displayed epitopes. In some embodiments, the immunogenic complex further includes an adjuvant or other immunomodulatory compounds attached to the exterior and/or encapsulated in the cage interior to help tailor the type of immune response generated for each pathogen.

In some embodiments, the nanostructure includes a single assembly including a plurality of identical first nanostructure-related polypeptides.

In alternative embodiments, the nanostructure includes a plurality assembly, including a plurality of identical first nanostructure-related polypeptides and a plurality of second assemblies, each second assembly comprising a plurality of identical second nanostructure-related polypeptides.

Various nanostructure platforms can be employed in generating the immunogenic compositions described herein. In some embodiments, the nanostructures employed are formed by multiple copies of a single subunit. In some embodiments, the nanostructures employed are formed by multiple copies of multiple different subunits.

The nanostructures are typically ball-like shaped, and/or have rotational symmetry (e.g., with 3-fold and 5-fold axis), e.g., with an icosahedral structure exemplified herein.

In some embodiments, the antigen is presented on self-assembling nanoparticles such as self-assembling nanostructures derived from ferritin (FR), E2p, Qβ, and I3-01. E2p is a redesigned variant of dihydrolipoyl acyltransferase from Bacillus stearothermophilus. I3-01 is an engineered protein that may self-assemble into hyperstable nanoparticles. Sequences of the subunits of these proteins are known in the art. In a first aspect, disclosed herein is a nanostructure-related polypeptide comprising an amino acid sequence that is at least 75% identical over its length, and identical at least at one identified interface position, to the amino acid sequence of a nanostructure-related polypeptide selected from the group consisting of SEQ ID NOS: 66-105. The nanostructure-related polypeptides can be used, for example, to prepare the nanostructures. The nanostructure-related polypeptides were designed for their ability to self-assemble in pairs to form nanostructures, such as icosahedral nanostructures.

In some embodiments, the nanostructure includes (a) a plurality of first assemblies, each first assembly comprising a plurality of identical first nanostructure-related polypeptides, wherein the first nanostructure-related polypeptides comprise the amino acid sequence of a nanostructure-related polypeptide selected from the group consisting of SEQ ID NOS: 66-105; and (b) a plurality of second assemblies, each second assembly comprising a plurality of identical second nanostructure-related polypeptides, wherein the second nanostructure-related polypeptides comprise the amino acid sequence of a nanostructure-related polypeptide selected from the group consisting of SEQ ID NOS: 66-105, and wherein the second nanostructure-related polypeptide differs from the first nanostructure-related polypeptide; wherein the plurality of first assemblies non-covalently interact with the plurality of second assemblies to form a nanostructure;

The nanostructures include symmetrically repeated, non-natural, non-covalent polypeptide-polypeptide interfaces that orient a first assembly and a second assembly into a nanostructure, such as one with an icosahedral symmetry.

SEQ ID NOS: 66-105 provide the amino acid sequence of exemplary nanostructure-related polypeptides. The number of interface residues for the exemplary nanostructure-related polypeptides of SEQ ID NO:66-105 range from 4-13 residues. In various embodiments, the nanostructure-related polypeptides comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given nanostructure-related polypeptide), to the amino acid sequence of a nanostructure-related polypeptide selected from the group consisting of SEQ ID NOS: 66-105. In other embodiments, the nanostructure-related polypeptides comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a nanostructure-related polypeptide selected from the group consisting of SEQ ID NOS: 66-105. In further embodiments, the nanostructure-related polypeptides include a nanostructure-related polypeptide having the amino acid sequence of a nanostructure-related polypeptide selected from the group consisting of SEQ ID NOS: 66-105.

In one non-limiting embodiment, the nanostructure-related polypeptides can be modified to facilitate covalent linkage to a “cargo” of interest. In one non-limiting example, the nanostructure-related polypeptides can be modified, such as by introduction of various cysteine residues at defined positions to facilitate linkage to one or more antigens of interest, such that a nanostructure of the nanostructure-related polypeptides would provide a scaffold to provide a large number of antigens for delivery as a vaccine to generate an improved immune response.

In some embodiments, some or all native cysteine residues that are present in the nanostructure-related polypeptides but not intended to be used for conjugation may be mutated to other amino acids to facilitate conjugation at defined positions. In another non-limiting embodiment, the nanostructure-related polypeptides may be modified by linkage (covalent or non-covalent) with a moiety to help facilitate “endosomal escape.” For applications that involve delivering molecules of interest to a target cell, such as targeted delivery, a critical step can be escape from the endosome—a membrane-bound organelle that is the entry point of the delivery vehicle into the cell. Endosomes mature into lysosomes, which degrade their contents. Thus, if the delivery vehicle does not somehow “escape” from the endosome before it becomes a lysosome, it will be degraded and will not perform its function. There are a variety of lipids or organic polymers that disrupt the endosome and allow escape into the cytosol. Thus, in this embodiment, the nanostructure-related polypeptides can be modified, for example, by introducing cysteine residues that will allow chemical conjugation of such a lipid or organic polymer to the monomer or resulting assembly surface. In another non-limiting example, the nanostructure-related polypeptides can be modified, for example, by introducing cysteine residues that will allow chemical conjugation of fluorophores or other imaging agents that allow visualization of the nanostructures in vitro or in vivo.

Surface amino acid residues on the nanostructure-related polypeptides can be mutated in order to improve the stability or solubility of the protein subunits or the assembled nanostructures. As will be known to one of skill in the art, if the nanostructure-related polypeptide has significant sequence homology to an existing protein family, a multiple sequence alignment of other proteins from that family can be used to guide the selection of amino acid mutations at non-conserved positions that can increase protein stability and/or solubility, a process referred to as consensus protein design (9).

Surface amino acid residues on the nanostructure-related polypeptides can be mutated to positively charged (Arg, Lys) or negatively charged (Asp, Glu) amino acids in order to endow the protein surface with an overall positive or overall negative charge. In one non-limiting embodiment, surface amino acid residues on the nanostructure-related polypeptides can be mutated to endow the interior surface of the self-assembling nanostructure with a high net charge. Such a nanostructure can then be used to package or encapsulate a cargo molecule with the opposite net charge due to the electrostatic interaction between the nanostructure interior surface and the cargo molecule. In one non-limiting embodiment, surface amino acid residues on the nanostructure-related polypeptides can be mutated primarily to Arginine or Lysine residues in order to endow the interior surface of the self-assembling nanostructure with a net positive charge. Solutions containing the nanostructure-related polypeptides can then be mixed in the presence of a nucleic acid cargo molecule such as a dsDNA, ssDNA, dsRNA, ssRNA, cDNA, miRNA., siRNA, shRNA, piRNA, or other nucleic acid in order to encapsulate the nucleic acid inside the self-assembling nanostructure. Such a nanostructure could be used, for example, to protect, deliver, or concentrate nucleic acids.

In one embodiment, the nanostructure has icosahedral symmetry. In this embodiment, the nanostructure may comprise 60 copies of the first nanostructure-related polypeptide and 60 copies of the second nanostructure-related polypeptide. In one such embodiment, the number of identical first nanostructure-related polypeptides in each first assembly is different than the number of identical second nanostructure-related polypeptides in each second assembly. For example, in one embodiment, the nanostructure comprises twelve first assemblies and twenty second assemblies; in this embodiment, each first assembly may; for example, comprise five copies of the identical first nanostructure-related polypeptide, and each second assembly may, for example, comprise three copies of the identical second nanostructure-related polypeptide. In another embodiment, the nanostructure comprises twelve first assemblies and thirty second assemblies; in this embodiment, each first assembly may, for example, comprise five copies of the identical first nanostructure-related polypeptide, and each second assembly may, for example, comprise two copies of the identical second nanostructure-related polypeptide. In a further embodiment, the nanostructure comprises twenty first assemblies and thirty second assemblies; in this embodiment, each first assembly may, for example, comprise three copies of the identical first nanostructure-related polypeptide, and each second assembly may, for example, comprise two copies of the identical second nanostructure-related polypeptide. All of these embodiments are capable of forming synthetic nanomaterials with regular icosahedral symmetry.

### EXAMPLES

In order that the disclosure may be better understood, the following examples are set forth. These examples are for purposes of illustration only and are not to be construed as limiting the scope of the disclosure in any manner.

### Example 1: Antigen Design

Mutations in FimH_{LD} or FimH-DSG were designed to lock the FimH lectin domain in an open conformation with the goal of improving functional immunogenicity. Mutations were of different classes, described in the Tables 2-9 below. The amino acid sequences for the various mutated FimH polypeptides are shown in Table 1.

TABLE 2
Wild type FimH_{LD} constructs including introduction of naturally
occurring amino acid substitution commong among UTI clinical isolates

| SEQ ID NO: | Protein ID | Substitutions |
|---|---|---|
| 1 | FimHLD_WT | WT |
| | FimHLD_V27A | V27A |

TABLE 3
Substitutions in the ligand binding site of FimH_{LD}

| SEQ ID NO: | Protein ID | Substitutions |
|---|---|---|
| 3 | FimHLD_F1I | F1I |
| 4 | FimHLD_F1L | F1L |
| 5 | FimHLD_F1V | F1V |
| 6 | FimHLD_F1M | F1M |
| 7 | FimHLD_F1Y | F1Y |
| 8 | FimHLD_F1W | F1W |
| 9 | FimHLD_Q133K | Q133K |

TABLE 4
Glycine switch mutations in the FimH_{LD}

| SEQ ID NO: | Protein ID | Substitutions |
|---|---|---|
| 10 | FimHLD_G15A | G15A |
| 11 | FimHLD_G15P | G15P |
| 12 | FimHLD_G16A | G16A |
| 13 | FimHLD_G16P | G16P |
| 14 | FimHLD_G15A_G16A | G15A G16A |
| 15 | FimHLD_R60P | R60P |
| 16 | FimHLD_G65A | G65A |

TABLE 5
Cysteine pairs for disulfide bond stabilization in the
FimH_{LD}

| SEQ ID NO: | Protein ID | Substitutions |
|---|---|---|
| 17 | FimHLD_ P12C_A18C | P12C A18C |
| 18 | FimHLD_G14C_F144C | G14C F144C |
| 19 | FimHLD_ P26C_V35C | P26C V35C |
| 20 | FimHLD_P26C_V154C | P26C V154C |
| 21 | FimHLD_P26C_V156C | P26C V156C |
| 22 | FimHLD_V27C_L34C | V27C L34C |
| 23 | FimHLD_V28C_N33C | V28C N33C |
| 24 | FimHLD_V28C_P157C | V28C P157C |
| 25 | FimHLD_Q32C_Y108C | Q32C Y108C |
| 26 | FimHLD_N33C_L109C | N33C L109C |
| 27 | FimHLD_N33C_P157C | N33C P157C |
| 28 | FimHLD_V35C_L107C | V35C L107C |
| 29 | FimHLD_V35C_L109C | V35C L109C |
| 30 | FimHLD_S62C_T86C | S62C T86C |
| 31 | FimHLD_S62C_L129C | S62C L129C |
| 32 | FimHLD_Y64C_L68C | Y64C L68C |
| 33 | FimHLD_Y64C_A127C | Y64C A127C |
| 34 | FimHLD_L68C_F71C | L68C F71C |
| 35 | FimHLD_V112C_T158C | V112C T158C |
| 36 | FimHLD_S113C_G116C | S113C G116C |
| 37 | FimHLD_S113C_T158C | S113C T158C |
| 38 | FimHLD_V118C_V156C | V118C V156C |
| 39 | FimHLD_A119C_V155C | A119C V155C |

TABLE 6
Nonpolar-to-polar mutations in FimH_{LD}

| SEQ ID NO: | Protein ID | Substitutions |
|---|---|---|
| 40 | FimHLD_L34N_V27A | V27A L34N |
| 41 | FimHLD_L34S_V27A | V27A L34S |
| 42 | FimHLD_L34T_V27A | V27A L34T |
| 43 | FimHLD_A119N_V27A | V27A A119N |
| 44 | FimHLD_A119S_V27A | V27A A119S |
| 45 | FimHLD_A119T_V27A | V27A A119T |
| | FimHLD_L34D_V27A | V27A L34D |
| | FimHLD_L34E_V27A | V27A L34E |
| | FimHLD_L34K_V27A | V27A L34K |
| | FimHLD_L34R_V27A | V27A L34R |
| | FimHLD_A119D_V27A | V27A A119D |
| | FimHLD_A119E_V27A | V27A A119E |
| | FimHLD_A119K_V27A | V27A A119K |
| | FimHLD_A119R_V27A | V27A A119R |

TABLE 7
Cavity-filling mutations at the Pilin-Lectin
interface of FimH-DSG

| SEQ ID NO: | Protein ID | Substitutions |
|---|---|---|
| 46 | FimH-DSG_A115V | A115V |
| 47 | FimH-DSG_V163I | V163I |
| 48 | FimH-DSG_V185I | V185I |
| 49 | FimH-DSG_DSG_V3I | DSG V3I |

TABLE 8
Combination of representative mutations in FimH_{LD}

| SEQ ID NO: | Protein ID | Substitutions |
|---|---|---|
| 50 | FimHLD_G15A_V27A | G15A V27A |
| 51 | FimHLD_G16A_V27A | G16A V27A |
| 52 | FimHLD_G15P_V27A | G15P V27A |
| 53 | FimHLD_G16P_V27A | G16P V27A |
| 54 | FimHLD_G15A_G16A_V27A | G15A G16A |
| | | V27A |
| 55 | FimHLD_V27A_R60P | V27A R60P |
| 56 | FimHLD_V27A_G65A | V27A G65A |
| 57 | FimHLD_V27A_Q133K | V27A Q133K |
| 58 | FimHLD_G15A_G16A_V27A_Q133K | G15A G16A |
| | | V27A Q133K |

TABLE 9
Combination of representative mutations in FimH-DSG

| SEQ ID NO: | Protein ID | Substitutions |
|---|---|---|
| 59 | FimH-DSG_WT | WT/none |
| 60 | FimH-DSG_V27A | V27A |
| 61 | FimH-DSG_G15A_V27A | G15A V27A |
| 62 | FimH-DSG_G15A_G16A_V27A | G15A G16A |
| | | V27A |
| 63 | FimH-DSG_V27A_Q133K | V27A Q133K |
| 64 | FimH- | G15A G16A |
| | DSG_G15A_G16A_V27A_Q133K | V27A |
| | | Q133K |

### Example 2: Antigen Expression and Purification

DNA encoding FimH_{LD} and FimH-DSG mutants was cloned into pcDNA3.1 containing a mouse IgK signal peptide and expressed in Expi293™ cells as previously described (PCT Intl. Publication No. WO2021/084429, published May 6, 2021). For protein characterization and immunogenicity studies, proteins were isolated using nickel affinity resin and size exclusion chromatography as described in PCT Intl. Publication No. WO2021/084429, published May 6, 2021.

### Example 3: Fluorescence Polarization Assay

To determine dissociation constants of FimH mutants for mannoside ligands, a fluorescence polarization assay was developed based on methods described by Rabbani et al. (J. Biol. Chem. 293:1835-1849 (2018)) using fluorescein conjugated to mannoside ligands with a high affinity for FimH. FimH proteins were diluted in 20 mM HEPES pH 7.4, 150 mM NaCl, 0.05 mg/mL plus BSA 0.05% in an 11-point threefold titration in a black flat bottom 96-well polypropylene plate (Greiner) with a final volume of 50 μL. 50 μL of fluorescein octylbiphenylmannopyranoside ligand at 0.7 nM in same buffer was added to each well. Plates were incubated overnight at room temperature, shaking at 100 rpm. After 20-24 hr, plates were read in a ClarioStar Plus plate reader with fluorescein excitation at 488 nm and emission at 530 nm.

### Example 4: Thermal Stability Assay (ThermoFluor Assay)

A 384-well thermal stability assay using SYPRO orange was developed to determine the melting temperatures of isolated proteins in APO (unbound) form and in the presence of ligand. Mannoside compounds (methyl α-D-mannopyranoside (Sigma M6882) mimicking the natural ligand of FimH (mannose) were used to analyze association to the protein. FimH protein stock solutions were prepared by diluting proteins in 40 mM Tris pH 8, 400 mM NaCl (Assay Buffer) to 4 μM; SYPRO orange dye (Invitrogen S6650) was diluted 1:10 in Assay Buffer. 4 μM FimH mutants (5 μL) were mixed with 1:10 SYPRO orange dye (0.1 μL) and either Assay Buffer or ligand diluted in Assay Buffer (5 μL) for 10 μL final reaction volume in a MicroAmp EnduraPlate Optical 384-well plate (Applied Biosystems 4483285). The plate was subjected to melt curve analysis in a QuantStudio 5 Real-time PCR system (ThermoFisher) using a dissociation protocol from 20° C. to 98° C., at 0.05° C./second. TAMRA was specified as the target and reporter, ROX as a passive reference (not used for any analysis however). Data was plotted as a Maxwell-Boltzmann distribution, with Temperature (from 20° C. to 98° C.) on the X-axis, and fluorescence from the TAMRA channel charted on the Y-axis (each temperature point read during the melt curve being assigned a specific fluorescence excitation value for the TAMRA reporter). A normalization algorithm was established to equalize fluorescence intensity between wells and samples, so the Y-axis of fluorescence could be compared from plate to plate, on a scale from 0 (no fluorescence) to 1 (highest recorded fluorescence). This equation is shown below. Using a search function in Microsoft Excel (also below), a relative fluorescence (after normalization) value of 0.5 (indicating approximately half of the protein has disassociated) was recorded, correlating to a specific temperature. This temperature, the taken melting temperature (Tₘ) of the protein was thus calculated. The shift in melting temperature (ΔTₘ) was calculated by subtracting the Tₘ of the protein+ligand from the apo condition. Pivot tables in Microsoft Excel were used to organize Tₘ from plate layout.

$$\begin{matrix}Equation to normalize TAMRA fluorescence signal : & \\ Normalization value ( between 0 \& 1 ) = \frac{\begin{matrix}raw fluorescence value - \\ minimum fluorescence value from entire well \\ ( from 20 ° C . to 98 ° C . )\end{matrix}}{\begin{matrix}Maximum fluorescence value from entire well - \\ minimum fluorescence value from entire well\end{matrix}} & \end{matrix}$$

$$Excel search function to Identify \text{} {T}_{m} ( 0.5 normalized fluorescence , \\ or 50 \% protein melting ) := LOOKUP ( 0.5 , \\ beginning of normalized fluorescence values : end of values , \\ \$ beginning of temperature values : \$ end of values )$$

### Example 5: Confirmation of Conformational State of FimH Mutants with FimH-Specific Neutralizing Monoclonal Antibodies

Neutralizing monoclonal antibodies 299-3, 304-1 and 440-2 (developed in-house) were used to confirm the conformational state of FimH mutants; 229-3 and 304-1 bind to similar epitopes as Mab 475 and 926 (Kisiela, D. I. et al. Proc Natl Acad Sci USA 110, 19089-19094 (2013)) while 440-2 recognizes a different epitope and appears to preferentially bind FimH_{LD} in an open conformational state. Variants maintaining structural integrity similar to wildtype are expected to bind all antibodies. Octet HTX from ForteBio was used for all the kinetic real-time biomolecular interaction experiments to measure antibody reactivity with each mutant. Experiments were carried out at 30° C. with 1000 rpm agitation in 96-well black plates containing 240 μL per well. Ni-NTA biosensors were equilibrated in buffer containing 1×PBS buffer containing 0.5% BSA and 0.05% TWEEN 20 (PBT) before allowing them to load His-tagged FimH mutant proteins at 5 μg/mL for 5 minutes. FimH loaded biosensors were allowed to reestablish baseline in PBT for 3 minutes before allowing them to associate with antibodies from different bins at 5 μg/mL for 5 minutes. Octet data analysis software was used for kinetic analysis of association step and obtain response in nm shift (tabulated).

### Example 6: Circular Dichroism Spectroscopy

Far-UV (320-250 nm) and near-UV (260-200 nm) circular dichroism spectra were recorded for FimH_{LD} and FimH-DSG mutants using JASCO J-810 Spectropolaromiter (Jasco), equipped with JASCO PTC-424S/15 (Jasco) temperature control and Isotemp water bath (Fisher Scientific) units. For far-UV, a 1 mm cell was used, and for near-UV a 10 mm cell was used. Proteins were diluted to 0.3 mg/mL in PBS and spectra were recorded at 20° C. using a cell with 1 mm (far-UV) or 10 mm (near-UV) path length. Scans were performed at 100 nm/min, DIT was set to 1s, bandwidth to 3s, and data pitch to 0.1 nm. Sensitivity was set to standard. Ten spectra were accumulated and averaged for near-UV and five for far-UV measurements respectively. Spectra were corrected to background manually using CD spectra arising from blank PBS runs and were converted to mean residue ellipticity using EQ. 1. Where θMRE is the calculated mean residue ellipticity, θEXP is experimentally measured CD signal, MW is protein molecular weight, N is the number of amino acid residues, C is protein concentration in mg/mL, I is the optical path length in cm.

$${θ}_{MRE} = ( {θ}_{EXP} \cdot MW ) / ( 10 \cdot N \cdot C \cdot l ) \qquad EQ . 1$$

### Example 7: Animal Immunogenicity Study EC-1678

6-8 week old CD-1 mice were obtained from Charles River Laboratories. For each group, 20 animals were immunized subcutaneously 0, 4 and 8 weeks with 10 μg FimH protein mixed with 20 μg Quillaja saponaria-21 (QS-21) from a 5.1 mg/mL QS-21 stock solution containing 5 mM Succinate, 60 mM NaCl, 0.1% PS80, pH 5.6.

### Example 8: FimH Whole Cell Neutralization Assays

To evaluate the ability of serum from vaccinated animals to inhibit binding of fimbriated E. coli to mannosylated substrates, a whole cell neutralization assay using yeast mannan was employed as described in PCT Intl. Publication No. WO2021/084429, published May 6, 2021.

### Example 9: Purification of FimH-DSG WT and FimH-DSG G15A G16A V27A Mutant from CHO Cells

Proteins were expressed in CHO cells as secreted proteins with C-terminal His tags. Cell culture supernatant was harvested and 1 M Tris pH 7.4 and 5 M NaCl were added to final concentrations of 20 mM and 150 mM final concentrations respectively. A 5 kDa TFF cassette buffer was rinsed and equilibrated in 20 mM Tris pH 7.5 with 500 mM NaCl and 40 mM imidazole. Supernatant was concentrated 2-fold and diafiltered against 6 volumes of 20 mM Tris pH 7.5 500 mM NaCl 40 mM imidazole. Retentate was collected and rinsed with 50-100 mL of 20 mM Tris pH 7.5 500 mM NaCl 40 mM imidazole. Retentate was filtered and rinsed with a 0.2 μm bottletop filter. An XK26/20 column was packed with Ni-SEPHAROSE 6 Fast Flow resin (Cytiva Life Sciences) and equilibrated with 5 column volumes of 20 mM Tris pH 7.5 500 mM NaCl 40 mM imidazole. Retentate was applied at half flow rate and washed until a stable baseline was reached (approximately 55 column volumes). Bound protein was eluted with 20 mM Tris, 500 mM NaCl, 500 mM imidazole, pH 7.5. Fractions containing the protein of interest were pooled and dialyzed in a 2 kDa dialysis cassette against 20 mM sodium acetate, pH 4.3 at 4° C. with two buffer changes. Protein was applied to a SP-SEPHAROSE cation exchange column (Cytiva Life Sciences) that had been equilibrated with the same buffer. Material bound to the cation-exchange resin was eluted with a linear gradient of NaCl using 20 mM sodium acetate, pH 4.3, 1 M NaCl buffer. Fractions were pooled, and dialyzed against TBS, pH 7.4.

### Example 10: Analytical Size Exclusion Chromatography (SEC) on FimH-DSG WT and FimH-DSG G15A G16A V27A Mutant

Analytical SEC was conducted using Waters X bridge Protein BEH SEC 125 Å 2.5 μm, 4.6×300 mm column in TBS, pH 7.4 buffer containing 10 mM EDTA at 25° C. The injection volume was 10 μl and the flow rate 0.5 mL/min.

### Example 11: Monosaccharide Analysis by High pH Anion Exchange Chromatography Using Pulsed Amperometric Detection (HPAEC-PAD)

Aqueous samples of FimH-DSG wild type and FimH-DSG triple mutant (G15A, G16A, V27A) were digested for 2 hr at 120° C. in 2N Trifluoroacetic acid. After this time samples were evaporated to dryness under vacuum at 45° C. for 6 hrs. Samples were re-constituted in Milli-Q H₂O and were evaluated by HPAEC-PAD on a DIONEX ICS 3000 ion chromatography system. A Dionex CarboPac PA1 column (4×250 mm) was used with an isocratic elution using a mixture of H₂O and 200 mM NaOH. Monosaccharide composition was confirmed by comparing retention times of peaks detected in FimH samples with solutions of known monosaccharide standards.

### Example 12: Detection of O-Antigen Sugar Moiety Binding to FimH-DSG WT and FimH-DSG G15A G16A V27A Mutant

Octet HTX from ForteBio was used for all the kinetic real-time biomolecular interaction experiments to measure possible O-antigen interactions with FimH-DSG WT and FimH-DSG G15A G16A V27A mutant. Experiments were carried out at 30° C. with 1000 rpm agitation in 96-well black plates containing 240 μl per well. Ni-NTA biosensors were equilibrated in buffer containing 1×PBS buffer with 0.5% BSA and 0.05% Tween 20 (PBT) before allowing them to load His-tagged FimH-DSG WT or FimH-DSG G15A G16A V27A mutant at 5 μg/ml for 5 min. FimH-DSG WT or FimH-DSG G15A G16A V27A-loaded biosensors were allowed to reestablish baseline in PBT for 3 min before allowing them to load with 2-fold titrations (200-3.125 μg/ml) of O-antigen polysaccharide CRM conjugates, O9 or O25b or O1a or O2. An antigen loaded biosensor without any polysaccharide was used as reference. FimH and O-antigen titration loaded biosensors were immersed in PBT for 3 min for new baseline to be established. Detection of O-antigen binding to the mutant was tested in an association step with 5 μg/mL O-antigen specific mAb for 5 min (MAb 601 for O9, MAb ECO-80-11 for O25b, MAb ECO-48-2 for O1a and MAb ECO-172-13 for O2). Octet data analysis software was used for kinetic analysis of reference subtracted association step and obtain response in nm shift (tabulated).

### Example 13: Protein Expression and Purification

FimH_{LD} and FimH-DSG mutants were expressed in Expi293 cells and isolated from supernatants by nickel affinity capture followed by size exclusion chromatography. Note, some mutants had poor expression levels and were not progressed to biochemical or biophysical evaluation (e.g. FimH_{LD} P26C V35C, N33C P157C, N33C L109C, V35C L107C, V35C L109C, S113C T158C). Mutants for which sufficient yields could be obtained were evaluated in thermal stability and ligand binding assays.

### Example 14: Identification of FimH_{LD} and FimH-DSG Mutants with Improved Thermal Stability and Reduced Shift in Melting Temperature in the Presence of Mannoside Ligand

Melting temperatures of FimH mutant proteins were determined using a SYPRO orange thermal shift-based differential scanning fluorimetry assay, wherein Tₘ designates the temperature at which 50% of the protein is unfolded. Non-covalent ligands often stabilize protein targets upon specific binding, resulting in an increase in protein melting temperature. Melting temperatures were therefore determined in the presence of methyl alpha-D-mannopyranoside, which is a derivative of alpha-D-mannose and has a micromolar affinity for FimH (Bouckaert, J. et al. Mol Microbiol. 55, 441-455 (2005)) and the difference in melting temperature (ΔTₘ) of protein in the presence of ligand relative to apo form was calculated.

Wild type (WT) FimH-DSG proteins exhibited significantly higher melting temperatures compared to FimH_{LD} WT and had lower ΔTₘ in the presence of ligand (Table 10, Table 11). FimH-DSG WT had a melting temperature of 71.66° C. while the melting temperature of FimH_{LD} WT was significantly lower (61.54° C.). In the presence of methyl alpha-D-mannopyranoside, the melting temperature of FimH_{LD} WT shifted by 10.99° C., while the temperature for FimH-DSG in the presence of ligand shifted only by 2.13° C. This suggests that FimH_{LD} is more efficiently stabilized by the ligand compared to FimH-DSG which may reflect reduced ligand binding by FimH-DSG.

Mutations impacted the melting temperature of FimH proteins in the apo state and in the presence of ligand. The FimH_{LD} lock mutant V27C L34C described previously (Kisiela, D. I. et al. Proc Natl Acad Sci USA 110, 19089-19094 (2013); Rodriguez, V. B. et al. J Biol Chem 288, 24128-24139 (2013)) exhibited a lower melting temperature (51.42° C.) compared to wild type FimH_{LD} (61.54° C.), consistent with published data (Kisiela, D. I. et al. Proc Natl Acad Sci USA 110, 19089-19094 (2013); Rodriguez, V. B. et al. J Biol Chem 288, 24128-24139 (2013)). Incubation of FimH_{LD} V27C L34C with methyl alpha-D-mannopyranoside increased the melting temperature by 7.27° C. compared to the apo form, suggesting that this mutant is partly stabilized by the ligand and may have residual ligand binding efficiency. In the context of FimH-DSG, the V27C L34C mutant was less thermostable compared to WT (Tₘ=63.29° C.) and the temperature shift in the presence of ligand was slightly reduced in the V27C L34C mutant compared to WT (ΔTₘ=1.29° C.). Five of the six Phe1 FimH_{LD} mutants had decreased melting temperatures compared to WT FimH_{LD}, except F1 L which had a similar melting temperature. In the presence of ligand, four of six FimH_{LD} Phe1 mutants showed a small ΔTm, suggesting poor stabilization by the ligand. In contrast, the most conservative amino acid substitutions F1W and F1Y, exhibited intermediate and comparable ΔTm values respectively, versus the Phe1 wildtype FimH_{LD}. With regard to overall thermal stability, the R60P reference mutation (described previously—Rabbani et al. J. Biol. Chem. 293:1835-1849 (2018); Rodriguez, V. B. et al. J Biol Chem 288, 24128-24139 (2013)) and several novel mutations designed in-house had significantly increased melting temperatures relative to V27C L34C. Interestingly, FimH_{LD} V28C N33C (both sites shifted just one residue away from the reference FimH_{LD} V27C L34C) had the highest melting temperature of any FimH_{LD} mutant (Tₘ=65.77° C.) and had a ΔTₘ of 2.81° C. in the presence of methyl alpha-D-mannopyranoside, suggesting reduced affinity for ligand. Mutations in the glycine loop region (G15A, G16A) in FimH_{LD} significantly increased thermal stability relative to V27C L34C and a very low shift in melting temperature was observed in the presence of ligand. The glycine loop mutations also slightly increased the thermal stability of FimH-DSG and no temperature shift was observed with ligand, together suggesting that FimH_{LD} and FimH-DSG mutants are not stabilized by the ligand and therefore may have reduced binding efficiency relative to wild type.

The sequence of FimH_{LD} WT is derived from E. coli UTI isolate J96 (Hull, R. A. et al., Infect Immun 33, 933-938 (1981)). V27A is a natural variant that is associated with virulent UTI isolates and isolates associated with Crohn's Disease (Schwartz, D. J. et al., Proc Natl Acad Sci USA 110, 15530-15537 (2013); Cespedes et al., Front Microbiol 8:639 (2017)). Incorporation of V27A into FimH_{LD} slightly reduced the melting temperature of FimH_{LD} WT and a smaller shift was observed with the V27A in the presence of methyl alpha-D-mannopyranoside compared to WT. On the other hand, V27A appeared to have a stabilizing effect in the context of glycine loop mutants G15A, G16A, G15P, G16P in FimH_{LD}, which all had a higher melting temperature with V27A compared to without, and had a ΔTm of <2° C. in the presence of V27A compared to up to 6.05° C. (G16P) without this mutation. In addition, FimH-DSG mutants containing V27A had slightly increased thermal stability and there was no detectable temperature shift in the presence of ligand. Together, this suggests that V27A has a stabilizing effect on the melting temperature of FimH and reduces ligand binding efficiency.

Several FimH_{L} disulfide and nonpolar to polar residue mutants were expressed at low levels and were either poorly thermostable or exhibited significant temperature shifts in the presence of mannoside compound suggesting that they retained ligand binding efficiency (Table 12). These were tested in single replicates and were excluded from further analysis. Similarly, the thermal stability of several other FimH-DSG mutants was analyzed, two of which had improved thermal stability and reduced shift with ligand (FimH-DSG V27A Q133K and FimH-DSG G15A G16A V27A Q133K) (Table 13). The Q133K mutation is a mutation in the binding pocket of FimH that eliminates ligand binding, which was described previously (Schwartz et al., Proc Nat Acad Sci USA 110:15530-15537 (2013)). These mutants were not analyzed further.

TABLE 10
Melting temperature of FimH_{LD} mutants in apo state and in the
presence of methyl alpha-D-mannopyranoside

| | | Tm | St. Dev. | ΔTm | St. Dev. |
|---|---|---|---|---|---|
| | Repli- | (Average)/ | (Tm)/ | (Average)/ | (ΔTm)/ |
| FimH variant | cates | ° C. | ° C. | ° C. | ° C. |
| FimH_{LD} WT | 11 | 61.54 | 0.81 | 10.99 | 0.84 |
| FimH_{LD} V27C L34C | 7 | 51.42 | 0.81 | 7.27 | 0.57 |
| FimH_{LD} V27A | 6 | 59.87 | 0.88 | 9.95 | 0.57 |
| FimH_{LD} G15A | 3 | 56.16 | 0.13 | 3.81 | 0.39 |
| FimH_{LD} G15P | 2 | 55.99 | 1.05 | 2.86 | 0.06 |
| FimH_{LD} G16A | 2 | 54.59 | 0.25 | 6.05 | 0.33 |
| FimH_{LD} G16P | 2 | 55.34 | 0.62 | 1.53 | 0.25 |
| FimH_{LD} G15A V27A | 4 | 57.77 | 0.54 | 1.45 | 0.51 |
| FimH_{LD} G15P V27A | 2 | 58.2 | 0.56 | 1.26 | 0.55 |
| FimH_{LD} G16A V27A | 6 | 57.65 | 0.45 | 1.84 | 0.97 |
| FimH_{LD} G16P V27A | 5 | 58.19 | 0.52 | 0.81 | 0.76 |
| FimH_{LD} R60P | 2 | 56.25 | 0.21 | 8.4 | 1.7 |
| FimH_{LD} V27A R60P | 3 | 60.1 | 0.44 | 3.12 | 0.45 |
| FimH_{LD} G15A G16A | 3 | 59.14 | 0.43 | 0.42 | 0.5 |
| V27A | | | | | |
| FimH_{LD} V28C N33C | 5 | 65.77 | 1.13 | 2.81 | 0.21 |
| FimH_{LD} P26C | 4 | 60.41 | 1.79 | 5.74 | 1.05 |
| V154C | | | | | |
| FimH_{LD} F1I | 2 | 55.18 | 0.04 | 0.34 | 1.16 |
| FimH_{LD} F1L | 2 | 60.07 | 0.23 | 0.78 | 0.54 |
| FimH_{LD} F1M | 3 | 52.68 | 1.52 | 1.1 | 1.63 |
| FimH_{LD} F1V | 3 | 52.4 | 0.05 | 0.74 | 0.93 |
| FimH_{LD} F1W | 3 | 52.82 | 0.6 | 4.51 | 0.64 |
| FimH_{LD} F1Y | 3 | 54.17 | 0.18 | 10.09 | 0.32 |

TABLE 11
Melting temperature of FimH-DSG mutants in apo state and in the
presence of methyl alpha-D-mannopyranoside

| | | Tₘ | St. Dev. | | St. Dev. |
|---|---|---|---|---|---|
| | Repli- | (average)/ | (Tₘ)/ | ΔTₘ/ | (ΔTₘ)/ |
| FimH variant | cates | ° C. | ° C. | ° C. | ° C. |
| FimH-DSG WT | 11 | 71.66 | 0.48 | 2.13 | 0.17 |
| FimH-DSG V27C L34C | 6 | 63.29 | 1.24 | 1.29 | 0.49 |
| FimH-DSG V27A | 5 | 72.56 | 0.55 | −0.35 | 0.22 |
| FimH-DSG G15A V27A | 5 | 73.02 | 0.64 | −0.14 | 0.29 |
| FimH-DSG G16A V27A | 5 | 72.27 | 0.48 | −0.04 | 0.06 |
| FimH-DSG G15A G16A | 10 | 73.42 | 0.52 | 0.04 | 0.12 |
| V27A | | | | | |

TABLE 12
Melting temperature of FimH_{LD} mutants in apo
state and in the presence of methyl alpha-D-
mannopyranoside, single replicates

| FimH variant | Tₘ/° C. | ΔTₘ/° C. |
|---|---|---|
| FimH_{LD} P26C V156C | 58.86 | 5.42 |
| FimH_{LD} Q32C Y108C | 61.09 | 9.77 |
| FimH_{LD} P26C V154C | 62.51 | 4.65 |
| FimH_{LD} V28C P157C | 59.61 | 7.16 |
| FimH_{LD} S62C T86C | 58.28 | 11.71 |
| FimH_{LD} S62C L129C | 57.12 | 12.77 |
| FimH_{LD} Y64C A127C | 60.22 | 12.87 |
| FimH_{LD} V112C T158C | 59.64 | 15.2 |
| FimH_{LD} V118C V156C | 56.7 | 12.89 |
| FimH_{LD} P12C A18C | 54.47 | 5.23 |
| FimH_{LD} G14C F144C | 49.24 | -0.1 |
| FimH_{LD} L68C F71C | 49.92 | 12.2 |
| FimH_{LD} S113C G116C | 59.8 | 9.29 |
| FimH_{LD} A119C V155C | 59.12 | 14.52 |
| FimH_{LD} L34S V27A | 48.86 | 12.78 |
| FimH_{LD} L34T V27A | 53.22 | 10.46 |
| FimH_{LD} L34N V27A | 47.31 | 13.07 |
| FimH_{LD} A119S V27A | 59.8 | 8.52 |
| FimH_{LD} A119T V27A | 59.51 | 9.19 |
| FimH_{LD} A119N V27A | 57.87 | 7.55 |
| FimH_{LD} V27A G65A | 59.8 | 10.85 |

TABLE 13
Melting temperature of FimH-DSG mutants in apo
state and in the presence of methyl alpha-D-
mannopyranoside, limited replicates

| | | Tₘ | St. Dev. | | St. Dev. |
|---|---|---|---|---|---|
| | Repli- | (average)/ | (Tₘ)/ | ΔTₘ/ | (ΔTₘ)/ |
| FimH variant | cates | ° C. | ° C. | ° C. | ° C. |
| FimH-DSG A115I | 2 | 68.51 | 0 | 4.07 | 0 |
| FimH-DSG V185I | 2 | 71.37 | 0.48 | 2.51 | 0.41 |
| FimH-DSG DSG V3I | 1 | 70.74 | N/A | 3.09 | N/A |
| FimH-DSG V163I | 1 | 70.36 | N/A | 3.48 | N/A |
| FimH-DSG Q133K | 1 | 71.61 | N/A | 1.74 | N/A |
| FimH-DSG V27A Q133K | 1 | 75.1 | N/A | 0.1 | N/A |
| FimH-DSG G15A G16A | 1 | 73.93 | N/A | 1.27 | N/A |
| V27A Q133K | | | | | |

### Example 15: Identification of FimH Mutants with Reduced Affinity for Mannoside Ligand

Dissociation constants (K_{d}) of FimH mutants for mannoside ligand were determined using a direct binding fluorescence polarization assay with a fluorescein-conjugated octylbiphenylmannopyranoside (BPMP) ligand. The K_{d} values of FimH_{LD} mutants relative to WT are shown in Table 14. FimH_{LD} WT and V27A showed similar high affinities for BPMP. The reference lock mutant FimH_{LD} V27C L34C (Kisiela, D. I. et al., Proc Natl Acad Sci USA 110, 19089-19094 (2013); Rodriguez, V. V. et al., J Biol Chem 288:24128-24139 (2013)) had a 91-fold lower affinity for the ligand relative to FimH_{LD} WT, while FimH_{LD} R60P V27A (Rabbani et al., J Biol Chem 293:1835-1849 (2018)) had a 179-fold lower affinity. The mutants disclosed herein were compared to reference lock mutants. No binding was detected for Glycine loop mutants FimH_{LD}G15A V27A, G15P V27A, G16P V27A and G16A G16A V27A, while G16A V27A had a 156-fold increase in K_{d} relative to WT. Glycine loop mutations combined with V27A all exhibited a significantly higher K_{d} than the Glycine loop mutants alone, suggesting that V27A has an indeterminate stabilizing effect, though it had little impact on the K_{d} of FimH_{LD} WT. Inclusion of V27A also further decreased the binding affinity of FimH_{LD} R60P.

Three novel disulfide lock mutants were tested in this assay, all of which had a modest reduction in ligand binding affinity relative to FimH_{LD} WT (33 to 43-fold lower affinity). FimH_{LD} mutants containing nonpolar to polar mutations (A119T V27A, A119N V27A, L34T V27A, L34N V27A) had a high affinity for BPMP, similar to that of FimH_{LD} WT. FimH_{LD} F1 mutants exhibited poor binding, except F1Y, which had a similar binding affinity to FimH_{LD} WT.

Ligand binding affinities of FimH-DSG constructs are shown in Table 15. The K_{d} of FimH-DSG WT was more than 100-fold higher than that of FimH_{LD} WT, likely reflecting the different conformational states of the two forms of FimH. FimH-DSG V27A also had a lower affinity relative to FimH-DSG WT. This is consistent with previous data showing that full length FimH with A27 in complex with FimC and FimG has a reduced binding affinity for mannoside relative to FimH V27 (Schwartz et al., Proc Nat Acad Sci USA 110:15530-15537 (2013)). Introduction of lock mutation V27C L34C into FimH-DSG reduced the affinity for BPMP 2.5-fold, while the Glycine loop mutant FimH-DSG G15A G16A V27A had a 28-fold lower affinity relative to FimH-DSG WT. K_{d} could not be calculated for FimH-DSG G15A V27A and FimH-DSG G16A V27A suggesting that these mutants cannot bind BPMP. Mutations designed to stabilize FimH-DSG in an open conformation via alteration of the pilin-lectin domain interface (A115I, V185I) improved binding affinity relative to FimH-DSG WT, as suggested from thermal stability data (Example 14).

In summary, Glycine loop mutations in either the FimH_{LD} or FimH-DSG protein were identified that had very low binding affinities for BPMP. Based on these and thermal stability data, Glycine mutants were selected for evaluation in functional immunogenicity studies in mice.

TABLE 14
Binding K_{d} of FimH_{LD} mutants to
octylbiphenylmannopyranoside ligand

| | | Average | StdDev/ |
|---|---|---|---|
| FimH variant | Replicates | K_{d}/nM | nM |
| FimH_{LD} WT | 10 | 0.193 | 0.041 |
| FimH_{LD} V27A | 3 | 0.213 | 0.034 |
| FimH_{LD} V27C L34C | 7 | 17.706 | 3.052 |
| FimH_{LD} G15P | 1 | >2000 | N/A |
| FimH_{LD} G15P V27A | 4 | >2000 | N/A |
| FimH_{LD} G15A | 1 | 46.74 | N/A |
| FimH_{LD} G15A V27A | 4 | >2000 | N/A |
| FimH_{LD} G16P | 1 | >2000 | N/A |
| FimH_{LD} G16P V27A | 4 | >2000 | N/A |
| FimH_{LD} G16A | 1 | 9.588 | N/A |
| FimH_{LD} G16A V27A | 3 | 30.177 | 0.8 |
| FimH_{LD} R60P | 1 | 4.46 | N/A |
| FimH_{LD} V27A R60P | 6 | 34.555 | 8.232 |
| FimH_{LD} G15A G16A | 2 | >2000 | N/A |
| V27A | | | |
| FimH_{LD} V27A G65A | 1 | 4.6 | N/A |
| FimH_{LD} V28C N33C | 2 | 6.465 | 2.128 |
| FimH_{LD} V28C P157C | 1 | 7.68 | N/A |
| FimH_{LD} P26C V154C | 1 | 8.4 | N/A |
| FimH_{LD} L34T V27A | 1 | 0.4 | N/A |
| FimH_{LD} L34N V27A | 1 | 1.7 | N/A |
| FimH_{LD} A119T V27A | 1 | 0.4 | N/A |
| FimH_{LD} A119N V27A | 1 | 1 | N/A |
| FimH_{LD} F1Y | 2 | 0.317 | N/A |
| FimH_{LD} F1W | 1 | 48.4 | N/A |
| FimH_{LD} F1M | 1 | >2000 | N/A |
| FimH_{LD} F1L | 1 | 534 | N/A |
| FimH_{LD} F1I | 1 | >2000 | N/A |
| FimH_{LD} F1V | 1 | 472 | N/A |

TABLE 15
Binding K_{d} of FimH-DSG mutants to
octylbiphenylmannopyranoside ligand

| | Repli- | Average | StdDev/ |
|---|---|---|---|
| FimH variant | cates | K_{d}/nM | nM |
| FimH-DSG WT | 10 | 23.384 | 8.197 |
| FimH-DSG V27A | 2 | 53.545 | 8.973 |
| FimH-DSG V27C L34C | 3 | 59.927 | 28.187 |
| FimH-DSG G15A V27A | 2 | >2000 | N/A |
| FimH-DSG G16A V27A | 2 | >2000 | N/A |
| FimH-DSG G15A G16A V27A | 1 | 667.9 | N/A |
| FimH-DSG A115I | 1 | 9.8 | N/A |
| FimH-DSG V185I | 1 | 21 | N/A |

### Example 16: Confirmation of Conformational State of FimH Mutants by Circular Dichroism Spectroscopy

FimH_{LD} and FimH-DSG mutants that exhibited improved thermal stability and reduced binding affinity for mannoside ligand (Examples 14 and 15) were subjected to secondary and tertiary structure analysis by circular dichroism (CD). Wild type and conformationally locked FimH_{LD} mutants have distinct tertiary CD profiles (Rabbani et al., J Biol Chem 293:1835-1849 (2018)). The secondary and tertiary structures of selected FimH_{LD} and FimH-DSG wild type and mutant proteins were examined by far-UV CD (secondary structure) and near-UV CD (tertiary structure) (see FIG. 1). The far-UV CD spectrum of FimH_{LD} is consistent with previously published data (Rabbani et al. J Biol Chem 293:1835-1849 (2018)) and the far-UV spectrum of both FimH_{LD} and FimH-DSG is characteristic of a protein with high beta-sheet content. FimH_{LD} V27C L34C had a slightly different far-UV spectrum compared to wild type FimH_{LD}, as observed by others (Rabbani et al. J Biol Chem 293:1835-1849 (2018)) reflecting an open conformational state. The secondary structure profile of the naturally occurring FimH_{LD} V27A mutant also varied somewhat. Overall, the secondary structures of the FimH_{LD} or FimH-DSG mutants are highly similar to wild type proteins (FIG. 1), suggesting that the overall secondary structure is not altered in these mutants. The tertiary structure profiles of FimH_{LD} mutants closely resemble in-house and published CD spectra of FimH_{LD} V27 L34C and V27A R60P, which are stabilized in an open conformational state (Rabbani et al. J Biol Chem 293:1835-1849 (2018)). The profiles of the mutants described here also differ significantly compared to wild type FimH_{LD} or FimH_{LD} V27A. Together, these data suggest that the mutations that were introduced shift the conformation of FimH_{LD} to an open conformation, while FimH-DSG tertiary structure remains largely unchanged upon introduction of conformation stabilizing mutations assessed herein.

### Example 17: Characterization of FimH Mutants Using Neutralizing Monoclonal Antibodies

The conformations of several selected FimH antigens were characterized by biolayer interferometry assay using lectin domain-specific monoclonal antibodies 299-3, 304-1 and 440-2. Competition experiments (not shown) demonstrated that antibodies 229-3 and 304-1 bind to similar ligand binding site epitopes as MAb 475 and 926 (Kisiela et al., Proc Natl Acad Sci USA 110:19089-19094 (2013)). Monoclonal antibody 440-2 binds to a different epitope and appears to preferentially bind to FimH_{LD} in the open conformation. Antibodies 229-3 and 304-1 were able to recognize all FimH_{LD} (Table 16) and FimH-DSG (Table 17) variants, though binding was reduced for FimH_{LD} V27A. In contrast, responses to antibody 440-2 were higher in all of the FimH_{LD} or FimH-DSG mutants relative to WT or V27A. This is consistent with CD spectroscopy profiles shown in FIG. 1, suggesting that FimH_{LD} mutants are in an open conformation. The response with 440-2 was also increased in FimH-DSG WT, which combined with the overlapping CD spectroscopy profiles of FimH-DSG mutants and WT (Example 16) suggests that this protein is in an open conformational state regardless of the presence or absence of stabilizing mutations.

TABLE 16
MAb binding to FimH_{LD} variants

| | Response (nm) | | |
|---|---|---|---|
| | Monoclonal antibody | | |
| FimH variant | 299-3 | 304-1 | 440-2 |
| FimH_{LD} WT | 3.1034 | 3.0195 | 0.0427 |
| FimH_{LD} V27A | 0.4378 | 0.5354 | 0.0595 |
| FimH_{LD} V27A R60P | 3.1921 | 3.0254 | 0.8483 |
| FimH_{LD} V27A G15A | 3.3127 | 3.0286 | 0.7799 |
| FimH_{LD} V27A G15P | 3.0686 | 2.9482 | 0.6242 |
| FimH_{LD} V27A G16A | 3.438 | 3.1974 | 0.7486 |
| FimH_{LD} V27A G16P | 3.255 | 3.1626 | 0.8816 |
| FimH_{LD} G15A G16A V27A | 3.3359 | 3.0596 | 0.7854 |
| FimH_{LD} V27C L34C | 3.107 | 2.7702 | 0.8063 |
| FimH_{LD} V28C N33C | 3.0536 | 2.7444 | 0.7231 |
| FimH_{LD} P26C V154C | 3.1056 | 2.8871 | 0.8254 |

TABLE 17
MAb binding to FimH-DSG variants

| | Response (nm) | | |
|---|---|---|---|
| | Monoclonal antibody | | |
| FimH variant | 299-3 | 304-1 | 440-2 |
| FimH-DSG WT | 2.4379 | 2.2184 | 0.3763 |
| FimH-DSG V27A | 2.4802 | 2.1687 | 0.3516 |
| FimH-DSG V27C L34C | 2.3477 | 2.2249 | 0.3329 |
| FimH-DSG V27A G15A | 2.5204 | 2.2999 | 0.3707 |
| FimH-DSG V27A G16A | 2.6876 | 2.4157 | 0.4183 |
| FimH-DSG G15A G16A V27A | 2.6765 | 2.3015 | 0.4926 |

### Example 18: FimH Mutant Neutralization Data

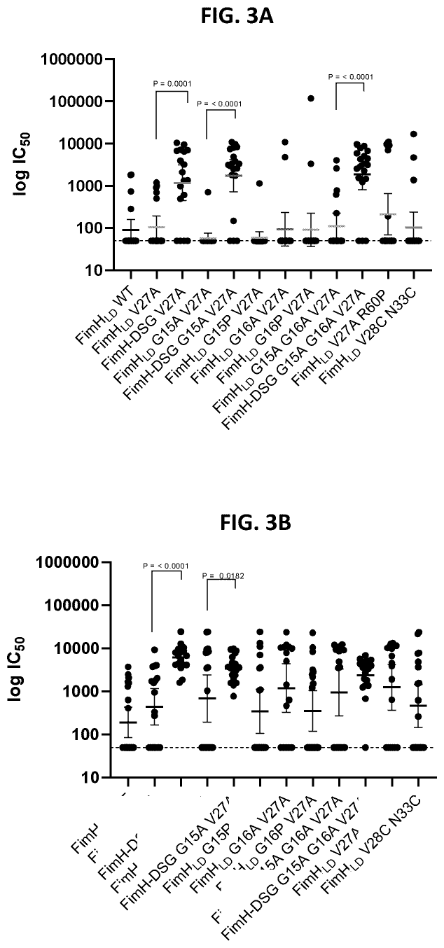

To evaluate the relative immunogenicity of selected mutants, mice were vaccinated with FimH mutants. The potency of FimH mutants to elicit functional antibody titers was quantified using a whole cell yeast mannan neutralization assay described above and previously (POT Intl. Publication No. WO2021/084429, published May 6, 2021). Briefly, fimbriated E. coli were incubated with serum and allowed to bind to a yeast mannan coated microtiter plate. The plate was washed and the number of viable E. coli bound to the plate was detected using a luminescent probe. Serum neutralization titers that inhibit binding of fimbriated bacteria to yeast mannan were determined from an eight-point two-fold dilution series of sera from vaccinated mice. Titers represent the reciprocal of the dilution of serum at which 50% of bacteria remain bound to the plate. A summary of mean titers and responses is shown in Table 18. Plots of individual mouse IC₅₀ responses at post dose 2 and 3 are shown in FIG. 2 and FIG. 3.

TABLE 18
VAC-2020-PRL-EC-1678 FimH_{LD} and FimH-DSG mutant yeast
mannan binding neutralization assay responder rates and GMTs

| | IC₅₀ | | Responder | | Responders | | | |
|---|---|---|---|---|---|---|---|---|
| | GMTs | | rate (%) | | (n) | | Mice (n) | |
| Protein | PD2 | PD3 | PD2 | PD3 | PD2 | PD3 | PD2 | PD3 |
| FimH_{LD} WT | 89 | 191 | 20 | 40 | 4 | 8 | 20 | 20 |
| FimH_{LD} V27A | 104 | 439 | 26 | 61 | 5 | 11 | 19 | 18 |
| FimH-DSG V27A | 1175 | 6102 | 78 | 100 | 14 | 18 | 18 | 18 |
| FimH_{LD} G15A | 57 | 683 | 5 | 53 | 1 | 10 | 20 | 19 |
| V27A | | | | | | | | |
| FimH-DSG | 1740 | 3400 | 84 | 100 | 16 | 19 | 19 | 19 |
| G15A V27A | | | | | | | | |
| FimH_{LD} G15P | 58 | 346 | 5 | 42 | 1 | 8 | 20 | 19 |
| V27A | | | | | | | | |
| FimH_{LD} G16A | 93 | 1193 | 13 | 69 | 2 | 11 | 16 | 16 |
| V27A | | | | | | | | |
| FimH_{LD} G16P | 91 | 352 | 10 | 45 | 2 | 9 | 20 | 20 |
| V27A | | | | | | | | |
| FimH_{LD} G15A | 111 | 1307 | 26 | 63 | 5 | 12 | 19 | 19 |
| G16A V27A | | | | | | | | |
| FimH-DSG G15A | 1869 | 2386 | 84 | 95 | 16 | 18 | 19 | 19 |
| G16A V27A | | | | | | | | |
| FimH_{LD} V27A | 212 | 1056 | 32 | 63 | 6 | 12 | 19 | 19 |
| R60P | | | | | | | | |
| FimH_{LD} V280 | 103 | 461 | 16 | 53 | 3 | 10 | 19 | 19 |
| N33C | | | | | | | | |

Previous work (POT Intl. Publication No. WO2021/084429, published May 6, 2021) showed that the previously described disulfide lock mutant FimH_{LD} V27C L34C (Kisiela et al., Proc Natl Acad Sci USA 110:19089-19094 (2013)) did not improve functional immunogenicity relative to FimH_{LD} WT. The functional immunogenicity of novel FimH_{LD} mutants and another previously described conformationally constrained mutant FimH_{LD} V27A R60P (Rabbani et al., J Biol Chem 293:1835-1849 (2018) are directly compared in FIG. 2. Mutants FimH_{LD} G16A V27A, FimH_{LD} G15A G16A V27A and FimH_{LD} V27A R60P yielded higher numbers of responders and higher titers (p value <0.05) than FimH_{LD} WT. Other mutations (G15A V27A, G16P V27A, V28C N33C) did not significantly enhance functional immunogenicity, although high titers were observed for mice that did respond, the number of responders in these groups was similar to that of the FimH_{LD} WT group. Thus, several mutants designed to enhance functional immunogenicity of FimH_{LD} by locking FimH_{LD} in an open conformation improved functional immunogenicity relative to FimH_{LD} WT. Following vaccination with 2 doses of FimH_{LD} and FimH-DSG mutants, significantly more animals yielded neutralizing titers in the groups vaccinated with FimH-DSG compared to FimH_{LD} (FIG. 3). This trend was sustained at post dose 3, where 95%-100% of mice responded in groups vaccinated with FimH-DSG V27A, FimH-DSG G15A V27A and FimH-DSG G15A G16A V27A. IC₅₀ Geometric mean titers (GMT) were also significantly higher in all groups vaccinated with FimH-DSG mutants at post dose 3. Analogous FimH-DSG mutants (V27A, G15A V27A, G15A G16A V27A) generated higher GMTs relative to FimH_{LD} mutants (p value of <0.05).

### Example 19: FimH-DSG G15A G16A V27A does not Bind Host Glycans and can be Isolated to Homogeneity

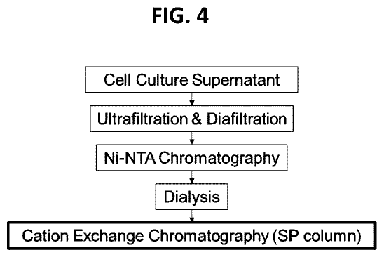

FimH-DSG WT and FimH-DSG G15A G16A V27A mutant proteins were expressed in CHO cells as secreted proteins containing C-terminal His tags. Purification of recombinant His-tagged forms of FimH-DSG was performed as shown in FIG. 4.

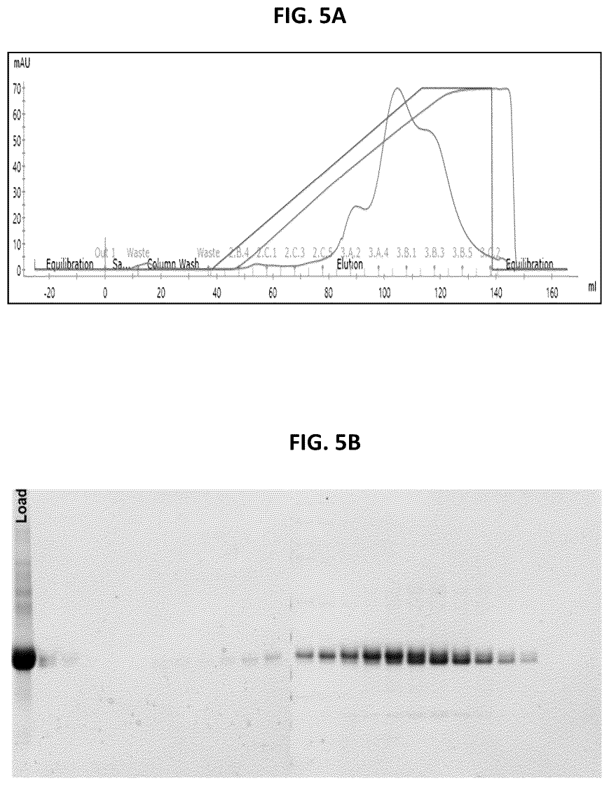

Following ultrafiltration and diafiltration, cell-free culture media containing FimH-DSG WT was isolated on nickel affinity resin and subjected to cation exchange chromatography. The eluted peak was rather broad and exhibited several distinctive shoulders suggesting possible heterogeneity of the FimH-DSG WT species (FIG. 5). Interestingly, when eluted fractions were analyzed by SDS-PAGE only single bands corresponding to the FimH-DSG WT were detected in each fraction.

During the purification process, FimH-DSG exhibited properties suggesting that it has poor solubility. Particularly, FimH-DSG WT Ni-SEPHAROSE eluates always appeared hazy by visual inspection. Shift to acidic pH (4.3) for subsequent purification on SP-SEPHAROSE resulted in clarification of the protein solution. However, poor solubility of FimH-DSG WT preparations and the tendency for aggregation was observed again after the transfer (dialysis) of isolated protein into TBS, pH 7.4. This resulted in progressive loss of the protein due to aggregation and precipitation. Removal of precipitates by centrifugation did not terminate or slow down the aggregation process, even though the protein concentration at this point would be typically reduced down to 0.2-0.4 mg/mL. Loss of protein due to aggregation could be controlled in the presence of 10% glycerol incorporated into the storage (TBS, pH 7.4) buffer. However, the presence of glycerol did not prevent the formation of HMW soluble aggregates that were detected spectrophotometrically by monitoring light scattering at 350 nm.

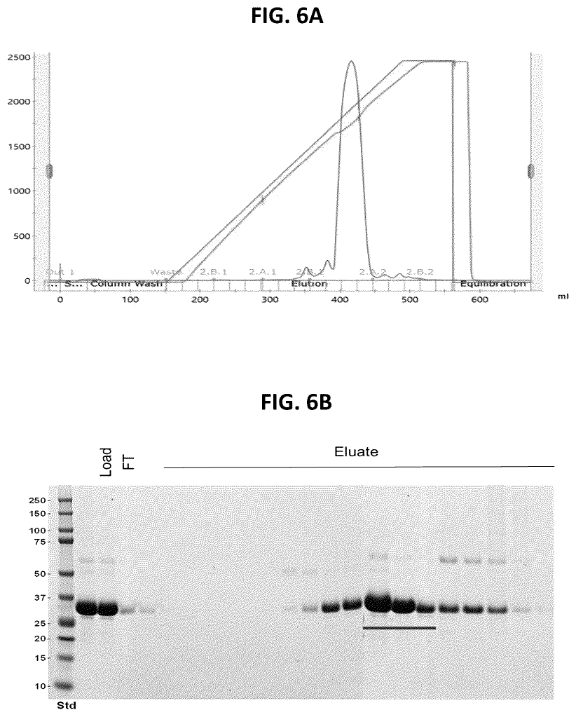

When the same process was utilized for isolation of FimH-DSG G15A G16A V27A mutant, several differences were observed. Firstly, these include the lack of any signs of haziness upon elution of the protein from the Ni-SEPHAROSE column. Secondly, the profile of eluted from SP-SEPHAROSE column peak was not as broad as the one observed with the WT FimH-DSG FIG. 6. And finally, FimH-DSG G15A G16A V27A mutant remained fully soluble after transfer into physiological pH buffer (TBS pH 7.4). At concentrations up to 5-6 mg/mL, FimH-DSG G15A G16A V27A mutant did not show any signs of aggregation or precipitation.

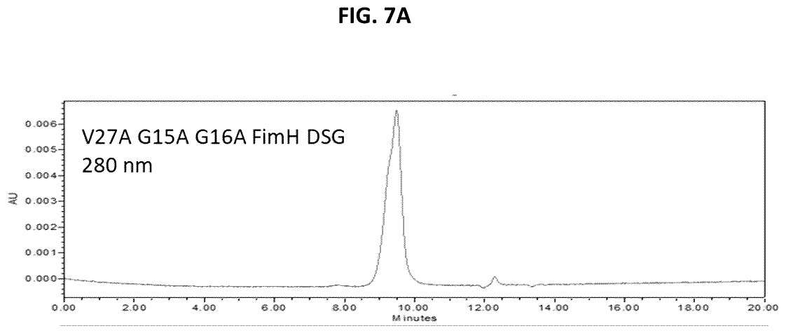

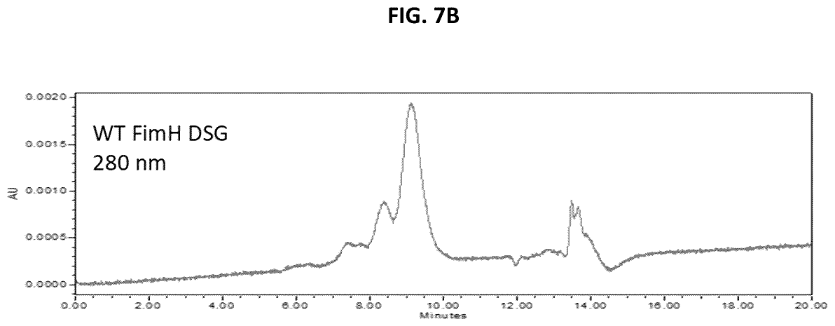

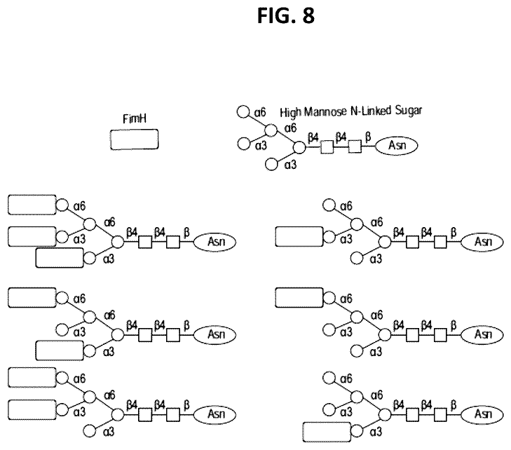

Analysis of isolated FimH-DSG WT and the FimH-DSG G15A G16A V27A mutant further revealed distinctive differences between these two variants of FimH-DSG. Analytical size exclusion chromatography (SEC) demonstrated that FimH-DSG G15A G16A V27A mutant eluted as a single peak with a retention time consistent with its molecular weight. In contrast, the elution profile of the wild type FimH-DSG was composed of several peaks where the retention time of the major peak was less than that of the mutant shown in FIG. 7. These data clearly demonstrate that the FimH-DSG WT forms high molecular mass complexes detectable by SEC. The presence of HMW complexes formed by the FimH-DSG WT and the tendency to aggregate could be linked to the functional activity of its N-terminal lectin-binding domain. We hypothesized that during CHO fermentation and upon secretion into the culture media the FimH-DSG WT binds glycan molecule(s) released from the surface of the host CHO cells. Due to the branched nature of the glycans, more than one copy of the FimH-DSG molecule could be accommodated by each glycan. Continuous “decoration” of the glycan by increasing numbers of FimH-DSG would result in formation of the various HMW complexes and eventually lead to the loss of solubility and precipitation (see FIG. 8). To test this hypothesis, isolated wild type and mutant FimH-DSG (and FimH_{LD}) species were subjected to High pH Anion-Exchange Chromatography with Pulsed Amperometric (electrochemical) Detection (HPAEC-PAD) analysis. This method allows the identification of oligosaccharides or glycans in a protein sample as well as providing information on the composition of these oligosaccharides. Briefly, acid hydrolysis is performed to release monosaccharides, followed by analysis of peaks relative to a monosaccharide standard. The results of HPAEC-PAD analysis revealed that isolated FimH-DSG WT (glycosylated) and FimH_{LD} (not glycosylated, data not shown) preparations contain significant amounts of monosaccharides. The summary of identified monosaccharides in the FimH-DSG WT and FimH-DSG G15A G16A V27A mutant is shown in Table 19. The content of monosaccharides in FimH-DSG G15A G16A V27A mutant was significantly less than that of FimH-DSG WT. Furthermore, it is entirely possible that the low monosaccharide content that was detected represents sugar moieties of the N-glycan predicted to modify N235.

TABLE 19
Normalized amounts of monosaccharides (μg/mg protein) detected
by HPAEC-PAD in various SP-Sepharose fractions from FimH-DSG
WT and the main peak of FimH-DSG G15A G16A V27A mutant

| Sugar | FimH-DSG WT | | | | | | | | | Total |
|---|---|---|---|---|---|---|---|---|---|---|
| Rhamnose | 0 | 0 | 0 | 0 | 0 | 0 | 0 | 0 | 0 | 0 |
| Nac Gal | 0 | 0 | 0 | 99 | 73 | 0 | 52 | 0 | 177 | 401 |
| Nac Glu | 133 | 190 | 9 | 0 | 0 | 0 | 0 | 44 | 0 | 376 |
| Galactose | 24 | 21 | 6 | 0 | 0 | 0 | 41 | 33 | 35 | 160 |
| Glucose | 8 | 3 | 0 | 19 | 25 | 62 | 34 | 52 | 33 | 236 |
| Mannose | 47 | 35 | 10 | 0 | 0 | 0 | 34 | 59 | 56 | 241 |
| Sugar | FimH-DSG G15A G16A V27A | | | | | | | | | Total |
| Nac- | 0 | 0 | 20 | 0 | 0 | 0 | 0 | 0 | 0 | 20 |
| Glucose | | | | | | | | | | |
| Galacose | 0 | 0 | 7 | 0 | 0 | 0 | 0 | 0 | 0 | 7 |
| Mannose | 0 | 0 | 17 | 0 | 0 | 0 | 0 | 0 | 0 | 17 |

### Example 20: FimH_{LD} and FimH-DSG G15A G16A V27A Mutants do not Bind to E. coli O-Antigens

The repeat units of E. coli O-antigens O8 and O9 are composed of polymannose residues. This raised the question of whether FimH might bind O-antigen conjugates in a FimH-O-antigen conjugate combination vaccine. Biolayer interferometry experiments were designed to test whether FimH_{LD} WT could bind to either free O9 polysaccharide or CRM-conjugated O9 polysaccharide, and whether the FimH_{LD} G15A G16A V27A mutant, which in binding assays was shown to have an undetectable affinity for mannoside ligand, could bind. O-antigen binding data is shown in Table 20. At high concentrations of free polysaccharide, a response was observed with FimH_{LD} WT. Higher responses were observed with CRM-conjugated polysaccharide. Detectable binding, however, was eliminated with the mutant protein FimH_{LD} G15A G16A V27A. Similar experiments were set up for FimH-DSG, wherein the ability of FimH-DSG WT or FimH-DSG G15A G16A V27A mutant to bind to free or CRM-conjugated O-antigens of different serotypes (O9, O25b, O1a and O2) was tested Table 21). As expected from their mannoside ligand binding properties, the overall binding affinities of the FimH-DSG WT was significantly lower than the corresponding FimH_{LD} variant. At the highest concentration of CRM conjugate, some titratable binding was observed with FimH-DSG WT with only slightly above background levels of binding seen for the free polysaccharide. As with the FimH_{LD} G15A G16A V27A mutant, the FimH-DSG G15A G16A V27A protein did not bind to any of the free or CRM-conjugated polysaccharides. In conclusion, unlike the parental WT FimH_{LD} or WT FimH-DSG antigens, the derived G15A G16A V27A mutants fail to bind O-antigens, providing a potential path forward for development of a combined FimH and O-antigen vaccine.

TABLE 20
FimH_{LD} G15A G16A V27A mutant does not bind
free or CRM-conjugated O-antigen O9 polysaccharide

| | Response (nm) | | | |
|---|---|---|---|---|
| | FimH_{LD} WT | | FimH_{LD} G15A G16A V27A | |
| O-antigen | Free O9 | O9- | Free O9 | O9- |
| (μg/ml) | poly | CRM | poly | CRM |
| 200 | 0.765 | 0.988 | 0.019 | 0.017 |
| 100 | 0.349 | 0.466 | −0.007 | 0.020 |
| 50 | 0.162 | 0.253 | 0.023 | 0.018 |
| 25 | 0.074 | 0.131 | 0.024 | 0.022 |
| 12.5 | 0.035 | 0.074 | 0.024 | 0.005 |
| 6.25 | 0.013 | 0.036 | 0.021 | 0.003 |
| 3.13 | 0.004 | 0.02 | 0.027 | 0.013 |

TABLE 21
FimH-DSG G15A G16A V27A mutant does not bind free or
CRM-conjugated O-antigen polysaccharides

| | | Response (nm) | | | |
|---|---|---|---|---|---|
| | | FimH-DSG | | FimH-DSG | |
| O- | O- | WT | | G15A G16A V27A | |
| antigen | antigen | Free | CRM | Free | CRM |
| serotype | (μg/ml) | poly | conjugate | poly | conjugate |
| O9 | 200 | 0.039 | 0.248 | −0.008 | −0.028 |
| O9 | 100 | 0.037 | 0.134 | −0.002 | −0.028 |
| O9 | 50 | 0.033 | 0.094 | −0.019 | −0.021 |
| O9 | 25 | 0.050 | 0.062 | −0.009 | −0.029 |
| O9 | 12.5 | 0.039 | 0.06 | −0.021 | −0.027 |
| O9 | 6.25 | 0.030 | 0.048 | −0.012 | −0.023 |
| O9 | 3.13 | 0.032 | 0.046 | −0.018 | −0.029 |
| O25b | 200 | −0.009 | 0.179 | −0.051 | −0.043 |
| O25b | 100 | −0.006 | 0.072 | −0.025 | −0.045 |
| O25b | 50 | −0.007 | 0.039 | −0.027 | −0.045 |
| O25b | 25 | −0.004 | 0.026 | −0.029 | −0.043 |
| O25b | 12.5 | −0.002 | 0.019 | −0.028 | −0.046 |
| O25b | 6.25 | 0.000 | 0.013 | −0.028 | −0.045 |
| O25b | 3.13 | −0.006 | 0.011 | −0.03 | −0.048 |
| O1a | 200 | 0.011 | 0.401 | −0.02 | −0.048 |
| O1a | 100 | 0.018 | 0.160 | −0.021 | −0.051 |
| O1a | 50 | 0.009 | 0.072 | −0.025 | −0.042 |
| O1a | 25 | 0.014 | 0.031 | −0.024 | −0.050 |
| O1a | 12.5 | 0.009 | 0.006 | −0.029 | −0.047 |
| O1a | 6.25 | 0.013 | −0.005 | −0.032 | −0.05 |
| O1a | 3.13 | 0.008 | −0.008 | −0.030 | −0.053 |
| O2 | 200 | 0.032 | 0.510 | −0.011 | −0.039 |
| O2 | 100 | 0.029 | 0.240 | −0.046 | −0.051 |
| O2 | 50 | 0.029 | 0.119 | −0.032 | −0.048 |
| O2 | 25 | 0.025 | 0.066 | −0.032 | −0.048 |
| O2 | 12.5 | 0.028 | 0.035 | −0.023 | −0.040 |
| O2 | 6.25 | 0.029 | 0.017 | −0.024 | −0.052 |
| O2 | 3.13 | 0.028 | 0.006 | −0.023 | −0.049 |

### Example 21: Non-Human Primates Vaccinated with FimH-DSG G15A G16A V27A Mutant with and without O-Antigens

### A. Methods

1. FimH IgG dLIA

E. coli mutant fimbrial antigen FimH-DSG G15A G16A V27A coupled to spectrally distinct MagPlex-C microspheres (Luminex) were diluted in blocking buffer to the concentration 50,000 beads/mL for 1-2 hours at room temperature while shaking immediately prior to assay primary incubation. The diluted microsphere mixture was added to assay plates containing appropriately diluted non-human primate serum samples, controls and the reference standard, a humanized in-house monoclonal antibody (FimH Y202) that binds the pilin domain of FimH-DSG, for incubation overnight at 2-8° C. while shaking. After washing off non-bound components, a purified R-Phycoerythrin goat anti-human IgG, Fcγ Fragment Specific secondary antibody (Jackson ImmunoResearch Laboratories, 109-116-170) was added to the microsphere mixture and incubated for 90 minutes at room temperature while shaking. The magnitude of the fluorescent PE signal measured by a Luminex FLEXMAP 3D reader is directly proportional to the amount of anti-FimH-DSG IgG bound to the protein coupled microspheres. The data was analyzed using a custom SAS application, which uses a log/log linear regression model of the standard curve to interpolate antigen-specific antibody concentrations (μg/mL) from median fluorescent intensity. A lower limit of quantitation (LLOQ) of 0.763 μg/mL was calculated from standard curve bias.

2. 4-Valent O-Ag IgG dLIA

E. coli long O-antigen polysaccharides of serotypes O25b, O1a, O2 and O6 were covalently conjugated to poly-L-lysine, and the derived conjugates were coupled to spectrally distinct MagPlex-C microspheres (Luminex) with a standard EDC/NHS mediated coupling protocol. Microspheres were incubated with serially diluted non-human primate serum samples, controls and polyclonal standard, for incubation overnight at 2-8° C. while shaking. After washing, bound serotype-specific IgG was detected with a PE-conjugated goat anti-human IgG, Fcγ Fragment Specific secondary antibody (Jackson ImmunoResearch Laboratories, 109-115-098) following incubation for 90 minutes at room temperature while shaking. Fluorescence was measured by a Luminex 200 reader for each of the four spectrally distinct regions and expressed as median fluorescent intensities. A standard curve plot of the polyclonal standard titration yielded a linear slope profile with arbitrary assignment from which signals could be interpolated as serotype-specific antibody levels (U/mL).

### 3. Non-Human Primates (NHPs)

Female Cynomolgus Macaques (Macaca fasicularis) were originally obtained from Charles River Laboratories (Houston, TX) before being transferred to Pfizer, Pearl River, NY (age range: 4-5 years, weight range: 3.1-5.9 kg). NHPs were housed in standard quad caging with water and food provided ad libitum. Animals were microchipped subcutaneously to monitor internal temperature. Only NHP free of E. coli infection were enrolled based on negative urine qPCR results (see method section 10 below).

### 4. Vaccination and Blood Collection

Cynomolgus macaques were immunized intramuscularly (0.55 mL) at week 0, 4 and 14 either with vehicle control (PBS, pH 6.2), a monomeric fimbrial antigen FimH-DSG G15A G16A V27A (50 μg/dose), or with a mixture of 4-valent O25b, O1a, O2 and O6 O-antigen polysaccharides conjugates (1 μg/dose) in combination with a monomeric fimbrial antigen FimH-DSG G15A G16A V27A (50 μg/dose). Vaccine antigens were adjuvanted with AS01b (50 μg of MPL and 50 μg of QS-21 per dose).

On week 0, 6 and 16, 10 mL of blood was collected via the femoral vein into 1 serum separator tube (BD Vacutainer), using a 21 g safety needled/vacutainer. Collection tubes were left at room temperature for 30 min and centrifuged at 3000 g for 10 min. Serum in the supernatant was collected, aliquoted and stored at −80° C.

5. E. coli Clinical Isolate

One representative ST131 O25b clinical isolate was selected based on patient's age and origin of sample collection (PFEEC0578, Male, age 38, bladder origin) from UPEC strains collected as part of the Pfizer-sponsored Antimicrobial Testing Leadership and Surveillance (ATLAS) database which is maintained by the International Health Management associates (IHMA) clinical lab. strain carries genes that encode the production of capsular polysaccharides of an unknown type.

### 6. UPEC Strain Stock Preparation

E. coli stock was prepared by inoculation of 12 mL of LB broth (Teknova, #L8198) followed by an overnight incubation at 37° C. under agitation at 275 rpm. After 18 hrs, the 12 mL culture was diluted into 113 mL of LB broth in a 250 mL flask (Corning, #431407). The culture was incubated for 2 to 3 hrs at 37° C. at −275 rpm until an OD₆₀₀ between 2.1 and 2.7. Twenty-five mL of glycerol (80%, MP, #3055-044) was mixed to the culture. Aliquots of 5 mL were frozen at −80° C. for long term storage. The concentration of viable bacteria per vial was confirmed by plating serial dilutions of the stock onto TSA plates (BD, BBL Trypticase Soy Agar (Soybean Casein Digest Agar) Catalog #B21283X) and analyzed after 18 hrs incubation at 30° C.

### 7. Nonhuman Primate Model of Cystitis

NHPs were anesthetized with a Ketamine/Dendrometer mixture administered intramuscularly. To prevent bladder contamination from urethral catheterization, the anogenital area was wiped down with sterile gauze moistened with sterile saline and/or antiseptic wipes containing Benzalkonium Chloride. A sterile 5 French red rubber catheter, previously coated with Surgi Lube to prevent tissue irritation, was gently introduced through the urethra into the bladder. The bladder was then voided of any urine either via natural flow or by aspiration with a syringe. Through the catheter, a volume of 1 mL containing 10⁸ CFU of UPEC strain PFEEC0578 was administered directly into the bladder.

### 8. Post Challenge Animal Monitoring

After challenge, animals were monitored twice a day during week 1 and once a day during subsequent weeks. NHPs were monitored up to 30 days post challenge. Monitoring included observation of the appearance of urine output, changes in behavior or appetite, signs of pain/discomfort and body temperature measurement.

### 9. Urine Collection Via Catheter Placement

To allow collection of clean urine samples, the bladder of anesthetized NHPs were catheterized as described above. After catheter placement, the bladder was voided of urine either via by natural flow or by aspiration with a syringe. When bladders contained no urine, 10 mL of saline was infused and aspiration through the catheter was repeated. All samples collected were immediately stored on ice.

### 10. DNA Extraction

Extraction of E. coli DNA from up to three replicates of NHP urine samples was performed using Qiagen Minelute DNA Extraction kits (Qiagen, Ref #51306, quantity was sometimes limited by sample volume collected). The manufacturer's Blood and Body Fluid Spin protocol was followed with the following modifications: sample starting volume was increased to 500 μL (if sample volume allowed), Buffer AL volume was increased to 500 μL, Proteinase K volume was increased to 50 μL, 50 μL of Molecular Grade Water (Corning Inc., Ref #46-000-CM) was warmed to 37° C. and used in place of elution buffer EB. Finally, after addition of 37° C. Molecular Grade Water, spin columns were incubated for 5 min at room temperature prior to final spin.

11. Quantitative Real-Time PCR (qPCR)

Quantitative real-time PCR (qPCR) was used to assess bacterial load in NHP urine samples. E. coli O25b serotype specific DNA was amplified with qPCR using the following primers: forward, TTGAAAGTGATGGTTTGGTAAGAAAT (SEQ ID NO: 109); reverse, TGCAGCACGTATGATAACTTCAAAG (SEQ ID NO: 110), and a probe with Fam fluorescent reporter and sequence AGGATATTTTACCCAGCAGTGCCCCGT (SEQ ID NO: 111) was used to quantify replication.

The O25b serotype specific amplicons correspond to portions of the O25b serotype orf10 region. Primers and probe were custom designed, purchased lyophilized from Integrated DNA Technologies, and reconstituted in Buffer TE (Corning Inc., Ref #46-009-CM) to a concentration of 100 nmol/mL.

DNA samples from the DNA extraction procedure described above were assayed in 96 well Applied Biosystems MicroAmp Optical 96 Well Reaction Plates (Applied Biosystems, Ref #N8010560). The qPCR reaction was performed in a total volume of 25 μl using 12.5 μL Applied Biosystems 2× Taqman Fast Advanced Master Mix (Applied Biosystems, Ref #4444554), 0.125 μL of each reconstituted Primer, 0.5 μL Probe, 1.75 μL Molecular Biology Grade Water (Corning Inc., Ref #46-000-CM), and 10 μL of sample per well.

The reaction conditions for amplification of DNA were 50° C. for 2 minutes then 95° C. for 2 min, and 40 cycles of 95° C. for 3 seconds and 60° C. for 30 seconds, run on either an Applied Biosystems 7500 Real Time-PCR System or an Applied Biosystems QuantStudio 6 Real Time-PCR System (Applied Biosystems).

To enable quantitation, a linear standard curve was developed. Aliquots of the same frozen stock of E. coli as used in each challenge experiment (preparation described above) were diluted to 1×109 CFU/mL in Sterile PBS (Corning, 21-040-CM). Subsequent serial dilution was performed to generate solutions containing E. coli at concentrations of 1×10⁸, 1×10⁷, 1×10⁶, 1×10⁵, 1×10⁴, 1000, 100, and 10 CFU/mL. Serial dilutions were prepared in sterile PBS (Corning, 21-040-CM) or pooled, twice filtered, NHP urine collected from subjects prior to inoculation. Dilutions in PBS and pooled, twice filtered, NHP urine were later found to be equivalent. The quantity of viable bacteria present at each dilution was confirmed by plating on TSA plates (BD, BBL Trypticase Soy Agar (Soybean Casein Digest Agar) Catalog #B21283X). DNA extraction was performed on each serial dilution using the same methods employed to extract DNA from samples, as described above. These qPCR standards were run on every qPCR assay plate, in duplicate.

Linear Regression analysis was performed using Applied Biosystems QuantStudio software. Statistical analysis determined that the standard curve generated behaved linearly between 100 and 1×10⁸ bacteria/mL. Consequently, the lower limit of quantitation (LLOQ) was determined to be 100 bacteria/mL. In some instances, samples reached the fluorescence threshold, but at a cycle corresponding to a quantity below the LLOQ, in other instances, the fluorescence threshold was not reached at all (undetermined value). When either of these conditions occurred, the values are reported here as the value of the LLOQ (100 bacteria/mL).

### 12. Myeloperoxidase (MPO) ELISA

Invitrogen Myeloperoxidase Instant ELISA Kits (Invitrogen, Ref #BMS20381NST) were used to quantify Myeloperoxidase (MPO) in NHP urine. PIPES Buffer was added to neat urine samples to a final concentration of 5% 0.5M PIPES buffer pH 6.8 (Alfa Aesar, Ref #J61786-AK). Samples were vortexed for 15 seconds and then diluted 1:1 with manufacturer supplied Sample Diluent. Samples were assayed in duplicate and manufacturer's instructions were followed for the remainder of the assay. At final endpoint, color intensity at 450 nm was measured on a Spectramax Plus instrument (Molecular Devices). The assay kit's included standards were used to generate a standard curve. For analysis, the assay kit's low standard (156.25 pg/mL) was understood to be the lower limit of detection (LLOD). The Spectramax instrument's companion software (Softmax, Molecular Devices) may extrapolate beyond the value of the low standard. The value of one-half of the LLOD (78.125 pg/mL) was substituted for any values extrapolated below that value or when any assay result fell entirely below the limit of detection.

### 13. IL-8 Luminex Assay

Interleukin-8 (IL-8) was measured using a Custom Bio-Rad IL-8 Human Cytokine Screening Panel Luminex Assay kit (BioRad Laboratories Inc., REF #17005177). PIPES Buffer was added to neat urine samples to a final concentration of 5% 0.5M PIPES buffer pH 6.8 (Alfa Aesar, Ref #J61786-AK). Samples were vortexed for 15 seconds and then diluted 1:1 with 50% of a modified LXA-4 Buffer (PBS 1×, 0.5% BSA, 0.025% sodium azide). Samples were assayed in duplicate and manufacturer's instructions were followed for the remainder of the assay. Assay plate was read on a BioPlex 200 Luminex instrument (BioRad Laboratories Inc.). The Bio-Plex 200 Luminex instrument's companion software, “BioPlex Manager”, and the assay kit's included standards were used to generate a standard curve and to extrapolate sample concentration from fluorescence intensity. The BioPlex Manager software determines the lower limit of quantitation (LLOQ).

### 14. Total Nucleated Cell Count and Light Microscopy Analysis of Urine Sediments

Within 1 hour of collection, urine samples (500 μl to 1 mL) were fixed with formalin to a final concentration of 1% and sent overnight on ice to Pfizer Groton, CT. Upon receipt, total nucleated cells (epithelial cells and polymorphonuclear cells) were counted on a Hemocytometer. Approximately 300 μL total was loaded into the Thermo Scientific Shandon EZ double cyto funnel, 100 μL into one funnel and 200 μL into the other funnel. Samples were cytocentrifuged for 5 min at 750 rpm onto Shandon Double Cytoslide Microscope Coated glass slides using a Thermo Scientific CytoSpin 4 cytocentrifuge. For samples with high cell counts, urine was first diluted 1:10 with 0.9% saline before cytocentrifugation. Using a Sysmex SP-10 instrument, cytoprep slides were then briefly methanol fixed and stained with Giemsa and May-Grunwald stain. For each urine sample, one slide per urine sample was prepared with the two sample volumes listed above.

Cytospin slides containing concentrated and stained urine sediment were evaluated by light microscopy for the presence or absence of increased polymorphonuclear cells (PMN, i.e. granulocytes with segmented or reniform nuclei, generally comprised of neutrophils but also including eosinophils and basophils). The absence of increased PMN cells was determined based on the observation of no PMN or rare PMN. The presence of increased PMN was determined based on the observation of more than rare PMN, relative to the background epithelial cell population.

### B. Results

1. Vaccination with FimH-DSG G15A G16A V27A Mutant with and without O-Antigens Elicits Potent Total and Neutralizing Antibodies in Non-Human Primates

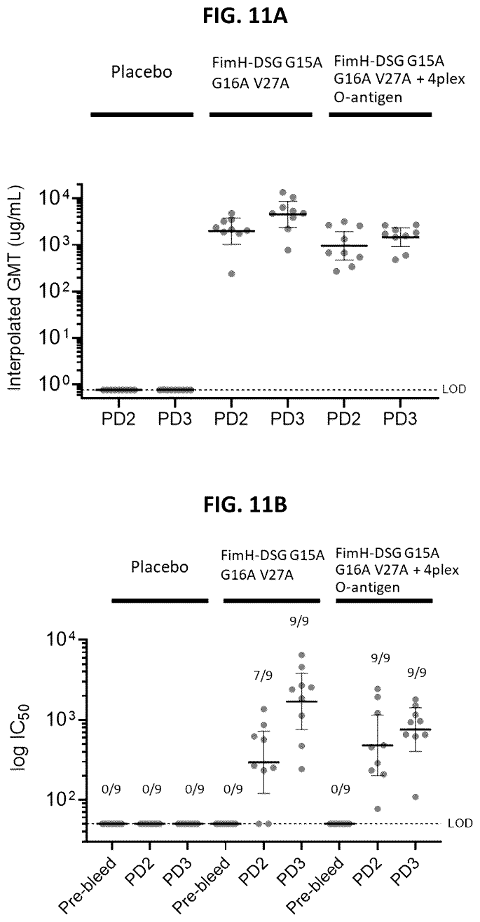

Non-human primates were vaccinated with FimH-DSG G15A G16A V27A mutant with or without a 4-valent O-antigen conjugate (O25b, O6, O1a, O2) and AS01b adjuvant (FIG. 9). In the FimH-DSG G15A G16A V27A and 4-valent O-antigen vaccinated group, O-antigen serotype-specific antibody titers rose ˜400-fold at 6 weeks post vaccination (FIG. 10 and Table 22). Total anti-FimH antibody titers were quantified using a direct Luminex immunoassay (dLIA) (FIG. 11A and Table 23). Animals in the placebo group had titers below the assay limit of quantification. In both vaccinated groups, titers rose after two doses and could be boosted with a third dose. Overall, titers were slightly lower in animals vaccinated with FimH-DSG G15A G16A V27A with O-antigens compared to FimH-DSG G15A G16A V27A alone.

TABLE 22
O-antigen serotype-specific titers in non-human primates
vaccinated with FimH-DSG G15A G16A V27A with
4-valent O-antigen

| Serotype | Week | GMT |
|---|---|---|
| O1a | 0 | 3.842 |
| | 6 | 2903.324 |
| | 16 | 3446.427 |
| O2 | 0 | 4.318 |
| | 6 | 5449.571 |
| | 16 | 5696.599 |
| O6 | 0 | 5.093 |
| | 6 | 2412.359 |
| | 16 | 1686.817 |
| O25b | 0 | 2.636 |
| | 6 | 3483.843 |
| | 16 | 4028.502 |

TABLE 23
FimH IgG geometric mean titers in non-human primates

| Group | | | FimH-DSG G15A | | FimH-DSG G15A G16A | |
|---|---|---|---|---|---|---|
| Time | Placebo | | G16A V27A | | V27A + 4-4-valent O-antigen | |
| point | PD2 | PD3 | PD2 | PD3 | PD2 | PD3 |
| GMT | 0.76 | 0.76 | 1965.69 | 4500.47 | 951.61 | 1457.49 |

Sera were evaluated in the E. coli binding inhibition assay in order to assess ability of anti-FimH antibodies to block binding of E. coli to yeast mannan (FIG. 11B and Table 24). The mean IC₅₀ of sera from animals vaccinated with FimH-DSG G15A G16A V27A alone rose to 293.65 post dose 2 and 1698.39 post dose 3, while the mean IC₅₀ of animals vaccinated with FimH-DSG G15A G16A V27A in combination with O-antigens was 480.12 after dose 2 and 756.45 after dose 3.

TABLE 24
E. coli neutralization assay IC₅₀ for non-human primate sera

| Group | | | | | | | FimH-DSG G15A | | |
|---|---|---|---|---|---|---|---|---|---|
| Time | Placebo | | | FimH-DSG G15A G16A V27A | | | G16A V27A + 4-valent O-antigen | | |
| point | Pre-bleed | PD2 | PD3 | Pre-bleed | PD2 | PD3 | Pre-bleed | PD2 | PD3 |
| Geomean | 50 | 50 | 50 | 50 | 293.65 | 1698.39 | 50 | 480.12 | 756.45 |
| IC₅₀ | | | | | | | | | |

Together, these data show that FimH-DSG G15A G16A V27A elicits a potent antibody response in non-human primates, and combining with O-antigens results in high titers though slightly lower than with FimH alone.

2. Vaccination with FimH-DSG G15A G16A V27A Mutant with and without O-Antigens Reduces Bacteriuria and Biomarkers of Infection in a Non-Human Primate Model

Five weeks after the final boost, vaccinated and placebo treated NHPs were inoculated via intravesical catherization with 108 CFU of UPEC isolate PFEEC0578. Bacteriuria was monitored in catheter collected urine over a period of 28 days. In all placebo treated animals, the instillation of live bacteria led to a high level of bacteriuria on day 2 and 7 post-challenge (geometric mean of approximatively 106 bacteria/mL of urine). Compared to placebo group, animals vaccinated with FimH-DSG G15A G16A V27A or FimH-DSG G15A G16A V27A+4-valent O-antigen exhibited a 300-fold or a 1000-fold reduction in geo mean bacteriuria respectively at day 2 and 7 post-infection.

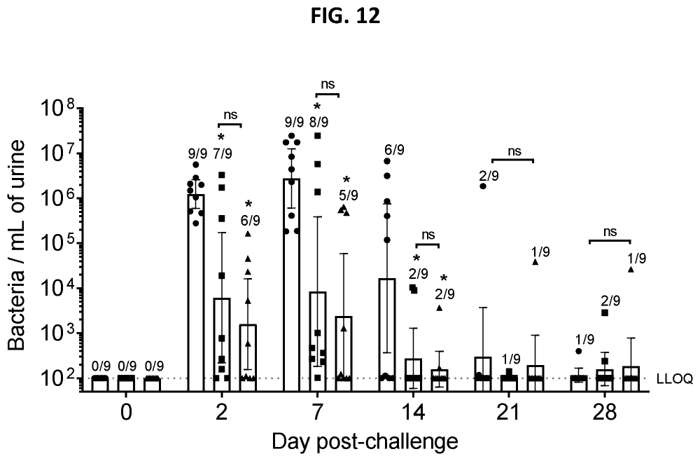

On day 14, approximately 50% of placebo vaccinated animals still exhibited bacteriuria >105 bacteria/mL of urine. Finally, the majority of placebo NHPs cleared the infection on day 21 and 28. In contrast, most FimH-DSG G15A G16A V27A or FimH-DSG G15A G16A V27A+4-valent O-antigen vaccinated animals cleared the infection by day 14 (FIG. 12).

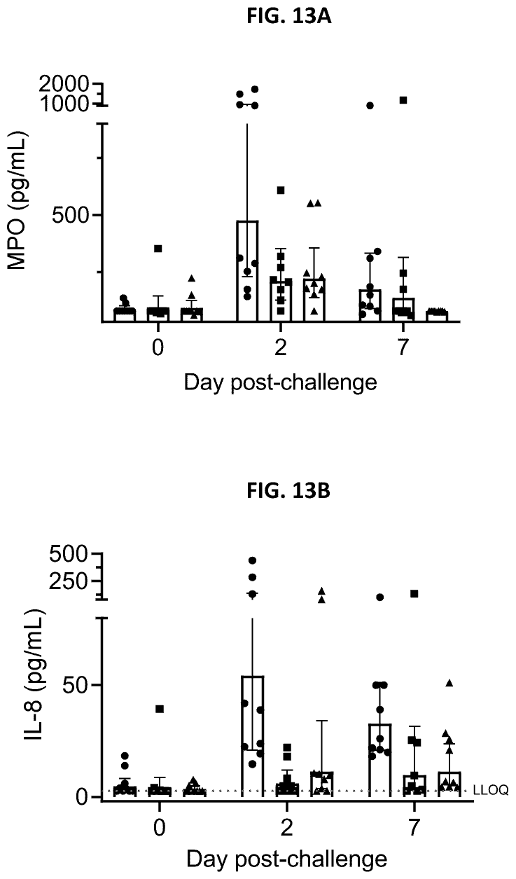

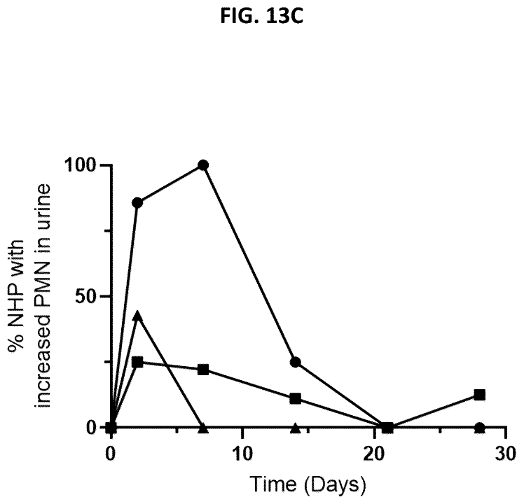

Next, various inflammatory biomarkers were monitored in the urine of challenged NHPs. At day 7 post-challenge, all placebo treated animals exhibited elevated levels of polymorphonuclear (PMN) cells in urine sediment as confirmed by cytology analysis. In contrast, less than 25% of FimH-DSG G15A G16A V27A vaccinated NHPs and no FimH-DSG G15A G16A V27A+4-valent O-antigen immunized animals had increased levels of PMN cells in urine sediment (FIG. 13C). In parallel, we measured levels of myeloperoxidase (MPO) and Interleukin 8 (IL-8) in urine samples over a 7-day period. On day 2 post-challenge, both vaccinated groups exhibited a 2-fold reduction in MPO levels (geometric mean of ˜200 pg/mL) compared to placebo group (geometric mean of 470 pg/mL) (FIG. 13A).

In addition, on day 2 and day 7 post-infection, urine concentrations of IL-8 in FimH-DSG G15A G16A V27A vaccinated animals were decreased by approximatively 10- and 5-fold respectively (geometric mean of 5.9 pg/mL and 9.8 pg/mL) compared to levels measured in urine of placebo treated NHPs (geometric mean of 54.2 pg/mL and 32.7 pg/mL). With a similar trend, urine levels of IL-8 on day 2 and day 7 in FimH-DSG G15A G16A V27A in combination with O-antigens immunized NHPs were reduced by approximatively 5- and 3-fold respectively (geometric mean of 11.3 pg/mL) compared to IL-8 concentrations observed in placebo treated animals (FIG. 13B).

### C. Conclusions

FimH-DSG G15A G16A V27A mutant induces high anti-FimH IgG titers in NHPs that can be boosted with a 3^{rd} dose. Animals vaccinated with the combination of FimH-DSG G15A G16A V27A mutant and the 4-valent O-antigens showed high O-antigen IgG titers.

FimH-DSG G15A G16A V27A elicits potent neutralizing antibodies in non-human primates. Combination with the 4-valent O-antigens is similarly immunogenic.

FimH-DSG G15A G16A V27A mutant with or without the 4-valent O-antigens reduces bacteriuria and biomarkers of infection in a urinary tract infection model in Cynomolgus macaques.

The following clauses describe additional aspects of the disclosure:
C1. A mutated FimH polypeptide, which comprises at least one amino acid mutation relative to the amino acid sequence of the wild-type FimH polypeptide, wherein the mutation position is selected from the group consisting of: F1, P12, G14, G15, G16, A18, P26, V27, V28, Q32, N33, L34, V35, R60, S62, Y64, G65, L68, F71, T86, L107, Y108, L109, V112, S113, A115, G116, V118, A119, A127, L129, Q133, F144, V154, V155, V156, P157, T158, V163, and V185, wherein the amino acid positions are numbered according to SEQ ID NO:59.
C2. A mutated FimH polypeptide according to clause C1, comprising at least one mutation selected from the group consisting of: F1I; F1L; F1V; F1M; F1Y; F1W; P12C; G14C; G15A; G15P; G16A; G16P; A18C; P26C; V27A; V27C; V28C; Q32C; N33C; L34C; L34N; L34S; L34T; L34D; L34E; L34K; L34R; V35C; R60P; S62C; Y64C; G65A; L68C; F71C; T86C; L107C; Y108C; L109C; V112C; S113C; A115V; G116C; V118C; A119C; A119N; A119S; A119T; A119D; A119E; A119K; A119R; A127C; L129C; Q133K; F144C; V154C; V156C; P157C; T158C; V1631; and V185I, or any combination thereof.
C3. A mutated FimH polypeptide according to clause C2, comprising the mutations G15A and G16A.
C4. A mutated FimH polypeptide according to clause C2, comprising the mutations P12C and A18C.
C5. A mutated FimH polypeptide according to clause C2, comprising the mutations G14C and F144C.
C6. A mutated FimH polypeptide according to clause C2, comprising the mutations P26C and V35C.
C7. A mutated FimH polypeptide according to clause C2, comprising the mutations P26C and V154C.
C8. A mutated FimH polypeptide according to clause C2, comprising the mutations P26C and V156C.
C9. A mutated FimH polypeptide according to clause C2, comprising the mutations V27C and L34C.
C10. A mutated FimH polypeptide according to clause C2, comprising the mutations V28C and N33C.
C11. A mutated FimH polypeptide according to clause C2, comprising the mutations V28C and P157C.
C12. A mutated FimH polypeptide according to clause C2, comprising the mutations Q32C and Y108C.
C13. A mutated FimH polypeptide according to clause C2, comprising the mutations N33C and L109C.
C14. A mutated FimH polypeptide according to clause C2, comprising the mutations N33C and P157C.
C15. A mutated FimH polypeptide according to clause C2, comprising the mutations V35C and L107C.
C16. A mutated FimH polypeptide according to clause C2, comprising the mutations V35C and L109C.
C17. A mutated FimH polypeptide according to clause C2, comprising the mutations S62C and T86C.
C18. A mutated FimH polypeptide according to clause C2, comprising the mutations S62C and L129C.
C19. A mutated FimH polypeptide according to clause C2, comprising the mutations Y64C and L68C.
C20. A mutated FimH polypeptide according to clause C2, comprising the mutations Y64C and A127C.
C21. A mutated FimH polypeptide according to clause C2, comprising the mutations L68C and F71C.
C22. A mutated FimH polypeptide according to clause C2, comprising the mutations V112C and T158C.
C23. A mutated FimH polypeptide according to clause C2, comprising the mutations S113C and G116C.
C24. A mutated FimH polypeptide according to clause C2, comprising the mutations S113C and T158C.
C25. A mutated FimH polypeptide according to clause C2, comprising the mutations V118C and V156C.
C26. A mutated FimH polypeptide according to clause C2, comprising the mutations A119C and V155C.
C27. A mutated FimH polypeptide according to clause C2, comprising the mutations L34N and V27A.
C28. A mutated FimH polypeptide according to clause C2, comprising the mutations L34S and V27A.
C29. A mutated FimH polypeptide according to clause C2, comprising the mutations L34T and V27A.
C30. A mutated FimH polypeptide according to clause C2, comprising the mutations L34D and V27A.
C31. A mutated FimH polypeptide according to clause C2, comprising the mutations L34E and V27A.
C32. A mutated FimH polypeptide according to clause C2, comprising the mutations L34K and V27A.
C33. A mutated FimH polypeptide according to clause C2, comprising the mutations L34R and V27A.
C34. A mutated FimH polypeptide according to clause C2, comprising the mutations A119N and V27A.
C35. A mutated FimH polypeptide according to clause C2, comprising the mutations A119S and V27A.
C36. A mutated FimH polypeptide according to clause C2, comprising the mutations A119T and V27A.
C37. A mutated FimH polypeptide according to clause C2, comprising the mutations A119D and V27A.
C38. A mutated FimH polypeptide according to clause C2, comprising the mutations A119E and V27A.
C39. A mutated FimH polypeptide according to clause C2, comprising the mutations A119K and V27A.
C40. A mutated FimH polypeptide according to clause C2, comprising the mutations A119R and V27A.
C41. A mutated FimH polypeptide according to clause C2, comprising the mutations G15A and V27A.
C42. A mutated FimH polypeptide according to clause C2, comprising the mutations G16A and V27A.
C43. A mutated FimH polypeptide according to clause C2, comprising the mutations G15P and V27A.
C44. A mutated FimH polypeptide according to clause C2, comprising the mutations G16P and V27A.
C45. A mutated FimH polypeptide according to clause C2, comprising the mutations G15A, G16A, and V27A.
C46. A mutated FimH polypeptide according to clause C2, comprising the mutations G65A and V27A.
C47. A mutated FimH polypeptide according to clause C2, comprising the mutations V27A and Q133K.
C48. A mutated FimH polypeptide according to clause C2, comprising the mutations G15A, G16A, V27A, and Q133K.
C49. A mutated FimH polypeptide according to clause C2, comprising the sequence of any one of SEQ ID NOs: 2-58, and 60-64.
C50. A mutated FimH polypeptide according to any of clauses C1-C49, wherein the polypeptide is isolated.
C51. A pharmaceutical composition comprising (i) a mutated FimH polypeptide according to any one of clauses C1-C50 and (ii) a pharmaceutically acceptable carrier.
C52. An immunogenic composition comprising a mutated FimH polypeptide according to any one of clauses C1-C50.
C53. An immunogenic composition according to clause C52, further comprising at least one additional antigen.
C54. An immunogenic composition according to clause C53, wherein the at least one additional antigen is a saccharide, or a polysaccharide, or a glycoconjugate, or a protein.
C55. An immunogenic composition according to clause C52, further comprising at least one adjuvant.
C56. A nucleic acid molecule comprising a nucleotide sequence that encodes an amino acid sequence of a mutated FimH polypeptide according to any one of clauses C1-C49.
C57. A mutated FimH polypeptide according to any of clauses C1-C50, wherein the polypeptide is immunogenic.
C58. A recombinant mammalian cell, comprising a polynucleotide encoding a mutated FimH polypeptide according to any one of clauses C1-C50.
C59. A culture comprising the recombinant cell of clause C58, wherein said culture is at least 5 liters in size.
C60. A method for producing a mutated FimH polypeptide according to any one of clauses C1-C50, comprising culturing a recombinant mammalian cell according to clause C58 under suitable conditions, thereby expressing the polypeptide; and harvesting the polypeptide.
C61. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of a composition according to any one of clauses C51 to C55.
C62. A method according to clause C61, wherein the subject is at risk of developing a urinary tract infection.
C63. A method according to clause C61, wherein the subject is at risk of developing bacteremia.
C64. A method according to clause C61, wherein the subject is at risk of developing sepsis.
C65. A method of eliciting an immune response against E. coli in a mammal, comprising administering to the mammal an effective amount of a composition according to any one of clauses C51-C55.
C66. A method according to clause C65, wherein the immune response comprises opsonophagocytic and/or neutralizing antibodies against E. coli.
C67. A method according to clause C65, wherein the immune response protects the mammal from an E. coli infection.
C68. A method of preventing, treating or ameliorating a bacterial infection, disease or condition in a subject, comprising administering to the subject an immunologically effective amount of a composition according to any one of clauses C51-C55.
C69. An immunogenic composition according to clause C54, wherein the additional antigen is a saccharide comprising a structure selected from any one of Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O8, Formula O9, Formula O10, Formula O11, Formula O12, Formula O13, Formula O14, Formula O15, Formula O16, Formula O17, Formula O18 (e.g., Formula O18A, Formula O18ac, Formula O18A1, Formula O18B, and Formula O18B1), Formula O19, Formula O20, Formula O21, Formula O22, Formula O23 (e.g., Formula O23A), Formula O24, Formula O25 (e.g., Formula O25a and Formula O25b), Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45 (e.g., Formula O45 and Formula O45rel), Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D₁, Formula O63, Formula O64, Formula O65, Formula O66, Formula O68, Formula O69, Formula O70, Formula O71, Formula O73 (e.g., Formula O73 (strain 73-1)), Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, and Formula O187, wherein n is an integer from 1 to 100.
C70. The immunogenic composition according to clause C69, wherein the saccharide comprises a structure selected from Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O10, Formula O16, Formula O17, Formula O18 (e.g., Formula O18A, Formula O18ac, Formula O18A1, Formula O18B, and Formula O18B1), Formula O21, Formula O23 (e.g., Formula O23A), Formula O24, Formula O25 (e.g., Formula O25a and Formula O25b), Formula O26, Formula O28, Formula O44, Formula O45 (e.g., Formula O45 and Formula O45rel), Formula O55, Formula O56, Formula O58, Formula O64, Formula O69, Formula O73 (e.g., Formula O73 (strain 73-1)), Formula O75, Formula O77, Formula O78, Formula O86, Formula O88, Formula O90, Formula O98, Formula O104, Formula O111, Formula O113, Formula O114, Formula O119, Formula O121, Formula O124, Formula O125, Formula O126, Formula O127, Formula O128, Formula O136, Formula O138, Formula O141, Formula O142, Formula O143, Formula O147, Formula O149, Formula O152, Formula O157, Formula O158, Formula O159, Formula O164, Formula O173, Formula 62D₁, Formula O22, Formula O35, Formula O65, Formula O66, Formula O83, Formula O91, Formula O105, Formula O116, Formula O117, Formula O139, Formula O153, Formula O167, and Formula O172, wherein n is an integer from 31 to 100.
C71. The immunogenic composition according to clause C70, wherein the saccharide comprises a structure selected from Formula O1 (e.g., Formula O1A, Formula O1B, and Formula O1C), Formula O2, Formula O3, Formula O4 (e.g., Formula O4:K52 and Formula O4:K6), Formula O5 (e.g., Formula O5ab and Formula O5ac (strain 180/C3)), Formula O6 (e.g., Formula O6:K2; K13; K15 and Formula O6:K54), Formula O7, Formula O10, Formula O16, Formula O17, Formula O18 (e.g., Formula O18A, Formula O18ac, Formula O18A1, Formula O18B, and Formula O18B1), Formula O21, Formula O23 (e.g., Formula O23A), Formula O24, Formula O25 (e.g., Formula O25a and Formula O25b), Formula O26, Formula O28, Formula O44, Formula O45 (e.g., Formula O45 and Formula O45rel), Formula O55, Formula O56, Formula O58, Formula O64, Formula O69, Formula O73 (e.g., Formula O73 (strain 73-1)), Formula O75, Formula O77, Formula O78, Formula O86, Formula O88, Formula O90, Formula O98, Formula O104, Formula O111, Formula O113, Formula O114, Formula O119, Formula O121, Formula O124, Formula O125, Formula O126, Formula O127, Formula O128, Formula O136, Formula O138, Formula O141, Formula O142, Formula O143, Formula O147, Formula O149, Formula O152, Formula O157, Formula O158, Formula O159, Formula O164, Formula O173, and Formula 62D₁, wherein n is an integer from 31 to 100.
C72. The immunogenic composition according to clause C70, comprising a structure selected from Formula O1 (e.g., Formula O1A, Formula O1B, and Formula O1C), Formula O2, Formula O6 (e.g., Formula O6:K2; K13; K15 and Formula O6:K54), Formula O15, Formula O16, Formula O21, Formula O25 (e.g., Formula O25a and Formula O25b), and Formula O75.
C73. The immunogenic composition according to clause C70, comprising a structure selected from Formula O4, Formula O11, Formula O21, and Formula O75.
C74. The immunogenic composition according to clause C69, wherein the saccharide does not comprise a structure selected from Formula O8, Formula O9a, Formula O9, Formula O20ab, Formula O20ac, Formula O52, Formula O97, and Formula O101.
C75. The immunogenic composition according to clause C69, wherein the saccharide does not comprise a structure selected from Formula O12.
C76. The immunogenic composition according to clause C72, wherein the saccharide is produced by expressing a wzz family protein in a Gram-negative bacterium to generate said saccharide.
C77. The immunogenic composition according to clause C76, wherein the wzz family protein is selected from the group consisting of wzzB, wzz, WZZ_{SF}, WZZ_{ST}, fepE, WZZ_{fepE}, wzz1 and wzz2.
C78. The immunogenic composition according to clause C76, wherein the wzz family protein is wzzB.
C79. The immunogenic composition according to clause C76, wherein the wzz family protein is fepE.
C80. The immunogenic composition according to clause C76, wherein the wzz family protein is wzzB and fepE.
C81. The immunogenic composition according to clause C76, wherein the wzz family protein is derived from Salmonella enterica.
C82. The immunogenic composition according to clause C76, wherein the wzz family protein comprises a sequence selected from any one of SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, and SEQ ID NO: 121.
C83. The immunogenic composition according to clause C76, wherein the wzz family protein comprises a sequence having at least 90% sequence identity to any one of 112, SEQ ID NO: 113, SEQ ID NO: 114, SEQ ID NO: 115, and SEQ ID NO: 116.
C84. The immunogenic composition according to clause C76, wherein the wzz family protein comprises a sequence selected from any one of SEQ ID NO: 117, SEQ ID NO: 118, SEQ ID NO: 119, SEQ ID NO: 120, and SEQ ID NO: 121.
C85. The immunogenic composition according to clause C69, wherein the saccharide is synthetically synthesized.
C86. The immunogenic composition according to any one of clauses C69 to C85, wherein the saccharide further comprises an E. coli R1 moiety.
C87. The immunogenic composition according to any one of clauses C69 to C85, wherein the saccharide further comprises an E. coli R2 moiety.
C88. The immunogenic composition according to any one of clauses C69 to C85, wherein the saccharide further comprises an E. coli R3 moiety.
C89. The immunogenic composition according to any one of clauses C69 to C85, wherein the saccharide further comprises an E. coli R4 moiety.
C90. The immunogenic composition according to any one of clauses C69 to C85, wherein the saccharide further comprises an E. coli K-12 moiety.
C91. The immunogenic composition according to any one of clauses C69 to C90, wherein the saccharide further comprises a 3-deoxy-d-manno-oct-2-ulosonic acid (KDO) moiety.
C92. The immunogenic composition according to any one of clauses C69 to C85, wherein the saccharide does not further comprise an E. coli R1 moiety.
C93. The immunogenic composition according to any one of clauses C69 to C85, wherein the saccharide does not further comprise an E. coli R2 moiety.
C94. The immunogenic composition according to any one of clauses C69 to C85, wherein the saccharide does not further comprise an E. coli R3 moiety.
C95. The immunogenic composition according to any one of clauses C69 to C85, wherein the saccharide does not further comprise an E. coli R4 moiety.
C96. The immunogenic composition according to any one of clauses C69 to C85, wherein the saccharide does not further comprise an E. coli K-12 moiety.
C97. The immunogenic composition according to any one of clauses C69 to C90, wherein the saccharide does not further comprise a 3-deoxy-d-manno-oct-2-ulosonic acid (KDO) moiety.
C98. The immunogenic composition according to any one of clauses C69 to C91, wherein the saccharide does not comprise a Lipid A.
C99. The immunogenic composition according to any one of clauses C69 to C98, wherein the polysaccharide has a molecular weight of between 10 kDa and 2,000 kDa, or between 50 kDa and 2,000 kDa.
C100. The immunogenic composition according to any one of clauses C69 to C99, wherein the saccharide has an average molecular weight of 20-40 kDa.
C101. The immunogenic composition according to any one of clauses C69 to C100, wherein the saccharide has an average molecular weight of 40,000 to 60,000 kDa.
C102. The immunogenic composition according to any one of clauses C69 to C101, wherein n is an integer 31 to 90.
C103. An immunogenic composition comprising a mutated FimH polypeptide according to any one of clauses C69 to C50, and a conjugate comprising a saccharide covalently bound a carrier protein, wherein the saccharide is derived from E. coli.
C104. An immunogenic composition comprising a mutated FimH polypeptide according to any one of clauses C69 to C50, and a conjugate comprising a saccharide according to any one of clauses C69 to C102, covalently bound to a carrier protein.
C105. An immunogenic composition comprising a mutated FimH polypeptide according to any one of clauses C69 to C50, or fragment thereof; and a conjugate according to any one of clause C69 to clause C102, wherein the carrier protein is selected from any one of poly(L-lysine), CRM₁₉₇, diphtheria toxin fragment B (DTFB), DTFB C8, Diphtheria toxoid (DT), tetanus toxoid (TT), fragment C of TT, pertussis toxoid, cholera toxoid, or exotoxin A from Pseudomonas aeruginosa; detoxified Exotoxin A of P. aeruginosa (EPA), maltose binding protein (MBP), detoxified hemolysin A of S. aureus, clumping factor A, clumping factor B, Cholera toxin B subunit (CTB), Streptococcus pneumoniae Pneumolysin and detoxified variants thereof, C. jejuni AcrA, C. jejuni natural glycoproteins and Streptococcal C5a peptidase (SCP).
C106. The immunogenic composition according to any one of clause C103 to clause C105, wherein the carrier protein is CRM₁₉₇.
C107. The immunogenic composition according to any one of clause C103 to clause C105, wherein the carrier protein is tetanus toxoid (TT).
C108. The immunogenic composition according to any one of clause C103 to clause C105, wherein the carrier protein is poly(L-lysine).
C109. The immunogenic composition according to any one of clause C103 to clause C107, wherein the conjugate is prepared by reductive amination.
C110. The immunogenic composition according to any one of clause C103 to clause C107, wherein the conjugate is prepared by CDAP chemistry.
C111. The immunogenic composition according to any one of clause C103 to clause C107, wherein the conjugate is a single-end linked conjugated saccharide.
C112. The immunogenic composition according to any one of clause C103 to clause C107, wherein the saccharide is conjugated to the carrier protein through a (2-((2-oxoethyl)thio)ethyl)carbamate (eTEC) spacer.
C113. The immunogenic composition according to clause C112, wherein the saccharide is conjugated to the carrier protein through a (2-((2-oxoethyl)thio)ethyl)carbamate (eTEC) spacer, wherein the saccharide is covalently linked to the eTEC spacer through a carbamate linkage, and wherein the carrier protein is covalently linked to the eTEC spacer through an amide linkage.
C114. The immunogenic composition according to any one of clause C112 to clause C113, wherein the CRM₁₉₇ comprises 2 to 20, or 4 to 16, lysine residues covalently linked to the polysaccharide through an eTEC spacer.
C115. The immunogenic composition according to any one of clause C103 to clause C114, wherein the saccharide:carrier protein ratio (w/w) is between 0.2 and 4.
C116. The immunogenic composition according to any one of clause C103 to clause C114, wherein the ratio of saccharide to protein is at least 0.5 and at most 2.
C117. The immunogenic composition according to any one of clause C103 to clause C114, wherein the ratio of saccharide to protein is between 0.4 and 1.7
C118. The immunogenic composition according to any one of clause C111 to clause C117, wherein the saccharide is conjugated to the carrier protein through a 3-deoxy-d-manno-oct-2-ulosonic acid (KDO) residue.
C119. The immunogenic composition according to clause C69, wherein the conjugate comprises a saccharide covalently bound to a carrier protein, wherein the saccharide comprises a structure selected from Formula O8, Formula O9a, Formula O9, Formula O20ab, Formula O20ac, Formula O52, Formula O97, and Formula O101, wherein n is an integer from 1 to 10.
C120. An immunogenic composition comprising a mutated FimH polypeptide, and a saccharide according to any one of clause C69 to clause C102, and a pharmaceutically acceptable diluent.
C121. An immunogenic composition comprising a mutated FimH polypeptide, and a glycoconjugate according to any one of clause C103 to clause C119, and a pharmaceutically acceptable diluent.
C122. The immunogenic composition according to clause C121, comprising at most about 25% free saccharide as compared to the total amount of saccharide in the composition.
C123. The immunogenic composition according to any one of clause C120 to clause C121, further comprising an adjuvant.
C124. The immunogenic composition according to any one of clause C120 to clause C121, further comprising aluminum.
C125. The immunogenic composition according to any one of clause C120 to clause C121, further comprising QS-21.
C126. The immunogenic composition according to any one of clause C120 to clause C121, further comprising a CpG oligonucleotide.
C127. The immunogenic composition according to any one of clause C120 to clause C121, wherein the composition does not include an adjuvant.
C128. An immunogenic composition comprising a mutated FimH polypeptide according to any of clauses C69 to C118, and a saccharide derived from E. coli, conjugated to a carrier protein through a (2-((2-oxoethyl)thio)ethyl)carbamate (eTEC) spacer, wherein the polysaccharide is covalently linked to the eTEC spacer through a carbamate linkage, and wherein the carrier protein is covalently linked to the eTEC spacer through an amide linkage.
C129. The immunogenic composition according to clause C128, wherein the saccharide is an O-antigen derived from E. coli.
C130. The immunogenic composition according to clause C128, further comprising a pharmaceutically acceptable excipient, carrier or diluent.
C131. The immunogenic composition according to clause C128, wherein the saccharide is an O-antigen derived from E. coli.
C132. An immunogenic composition comprising a mutated FimH polypeptide, and a saccharide according to any one of clause C69 to clause C85, conjugated to a carrier protein through a (2-((2-oxoethyl)thio)ethyl)carbamate (eTEC) spacer, wherein the polysaccharide is covalently linked to the eTEC spacer through a carbamate linkage, and wherein the carrier protein is covalently linked to the eTEC spacer through an amide linkage.
C133. An immunogenic composition comprising a mutated FimH polypeptide, and (i) a conjugate of an E. coli O25B antigen covalently coupled to a carrier protein, (ii) a conjugate of an E. coli O1A antigen covalently coupled to a carrier protein, (iii) a conjugate of an E. coli O2 antigen covalently coupled to a carrier protein, and (iv) a conjugate of an O6 antigen covalently coupled to a carrier protein, wherein the E. coli O25B antigen comprises the structure of Formula O25B, wherein n is an integer greater than 30.
C134. The immunogenic composition of clause C133, wherein the carrier protein is selected from any one of poly(L-lysine), CRM₁₉₇, diphtheria toxin fragment B (DTFB), DTFB C8, Diphtheria toxoid (DT), tetanus toxoid (TT), fragment C of TT, pertussis toxoid, cholera toxoid, or exotoxin A from Pseudomonas aeruginosa; detoxified Exotoxin A of P. aeruginosa (EPA), maltose binding protein (MBP), detoxified hemolysin A of S. aureus, clumping factor A, clumping factor B, Cholera toxin B subunit (CTB), Streptococcus pneumoniae Pneumolysin and detoxified variants thereof, C. jejuni AcrA, C. jejuni natural glycoproteins and Streptococcal C5a peptidase (SCP).
C135. An immunogenic composition comprising a mutated FimH polypeptide according to any one of clauses C69 to C118, and (i) a conjugate of an E. coli O25B antigen covalently coupled to a carrier protein, (ii) a conjugate of an E. coli O4 antigen covalently coupled to a carrier protein, (iii) a conjugate of an E. coli O11 antigen covalently coupled to a carrier protein, and (iv) a conjugate of an O21 antigen covalently coupled to a carrier protein, wherein the E. coli O25B antigen comprises the structure of Formula O75, wherein n is an integer greater than 30.
C136. The immunogenic composition of clause C135, wherein the carrier protein is selected from any one of poly(L-lysine), CRM₁₉₇, diphtheria toxin fragment B (DTFB), DTFB C8, Diphtheria toxoid (DT), tetanus toxoid (TT), fragment C of TT, pertussis toxoid, cholera toxoid, or exotoxin A from Pseudomonas aeruginosa; detoxified Exotoxin A of P. aeruginosa (EPA), maltose binding protein (MBP), detoxified hemolysin A of S. aureus, clumping factor A, clumping factor B, Cholera toxin B subunit (CTB), Streptococcus pneumoniae Pneumolysin and detoxified variants thereof, C. jejuni AcrA, C. jejuni natural glycoproteins and Streptococcal C5a peptidase (SCP).
C137. A method of making an immunogenic composition comprising a mutated FimH polypeptide, and a conjugate comprising a saccharide conjugated to a carrier protein through a (2-((2-oxoethyl)thio)ethyl)carbamate (eTEC) spacer, comprising the steps of a) reacting a saccharide with 1,1′-carbonyl-di-(1,2,4-triazole) (CDT) or 1,1′-carbonyldiimidazole (CDI), in an organic solvent to produce an activated saccharide; b) reacting the activated saccharide with cystamine or cysteamine or a salt thereof, to produce a thiolated saccharide; c) reacting the thiolated saccharide with a reducing agent to produce an activated thiolated saccharide comprising one or more free sulfhydryl residues; d) reacting the activated thiolated saccharide with an activated carrier protein comprising one or more α-haloacetamide groups, to produce a thiolated saccharide-carrier protein conjugate; and e) reacting the thiolated saccharide-carrier protein conjugate with (i) a first capping reagent capable of capping unconjugated α-haloacetamide groups of the activated carrier protein; and/or (ii) a second capping reagent capable of capping unconjugated free sulfhydryl residues; whereby an eTEC linked glycoconjugate is produced, wherein the saccharide is derived from E. coli; further comprising expressing a polynucleotide encoding a polypeptide derived from FimH or fragment thereof in a recombinant mammalian cell, and isolating said polypeptide or fragment thereof.
C138. The method according to clause C137, comprising making the immunogenic composition according to any one of clause C69 to clause C102.
C139. The method according to any of one clause C137 to clause C138, wherein the capping step e) comprises reacting the thiolated saccharide-carrier protein conjugate with (i) N-acetyl-L-cysteine as a first capping reagent, and/or (ii) iodoacetamide as a second capping reagent.
C140. The method according to any of one clause C137 to clause C139, further comprising a step of compounding the saccharide by reaction with triazole or imidazole to provide a compounded saccharide, wherein the compounded saccharide is shell frozen, lyophilized and reconstituted in an organic solvent prior to step a).
C141. The method according to any of one clause C137 to clause C140, further comprising purification of the thiolated polysaccharide produced in step c), wherein the purification step comprises diafiltration.
C142. The method according to any of one clause C137 to clause C141, wherein the method further comprises purification of the eTEC linked glycoconjugate by diafiltration.
C143. The method according to any of one clause C137 to clause C142, wherein the organic solvent in step a) is a polar aprotic solvent selected from any one of dimethyl sulfoxide (DMSO), dimethylformamide (DMF), dimethylacetamide (DMA), N-methyl-2-pyrrolidone (NMP), acetonitrile, 1,3-Dimethyl-3,4,5,6-tetrahydro-2(1H)-pyrimidinone (DMPU) and hexamethylphosphoramide (HMPA), or a mixture thereof.
C144. The method according to any of one clause C137 to clause C142, wherein the medium comprises an element selected from any one of KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O.
C145. The medium according to clause C144, wherein the medium is used for culturing E. coli.
C146. A method for producing a saccharide according to any one of clause C69 to clause C102, comprising culturing a recombinant E. coli in a medium; producing said saccharide by culturing said cell in said medium; whereby said cell produces said saccharide.
C147. The method according to clause C146, wherein the medium comprises an element selected from any one of KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O.
C148. The method according to clause C146, wherein the medium comprises soy hydrolysate.
C149. The method according to clause C146, wherein the medium comprises yeast extract.
C150. The method according to clause C146, wherein the medium does not further comprise soy hydrolysate and yeast extract.
C151. The method according to clause C146, wherein the E. coli cell comprises a heterologous wzz family protein selected from any one of wzzB, wzz, wzz_{SF}, wzz_{ST}, fepE, wzz_{fepE}, wzz1 and wzz2.
C152. The method according to clause C146, wherein the E. coli cell comprises a Salmonella enterica wzz family protein selected from any one of wzzB, wzz, wzz_{SF}, wzz_{ST}, fepE, wzz_{fepE}, wzz1 and wzz2.
C153. The method according to clause C152, wherein the wzz family protein comprises a sequence selected from any one of SEQ ID NO: 112, SEQ ID NO: 113, SEQ ID NO: 114, SEQ ID NO: 115, and SEQ ID NO: 116.
C154. The method according to clause C146, wherein the culturing produces a yield of >120 OD₆₀₀/mL.
C155. The method according to clause C146, further comprising purifying the saccharide.
C156. The method according to clause C146, wherein the purifying step comprises any one of the following: dialysis, concentration operations, diafiltration operations, tangential flow filtration, precipitation, elution, centrifugation, precipitation, ultra-filtration, depth filtration, and column chromatography (ion exchange chromatography, multimodal ion exchange chromatography, DEAE, and hydrophobic interaction chromatography).
C157. A method for inducing an immune response in a subject comprising administering to the subject a composition according to any one of clause C69 to clause C136.
C158. The method according to clause C157, wherein the immune response comprises induction of an anti-E. coli O-specific polysaccharide serum antibody.
C159. The method according to clause C157, wherein the immune response comprises induction of an anti-E. coli IgG antibody.
C160. The method according to clause C157, wherein the immune response comprises induction of bactericidal activity against E. coli.
C161. The method according to clause C157, wherein the immune response comprises induction of opsonophagocytic antibodies against E. coli.
C162. The method according to clause C157, wherein the immune response comprises a geometric mean titer (GMT) level of at least 1,000 to 200,000 after initial dosing.
C163. The method according to clause C157, wherein the composition comprises a saccharide comprising the Formula O25, wherein n is an integer 40 to 100, wherein the immune response comprises a geometric mean titer (GMT) level of at least 1,000 to 200,000 after initial dosing.
C164. The method according to clause C157, wherein the subject is at risk of any one of the conditions selected from urinary tract infection, cholecystitis, cholangitis, diarrhea, hemolytic uremic syndrome, neonatal meningitis, urosepsis, intra-abdominal infection, meningitis, complicated pneumonia, wound infection, post-prostate biopsy-related infection, neonatal/infant sepsis, neutropenic fever, and other blood stream infection; pneumonia, bacteremia, and sepsis.
C165. The method according to clause C157, wherein the subject is a mammal.
C166. A method for (i) inducing an immune response in a subject against extra-intestinal pathogenic E. coli, or (ii) inducing the production of opsonophagocytic antibodies in a subject that are specific to extra-intestinal pathogenic E. coli, wherein the method comprises administering to the subject an effective amount of the composition according to any one of clause C69 to clause C136.
C167. The method of clause C166, wherein the subject is at risk of developing a urinary tract infection.
C168. The method of clause C166, wherein the subject is at risk of developing bacteremia.
C169. The method of clause C166, wherein the subject is at risk of developing sepsis.
C170. A method for inducing an immune response in a subject comprising administering to the subject a composition according to any one of clauses C69 to clause C136.
C171. The method according to clause C170, wherein the immune response comprises induction of an anti-E. coli O-specific polysaccharide serum antibody.
C172. The method according to clause C170, wherein the anti-E. coli O-specific polysaccharide serum antibody is an IgG antibody.
C173. The method according to clause C170, wherein the anti-E. coli O-specific polysaccharide serum antibody is an IgG antibody has bactericidal activity against E. coli.
C174. The immunogenic composition of clause C69, wherein n is greater than the number of repeat units in the corresponding wild-type E. coli polysaccharide.
C175. The composition according to clause C174, wherein n is an integer from 31 to 100.
C176. The composition according to clause C174, wherein the saccharide comprises a structure according to any one of Formula O1A, Formula O1B, and Formula O1C, Formula O2, Formula O6, and Formula O25B.
C177. The composition according to clause C174, wherein the saccharide is produced in a recombinant host cell that expresses a wzz family protein having at least 90% sequence identity to any one of SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, and SEQ ID NO: 121.
C178. The composition according to clause C177, wherein the protein comprises any one of SEQ ID NO: 112, SEQ ID NO: 113, SEQ ID NO: 114, SEQ ID NO: 115, and SEQ ID NO: 116.
C179. The saccharide according to clause C174, wherein the saccharide is synthetically synthesized.
C180. An immunogenic composition comprising a mutated FimH polypeptide according to any one of clauses C1 to C55, and (a) a conjugate comprising a carrier protein covalently bound to a saccharide comprising Formula O25b, wherein n is an integer from 31 to 90, (b) a conjugate comprising a carrier protein covalently bound to a saccharide comprising Formula O1A, wherein n is an integer from 31 to 90, (c) a conjugate comprising a carrier protein covalently bound to a saccharide comprising Formula O2, wherein n is an integer from 31 to 90, and (d) a conjugate comprising a carrier protein covalently bound to a saccharide comprising and Formula O6, wherein n is an integer from 31 to 90.
C181. The immunogenic composition according to clause C180, further comprising a conjugate comprising a carrier protein covalently bound to a saccharide comprising a structure selected from any one of the following: Formula O15, Formula O16, Formula O17, Formula O18 and Formula O75, wherein n is an integer from 31 to 90.
C182. The immunogenic composition according to clause C180, comprising at most 25% free saccharide as compared to the total amount of saccharide in the composition.
C183. A method of eliciting an immune response against E. coli in a mammal, comprising administering to the mammal an effective amount of the composition according to any one of clauses C180 to C182.
C184. The method according to clause C183, wherein the immune response comprises opsonophagocytic antibodies against E. coli.
C185. The method according to clause C183, wherein the immune response protects the mammal from an E. coli infection.
C186. A recombinant mammalian cell comprising (a) a first gene of interest encoding a mutated FimH polypeptide of any one of clauses C1 to C55, wherein the gene is integrated between at least two recombination target sites (RTS).
C187. The embodiment of clause C186, wherein the two RTS are chromosomally-integrated within the NL1 locus or the NL2 locus.
C188. The embodiment of clause C186, wherein the first gene of interest further comprises a reporter gene, a gene encoding a difficult to express protein, an ancillary gene or a combination thereof.
C189. The embodiment of clause C186, further comprising a second gene of interest that is integrated within a second chromosomal locus distinct from the locus of (a), wherein the second gene of interest comprises a reporter gene, a gene encoding a difficult to express protein, an ancillary gene or a combination thereof.
C190. The recombinant cell according to C186, wherein the polynucleotide sequence is integrated into the genomic DNA of said mammalian cell.
C191. The recombinant cell according to C186, wherein the polynucleotide sequence is codon optimized for expression in the cell.
C192. The recombinant cell according to C186, wherein the cell is a human embryonic kidney cell.
C193. The recombinant cell according to C192, wherein the human embryonic kidney cell comprises a HEK293 cell.
C194. The recombinant cell according to C193, wherein the HEK293 cell is selected from any one of HEK293T cells, HEK293TS cells, and HEK293E cells.
C195. The recombinant cell according to C186, wherein the cell is a CHO cell.
C196. The recombinant cell according to C195, wherein said CHO cell is a CHO-K1 cell, CHO-DUXB11, CHO-DG44 cell, or CHO-S cell.
C197. The recombinant cell according to C186, wherein the polypeptide is soluble.
C198. The recombinant cell according to C186, wherein the polypeptide is secreted from the cell.
C199. A culture comprising the recombinant cell of C186, wherein said culture is at least 5 liter in size.
C200. The culture according to C199, wherein the yield of the polypeptide or fragment thereof is at least 0.05 g/L.
C201. The culture according to C199, wherein the yield of the polypeptide or fragment thereof is at least 0.10 g/L.
C202. A method for producing a polypeptide derived from E. coli or a fragment thereof, comprising culturing a recombinant mammalian cell according to C186 under a suitable condition, thereby expressing the polypeptide or fragment thereof; and harvesting the polypeptide or fragment thereof.
C203. The method according to C202, further comprising purifying the polypeptide or fragment thereof.
C204. The immunogenic composition according to Clause C54, further comprising at least one saccharide derived from any one K. pneumoniae type selected from the group consisting of O1, O2, O3, and O5.
C205. The immunogenic composition according to Clause C204, further comprising a saccharide derived from K. pneumoniae type O1.
C206. The immunogenic composition according to Clause C204, further comprising a saccharide derived from K. pneumoniae type O2.
C207. The composition according to Clause C204, further comprising a saccharide derived from K. pneumoniae type O3.
C208. The immunogenic composition according to Clause C204, further comprising a saccharide derived from K. pneumoniae type O5.
C209. The immunogenic composition according to Clause C204, further comprising a saccharide derived from K. pneumoniae type O1 and a saccharide derived from K. pneumoniae type O2.
C210. The immunogenic composition according to any one of Clauses C204 to C209, further comprising at least one saccharide comprising a structure selected from any one of Formula O1, Formula O1A, Formula O1B, Formula O1C, Formula O2, Formula O3, Formula O4, Formula O4:K52, Formula O4:K6, Formula O5, Formula O5ab, Formula O5ac, Formula O6, Formula O6:K2; K13; K15, Formula O6:K54, Formula O7, Formula O8, Formula O9, Formula O10, Formula O11, Formula O12, Formula O13, Formula O14, Formula O15, Formula O16, Formula O17, Formula O18, Formula O18A, Formula O18ac, Formula O18A1, Formula O18B, Formula O18B1, Formula O19, Formula O20, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186, Formula O187, wherein n is an integer from 1 to 100, more preferably from 31 to 90.
C211. The immunogenic composition according to Clause C210, wherein the saccharide derived from K. pneumoniae is conjugated to a carrier protein; and the saccharide derived from E. coli is conjugated to a carrier protein.
C212. A method of eliciting an immune response against E. coli in a mammal, comprising administering to the mammal an effective amount of the composition according to any one of Clauses C204-C211.
C213. The method according to Clause C212, wherein the immune response comprises opsonophagocytic antibodies against E. coli.
C214. The method according to Clause C212, wherein the immune response protects the mammal from an E. coli infection.
C215. A method of eliciting an immune response against K. pneumoniae in a mammal, comprising administering to the mammal an effective amount of the composition according to any one of Clauses C204-C211.
C216. The method according to Clause C215, wherein the immune response comprises opsonophagocytic antibodies against K. pneumoniae.
C217. The method according to Clause C215, wherein the immune response protects the mammal from a K. pneumoniae infection.
C218. The compositions and methods of any of Clauses C204 to C217, wherein the K. pneumoniae serotype O1 comprises variant O1V1 or O1V2.
C219. The compositions and methods of any of Clauses C204 to C217, wherein the K. pneumoniae serotype O2 comprises variant O2V1 or O2V2.
C220. Use of the compositions set forth in any one of Clauses C1-C219 as set forth herein.
C221. The composition of clause C211, wherein the K. pneumoniae O-antigen is selected from the group consisting of a) serotype O1 subtype v1 (O1vi), b) serotype O1 subtype v2 (O1v2), c) serotype O2 subtype v1 (O2v1), and d) serotype O2 subtype v2 (O2v2).
C222. A nucleic acid comprising nucleotides encoding the polypeptides of any one of clauses C1-C221.
C223. The nucleic acid of clause C222, wherein the nucleic acid is RNA.
C224. A nanoparticle comprising the nucleic acid of clause C222 or C223.
C225. The immunogenic composition of this invention, further comprising one or more conjugates having a saccharide selected from the group consisting of Formula O4, Formula O11, Formula O13, Formula O21 and Formula O86, wherein n is an integer from 1 to 100, preferably from 31 to 90.
C226. The immunogenic composition of this invention, further comprising one or more conjugates having a saccharide selected from the group consisting of Formula O1a, Formula O2, Formula O6, and Formula O25b, wherein n is an integer from 1 to 100, preferably from 31 to 90.

1-10. (canceled)

11. A nucleic acid molecule comprising a nucleotide sequence that encodes an amino acid sequence of a mutated FimH polypeptide, wherein the mutated FimH polypeptide comprises an amino acid substitution relative to the amino acid sequence of the wild-type FimH polypeptide, wherein the substitution is selected from the group consisting of: F1I; F1V; F1M; F1Y; F1W; P12C; G14C; G15A; G15P; G16A; G16P; A18C; P26C; V28C; Q32C; N33C; L34N; L34S; L34T; L34D; L34E; L34K; L34R; V35C; S62C; Y64C; G65A; L68C; F71C; T86C; L107C; Y108C; L109C; V112C; S113C; A115V; G116C; V118C; A119C; A119N; A119S; A119D; A119E; A119K; A119R; A127C; L129C; F144C; V154C; V155C; V156C; T158C; and V185I, and wherein the amino acid positions are numbered according to SEQ ID NO: 59.

12-23. (canceled)

24. The nucleic acid of claim 11, wherein the mutated FimH polypeptide comprises the amino acid substitution V27A.

25. The nucleic acid of claim 11, wherein the nucleic acid is RNA.

26. The nucleic acid of claim 25, wherein the RNA is nucleoside modified mRNA (modRNA).

27. A nanoparticle comprising the nucleic acid of claim 25.

28. An immunogenic composition comprising the nanoparticle of claim 27.

29. The immunogenic composition of claim 28, comprising an E. coli saccharide antigen, wherein the saccharide antigen comprises a structure selected from the group consisting of Formula O25b, Formula O1A, Formula O2, and Formula O6.

30. A nucleic acid molecule comprising a nucleotide sequence that encodes an amino acid sequence of a mutated FimH polypeptide, wherein the mutated FimH polypeptide comprises an amino acid substitution relative to the amino acid sequence of the wild-type FimH polypeptide, wherein the substitution is selected from the group consisting of:
a) G15A and G16A;
b) P12C and A18C;
c) G14C and F144C;
d) P26C and V35C;
e) P26C and V154C;
f) P26C and V156C;
g) V27A and Q133K;
h) V28C and N33C;
i) V28C and P157C;
j) Q32C and Y108C;
k) N33C and L109C;
I) N33C and P157C;
m) V35C and L107C;
n) V35C and L109C;
o) S62C and T86C;
p) S62C and L129C;
q) Y64C and L68C;
r) Y64C and A127C;
s) L68C and F71C;
t) V112C and T158C;
u) S113C and G116C;
v) S113C and T158C;
w) V118C and V156C;
x) A119C and V155C;
y) L34N and V27A;
z) L34S and V27A;
aa) L34T and V27A;
ab) L34D and V27A;
ac) L34E and V27A;
ad) L34K and V27A;
ae) L34R and V27A;
af) A119N and V27A;
ag) A119S and V27A;
ah) A119T and V27A;
ai) A119D and V27A;
aj) A119E and V27A;
ak) A119K and V27A;
al) A119R and V27A;
am) G15A and V27A;
an) G16A and V27A;
ao) G15P and V27A;
ap) G16P and V27A;
aq) G15A, G16A, and V27A;
ar) G65A and V27A; and
as) G15A, G16A, V27A, and Q133K, and wherein the amino acid positions are numbered according to SEQ ID NO: 59.

31. The nucleic acid of claim 30, wherein the nucleic acid is RNA.

32. The nucleic acid of claim 31, wherein the RNA is nucleoside modified mRNA (modRNA).

33. A nanoparticle comprising the nucleic acid of claim 31.

34. An immunogenic composition comprising the nanoparticle of claim 33.

35. The immunogenic composition of claim 34, comprising an E. coli saccharide antigen, wherein the saccharide antigen comprises a structure selected from the group consisting of Formula O25b, Formula O1A, Formula O2, and Formula O6.

36. A nucleic acid molecule comprising a nucleotide sequence that encodes an amino acid sequence of a mutated FimH polypeptide, wherein the mutated FimH polypeptide comprises the following amino acid substitutions relative to the amino acid sequence of the wild-type FimH polypeptide: G15A, G16A, and V27A, and wherein the amino acid positions are numbered according to SEQ ID NO: 59.

37. The nucleic acid of claim 36, wherein the nucleic acid is RNA.

38. The nucleic acid of claim 37, wherein the RNA is nucleoside modified mRNA (modRNA).

39. A nanoparticle comprising the nucleic acid of claim 37.

40. An immunogenic composition comprising the nanoparticle of claim 39.

41. The immunogenic composition of claim 40, comprising an E. coli saccharide antigen, wherein the saccharide antigen comprises a structure selected from the group consisting of Formula O25b, Formula O1A, Formula O2, and Formula O6.

42. A method of eliciting an immune response against E. coli in a mammal, comprising administering to the mammal an effective amount of the composition of claim 41.


SEQUENCE LISTING (147 sequences)

SEQ ID NO: 1 - protein, 160 aa, Escherichia coli [sequence omitted]

SEQ ID NO: 2 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 3 - protein, 160 aa, synthetic construct
  REGION 1..160: note = Synthetic
IACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 4 - protein, 160 aa, synthetic construct
  REGION 1..160: note = Synthetic
LACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 5 - protein, 160 aa, synthetic construct
  REGION 1..160: note = Synthetic
VACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 6 - protein, 160 aa, synthetic construct
  REGION 1..160: note = Synthetic
MACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 7 - protein, 160 aa, synthetic construct
  REGION 1..160: note = Synthetic
YACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 8 - protein, 160 aa, synthetic construct
  REGION 1..160: note = Synthetic
WACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 9 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 10 - protein, 160 aa, synthetic construct
  REGION 1..160: note = Synthetic
FACKTASGTAIPIGAGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 11 - protein, 160 aa, synthetic construct
  REGION 1..160: note = Synthetic
FACKTASGTAIPIGPGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 12 - protein, 160 aa, synthetic construct
  REGION 1..160: note = Synthetic
FACKTASGTAIPIGGASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 13 - protein, 160 aa, synthetic construct
  REGION 1..160: note = Synthetic
FACKTASGTAIPIGGPSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 14 - protein, 160 aa, synthetic construct
  REGION 1..160: note = Synthetic
FACKTASGTAIPIGAASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 15 - protein, 160 aa, synthetic construct
  REGION 1..160: note = Synthetic
FACKTASGTAIPIGGGSANVYVNLAPVVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 16 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 17 - protein, 160 aa, synthetic construct
  REGION 1..160: note = Synthetic
FACKTASGTAICIGGGSCNVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 18 - protein, 160 aa, synthetic construct
  REGION 1..160: note = Synthetic
FACKTASGTAIPICGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQCVWNIYANNDVVVPTGG

SEQ ID NO: 19 - protein, 160 aa, synthetic construct
  REGION 1..160: note = Synthetic
FACKTASGTAIPIGGGSANVYVNLACVVNVGQNLCVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 20 - protein, 160 aa, synthetic construct
  REGION 1..160: note = Synthetic
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDCVVPTGG

SEQ ID NO: 21 - protein, 160 aa, synthetic construct
  REGION 1..160: note = Synthetic
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVCPTGG

SEQ ID NO: 22 - protein, 160 aa, synthetic construct
  REGION 1..160: note = Synthetic
FACKTASGTAIPIGGGSANVYVNLAPCVNVGQNCVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 23 - protein, 160 aa, synthetic construct
  REGION 1..160: note = Synthetic
FACKTASGTAIPIGGGSANVYVNLAPVCNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 24 - protein, 160 aa, synthetic construct
  REGION 1..160: note = Synthetic
FACKTASGTAIPIGGGSANVYVNLAPVCNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 25 - protein, 160 aa, synthetic construct
  REGION 1..160: note = Synthetic
FACKTASGTAIPIGGGSANVYVNLAPVVNVGCNLVVDLSTQIFCHNDYPETITDYVTLQRGSAYGGVLSSFSGTVKYSGSSYPFPTTSETPRVVYNSRTDKPWPVALCLTPVSSAGGVAIKAGSLIAVLILRQTNNYNSDDFQFVWNIYANNDVVVPTGG

SEQ ID NO: 26 - protein, 160 aa, synthetic construct
  REGION 1..160: note = Synthetic
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 27 - protein, 160 aa, synthetic construct
  REGION 1..160: note = Synthetic
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 28 - protein, 160 aa, synthetic construct
  REGION 1..160: note = Synthetic
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVACYLTPVSSAGGVAIKAGSLIAVLILRQTNNYNSDDFQFVWNIYANNDVVVPTGG

SEQ ID NO: 29 - protein, 160 aa, synthetic construct
  REGION 1..160: note = Synthetic
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 30 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGCAYGGVLSSFSGTVKYSGSSYPFPCTSETPRVVYNSRTDKPWPVALYLTPVSSAGGVAIKAGSLIAVLILRQTNNYNSDDFQFVWNIYANNDVVVPTGG

SEQ ID NO: 31 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGCAYGGVLSSFSGTVKYSGSSYPFPTTSETPRVVYNSRTDKPWPVALYLTPVSSAGGVAIKAGSLIAVCILRQTNNYNSDDFQFVWNIYANNDVVVPTGG

SEQ ID NO: 32 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSACGGVCSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 33 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSACGGVLSSFSGTVKYSGSSYPFPTTSETPRVVYNSRTDKPWPVALYLTPVSSAGGVAIKAGSLICVLILRQTNNYNSDDFQFVWNIYANNDVVVPTGG

SEQ ID NO: 34 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVCSSCSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 35 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPCSSAGGVAIKAGSLIAVLILRQTNNYNSDDFQFVWNIYANNDVVVPCGG

SEQ ID NO: 36 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSACGVAIKAGSLIAVLILRQTNNYNSDDFQFVWNIYANNDVVVPTGG

SEQ ID NO: 37 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSAGGVAIKAGSLIAVLILRQTNNYNSDDFQFVWNIYANNDVVVPCGG

SEQ ID NO: 38 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGCAIKAGSLIAVLILRQTNNYNSDDFQFVWNIYANNDVVCPTGG

SEQ ID NO: 39 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVCIKAGSLIAVLILRQTNNYNSDDFQFVWNIYANNDVCVPTGG

SEQ ID NO: 40 - protein, 160 aa, synthetic construct
  REGION 1..160: note = Synthetic
FACKTASGTAIPIGGGSANVYVNLAPAVNVGQNNVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 41 - protein, 160 aa, synthetic construct
  REGION 1..160: note = Synthetic
FACKTASGTAIPIGGGSANVYVNLAPAVNVGQNSVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 42 - protein, 160 aa, synthetic construct
  REGION 1..160: note = Synthetic
FACKTASGTAIPIGGGSANVYVNLAPAVNVGQNTVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 43 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVNIKAGSLIAVLILRQTNNYNSDDFQFVWNIYANNDVVVPTGG

SEQ ID NO: 44 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVSIKAGSLIAVLILRQTNNYNSDDFQFVWNIYANNDVVVPTGG

SEQ ID NO: 45 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVTIKAGSLIAVLILRQTNNYNSDDFQFVWNIYANNDVVVPTGG

SEQ ID NO: 46 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSVGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 47 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDISARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 48 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTIYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 49 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADITITVNGKVVAK

SEQ ID NO: 50 - protein, 160 aa, synthetic construct
  REGION 1..160: note = Synthetic
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 51 - protein, 160 aa, synthetic construct
  REGION 1..160: note = Synthetic
FACKTASGTAIPIGGASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 52 - protein, 160 aa, synthetic construct
  REGION 1..160: note = Synthetic
FACKTASGTAIPIGPGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 53 - protein, 160 aa, synthetic construct
  REGION 1..160: note = Synthetic
FACKTASGTAIPIGGPSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 54 - protein, 160 aa, synthetic construct
  REGION 1..160: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 55 - protein, 160 aa, synthetic construct
  REGION 1..160: note = Synthetic
FACKTASGTAIPIGGGSANVYVNLAPAVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 56 - protein, 160 aa, synthetic construct; identical to SEQ ID NO: 2
  REGION 1..160: note = Synthetic

SEQ ID NO: 57 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 58 - protein, 160 aa, synthetic construct
  REGION 1..160: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 59 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 60 - protein, 300 aa, synthetic construct
  REGION 1..300: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 61 - protein, 300 aa, synthetic construct
  REGION 1..300: note = Synthetic
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 62 - protein, 300 aa, synthetic construct
  REGION 1..300: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 63 - protein, 300 aa, synthetic construct
  REGION 1..300: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 64 - protein, 300 aa, synthetic construct
  REGION 1..300: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 65 - protein, 19 aa, Mus musculus
ETDTLLLWVLLLWVPGSTG

SEQ ID NO: 66 - protein, 207 aa, synthetic construct
  REGION 1..207: note = Synthetic
MEGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 67 - protein, 156 aa, synthetic construct
  REGION 1..156: note = Synthetic
MTKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 68 - protein, 156 aa, synthetic construct
  REGION 1..156: note = Synthetic
MTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 69 - protein, 209 aa, synthetic construct
  REGION 1..209: note = Synthetic
MSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 70 - protein, 114 aa, synthetic construct
  REGION 1..114: note = Synthetic
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 71 - protein, 157 aa, synthetic construct
  REGION 1..157: note = Synthetic
MNQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 72 - protein, 205 aa, synthetic construct
  REGION 1..205: note = Synthetic
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 73 - protein, 157 aa, synthetic construct
  REGION 1..157: note = Synthetic
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 74 - protein, 177 aa, synthetic construct
  REGION 1..177: note = Synthetic
MFTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 75 - protein, 157 aa, synthetic construct
  REGION 1..157: note = Synthetic
MNQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 76 - protein, 201 aa, synthetic construct
  REGION 1..201: note = Synthetic
MGHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 77 - protein, 237 aa, synthetic construct
  REGION 1..237: note = Synthetic
MYEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 78 - protein, 138 aa, synthetic construct
  REGION 1..138: note = Synthetic
MGMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 79 - protein, 154 aa, synthetic construct
  REGION 1..154: note = Synthetic
MKYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 80 - protein, 164 aa, synthetic construct
  REGION 1..164: note = Synthetic
MAVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 81 - protein, 175 aa, synthetic construct
  REGION 1..175: note = Synthetic
MGANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 82 - protein, 208 aa, synthetic construct
  REGION 1..208: note = Synthetic
MTDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 83 - protein, 128 aa, synthetic construct
  REGION 1..128: note = Synthetic
MITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 84 - protein, 235 aa, synthetic construct
  REGION 1..235: note = Synthetic
MGSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 85 - protein, 162 aa, synthetic construct
  REGION 1..162: note = Synthetic
MSGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 86 - protein, 157 aa, synthetic construct
  REGION 1..157: note = Synthetic
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 87 - protein, 157 aa, synthetic construct
  REGION 1..157: note = Synthetic
MILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 88 - protein, 156 aa, synthetic construct
  REGION 1..156: note = Synthetic
MTKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 89 - protein, 209 aa, synthetic construct
  REGION 1..209: note = Synthetic
MDDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 90 - protein, 114 aa, synthetic construct
  REGION 1..114: note = Synthetic
MPIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 91 - protein, 114 aa, synthetic construct
  REGION 1..114: note = Synthetic
MPIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 92 - protein, 157 aa, synthetic construct
  REGION 1..157: note = Synthetic
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 93 - protein, 157 aa, synthetic construct
  REGION 1..157: note = Synthetic
MNQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 94 - protein, 205 aa, synthetic construct
  REGION 1..205: note = Synthetic
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 95 - protein, 205 aa, synthetic construct
  REGION 1..205: note = Synthetic
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 96 - protein, 205 aa, synthetic construct
  REGION 1..205: note = Synthetic
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 97 - protein, 157 aa, synthetic construct
  REGION 1..157: note = Synthetic
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 98 - protein, 157 aa, synthetic construct
  REGION 1..157: note = Synthetic
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 99 - protein, 157 aa, synthetic construct
  REGION 1..157: note = Synthetic
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 100 - protein, 156 aa, synthetic construct
  REGION 1..156: note = Synthetic
  SITE 70: note = MISC_FEATURE - Xaa is A or K
MTKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 101 - protein, 209 aa, synthetic construct
  REGION 1..209: note = Synthetic
  SITE 2: note = MISC_FEATURE - Xaa is S or D
  SITE 3: note = MISC_FEATURE - Xaa is T or D
  SITE 10: note = MISC_FEATURE - Xaa is A or R
  SITE 85: note = MISC_FEATURE - Xaa is T or D
  SITE 119: note = MISC_FEATURE - Xaa is A or Q
  SITE 163: note = MISC_FEATURE - Xaa is S or D
  SITE 189: note = MISC_FEATURE - Xaa is T or R
MXXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 102 - protein, 114 aa, synthetic construct
  REGION 1..114: note = Synthetic
  SITE 13: note = MISC_FEATURE - Xaa is T or D
  SITE 33: note = MISC_FEATURE - Xaa is K or E
  SITE 71: note = MISC_FEATURE - Xaa is S or D
  SITE 74: note = MISC_FEATURE - Xaa is R or E
  SITE 101: note = MISC_FEATURE - Xaa is N or D
  SITE 103: note = MISC_FEATURE - Xaa is N or D
MPIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 103 - protein, 157 aa, synthetic construct
  REGION 1..157: note = Synthetic
  SITE 9: note = MISC_FEATURE - Xaa is Y or H
  SITE 82: note = MISC_FEATURE - Xaa is N or D
  SITE 87: note = MISC_FEATURE - Xaa is R or D
  SITE 105: note = MISC_FEATURE - Xaa is S or D
  SITE 119: note = MISC_FEATURE - Xaa is R or E
  SITE 121: note = MISC_FEATURE - Xaa is R or E
  SITE 124: note = MISC_FEATURE - Xaa is A or D
  SITE 126: note = MISC_FEATURE - Xaa is H or D
  SITE 128: note = MISC_FEATURE - Xaa is R or E
  SITE 150: note = MISC_FEATURE - Xaa is A or N
MNQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 104 - protein, 205 aa, synthetic construct
  REGION 1..205: note = Synthetic
  SITE 126: note = MISC_FEATURE - Xaa is T or D
  SITE 139: note = MISC_FEATURE - Xaa is Q or E
  SITE 142: note = MISC_FEATURE - Xaa is K or E
  SITE 160: note = MISC_FEATURE - Xaa is N or D
  SITE 163: note = MISC_FEATURE - Xaa is N or D
  SITE 166: note = MISC_FEATURE - Xaa is E or K
  SITE 169: note = MISC_FEATURE - Xaa is D or K
  SITE 179: note = MISC_FEATURE - Xaa is S, D or K
  SITE 183: note = MISC_FEATURE - Xaa is K or E
  SITE 185: note = MISC_FEATURE - Xaa is T, D, or K
  SITE 192: note = MISC_FEATURE - Xaa is D or K
  SITE 195: note = MISC_FEATURE - Xaa is A, E, or K
  SITE 198: note = MISC_FEATURE - Xaa is E or K
  SITE 199: note = MISC_FEATURE - Xaa is E or K
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 105 - protein, 157 aa, synthetic construct
  REGION 1..157: note = Synthetic
  SITE 9: note = MISC_FEATURE - Xaa is Y or H
  SITE 38: note = MISC_FEATURE - Xaa is A or R
  SITE 82: note = MISC_FEATURE - Xaa is N or D
  SITE 87: note = MISC_FEATURE - Xaa is R or D
  SITE 97: note = MISC_FEATURE - Xaa is N or D
  SITE 105: note = MISC_FEATURE - Xaa is S, N, or D
  SITE 119: note = MISC_FEATURE - Xaa is R, E, or N
  SITE 121: note = MISC_FEATURE - Xaa is R, E, or D
  SITE 122: note = MISC_FEATURE - Xaa is K or D
  SITE 124: note = MISC_FEATURE - Xaa is K or D
  SITE 126: note = MISC_FEATURE - Xaa is H or D
MNQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 106 - DNA, 142 bp, synthetic construct
  misc_feature 1..142: note = Synthetic
  source 1..142: mol_type = other DNA
taatgcttaagtcgaacagaaagtaatcgtattgtacacggccgcataatcgaaattaatacgactcactataggggaattgtgagcggataacaattccccatcttagtatattagttaagtataagaaggagatatactt

SEQ ID NO: 107 - DNA, 18 bp, synthetic construct
  misc_feature 1..18: note = Synthetic
  source 1..18: mol_type = other DNA
taaagaaggagatatcat

SEQ ID NO: 108 - DNA, 17 bp, synthetic construct
  misc_feature 1..17: note = Synthetic
  source 1..17: mol_type = other DNA
tgagaaggagatatcat

SEQ ID NO: 109 - DNA, 26 bp, synthetic construct
  misc_feature 1..26: note = Synthetic
  source 1..26: mol_type = other DNA
ttgaaagtgatggtttggtaagaaat

SEQ ID NO: 110 - DNA, 25 bp, synthetic construct
  misc_feature 1..25: note = Synthetic
  source 1..25: mol_type = other DNA
tgcagcacgtatgataacttcaaag

SEQ ID NO: 111 - DNA, 27 bp, synthetic construct
  misc_feature 1..27: note = Synthetic
  source 1..27: mol_type = other DNA
aggatattttacccagcagtgccccgt

SEQ ID NO: 112 - protein, 325 aa, synthetic construct
  REGION 1..325: note = Synthetic
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQEQVTKPQVQQTEDVTQDTLFLLGSEALESMIKHEATRPLVFSSNYYQTRQNLLDIESLKVDDLDIHAYRYVMKPTLPIRRDSPKKAITLILAVLLGGMVGAGIVLGRNALRNYNAK

SEQ ID NO: 113 - protein, 326 aa, synthetic construct
  REGION 1..326: note = Synthetic
MRVENNNVSGQNNDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQDEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPTLPIRRDSPKKAITLILAVLLGGMVGAGIVLGRNALRNYNAK

SEQ ID NO: 114 - protein, 326 aa, synthetic construct
  REGION 1..326: note = Synthetic
MRVENNNVSGQNHDPEQIDLIDLLVQLWRGKMTIIISVVVAIALAIGYLAVAKEKWTSTAIITQPDVGQIAGYNNAMNVIYGQAAPKVSDLQETLIGRFSFAFSALAETLDNQKEPEKLTIEPSVKNQQLPLTVSYVGQTAEDAQMKLAQYIQQVDDKVNQELEKDLKDNLALGRKNLQDSLRTQEVVAQEQKDLRIRQIQEALQYANQAQVTKPQIQQTGEDITQDTLFLLGSEALESMIKHEATRPLVFSPNYYQTRQNLLDIENLKVDDLDIHAYRYVMKPTLPIRRDSPKKAITLILAVLLGGMVGAGIVLGRNALRNYNSK

SEQ ID NO: 115 - protein, 326 aa, synthetic construct
  REGION 1..326: note = Synthetic
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEREKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPMLPIRRDSPKKAITLILAVLLGGMVGAGIVLGRNALRNYNAK

SEQ ID NO: 116 - protein, 327 aa, synthetic construct
  REGION 1..327: note = Synthetic
MTVDSNTSSGRGNDPEQIDLIELLLQLWRGKMTIIVAVIIAILLAVGYLMIAKEKWTSTAIITQPDAAQVATYTNALNVLYGGNAPKISEVQANFISRFSSAFSALSEVLDNQKEREKLTIEQSVKGQALPLSVSYVSTTAEGAQRRLAEYIQQVDEEVAKELEVDLKDNITLQTKTLQESLETQEVVAQEQKDLRIKQIEEALRYADEAKITQPQIQQTQDVTQDTMFLLGSDALKSMIQNEATRPLVFSPAYYQTKQTLLDIKNLKVTADTVHVYRYVMKPTLPVRRDSPKTAITLVLAVLLGGMIGAGIVLGRNALRSYKPKAL

SEQ ID NO: 117 - protein, 377 aa, synthetic construct
  REGION 1..377: note = Synthetic
MSSLNIKQGSDAHFPDYPLASPSNNEIDLLNLISVLWRAKKTVMAVVFAFACAGLLISFILPQKWTSAAVVTPPEPVQWQELEKSFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISTLVVKESLENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKAHVNDVNFTPFKYQLSPSLPVKKDGPGKAIIVILSALIGGMVACGGVLLRYAMASRKQDAMMADHLV

SEQ ID NO: 118 - protein, 377 aa, synthetic construct
  REGION 1..377: note = Synthetic
MSSLNIKQGSEAHFPEYPLASPSNNEIDLLNLIEVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDPLDLHRAIVALSEKMKAVDDNASKKKDESALYTSWTLSFTAPTSEEAQKVLAGYIDYISALVVKESIENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKTNINDVNFTPFKYQLRPSLPVKKDGQGKAIIVILSALVGGMVACGGVLLRHAMASRKQDAMMADHLV

SEQ ID NO: 119 - protein, 377 aa, synthetic construct; identical to SEQ ID NO: 117
  REGION 1..377: note = Synthetic

SEQ ID NO: 120 - protein, 377 aa, synthetic construct
  REGION 1..377: note = Synthetic
MSSLNIKQGSDAHFPDYPLASPSNNEIDLLNLISVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISALVVKESIENVRNKLEIKTQFEKEKLAQDRIKMKNQLDANIQRLNYSLDIANAAGIKKPVYSNGQAVKDDPDFSISLGADGIERKLEIEKAVTDVAELNGELRNRQYLVEQLTKANINDVNFTPFKYQLSPSLPVKKDGPGKAIIVILSALIGGMVACGSVLLRYAMASRKQDAMMADHLV

SEQ ID NO: 121 - protein, 378 aa, synthetic construct
  REGION 1..378: note = Synthetic
MPSLNVKQEKNQSFAGYSLPPANSHEIDLFSLIEVLWQAKRRILATVFAFACVGLLLSFLLPQKWTSQAIVTPAESVQWQGLERTLTALRVLDMEVSVDRGSVFNLFIKKFSSPSLLEEYLRSSPYVMDQLKGAQIDEQDLHRAIVLLSEKMKAVDSNVGKKNETSLFTSWTLSFTAPTREEAQKVLAGYIQYISDIVVKETLENIRNQLEIKTRYEQEKLAMDRVRLKNQLDANIQRLHYSLEIANAAGIKRPVYSNGQAVKDDPDFSISLGADGISRKLEIEKGVTDVAEIDGDLRNRQYHVEQLAAMNVSDVKFTPFKYQLSPSLPVKKDGPGKAIIIILAALIGGMMACGGVLLRHAMVSRKMENALAIDERLV

SEQ ID NO: 122 - DNA, 22 bp, synthetic construct
  misc_feature 1..22: note = Synthetic
  source 1..22: mol_type = other DNA
gaagcaaaccgtacgcgtaaag

SEQ ID NO: 123 - DNA, 20 bp, synthetic construct
  misc_feature 1..20: note = Synthetic
  source 1..20: mol_type = other DNA
cgaccagctcttacacggcg

SEQ ID NO: 124 - DNA, 36 bp, synthetic construct
  misc_feature 1..36: note = Synthetic
  source 1..36: mol_type = other DNA
gaaataggaccactaataaatacacaaattaataac

SEQ ID NO: 125 - DNA, 21 bp, synthetic construct
  misc_feature 1..21: note = Synthetic
  source 1..21: mol_type = other DNA
ataattgacgatccggttgcc

SEQ ID NO: 126 - DNA, 27 bp, synthetic construct
  misc_feature 1..27: note = Synthetic
  source 1..27: mol_type = other DNA
gctatttacgccctgattgtcttttgt

SEQ ID NO: 127 - DNA, 22 bp, synthetic construct
  misc_feature 1..22: note = Synthetic
  source 1..22: mol_type = other DNA
attgagaacctgcgtaaacggc

SEQ ID NO: 128 - DNA, 24 bp, synthetic construct
  misc_feature 1..24: note = Synthetic
  source 1..24: mol_type = other DNA
tgaagagcggttcagataacttcc

SEQ ID NO: 129 - DNA, 21 bp, synthetic construct
  misc_feature 1..21: note = Synthetic
  source 1..21: mol_type = other DNA
cgatccggaaacctcctacac

SEQ ID NO: 130 - DNA, 26 bp, synthetic construct
  misc_feature 1..26: note = Synthetic
  source 1..26: mol_type = other DNA
gattattcgcgcaacgctaaacagat

SEQ ID NO: 131 - DNA, 23 bp, synthetic construct
  misc_feature 1..23: note = Synthetic
  source 1..23: mol_type = other DNA
tgatcattgacgatccggtagcc

SEQ ID NO: 132 - DNA, 70 bp, synthetic construct
  misc_feature 1..70: note = Synthetic
  source 1..70: mol_type = other DNA
cggtagctgtaaagccaggggcggtagcgtggtttaaacccaagcaacagatcggcgtcgtcggtatgga

SEQ ID NO: 133 - DNA, 78 bp, synthetic construct
  misc_feature 1..78: note = Synthetic
  source 1..78: mol_type = other DNA
agcttccataccgacgacgccgatctgttgcttgggtttaaaccacgctaccgcccctggctttacagctaccgagct

SEQ ID NO: 134 - DNA, 30 bp, synthetic construct
  misc_feature 1..30: note = Synthetic
  source 1..30: mol_type = other DNA
ggtagctgtaaagccaggggcggtagcgtg

SEQ ID NO: 135 - DNA, 30 bp, synthetic construct
  misc_feature 1..30: note = Synthetic
  source 1..30: mol_type = other DNA
ccataccgacgacgccgatctgttgcttgg

SEQ ID NO: 136 - protein, 23 aa, Homo sapiens
MGVPRPQPWALGLLLFLLPGSLG

SEQ ID NO: 137 - protein, 18 aa, Homo sapiens
MHSSALLCCLVLLTGVRA

SEQ ID NO: 138 - protein, 25 aa, Human respiratory syncytial virus A (strain A2)
MELLILKANAITTILTAVTFCFASG

SEQ ID NO: 139 - protein, 15 aa, Human respiratory syncytial virus A (strain A2)
MAIIYLILLFTAVRG

SEQ ID NO: 140 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 141 - protein, 62 aa, Homo sapiens
MGVPRPQPWALGLLLFLLPGSLGFACKTANGTAIPIGGGSANVYVNLAPVVNVGQNLVVDLS

SEQ ID NO: 142 - protein, 574 aa, Human respiratory syncytial virus A (strain A2)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 143 - protein, 64 aa, Human respiratory syncytial virus A (strain A2)
MELLILKANAITTILTAVTFCFASGFACKTANGTAIPIGGGSANVYVNLAPVVNVGQNLVVDLS

SEQ ID NO: 144 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 145 - protein, 57 aa, Homo sapiens
MHSSALLCCLVLLTGVRAFACKTANGTAIPIGGGSANVYVNLAPVVNVGQNLVVDLS

SEQ ID NO: 146 - protein, 562 aa, Influenza A virus (strain A/Japan/305/1957 H2N2)
MAIIYLILLFTAVRGDQICIGYHANNSTEKVDTNLERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKEGSDYPVAKGSYNNTSGEQMLIIWGVHHPIDETEQRTLYQNVGTYVSVGTSTLNKRSTPEIATRPKVNGQGGRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFGNLERRLENLNKRMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 147 - protein, 54 aa, Influenza A virus (strain A/Japan/305/1957 H2N2)
MAIIYLILLFTAVRGFACKTANGTAIPIGGGSANVYVNLAPVVNVGQNLVVDLS